(19) United States
(12) Patent Application Publication
(10) Pub. No.: US 2024/0091321 A1
(43) Pub. Date: Mar. 21, 2024

(54) VARIANT IGF2 CONSTRUCTS

(71) Applicant: Amicus Therapeutics, Inc., Philadelphia, PA (US)

(72) Inventors: Hung V. Do, Edmond, OK (US); Steven Tuske, Farmingdale, NJ (US); Russell Gotschall, Doylestown, PA (US); Ce Feng Liu, Brooklyn, NY (US)

(73) Assignee: Amicus Therapeutics, Inc., Philadelphia, PA (US)

(21) Appl. No.: 17/767,803

(22) PCT Filed: Oct. 12, 2020
(86) PCT No.: PCT/US20/55251

§ 371 Date: Apr. 8, 2022

Related U.S. Application Data
(60) Provisional application No. 62/929,054, filed on Oct. 31, 2019.
(60) Provisional application No. 62/913,677, filed on Oct. 10, 2019.

(51) Int. Cl. A61K 38/47; A61K 38/30; A61K 38/46; A61K 38/48; A61K 48/00

(52) U.S. Cl. CPC A61K 38/47; A61K 38/30; A61K 38/465; A61K 38/48; A61K 48/0041

(57) ABSTRACT

Provided herein are novel IGF2 peptides, fusion proteins, and nucleic acid sequences encoding novel IGF2 peptides and fusion proteins for the treatment of lysorsomal storage disorders, wherein the IGF2 peptides confer enhanced properties, such as enhanced expression, secretion and cellular uptake. The constructs provided herein are useful in treating lysosomal storage disorders by both enzyme replacement therapy and gene therapy.

### CROSS-REFERENCE TO RELATED APPLICATIONS

This application claims the benefit of priority to U.S. Provisional Application 62/913,677, filed on Oct. 10, 2019, and U.S. Provisional Application 62/929,054, filed on Oct. 31, 2019, each of which is incorporated by reference herein in its entirety.

### BACKGROUND

Genetic disorders arise via heritable or de novo mutations occurring in gene coding regions of the genome. In some cases, such genetic disorders are treated by administration of a protein that replaces a protein encoded by the gene mutated in the individual having the genetic disorder or by administration of a gene therapy vector encoding such a protein. Such treatment has challenges however, as the administered protein or the protein encoded by the gene therapy vector does not always result in the protein reaching the organs, cells, or organelle where it is needed. Proteins having improved intracellular targeting (e.g., to lysosomes), and gene therapy vectors encoding them, are desired.

### SUMMARY

In certain aspects, there are provided nucleic acid constructs comprising: (a) a nucleic acid sequence encoding a therapeutic protein, and (b) a nucleic acid sequence encoding a variant IGF2 (vIGF2) peptide. In some embodiments, the vIGF2 peptide has an amino acid sequence that is at least 90, 95, 96, 97, 98 or 99% identical to an IGF2 variant peptide of Table 3. In some embodiments, the vIGF2 peptide comprises an amino acid sequence that is at least 90, 95, 96, 97, 98 or 99% identical to an IGF2 variant peptide selected from the group consisting of SEQ ID NO:90-123 of Table 3. In some embodiments, the vIGF2 peptide further comprises a linker having a sequence that is at least 90, 95, 96, 97, 98 or 99% identical to a sequence selected from the group consisting of SEQ ID NOs: 181-188. In some embodiments, the vIGF2 peptide has decreased or no affinity for the insulin receptor and IGFR1 as compared to native IGF2 peptide. In some embodiments, the vIGF2 peptide has increased affinity for the CI-MPR as compared to native IGF2 peptide. In some embodiments, the vIGF2 peptide confers improved expression and/or secretion of a fusion protein, compared to a native IGF2 peptide. In some embodiments, the vIGF2 peptide is capable of facilitating uptake of the therapeutic protein into a lysosome in a cell. In some embodiments, the therapeutic protein is capable of replacing a defective or deficient protein associated with a genetic disorder in a subject having the genetic disorder. In some embodiments, the genetic disorder is a lysosomal storage disorder. In some embodiments, the genetic disorder is selected from the group consisting of aspartylglucosaminuria, CLN1, CLN2 C cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), chronic granulomatous disease (CGD), and neuronal ceroid lipofuscinosis. In some embodiments, the genetic disorder is Pompe disease. In some embodiments, the genetic disorder is neuronal ceroid lipofuscinosis. In some embodiments, the therapeutic protein comprises an enzyme selected from the group consisting of alpha-galactosidase (A or B), β-galactosidase, f3-hexosaminidase (A or B), galactosylceramidase, arylsulfatase (A or B), β-glucocerebrosidase, glucocerebrosidase, lysosomal acid lipase, lysosomal enzyme acid sphingomyelinase, formylglycine-generating enzyme, iduronidase (e.g., alpha-L), acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, N-sulfoglucosamine sulfohydrolase, glycosaminoglycan N—acetylgalactosamine 4-sulfatase β-glucuronidase, hyaluronidase, alpha-N-acetyl neuraminidase (sialidase), gangliosidesialidase, phosphotransferase, alpha-glucosidase, alpha-D-mannosidase, beta-D-mannosidase, aspartylglucosaminidase, alpha-L-fucosidase, battenin, palmitoyl protein thioesterases, and other Batten-related proteins (e.g., ceroid-lipofuscinosis neuronal protein 6), or an enzymatically active fragment thereof. In some embodiments, the therapeutic protein is alpha-glucosidase, or an enzymatically active fragment thereof. In some embodiments, the therapeutic protein is palmitoyl protein thioesterase 1 (PPT1). In some embodiments, the therapeutic protein is tripeptidyl peptidase 1 (TPP1). In some embodiments, the therapeutic protein is aspartylglucosaminidase. In some embodiments, the therapeutic protein is NAGLU (SEQ ID NO:54). In some embodiments, the therapeutic protein is the mature peptide of NAGLU, corresponding to amino acids 24-743 of SEQ ID NO:54 that remain after removal of the native signal peptide (SEQ ID NO:180). In some embodiments, the nucleic acid construct further comprises a translation initiation sequence. In some embodiments, the translation initiation sequence comprises a Kozak sequence. In some embodiments, the vIGF2 encoding nucleic acid sequence is 5′ to the nucleic acid sequence encoding a therapeutic protein. In some embodiments, the vIGF2 encoding nucleic acid sequence is 3′ to the nucleic acid sequence encoding a therapeutic protein. In some embodiments, the nucleic acid construct further comprises a linker sequence encoding a linker peptide between the vIGF2 nucleotide sequence and the nucleic acid sequence encoding a therapeutic protein. In some embodiments, the linker peptide comprises SEQ ID NO: 181-188. In some embodiments, the nucleic acid construct is a virus vector. In some embodiments, the virus vector is an adenovirus vector, an adeno-associated virus (AAV) vector, a retrovirus vector, a lentivirus vector, a pox virus vector, a vaccinia virus vector, an adenovirus vector, or a herpes virus vector.

In additional aspects, there are provided pharmaceutical compositions comprising a therapeutically effective amount of any one of the nucleic acid constructs provided herein a pharmaceutically acceptable carrier or excipient. In some embodiments, the excipient comprises a non-ionic, low-osmolar compound, a buffer, a polymer, a salt, or a combination thereof.

In further aspects, there are provided methods for treating a genetic disorder comprising administering to a subject in need thereof any one of the nucleic acid constructs provided herein or any one of the pharmaceutical compositions provided herein. In some embodiments, genetic disorder is a lysosomal storage disorder. In some embodiments, the genetic disorder is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), chronic granulomatous disease (CGD), and neuronal ceroid lipofuscinosis (Batten disease). In some embodiments, the genetic disorder is Pompe disease. In some embodiments, the genetic disorder is neuronal ceroid lipofuscinosis. In some embodiments, the genetic disorder is Aspartylglucosaminuria. In some embodiments, the administering is performed intrathecally, intraocularly, intravitreally, retinally, intravenously, intramuscularly, intraventricularly, intracerebrally, intracerebellarly, intracerebroventricularly, intraperenchymally, subcutaneously, or a combination thereof. In some embodiments, the administering is performed intrathecally.

In additional aspects, there are provided pharmaceutical compositions comprising any one of the gene therapy vectors provided herein and a pharmaceutically acceptable carrier or excipient for use in treating a genetic disorder. In further aspects, there are provided pharmaceutical composition comprising any one of the nucleic acid constructs provided herein and a pharmaceutically acceptable carrier or excipient for use in preparation of a medicament for treatment of a genetic disorder. In some embodiments, the genetic disorder is a lysosomal storage disorder. In some embodiments, the genetic disorder is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), chronic granulomatous disease (CGD), and neuronal ceroid lipofuscinosis. In some embodiments, the genetic disorder is Pompe disease. In some embodiments, the genetic disorder is neuronal ceroid lipofuscinosis. In some embodiments, the genetic disorder is Aspartylglucosaminuria. In some embodiments, the composition is formulated for administration intrathecally, intraocularly, intravitreally, retinally, intravenously, intramuscularly, intraventricularly, intracerebrally, intracerebellarly, or subcutaneously. In some embodiments, the composition is formulated for administration intrathecally.

In additional aspects there are provided nucleic acids encoding a fusion protein having an amino acid sequence at least 90, 95, 96, 97, 98 or 99% identical to a sequence selected from the group consisting of SEQ ID NO:47-53. In some embodiments, the nucleic acid is at least 85, 90, 95, 96, 97, 98 or 99% identical to a sequence selected from the group consisting of SEQ ID NO: 60-67.

In further aspects, there are provided pharmaceutical composition comprising any one of the above nucleic acids and a pharmaceutically acceptable carrier or excipient. In some embodiments, the excipient comprises a non-ionic, low-osmolar compound, a buffer, a polymer, a salt, or a combination thereof.

In further aspects pharmaceutical composition comprising the fusion protein having an amino acid sequence at least 90, 95, 96, 97, 98 or 99% identical to a sequence selected from the group consisting of SEQ ID NO: 47-53 and SEQ ID NO: 60-67, and a pharmaceutically acceptable carrier or excipient. In some embodiments, the excipient comprises a non-ionic, low-osmolar compound, a buffer, a polymer, a salt, or a combination thereof.

In additional aspects, there are provided gene therapy vectors comprising a nucleic acid encoding an amino acid sequence at least 90, 95, 96, 97, 98 or 99% identical to a sequence selected from the group consisting of SEQ ID NO: 47-53 and SEQ ID NO: 60-67; and a nucleic acid encoding an amino acid sequence at least 90, 95, 96, 97, 98 or 99% identical to a sequence selected from the group consisting of SEQ ID NO:106, 109, 111, 119, 120 and 121. In some embodiments, the gene therapy vector is a virus vector. In some embodiments, the virus vector is an adenovirus vector, an adeno-associated virus (AAV) vector, a retrovirus vector, a lentivirus vector, a pox virus vector, a vaccinia virus vector, an adenovirus vector, or a herpes virus vector and a pharmaceutically acceptable carrier or excipient. In some embodiments, the excipient comprises a non-ionic, low-osmolar compound, a buffer, a polymer, a salt, or a combination thereof.

In additional aspects, there are provided methods of treating CLN1/PPT1 disease or CLN2/TPP1 disease comprising administering to a subject in need thereof a therapeutically effective amount of any one of the nucleic acids herein, any one of the fusion proteins herein, any one of the gene therapy vectors herein, or any one of the pharmaceutical compositions herein. In some embodiments, the administering is performed intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In some embodiments, the nucleic acid has a nucleic acid sequence at least 85, 90, 95, 96, 97, 98 or 99% identical to a sequence selected from the group consisting of SEQ ID NO:189-250.

In additional aspects there are provided pharmaceutical compositions comprising any one of the nucleic acids herein a pharmaceutically acceptable carrier or excipient. In some embodiments, the excipient comprises a non-ionic, low-osmolar compound, a buffer, a polymer, a salt, or a combination thereof.

In some embodiments, there are provided a variant IGF2 (vIGF2) peptide that is at least 95, 96, 97, 98 or 99% identical to a sequence selected from the group consisting of SEQ ID NO: 90-103.

In some embodiments, the variant IGF2 (vIGF2) peptide is at least 98% identical to at least one sequence selected from SEQ ID NOs:106, 109, 111, 119, 120, 121. In some embodiments, the vIGF2 peptide is at least 95, 96, 97, 98 or 99% identical to SEQ ID NO:120 or 121.

In some embodiments, there are provided a fusion protein comprising a variant vIGF2 peptide and a therapeutic protein having an amino acid sequence at least 95, 96, 97, 98 or 99% identical to a sequence selected from the group consisting of SEQ ID NO:4, amino acid residues 21-306 of SEQ ID NO:4, amino acid residues 28-306 of SEQ ID NO:4, SEQ ID NO: 8, SEQ ID NO:46, and SEQ ID NO:54.

In some embodiments, the fusion protein has an amino acid sequence at least 95, 96, 97, 98 or 99% identical to a sequence selected from the group consisting of SEQ ID NO:60-67, SEQ ID NO:47-53 and SEQ ID NO:54-59. In some embodiments, the fusion protein further comprises a lysosomal cleavage peptide. In some embodiments, the lysosomal cleavage peptide has SEQ ID NO:188. In some embodiments the vIGF2 peptide is N terminal to the therapeutic protein. In some embodiments, the vIGF2 peptide is C terminal to the therapeutic protein.

In some embodiments, the fusion protein comprises a signal sequence. In some embodiments, the signal sequence has an amino acid sequence at least 95, 96, 97, 98 or 99% identical to a sequence selected from the group consisting of SEQ ID NO:169-180.

In some embodiments, the therapeutic protein is PPT1 or an enzymatically active fragment thereof, TPP1 or an enzymatically active fragment thereof, or NAGLU or enzymatically active fragment thereof.

In some embodiments, the fusion protein is taken up by target cells more efficiently than the corresponding protein lacking the vIGF2 peptide. In some embodiments, the fusion protein is taken up by cells in the brain. In some embodiments the fusion protein is taken up by neuronal cells. In some embodiments the fusion protein is taken up by glial cells.

Provided herein are also pharmaceutical composition comprising fusion proteins having a vIGF2 peptide and a therapeutic protein, along with a pharmaceutically acceptable carrier or excipient. Methods of treating a lysosomal storage disorder, comprising administering such pharmaceutical compositions to a subject in need thereof are also provided herein. In some embodiments, the lysosomal storage disorder is selected from the group consisting of CLN1/PPT1 disease, CLN2/TPP1 disease, and Sanfilippo Type B disease. In some embodiments, the fusion protein or pharmaceutical composition comprising the fusion protein is administered intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In some embodiments, administering the pharmaceutical composition prevents/reduces or reverses accumulation of autofluorescent storage material (ASM) in the brain. In some embodiments, administering the pharmaceutical composition prevents/reduces or reverses elevation of glial fibrillary acidic protein (GFAP) in the brain. In some embodiments, administering the pharmaceutical composition prevents/reduces or reverses accumulation of autofluorescent storage material (ASM) in the cortex or thalamus. In some embodiments, administering the pharmaceutical composition prevents/reduces or reverses elevation of glial fibrillary acidic protein (GFAP) brain cortex or thalamus.

Further provided herein are nucleic acids encoding a fusion protein comprising vIGF2 and a therapeutic protein, wherein the nucleic acid is at least 85, 90, 95, 96, 97. 98 or 99% identical to a sequence selected from the group consisting of SEQ ID NO:189-250.

In additional aspects, there are provided pharmaceutical compositions comprising any one of the fusion proteins herein and a pharmaceutically acceptable carrier or excipient. In some embodiments, the excipient comprises a non-ionic, low-osmolar compound, a buffer, a polymer, a salt, or a combination thereof.

In further aspects, there are provided gene therapy vectors comprising a nucleic acid encoding an amino acid sequence at least 90% identical to SEQ ID NO: 51. In some embodiments, the gene therapy vector is a virus vector. In some embodiments, the virus vector is an adenovirus vector, an adeno-associated virus (AAV) vector, a retrovirus vector, a lentivirus vector, a pox virus vector, a vaccinia virus vector, an adenovirus vector, or a herpes virus vector.

In additional aspects, there are provided pharmaceutical compositions comprising any one of the gene therapy vectors provided herein and a pharmaceutically acceptable carrier or excipient. In some embodiments, the excipient comprises a non-ionic, low-osmolar compound, a buffer, a polymer, a salt, or a combination thereof.

In another aspect, there are provided nucleic acid constructs comprising: (a) a nucleic acid sequence encoding a therapeutic protein, and (b) a nucleic acid sequence encoding a variant IGF2 (vIGF2) peptide that is at least 95, 96, 97. 98 or 99% identical to at least one sequence selected from SEQ ID NO: 90-103. In some aspects, the vIGF2 peptide has an amino acid sequence that is at least 95, 96, 97. 98 or 99% identical to an IGF2 variant peptide selected from SEQ ID NOs:106, 109, 111, 119, 120, 121. In some embodiments, the vIGF2 peptide comprises an amino acid sequence that is at least 95, 96, 97. 98 or 99% identical to an IGF2 variant peptide selected from the group consisting of SEQ ID NO:120 and SEQ ID NO:121.

In some aspects, the nucleic acid further comprises a sequence encoding a linker having a sequence that is at least 95, 96, 97. 98 or 99% identical to a sequence selected from the group consisting of SEQ ID NOs: 181-188. In some embodiments, the vIGF2 peptide is capable of increasing expression and/or secretion of a therapeutic protein compared to a vIGF2 peptide having the amino acid sequence of SEQ ID NO:80. In some embodiments, the vIGF2 peptide has increased affinity for the CI-MPR as compared to a vIGF2 peptide having the amino acid sequence of SEQ ID NO:80. In some embodiments, the vIGF2 peptide is capable of improving uptake of the therapeutic protein into a target cell, such as a human brain cell. In some embodiments, the human brain cell is a neuronal cell or a glial cell.

In certain aspects, the therapeutic protein is capable of replacing a defective or deficient protein associated with a genetic disorder in a subject having the genetic disorder. In some embodiments, the genetic disorder is a lysosomal storage disorder. In some embodiments, the genetic disorder is selected from the group consisting of aspartylglucosaminuria, neuronal ceroid lipofuscinosis, CLN1/PPT1 disease, CLN2/PPT1 disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), and neuronal ceroid lipofuscinosis. In some embodiments, the genetic disorder is selected from the group consisting of CLN1/PPT1 disease, CLN2/PPT1 disease, Pompe disease and MPS IIIB disease. In some aspects, the genetic disorder is CLN1/PPT1 disease or CLN2/PPT1 disease.

In some aspects, the therapeutic protein comprises a human enzyme selected from the group consisting of alpha-galactosidase (A or B), β-galactosidase, f3-hexosaminidase (A or B), galactosylceramidase, arylsulfatase (A or B), β-glucocerebrosidase, glucocerebrosidase, lysosomal acid lipase, lysosomal enzyme acid sphingomyelinase, formylglycine-generating enzyme, iduronidase (e.g., alpha-L), acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, N-sulfoglucosamine sulfohydrolase, glycosaminoglycan N-acetylgalactosamine 4-sulfatase, β-glucuronidase, hyaluronidase, alpha-N-acetyl neuraminidase (sialidase), gangliosidesialidase, phosphotransferase, alpha-glucosidase, alpha-D-mannosidase, beta-D-mannosidase, aspartylglucosaminidase, alpha-L-fucosidase, battenin, PPT1, TPP1, and other Batten-related proteins (e.g., ceroid-lipofuscinosis neuronal protein 6), or an enzymatically active fragment thereof. In some embodiments, the therapeutic protein is a human lysosomal enzyme or an enzymatically active fragment thereof. In some embodiments, the human lysosomal enzyme is alpha-glucosidase, PPT1, TPP1, or NAGLU.

In some aspects, the nucleic acid construct further comprises a sequence encoding a signal peptide. In some embodiments, the signal peptide is a sequence selected from the group consisting of SEQ ID NO:169-180. In some embodiments, the vIGF2 encoding nucleic acid sequence is 5′ to the nucleic acid sequence encoding a therapeutic protein. In other embodiments, the vIGF2 encoding nucleic acid sequence is 3′ to the nucleic acid sequence encoding a therapeutic protein.

Further provided herein are gene therapy vectors comprising the nucleic acids described herein. In some embodiments, the gene therapy vector is a virus vector. In some embodiments, the virus vector is an adenovirus vector, an adeno-associated virus (AAV) vector, a retrovirus vector, a lentivirus vector, a pox virus vector, a vaccinia virus vector, an adenovirus vector, or a herpes virus vector.

In some aspects, the nucleic acid constructs herein are in a plasmid or bacterial artificial chromosome. In some embodiments, the nucleic acids constructs described herein are in a host cell.

There are further provided pharmaceutical compositions, comprising a therapeutically effective amount of the nucleic acid constructs described herein, or gene therapy vectors comprising the nucleic acid constructs described herein, along with a pharmaceutically acceptable carrier or excipient. In some embodiments, the excipient comprises a non-ionic, low-osmolar compound, a buffer, a polymer, a salt, or a combination thereof.

Further provided herein are methods for treating a genetic disorder comprising administering to a subject in need thereof the nucleic acid constructs, gene therapy vectors and/or pharmaceutical composition described herein. In some embodiments, the genetic disorder is a lysosomal storage disorder. In some embodiments, the genetic disorder is selected from the group consisting of aspartylglucosaminuria, neuronal ceroid lipofuscinosis, CLN1/PPT1 disease, CLN2/PPT1 disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), and chronic granulomatous disease (CGD). In some embodiments, the genetic disorder is Batten's disease, such as CLN1/PPT1 disease or CLN2/TPP1 disease. In some embodiments, the genetic disorder is Pompe disease or Sanfilippo disease type B.

In some embodiments, the administering is performed intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In some aspects, administering the nucleic acid, gene therapy vector, fusion protein, or pharmaceutical composition prevents/reduces or reverses accumulation of autofluorescent storage material (ASM) in the brain. In some embodiments, administering the nucleic acid, gene therapy vector fusion protein, or pharmaceutical composition prevents/reduces or reverses elevation of glial fibrillary acidic protein (GFAP) in the brain. In some embodiments, administering the nucleic acid, gene therapy vector, fusion protein, or pharmaceutical composition prevents/reduces or reverses accumulation of autofluorescent storage material (ASM) in the cortex or thalamus. In some aspects, administering the nucleic acid, gene therapy vector, fusion protein, or pharmaceutical composition prevents/reduces or reverses elevation of glial fibrillary acidic protein (GFAP) brain cortex or thalamus.

In some aspects, the nucleic acid encodes a fusion protein having a sequence at least 95, 96, 97. 98 or 99% identical to a sequence selected from the group consisting of SEQ ID NO:60-67. In some embodiments, the nucleic acid encodes a fusion protein having a sequence at least 98% identical to a sequence selected from the group consisting of SEQ ID NO:47-53.

In some aspects, the nucleic acid encodes a fusion protein comprising: (a) an amino acid sequence at least 95, 96, 97, 98 or 99% identical to a sequence selected from the group consisting of SEQ ID NO:106, 109, 111, 119, 120 and 121; and (b) an amino acid sequence at least 95, 96, 97. 98 or 99% identical to a sequence selected from the group consisting of SEQ ID NO:4, residues 21-306 of SEQ ID NO:4, residues 28-306 of SEQ ID NO:4, SEQ ID NO: 8, and SEQ ID NO:46. In some embodiments, the nucleic acid encodes a vIGF2 at least 95, 96, 97, 98, or 99% identical to SEQ ID NO:120 and 121. In some embodiments, the nucleic acid encodes a fusion protein comprising: (a) at least one of SEQ ID NO:106, 109, 111, 119, 120 or 121; and (b) at least one of SEQ ID NO:4, residues 21-306 of SEQ ID NO:4, residues 28-306 of SEQ ID NO:4, SEQ ID NO: 8, and SEQ ID NO:46. residues 28-306 of SEQ ID NO:4, SEQ ID NO: 8, and SEQ ID NO:46.

In some embodiments, the nucleic acid further encodes a lysosomal cleavage peptide.

In some aspects, the fusion protein has a sequence at least 95, 96, 97, 98, or 99% identical to at least one of SEQ ID NO:60-67 and SEQ ID NO:47-53. In some embodiments, the fusion protein comprises at least one of SEQ ID NO:60-67 and SEQ ID NO:47-53. In some embodiments the fusion protein consists or consists essentially of SEQ ID NO:60-67 and SEQ ID NO:47-53.

In additional aspects, there are provided methods of treating a lysosomal storage disease comprising administering to a subject in need thereof a therapeutically effective amount of any one of the nucleic acids herein, any one of the fusion proteins herein, any one of the gene therapy vectors herein, or any one of the pharmaceutical compositions herein. In some embodiments, the administering is performed intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In further aspects, there are provided methods of treating Batten disease, including CLN1/PPT1 disease and CLN2/TPP1 disease comprising administering to a subject in need thereof a therapeutically effective amount of any one of the nucleic acids herein, any one of the fusion proteins herein, any one of the gene therapy vectors herein, or any one of the pharmaceutical compositions herein. In some embodiments, the administering is performed intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In additional aspects, there are provided pharmaceutical compositions comprising any one of the nucleic acids provided herein and a pharmaceutically acceptable carrier or excipient. In some embodiments, the excipient comprises a non-ionic, low-osmolar compound, a buffer, a polymer, a salt, or a combination thereof.

In additional aspects, there are provided pharmaceutical compositions comprising any one of the gene therapy vectors provided herein and a pharmaceutically acceptable carrier or excipient.

In additional aspects, there are provided fusion proteins comprising: (a) a lysosomal enzyme, and (b) a variant IGF2 (vIGF2) peptide, wherein the vIGF2 peptide comprises an amino acid sequence that is at least 95, 96, 97, 98, or 99% identical to an IGF2 variant peptide of Table 3. In some embodiments, the vIGF2 peptide comprises an amino acid sequence that is at least 95, 96, 97, 98, or 99% identical to an IGF2 variant peptide selected from the group consisting of SEQ ID NO: 69-131. In some embodiments, the vIGF2 peptide comprises an amino acid sequence that is at least 95, 96, 97, 98, or 99% identical to an IGF2 variant peptide selected from the group consisting of SEQ ID NO: 90-123. In some embodiments, the vIGF2 has been modified to replace residues 31-38 of wildtype IGF2 with four glycine residues (Δ 31-38GGGG). In some embodiments, the vIGF2 has been further modified by a V43L mutation. In some embodiments, the vIGF2 has been further modified to replace the serine in position 50 with an acidic residue (aspartic or glutamic acid). In some aspects, the vIGF2 has the sequence of SEQ ID NO:120 or 121.

In some embodiments, the vIGF2 peptide further comprises a linker having a sequence that is at least 95, 96, 97, 98, or 99% identical to a sequence selected from the group consisting of SEQ ID NOs: 181-188. In some embodiments, the linker is cleavable. In some embodiments, the vIGF2 peptide has decreased or no affinity for the insulin receptor and IGFR1 as compared to native IGF2 peptide. In some embodiments, the vIGF2 peptide has increased affinity for the CI-MPR as compared to native IGF2 peptide. In some embodiments, the vIGF2 peptide is capable of facilitating uptake of the lysosomal enzyme into a lysosome in a cell. In some embodiments, the lysosomal enzyme is capable of replacing a defective or deficient protein associated with a lysosomal storage disorder. In some embodiments, the lysosomal storage disorder is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), chronic granulomatous disease (CGD), and neuronal ceroid lipofuscinosis. In some embodiments, the lysosomal storage disorder is Pompe disease. In some embodiments, the lysosomal storage disorder is neuronal ceroid lipofuscinosis. In some embodiments, the lysosomal enzyme comprises an enzyme selected from the group consisting of alpha-galactosidase (A or B), β-galactosidase, f3-hexosaminidase (A or B), galactosylceramidase, arylsulfatase (A or B), β-glucocerebrosidase, glucocerebrosidase, lysosomal acid lipase, lysosomal enzyme acid sphingomyelinase, formylglycine-generating enzyme, iduronidase (e.g., alpha-L), acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-sulfoglucosamine sulfohydrolase, N-acetylgalactosamine-6-sulfatase, glycosaminoglycan N-acetylgalactosamine 4-sulfatase, β-glucuronidase, hyaluronidase, alpha-N-acetyl neuraminidase (sialidase), gangliosidesialidase, phosphotransferase, alpha-glucosidase, alpha-D-mannosidase, beta-D-mannosidase, aspartylglucosaminidase, alpha-L-fucosidase, battenin, palmitoyl protein thioesterases, and other Batten-related proteins (e.g., ceroid-lipofuscinosis neuronal protein 6), or an enzymatically active fragment thereof. In some embodiments, the lysosomal enzyme is alpha-glucosidase, or an enzymatically active fragment thereof. In some embodiments, the lysosomal enzyme is a palmitoyl protein thioesterase. In some embodiments, the lysosomal enzyme is tripeptidyl peptidase 1. In some embodiments, the lysosomal enzyme is aspartylglucosaminidase.

Additionally, provided herein are pharmaceutical compositions comprising a therapeutically effective amount of any one of the fusion proteins provided herein and a pharmaceutically acceptable carrier or excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent application file contains at least one drawing executed in color. Copies of this patent application with color drawing(s) will be provided by the Office upon request and payment of the necessary fee. An understanding of the features and advantages of the present disclosure will be obtained by reference to the following detailed description that sets forth illustrative embodiments, in which the principles of the disclosure are utilized, and the accompanying drawings of which:

FIG. 1 shows affinity chromatography using immobilized CI-MPR was used to determine the proportion of GAA that is able to interact with the CI-MPR through phosphorylated oligosaccharides. The first peak is the material that flows through column indicting that it does not have phosphorylated glycans. The later peak is the material able to bind the immobilized CI-MPR. It is eluted with an increasing gradient of M6P. M6P reveals that GAA contains both M6P-containing and -lacking fractions. Since binding the CI-MPR is the mandatory first step for receptor-mediated endocytosis, only the rhGAA fraction that binds the CI-MPR is capable of efficient cellular uptake.

FIG. 2 shows structure of the CI-MPR including the different binding domains for the IGF2 and for mono- and bis-phosphorylated oligosaccharides.

FIG. 3 shows the sequence and structure of the mature, human IGF2 peptide. Site specific amino acid substitutions are proposed to influence binding to other receptors and serum proteins.

FIG. 4 shows binding of the wild-type IGF2 (wtIGF2) peptide to CI-MPR as measured by surface plasmon resonance

FIG. 5 shows binding of the variant IGF2 (vIGF2) peptide binding to CI-MPR as measured by surface plasmon resonance.

FIG. 6 shows benefit of adding vIGF2 to alglucosidase alfa to increase the binding to the IGF2/CI-MPR.

FIG. 7 shows the benefit of adding a vIGF2 to recombinant human N-acetyl-α-D-glucosaminidase (rhNAGLU) to increase the binding to the IGF2/CI-MPR.

FIG. 8 shows binding of wildtype human IGF2 to insulin receptor.

FIG. 9 shows no detectable binding of vIGF2 to insulin receptor.

FIG. 10 shows binding of wildtype IGF2 to insulin-like growth factor 1 receptor.

FIG. 11 shows decreased binding of vIGF2 peptide to insulin-like growth factor 1 receptor, as compared to wildtype IGF2.

FIG. 12 shows two examples of gene therapy expression cassettes encoding Natural hGAA and Engineered hGAA. Natural hGAA is poorly phosphorylated, and unable to efficiently bind the CI-MPR. Engineered hGAA has element added for improved CIMPR binding (vIGF2), a 2GS linker is incorporate to allow for greater interaction ofvIGF2-GAA protein with CI-MPR, and a BiP signal peptide to improve secretion.

FIG. 13 shows a Western blot of palmitoyl-protein thioesterase 1 (PPT1) from cells expressing recombinant human PPT1 (PPT1-1), recombinant human PPT1 having a vIGF2 targeting domain (PPT1-2) and recombinant human PPT1 having a vIGF2 targeting domain and a BiP signal sequence (PPT1-29). Protein expression can be influenced by the variant of IGF used.

FIG. 14 shows binding of PPT1 constructs to CI-MPR.

FIG. 15 shows GAA activity in conditioned media of CHO cells expressing engineered or natural hGAA.

FIG. 16 shows the study design of a 4-week mouse study of gene therapy in a GAA knockout mouse.

FIG. 17 shows GAA plasma activity in untreated wild type (“Normal”) mice or GAA knockout mice treated with gene therapy vectors or vehicle as indicated.

FIG. 18 shows GAA levels measured in untreated wild type (“Normal”) mice or GAA knockout mice treated with gene therapy vectors or vehicle as indicated.

FIG. 19 shows cell surface receptor CI-MPR binding of rhGAA from plasma samples obtained from treated mice as indicated.

FIG. 20 shows GAA activity, and quad glycogen histopathology score for tibialis anterior of untreated wild type (“Normal”) mice or GAA knockout mice treated with gene therapy vectors or vehicle as indicated.

FIG. 21 shows glycogen PAS of tibialis anterior from untreated wild type mice or GAA knockout mice treated with gene therapy vectors or vehicle as indicated.

FIG. 22 shows hGAA immunohistochemistry of tibialis anterior from untreated wild type mice or GAA knockout mice treated with gene therapy vectors or vehicle as indicated.

FIG. 23 shows brain GAA activity, brain glycogen, and spinal cord glycogen histopathology scoring for brain and spinal cord from untreated wild type (“Normal”) mice or GAA knockout mice treated with gene therapy vectors or vehicle as indicated.

FIG. 24 shows glycogen PAS of brain from untreated wild type mice or GAA knockout mice treated with gene therapy vectors or vehicle as indicated.

FIG. 25 shows hGAA immunohistochemistry of brainstem and choroid plexus from untreated wild type (“Normal”) mice or GAA knockout mice treated with gene therapy vectors or vehicle as indicated.

FIG. 26 shows glycogen PAS of spinal cord from untreated wild type mice or GAA knockout mice treated with gene therapy vectors or vehicle as indicated.

FIG. 27 shows hGAA immunohistochemistry of spinal cord from untreated wild type mice or GAA knockout mice treated with gene therapy vectors or vehicle as indicated.

FIG. 28 shows quadriceps GAA activity and glycogen histopathology scoring from untreated wild type (“Normal”) mice or GAA knockout mice treated with gene therapy vectors or vehicle as indicated.

FIG. 29 shows glycogen luxol/PAS for quadriceps from untreated wild type mice or GAA knockout mice treated with gene therapy vectors or vehicle as indicated.

FIG. 30 shows hGAA immunohistochemistry of quadriceps from untreated wild type mice or GAA knockout mice treated with gene therapy vectors or vehicle as indicated.

FIG. 31 shows triceps GAA activity and histopathology scoring for untreated wild type (“Normal”) mice or GAA knockout mice treated with gene therapy vectors or vehicle as indicated.

FIG. 32 shows glycogen luxol/PAS of triceps from untreated wild type mice or GAA knockout mice treated with gene therapy vectors or vehicle as indicated.

FIG. 33 shows hGAA immunohistochemistry of triceps from untreated wild type mice or GAA knockout mice treated with gene therapy vectors or vehicle as indicated.

FIG. 34 shows engineered and wild type PPT1 binding to CIMPR.

FIG. 35 shows engineered and wild type TPP1 binding to CIMPR.

FIG. 36 shows engineered and wild type AGA binding to CIMPR.

FIG. 37 shows engineered and wild type GLA binding to CIMPR.

FIG. 38 shows a Western blot of GAA from cells expressing various mutant vIGF2-GAA constructs in conditioned media.

FIG. 39 shows secretion of new IGF2-GAA variants relative to vIGF2-GAA, constructs from Western blot of FIG. 38.

FIG. 40 shows CI-MPR binding of various vIGF2-GAA constructs.

FIG. 41 shows Bmax and Kd values for CIMPR binding of various vIGF2-GAA constructs.

FIG. 42 shows CI-MPR binding of various vIGF2-GAA constructs.

FIG. 43 shows Bmax and Kd values for CIMPR binding of various vIGF2-GAA constructs.

FIG. 44 shows CI-MPR binding of various vIGF2-GAA constructs.

FIG. 45 shows Bmax and Kd values for CIMPR binding of various vIGF2-GAA constructs.

FIG. 46 shows cell uptake for various vIGF2-GAA constructs.

FIG. 47 shows cell uptake for various vIGF2-GAA constructs.

FIG. 48 shows various vIGF2 peptides binding to CI-MPR or IGF2R.

FIG. 49 shows PPT1 in conditioned media quantified by Western blot.

FIG. 50 shows PPT1 in conditioned media quantified by Western blot.

FIG. 51 shows PPT1 in conditioned media quantified by activity.

FIG. 52 shows correlation between PPT1 Western blot quantification versus activity quantification.

FIG. 53 shows binding of PPT1 constructs to CI-MPR.

FIG. 54 shows a structure diagram of selected PPT1 constructs.

FIG. 55 shows Western blot of PPT1 secreted into conditioned media.

FIG. 56 shows processing of PPT1 in the cell by Western blot.

FIG. 57 shows PPT1 in conditioned media quantified by Western blot.

FIG. 58 shows relative PPT1 activity.

FIG. 59 shows binding of PPT1 constructs to CI-MPR.

FIG. 60 shows binding of PPT1 constructs to CI-MPR.

FIG. 61 shows an alignment of variants of IGF2-GAA (1: vIGF2; 2: vIGF2-17; 3: IGF2-20; and 4: IGF2-22).

FIG. 62 shows additional PPT1 constructs.

FIG. 63 (A) shows expression of PPT1 constructs, normalized to wild-type, untagged PPT1 (construct 100), as measured by the band intensity on a Western Blot. The average intensity for four replicate transfections is shown for each sample with standard deviation error bars. (B) shows PPT1 expression/secretion of PPT1 in media, normalized to wild-type, as measured by the band intensity on a Western Blot.

FIG. 64 shows uptake into rat cortical neurons of PPT1 constructs as measured by immunofluorescence. (A) shows neuronal uptake of purified PPT1-101 and PPT1-104. (B) shows neuronal uptake of PPT-1 constructs from media (not purified).

FIG. 65 shows additional NAGLU constructs.

FIG. 67 (A) shows expression of NAGLU constructs, normalized to wild-type, untagged PPT1 (construct 100), as measured by the band intensity on a Western Blot. The average intensity for four replicate transfections is shown for each sample with standard deviation error bars. (B) shows PPT1 expression/secretion of PPT1 in media, normalized to wild-type, as measured by the band intensity on a Western Blot.

FIG. 68 shows expression of TPP1 constructs, normalized to wild-type, untagged TPP1, as measured by the band intensity on a Western Blot.

FIG. 69 shows CIMPR binding of TPP1 constructs.

FIG. 70 shows human CLN1 transgene expression as detected by RT-qPCR.

FIG. 71-72 show brain autofluorescent storage material (ASM) accumulation, a correlate of lysosomal dysfunction.

FIG. 73 shows Glial Fibrillary Acidic Protein (GFAP), a correlate of astrogliosis and neuroinflammation.

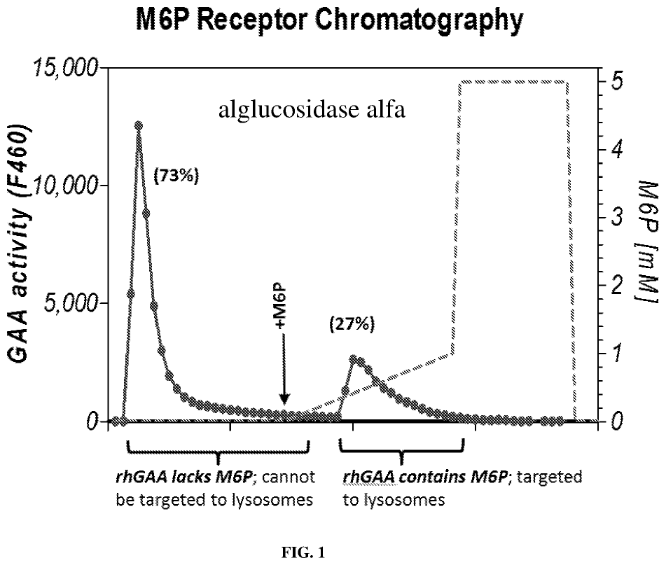

### DETAILED DESCRIPTION

Provided herein are novel, engineered IGF2 peptides with enhanced properties, including enhanced expression, secretion and CIMPR binding. Further provided herein are fusion proteins and nucleic acids encoding fusion proteins comprising novel IGF2 peptides and lysosomal enzymes with enhanced properties, such as increased CIMPR binding and improved expression and secretion. The fusion proteins and nucleic acid constructs provided herein are useful both for enzyme replacement therapies and for gene therapies to treat lysosomal storage disorders

Gene therapy for single gene genetic disorders presents a potential one-time treatment for diseases and disorders, some of which have devastating symptoms that can appear early in life and sometimes lead to life-long disability. Genetic disorders, such as neurological disorders or lysosomal storage disorders, are often treated with enzyme replacement therapies which administer to the patient a therapeutic protein that is an active form of the protein that is defective or deficient in the disease or disorder state. However, there are challenges for current therapies, including frequent treatments, development of an immune response to the therapeutic protein, and difficulty targeting the therapeutic protein to the affected tissue, cell, or subcellular compartment. Gene therapy offers advantages including a reduced number of treatments and long-lasting efficacy.

Provided herein are fusion proteins for administration as enzyme replacement therapy or encoded by vectors for gene therapy vectors that offer improvements to enzyme replacement therapy or gene therapy, such as providing more therapeutic protein where it is needed, thus improving treatment efficacy. Such challenges are addressed herein by improving expression and cellular uptake or delivery and intracellular or subcellular targeting of therapeutic proteins. Specific tools or components provided herein include but are not limited to signal peptides (e.g., binding immunoglobulin protein (BiP) and Gaussia signal peptides) for increasing secretion and peptides that increase endocytosis of the therapeutic protein (e.g., peptides that bind to the CI-MPR with high affinity for increasing cellular uptake and lysosomal delivery). Such peptides are fused to therapeutic proteins encoded by gene therapy vectors. In some embodiments, the peptides are IGF2 (Insulin Like growth factor 2) peptides or variants thereof. Gene therapy vectors provided herein are contemplated to comprise, in some embodiments, a nucleic acid encoding a therapeutic protein fused to a peptide that bind to the CI-MPR with high affinity for optimizing efficacy of gene therapy.

Gene therapy constructs for enzyme replacement gene therapy were designed. A translation initiation sequence, including, but not limited to a Kozak sequence or an IRES sequence, such as CrPV IRES, located at the 5′ end of the construct, followed by a nucleic acid encoding a signal peptide selected from one or more of a GAA signal peptide, a nucleic acid encoding an anti-trypsin inhibitor, and a nucleic acid encoding BiP sequence. These are followed by a nucleic acid encoding a cell targeting domain which can be a vIGF-2, a HIRMab, or a TfRMab or other cell targeting peptide or protein. The gene therapy construct further comprises a nucleic acid encoding a linker and a nucleic acid encoding a corrective enzyme or enzymatically active fragment thereof, wherein the linker connects the cell targeting domain to the corrective enzyme, or enzymatically active fragment thereof. Suitable corrective enzymes include but are not limited to alpha-glucosidase (GAA), alpha-galactosidase (GLA), iduronidase (IDUA), iduroniate-2-sulfatase (IDS), PPT1, TPP1, NAGLU, or enzymatically active fragments thereof, and other enzymes found deficient in an individual.

Intracellular Targeting of Therapeutic Proteins

N-linked carbohydrates of most lysosomal proteins are modified to contain a specialized carbohydrate structure called mannose 6-phosphate (M6P). M6P is the biological signal that enables transport of lysosomal proteins to lysosomes via membrane-bound M6P receptors. Enzyme replacement therapies for lysosomal storage disorders utilize M6P receptors for uptake and delivery of therapeutic proteins to lysosomes. Certain therapeutics do not utilize M6P receptors including Cerezyme® and other versions of recombinant human GCase, utilize the mannose receptor that is able to bind terminal mannose on protein glycans and deliver to the lysosome. A problem facing certain enzyme replacement therapeutics is there are low amounts of M6P present on the enzyme therapeutic which necessitate higher doses to reach therapeutic efficacy. This leads to substantially longer infusion times, higher probability of developing immune responses to the therapeutic, and higher drug demand, requiring increased protein manufacturing resulting in increased costs.

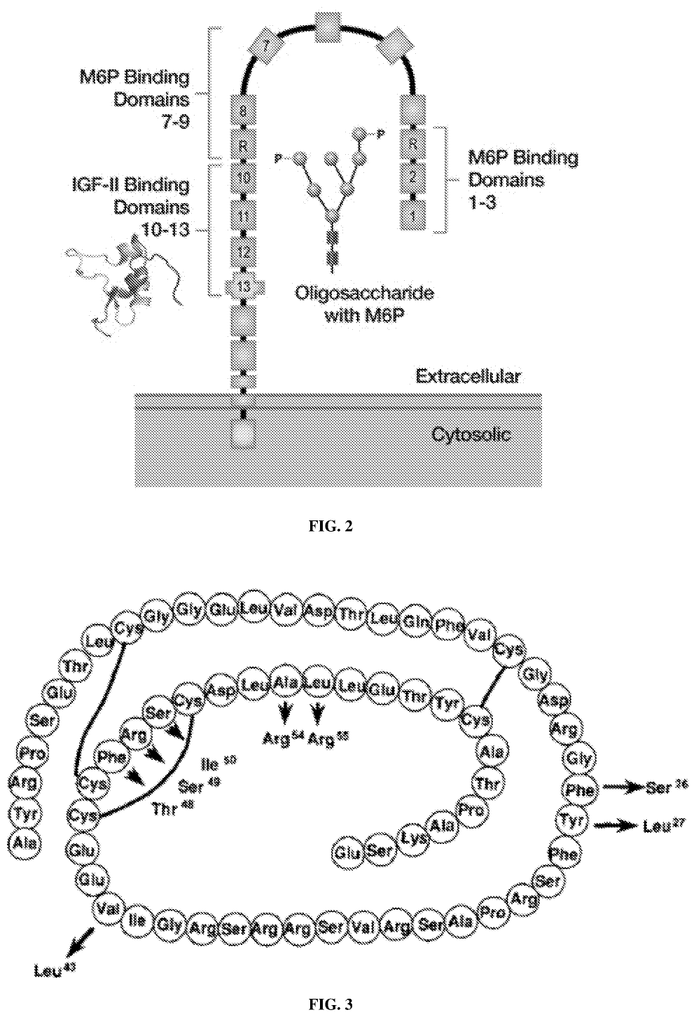

The CI-MPR captures M6P-containing lysosomal enzymes from circulation. The receptor has distinct binding domains for M6P and insulin-like growth factor (domains 1-3 and 7-9, see FIG. 2) and therefore is also known as the IGF2/Mannose-6-phosphate receptor or IGF2/CI-MPR. This receptor can be utilized for targeting M6P- or IGF2- or IGF2 variant-containing enzyme replacement therapeutics. Binding affinity of this receptor for these ligands including insulin-like growth factor is provided in Table 1. Notably, IGF2 peptide has a higher binding affinity for CI-MPR than mono- or bis-phosphorylated oligosaccharides.

TABLE 1
Ligands for CI-MPR

| Ligand | Binding Affinity (Apparent Kd; nM) |
|---|---|
| IGF2 | 0.03-0.2 |
| [Leu27]IGF2 | 0.05 |
| Bis-M6P | 2 |
| Beta-galactosidase | 20 |
| Pentamannose-M6P | 6,000 |
| Free M6P | 7,000 |

Accordingly, in some embodiments, it is desired to design improved variant IGF2 (vIGF2) peptides for making therapeutic fusion proteins that have increased stability, CI-MPR binding, cellular uptake and lysosomal localization, for example in treating diseases such as lysosomal storage diseases.

In some embodiments, the variant vIGF2 has improved binding to CI-MPR which is responsible for cellular uptake and delivery of IGF2 to lysosomes for degradation. Some variant IGF2 peptides have decreased affinity for insulin-like growth factor receptor 1 (IGF1R). In some embodiments, IGF2 has decreased or no affinity for integrins. In some embodiments, the IGF2 also has decreased or no affinity for at least one insulin-like growth factor binding proteins (IGFBP1-6). In some embodiments, the IGF2 variants have decreased or no binding to heparin. In some embodiments, the IGF2 variants

A goal in designing a vIGF2 peptide would be to improve the biophysical properties of the vIGF2 and enhance binding to CI-MPR/cellular uptake and lysosomal delivery, while minimizing the other functions. Accordingly, vIGF2 peptides may (1) improve stability/solubility of vIGF2; (2) attenuate binding affinity to IR/IGF1R/integrins; and (3) improve binding affinity to CI-MPR. In some embodiments, vIGF2 peptides are designed using structure guided rational design, identifying crucial versus dispensable residues, point mutations and truncations. In some embodiments, vIGF2 peptides are designed using in silico computational experiments comprising systemic mutational studies to determine if a given mutation affects stability and affinity to various binding partners, alanine scanning mutagenesis (NAMD), and/or improving IGF2 solubility, bioavailability, and/or reducing immunogenicity. In some embodiments, vIGF2 peptides are designed via directed evolution based on split-GFP assays. In some embodiments, vIGF2 peptides are designed via directed evolution based on phage display.

In some embodiments, vIGF2 peptides are designed using in silico computational experiments comprising systemic mutational studies to determine if a given mutation affects stability of the IGF2 peptide. In some embodiments, the stability of the peptide with the mutation is the same as or increased as compared to the wild type IGF2.

In some embodiments, vIGF2 peptides are designed to reduce binding to integrin. In some embodiments, vIGF2 peptides with reduced binding to integrin comprise mutations R24E/R34E, R24E/R37E/R38E, R34E/R37E/R38E, R24E/R37E, R24E/R38E, or R24E/R34E/R37E/R38E. In some embodiments, vIGF2 peptides have reduced binding to integrin and heparin, such as mutation of residues R37, R38, or R40.

In some embodiments, mutations T16I, T16V, T16L, T16F, T16Y, or T16W increase binding of vIGF2 to CI-MPR. In some embodiments, mutations T16V or T16Y increase binding of vIGF2 to CI-MPR. In some embodiments, mutations at D23, for example, D23K or D23R, increase binding of vIGF2 to CI-MPR. In some embodiments, mutations at F19, such as F19W, increase binding of vIGF2 to CI-MPR. In some embodiments, mutations at S50, such as S50D or S50E, increase binding of vIGF2 to CI-MPR. In some embodiments vIGF2 having mutations D23K and S50E have increased binding to CI-MPR. In some embodiments, vIGF2 having mutations A1-4, E6R, Y27L, and K65R have increased binding to CI-MPR. In some embodiments, vIGF2 having mutations A33-40, D23R, F26E, and S50E have increased binding to CI-MPR.

In some embodiments, vIGF2 peptides are designed to have reduced IGFR1 binding. In some embodiments, mutations that affect IGF1R binding are on the different face of IGF2 compared to mutations that affect CI-MPR binding. In some embodiments, F26, Y27, and V43 are important for binding to IGF1R. In some embodiments, vIGF2 peptides having a mutation of S29N, R34_GS, S39_PQ, R34_GS/S39_PQ, S29N/S39_PQ, or S29N/S39PQ, R43_GS have decreased binding to insulin receptor and IGF1R. In some embodiments, a vIGF2 peptide having a mutation of S39_PQ (PQ insertion after S39) has decreased binding to the insulin receptor and IGF1R. In some embodiments, vIGF2 peptides having mutations at G11, V14, L17, G25, F26, Y27, F28, S29, R30, P31, A32, S33, V35, S36, R37, S39, G41, 142, V43, E44, F48, T53, Y59, C60, or A61 have reduced binding to IGF1R. In some embodiments, vIGF2 peptides having mutations at G10, L13, V14, L17, F26, Y27, F28, S29, R30, P31, A32, S33, V35, G41, 142, V43, T58, or Y59 have reduced binding to IGF1R. In some embodiments, vIGF2 peptides having mutations V14D/F26A/F28R/V43D have reduced binding to IGF1R. In some embodiments, vIGF2 peptides having mutations F26S, Y27L, or V43L have reduced binding to IGF1R and/or insulin receptor.

In some embodiments, vIGF2 peptides have a deletion in the C domain (e.g., residues 32-41, SRVSRRSR) causing the vIGF2 peptides to have reduced binding to IGF1R, insulin receptor, heparin, and integrin. In some embodiments the vIGF2 peptides have the mutation Δ1-4, E6R, Δ30-39. In some embodiments, the vIGF2 peptides have the mutation Δ1-4, E6R, Δ33-40.

In some embodiments, vIGF2 peptides have mutations to decrease its instability index. In some embodiments, mutations of IGF2 peptides with increased stability include R38G, R38G/E45W, R38G/E45W/S50G, P31G/R38G/E45W/S50G, or L17N/P31G/R38G/E45W/S50G. In some embodiments, mutations of IGF2 peptides with increased stability include R38G, R38G/E45W, R38G/E45W/S50G, P31G/R38G/E45W/S50G, L17N/P31G/R38G/E45W/S50G, L17N/P31G/R38G/E45W/S50G/S66G, L17N/P31G/R38G/E45W/S50G/A64M/S66G, or S5L/L17N/P31G/R38G/E45W/S50G/A64M/S66G.

In some embodiments, vIGF2 peptides are mutated to reduce aggregation. In some embodiments, residues prone to aggregation include residues 17-21 (LQFVC), 41-49 (GIVEECCFR), or 53-62 (LALLETYCAT). In some embodiments, vIGF2 peptides are mutated at F26, Y59, Y27, V14, A1, or L8 to reduce aggregation.

In some embodiments, vIGF2 peptides are designed to have reduced binding to IGFBP. In some embodiments, vIGF2 peptides have the mutations L8A, V20A, or L56A. In some embodiments, vIGF2 peptides having mutations at E6, L8, R24, G25, F26, Y27, or F28 have reduced binding to IGFBP4. In some embodiments, vIGF2 peptides having mutations at T7, G10, V14, V43, E44, C47, or F48 have reduced binding to IGFBP4. In some embodiments, vIGF2 peptides having mutations at E6 or L8 have reduced binding to IGFBP4. In some embodiments, vIGF2 peptides having mutations E6Q or T7A have reduced binding to human serum binding protein. In some embodiments, vIGF2 peptides having mutations Q18Y or F19L have reduced binding to human serum binding protein. In some embodiments, vIGF2 peptides having mutations at E6Q, T7A, Q18Y, or F19L have reduced binding to human serum binding protein.

In some embodiments, vIGF2 peptides have been modified to replace residues 31-38 with GGGG (vIGF2 Δ 31-38GGGG), and some of these vIGF2 peptides further contain a V43L and an S50E or S50D mutation. (SEQ ID NO:s 120-121). In some embodiments, vIGF2 peptides that are at least 95% identical to SEQ ID NO:s 120-121 enhance expression and/or secretion of a therapeutic protein. In some embodiments, the therapeutic protein is PPT1 or TPP1 or an enzymatically active fragment thereof.

Therapeutic Fusion Proteins for Gene Therapy

Therapeutic fusion proteins produced from gene therapy vectors are provided herein. In some embodiments the fusion protein is secreted by cells transduced with the gene therapy vector encoding the fusion protein. In some embodiments, the transduced cells are within a tissue or organ (e.g., liver). Once secreted from a cell, the fusion protein is transported through a patient's vascular system and reaches the tissue of interest. In some embodiments, the therapeutic fusion protein is engineered to have improved secretion. In some embodiments, the fusion protein comprises a signal peptide for improving the secretion level as compared to the corresponding therapeutic protein or a fusion protein comprising the therapeutic protein having only a native signal peptide.

The provided gene therapy vectors are, in some embodiments, engineered to improve delivery of the therapeutic protein. For example, in some instances gene therapy may not achieve the intended treatment by merely generating a sufficient amount of a therapeutic protein in the body of the patient if an insufficient amount of the therapeutic protein is delivered into the cells in need of the therapeutic protein, due to, for example, physical and/or biological barriers that impede distribution of the therapeutic protein to the site where needed. As such, even if a gene therapy is capable of flooding blood or a tissue, to a point of saturation, with a high concentration of a therapeutic protein, the gene therapy may not be sufficiently therapeutic. Additionally, non-productive clearance pathways may remove the vast majority of the therapeutic protein. Even if the therapeutic protein is transported out of the vasculature to the interstitial space within the tissue (e.g., muscle fibers), adequate therapeutic effects are not assured. For effective treatment of lysosomal storage disorders, a therapeutically effective amount of the therapeutic protein must undergo cellular endocytosis and lysosomal delivery to result in a meaningful efficacy. The present disclosure addresses these issues by providing gene therapy vectors encoding fusion proteins comprising a peptide that enables endocytosis of the therapeutic protein into a target cell for treatment resulting in efficacious treatment. In some embodiments, the peptide that enables endocytosis is a peptide that binds the CI-MPR. In some embodiments, the peptide that binds the CI-MPR is a vIGF2 peptide. Recombinantly expressed GLA was known to be well phosphorylated and thus bind to the CIMPR, but surprisingly, GLA expressed in mice is under-phosphorylated and does not bind well to the CIMPR. Therefore, GLA for use in gene therapy unexpectedly requires additional engineering to enhance CIMPR binding (such as the IGF2 tag).

Provided herein are gene therapy vectors encoding fusion proteins comprising a peptide that enables endocytosis the therapeutic protein into a target cell for treatment. In some embodiments, the gene therapy vectors encode fusion proteins comprising a therapeutic protein and a peptide that binds the CI-MPR. Such fusion proteins when expressed from a gene therapy vector target therapeutic proteins, such as enzyme replacement therapeutics, to the cells where they are needed, increase delivery into or cellular uptake by such cells and target the therapeutic protein to a subcellular location (e.g., a lysosome). In some embodiments, the peptide is an IGF2 peptide or variant thereof, which can target a therapeutic protein to the lysosome. Fusion proteins herein also, in some embodiments, further comprise a signal peptide that increases secretion, such as a BiP signal peptide or a Gaussia signal peptide. In some embodiments, fusion proteins comprise a linker sequence. In some embodiments, nucleic acids encoding fusion proteins herein, comprise internal ribosomal entry sequences. Uh

Therapeutic Fusion Proteins for Enzyme Replacement Therapy

Therapeutic fusion proteins produced for enzyme replacement therapy are provided herein. The provided fusion proteins are, in some embodiments, engineered to improve delivery of the therapeutic protein. For example, in some instances fusion protein may not achieve the intended treatment if an insufficient amount of the therapeutic fusion protein is delivered into the cells in need of the therapeutic protein, due to, for example, physical and/or biological barriers that impede distribution of the therapeutic protein to the site where needed. Even if the therapeutic protein is transported out of the vasculature to the interstitial space within the tissue (e.g., muscle fibers), adequate therapeutic effects are not assured. For effective treatment of lysosomal storage disorders, a therapeutically effective amount of the therapeutic protein must undergo cellular endocytosis and lysosomal delivery to result in a meaningful efficacy. The present disclosure addresses these issues by providing fusion proteins comprising a peptide that enables endocytosis of the therapeutic protein into a target cell for treatment resulting in efficacious treatment. In some embodiments, the peptide that enables endocytosis is a peptide that binds the CI-MPR. In some embodiments, the peptide that binds the CI-MPR is a vIGF2 peptide.

Provided herein are fusion proteins comprising a peptide that enables endocytosis the therapeutic protein into a target cell for treatment. In some embodiments, the fusion proteins comprises a peptide that binds the CI-MPR. Such fusion proteins are used as enzyme replacement therapeutics, have increased delivery into or cellular uptake by cells needing such proteins and target the therapeutic protein to a subcellular location (e.g., a lysosome). In some embodiments, the peptide is an IGF2 peptide or variant thereof, which can target a therapeutic protein to the lysosome.

Therapeutic proteins for enzyme replacement therapy or gene therapy comprising a vIGF2 peptide are provided herein. Exemplary proteins are provided in Table 2 below.

TABLE 2
Amino Acid Sequences

| | | SEQ |
|---|---|---|
| | | ID |
| | | NO |
| Natural | MGVRHPPCSHRLLAVCALVSLATAALLGHILLHDFLLVPRELSGSSP | 1 |
| hGAA | VLEETHPAHQQGASRPGPRDAQAHPGRPRAVPTQCDVPPNSRFDCA | |
| | PDKAITQEQCEARGCCYIPAKQGLQGAQMGQPWCFFPPSYPSYKLE | |
| | NLSSSEMGYTATLTRTTPTFFPKDILTLRLDVMMETENRLHFTIKDP | |
| | ANRRYEVPLETPHVHSRAPSPLYSVEFSEEPFGVIVRRQLDGRVLLN | |
| | TTVAPLFFADQFLQLSTSLPSQYITGLAEHLSPLMLSTSWTRITLWNR | |
| | DLAPTPGANLYGSHPFYLALEDGGSAHGVFLLNSNAMDVVLQPSPA | |
| | LSWRSTGGILDVYIFLGPEPKSVVQQYLDVVGYPFMPPYWGLGFHL | |
| | CRWGYSSTAITRQVVENMTRAHFPLDVQWNDLDYMDSRRDFTFN | |
| | KDGFRDFPAMVQELHQGGRRYMMIVDPAISSSGPAGSYRPYDEGLR | |
| | RGVFITNETGQPLIGKVWPGSTAFPDFTNPTALAWWEDMVAEFHD | |
| | QVPFDGMWIDMNEPSNFIRGSEDGCPNNELENPPYVPGVVGGTLQA | |
| | ATICASSHQFLSTHYNLHNLYGLTEAIASHRALVKARGTRPFVISRST | |
| | FAGHGRYAGHWTGDVWSSWEQLASSVPEILQFNLLGVPLVGADVC | |
| | GFLGNTSEELCVRWTQLGAFYPFMRNHNSLLSLPQEPYSFSEPAQQ | |
| | AMRKALTLRYALLPHLYTLFHQAHVAGETVARPLFLEFPKDSSTWT | |
| | VDHQLLWGEALLITPVLQAGKAEVTGYFPLGTWYDLQTVPVEALG | |
| | SLPPPPAAPREPAIHSEGQWVTLPAPLDTINVHLRAGYIIPLQGPGLT | |
| | TTESRQQPMALAVALTKGGEARGELFWDDGESLEVLERGAYTQVIF | |
| | LARNNTIVNELVRVTSEGAGLQLQKVTVLGVATAPQQVLSNGVPVS | |
| | NFTYSPDTKVLDICVSLLMGEQFLVSWC | |
| Engineered | MKLSLVAAMLLLLSAARASRTLCGGELVDTLQFVCGDRGFLFSRPA | 2 |
| hGAA (BiP- | SRVSRRSRGIVEECCFRSCDLALLETYCATPARSEGGGGSGGGGSRP | |
| vIGF2- | GPRDAQAHPGRPRAVPTQCDVPPNSRFDCAPDKAITQEQCEARGCC | |
| GAA) | YIPAKQGLQGAQMGQPWCFFPPSYPSYKLENLSSSEMGYTATLTRT | |
| | TPTFFPKDILTLRLDVMMETENRLHFTIKDPANRRYEVPLETPHVHS | |
| | RAPSPLYSVEFSEEPFGVIVRRQLDGRVLLNTTVAPLFFADQFLQLST | |
| | SLPSQYITGLAEHLSPLMLSTSWTRITLWNRDLAPTPGANLYGSHPF | |
| | YLALEDGGSAHGVFLLNSNAMDVVLQPSPALSWRSTGGILDVYIFL | |
| | GPEPKSVVQQYLDVVGYPFMPPYWGLGFHLCRWGYSSTAITRQVV | |
| | ENMTRAHFPLDVQWNDLDYMDSRRDFTFNKDGFRDFPAMVQELH | |
| | QGGRRYMMIVDPAISSSGPAGSYRPYDEGLRRGVFITNETGQPLIGK | |
| | VWPGSTAFPDFTNPTALAWWEDMVAEFHDQVPFDGMWIDMNEPS | |
| | NFIRGSEDGCPNNELENPPYVPGVVGGTLQAATICASSHQFLSTHYN | |
| | LHNLYGLTEAIASHRALVKARGTRPFVISRSTFAGHGRYAGHWTGD | |
| | VWSSWEQLASSVPEILQFNLLGVPLVGADVCGFLGNTSEELCVRWT | |
| | QLGAFYPFMRNHNSLLSLPQEPYSFSEPAQQAMRKALTLRYALLPH | |
| | LYTLFHQAHVAGETVARPLFLEFPKDSSTWTVDHQLLWGEALLITP | |
| | VLQAGKAEVTGYFPLGTWYDLQTVPVEALGSLPPPPAAPREPAIHSE | |
| | GQWVTLPAPLDTINVHLRAGYIIPLQGPGLTTTESRQQPMALAVALT | |
| | KGGEARGELFWDDGESLEVLERGAYTQVIFLARNNTIVNELVRVTS | |
| | EGAGLQLQKVTVLGVATAPQQVLSNGVPVSNFTYSPDTKVLDICVS | |
| | LLMGEQFLVSWC | |
| hGAA Δ1- | SRPGPRDAQAHPGRPRAVPTQCDVPPNSRFDCAPDKAITQEQCEAR | 3 |
| 60 | GCCYIPAKQGLQGAQMGQPWCFFPPSYPSYKLENLSSSEMGYTATL | |
| | TRTTPTFFPKDILTLRLDVMMETENRLHFTIKDPANRRYEVPLETPH | |
| | VHSRAPSPLYSVEFSEEPFGVIVRRQLDGRVLLNTTVAPLFFADQFL | |
| | QLSTSLPSQYITGLAEHLSPLMLSTSWTRITLWNRDLAPTPGANLYG | |
| | SHPFYLALEDGGSAHGVFLLNSNAMDVVLQPSPALSWRSTGGILDV | |
| | YIFLGPEPKSVVQQYLDVVGYPFMPPYWGLGFHLCRWGYSSTAITR | |
| | QVVENMTRAHFPLDVQWNDLDYMDSRRDFTFNKDGFRDFPAMVQ | |
| | ELHQGGRRYMMIVDPAISSSGPAGSYRPYDEGLRRGVFITNETGQPL | |
| | IGKVWPGSTAFPDFTNPTALAWWEDMVAEFHDQVPFDGMWIDMN | |
| | EPSNFIRGSEDGCPNNELENPPYVPGVVGGTLQAATICASSHQFLST | |
| | HYNLHNLYGLTEAIASHRALVKARGTRPFVISRSTFAGHGRYAGHW | |
| | TGDVWSSWEQLASSVPEILQFNLLGVPLVGADVCGFLGNTSEELCV | |
| | RWTQLGAFYPFMRNHNSLLSLPQEPYSFSEPAQQAMRKALTLRYAL | |
| | LPHLYTLFHQAHVAGETVARPLFLEFPKDSSTWTVDHQLLWGEALL | |
| | ITPVLQAGKAEVTGYFPLGTWYDLQTVPVEALGSLPPPPAAPREPAI | |
| | HSEGQWVTLPAPLDTINVHLRAGYIIPLQGPGLTTTESRQQPMALAV | |
| | ALTKGGEARGELFWDDGESLEVLERGAYTQVIFLARNNTIVNELVR | |
| | VTSEGAGLQLQKVTVLGVATAPQQVLSNGVPVSNFTYSPDTKVLDI | |
| | CVSLLMGEQFLVSWC | |
| wt- | MASPGCLWLLAVALLPWTCASRALQHLDPPAPLPLVIWHGMGDSC | 4 |
| palmitoyl- | CNPLSMGAIKKMVEKKIPGIYVLSLEIGKTLMEDVENSFFLNVNSQV | |
| protein | TTVCQALAKDPKLQQGYNAMGFSQGGQFLRAVAQRCPSPPMINLIS | |
| thioesterase | VGGQHQGVFGLPRCPGESSHICDFIRKTLNAGAYSKVVQERLVQAE | |
| 1 (PPT1) | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLG | |
| PPT1-2 | MASPGCLWLLAVALLPWTCASRALQHLSRTLCGGELVDTLQFVCG | 5 |
| (vIGF2- | DRGFLFSRPASRVSRRSRGIVEECCFRSCDLALLETYCATPARSEGG | |
| PPT1) | GGSGGGGSRPRAVPTQDPPAPLPLVIWHGMGDSCCNPLSMGAIKK | |
| | MVEKKIPGIYVLSLEIGKTLMEDVENSFFLNVNSQVTTVCQALAKDP | |
| | KLQQGYNAMGFSQGGQFLRAVAQRCPSPPMINLISVGGQHQGVFG | |
| | LPRCPGESSHICDFIRKTLNAGAYSKVVQERLVQAEYWHDPIKEDV | |
| | YRNHSIFLADINQERGINESYKKNLMALKKFVMVKFLNDSIVDPVD | |
| | SEWFGFYRSGQAKETIPLQETSLYTQDRLGLKEMDNAGQLVFLATE | |
| | GDHLQLSEEWFYAHIIPFLG | |
| PPT1-29 | MKLSLVAAMLLLLWVALLLLSAARAAASRTLCGGELVDTLQFVCG | 6 |
| (BiP2aa- | DRGFLFSRPASRVSRRSRGIVEECCFRSCDLALLETYCATPARSEGG | |
| vIGF2- | GGSGGGGSRPRAVPTQDPPAPLPLVIWHGMGDSCCNPLSMGAIKK | |
| PPT1) | MVEKKIPGIYVLSLEIGKTLMEDVENSFFLNVNSQVTTVCQALAKDP | |
| | KLQQGYNAMGFSQGGQFLRAVAQRCPSPPMINLISVGGQHQGVFG | |
| | LPRCPGESSHICDFIRKTLNAGAYSKVVQERLVQAEYWHDPIKEDV | |
| | YRNHSIFLADINQERGINESYKKNLMALKKFVMVKFLNDSIVDPVD | |
| | SEWFGFYRSGQAKETIPLQETSLYTQDRLGLKEMDNAGQLVFLATE | |
| | GDHLQLSEEWFYAHIIPFLG | |
| PPT1 | MASPGCLWLLAVALLPWTCASRALQHLDPPAPLPLVIWHGMGDSC | 7 |
| engineered | CNPLSMGAIKKMVEKKIPGIYVLSLEIGKTLMEDVENSFFLNVNSQV | |
| | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLGRPRAVPTQGGSGSGSTS | |
| | SSRTLCGGELVDTLQFVCGDRGFLFSRPASRVSRRSRGIVEECCFRE | |
| | CDLALLETYCATPARSE | |
| TPP1 | MGLQACLLGLFALILSGKCSYSPEPDQRRTLPPGWVSLGRADPEEEL | 8 |
| wildtype | SLTFALRQQNVERLSELVQAVSDPSSPQYGKYLTLENVADLVRPSPL | |
| | TLHTVQKWLLAAGAQKCHSVITQDFLTCWLSIRQAELLLPGAEFHH | |
| | YVGGPTETHVVRSPHPYQLPQALAPHVDFVGGLHRFPPTSSLRQRPE | |
| | PQVTGTVGLHLGVTPSVIRKRYNLTSQDVGSGTSNNSQACAQFLEQ | |
| | YFHDSDLAQFMRLFGGNFAHQASVARVVGQQGRGRAGIEASLDVQ | |
| | YLMSAGANISTWVYSSPGRHEGQEPFLQWLMLLSNESALPHVHTVS | |
| | YGDDEDSLSSAYIQRVNTELMKAAARGLTLLFASGDSGAGCWSVS | |
| | GRHQFRPTFPASSPYVTTVGGTSFQEPFLITNEIVDYISGGGFSNVFPR | |
| | PSYQEEAVTKFLSSSPHLPPSSYFNASGRAYPDVAALSDGYWVVSN | |
| | RVPIPWVSGTSASTPVFGGILSLINEHRILSGRPPLGFLNPRLYQQHG | |
| | AGLFDVTRGCHESCLDEEVEGQGFCSGPGWDPVTGWGTPNFPALL | |
| | KTLLNP | |
| TPP1 | MGLQACLLGLFALILSGKCSRTLCGGELVDTLQFVCGDRGFLFSRPA | 9 |
| engineered | SRVSRRSRGIVEECCFRSCDLALLETYCATPARSEGGGGSGGGGSRP | |
| | RAVPTQSYSPEPDQRRTLPPGWVSLGRADPEEELSLTFALRQQNVER | |
| | LSELVQAVSDPSSPQYGKYLTLENVADLVRPSPLTLHTVQKWLLAA | |
| | GAQKCHSVITQDFLTCWLSIRQAELLLPGAEFHHYVGGPTETHVVR | |
| | SPHPYQLPQALAPHVDFVGGLHRFPPTSSLRQRPEPQVTGTVGLHLG | |
| | VTPSVIRKRYNLTSQDVGSGTSNNSQACAQFLEQYFHDSDLAQFMR | |
| | LFGGNFAHQASVARVVGQQGRGRAGIEASLDVQYLMSAGANISTW | |
| | VYSSPGRHEGQEPFLQWLMLLSNESALPHVHTVSYGDDEDSLSSAY | |
| | IQRVNTELMKAAARGLTLLFASGDSGAGCWSVSGRHQFRPTFPASS | |
| | PYVTTVGGTSFQEPFLITNEIVDYISGGGFSNVFPRPSYQEEAVTKFL | |
| | SSSPHLPPSSYFNASGRAYPDVAALSDGYWVVSNRVPIPWVSGTSAS | |
| | TPVFGGILSLINEHRILSGRPPLGFLNPRLYQQHGAGLFDVTRGCHES | |
| | CLDEEVEGQGFCSGPGWDPVTGWGTPNFPALLKTLLNP | |
| AGA | MARKSNLPVLLVPFLLCQALVRCSSPLPLVVNTWPFKNATEAAWR | 10 |
| wildtype | ALASGGSALDAVESGCAMCEREQCDGSVGFGGSPDELGETTLDAMI | |
| | MDGTTMDVGAVGDLRRIKNAIGVARKVLEHTTHTLLVGESATTFA | |
| | QSMGFINEDLSTTASQALHSDWLARNCQPNYWRNVIPDPSKYCGPY | |
| | KPPGILKQDIPIHKETEDDRGHDTIGMVVIHKTGHIAAGTSTNGIKFK | |
| | IHGRVGDSPIPGAGAYADDTAGAAAATGNGDILMRFLPSYQAVEY | |
| | MRRGEDPTIACQKVISRIQKHFPEFFGAVICANVTGSYGAACNKLST | |
| | FTQFSFMVYNSEKNQPTEEKVDCI | |
| AGA | MARKSNLPVLLVPFLLCQALVRCSRTLCGGELVDTLQFVCGDRGFL | 11 |
| engineered | FSRPASRVSRRSRGIVEECCFRSCDLALLETYCATPARSEGGGGSGG | |
| (N-terminal | GGSRPRAVPTQSSPLPLVVNTWPFKNATEAAWRALASGGSALDAV | |
| fusion) | ESGCAMCEREQCDGSVGFGGSPDELGETTLDAMIMDGTTMDVGAV | |
| | GDLRRIKNAIGVARKVLEHTTHTLLVGESATTFAQSMGFINEDLSTT | |
| | ASQALHSDWLARNCQPNYWRNVIPDPSKYCGPYKPPGILKQDIPIH | |
| | KETEDDRGHDTIGMVVIHKTGHIAAGTSTNGIKFKIHGRVGDSPIPG | |
| | AGAYADDTAGAAAATGNGDILMRFLPSYQAVEYMRRGEDPTIACQ | |
| | KVISRIQKHFPEFFGAVICANVTGSYGAACNKLSTFTQFSFMVYNSE | |
| | KNQPTEEKVDCI | |
| GLA | MQLRNPELHLGCALALRFLALVSWDIPGARALDNGLARTPTMGWL | 12 |
| wildtype | HWERFMCNLDCQEEPDSCISEKLFMEMAELMVSEGWKDAGYEYL | |
| | CIDDCWMAPQRDSEGRLQADPQRFPHGIRQLANYVHSKGLKLGIYA | |
| | DVGNKTCAGFPGSFGYYDIDAQTFADWGVDLLKFDGCYCDSLENL | |
| | ADGYKHMSLALNRTGRSIVYSCEWPLYMWPFQKPNYTEIRQYCNH | |
| | WRNFADIDDSWKSIKSILDWTSFNQERIVDVAGPGGWNDPDMLVIG | |
| | NFGLSWNQQVTQMALWAIMAAPLFMSNDLRHISPQAKALLQDKD | |
| | VIAINQDPLGKQGYQLRQGDNFEVWERPLSGLAWAVAMINRQEIG | |
| | GPRSYTIAVASLGKGVACNPACFITQLLPVKRKLGFYEWTSRLRSHI | |
| | NPTGTVLLQLENTMQMSLKDLL | |
| GLA | MQLRNPELHLGCALALRFLALVSWDIPGARALDNGLARTPTMGWL | 13 |
| engineered | HWERFMCNLDCQEEPDSCISEKLFMEMAELMVSEGWKDAGYEYL | |
| | CIDDCWMAPQRDSEGRLQADPQRFPHGIRQLANYVHSKGLKLGIYA | |
| | DVGNKTCAGFPGSFGYYDIDAQTFADWGVDLLKFDGCYCDSLENL | |
| | ADGYKHMSLALNRTGRSIVYSCEWPLYMWPFQKPNYTEIRQYCNH | |
| | WRNFADIDDSWKSIKSILDWTSFNQERIVDVAGPGGWNDPDMLVIG | |
| | NFGLSWNQQVTQMALWAIMAAPLFMSNDLRHISPQAKALLQDKD | |
| | VIAINQDPLGKQGYQLRQGDNFEVWERPLSGLAWAVAMINRQEIG | |
| | GPRSYTIAVASLGKGVACNPACFITQLLPVKRKLGFYEWTSRLRSHI | |
| | NPTGTVLLQLENTMQMSLKDLLYIPAKQGLQGAQMGQPGGGGSGG | |
| | GGSRTLCGGELVDTLQFVCGDRGFLFSRPASRVSRRSRGIVEECCFR | |
| | SCDLALLETYCATPARSE | |
| BiP-vIGF2- | MKLSLVAAMLLLLSAARASRTLCGGELVDTLQFVCGDRGFLFSRPA | 14 |
| 17-2GS- | SRVSRRSRGIVEECCFRECDLALLETYCATPARSEGGGGSGGGGSRP | |
| GAA | GPRDAQAHPGRPRAVPTQCDVPPNSRFDCAPDKAITQEQCEARGCC | |
| | YIPAKQGLQGAQMGQPWCFFPPSYPSYKLENLSSSEMGYTATLTRT | |
| | TPTFFPKDILTLRLDVMMETENRLHFTIKDPANRRYEVPLETPHVHS | |
| | RAPSPLYSVEFSEEPFGVIVRRQLDGRVLLNTTVAPLFFADQFLQLST | |
| | SLPSQYITGLAEHLSPLMLSTSWTRITLWNRDLAPTPGANLYGSHPF | |
| | YLALEDGGSAHGVFLLNSNAMDVVLQPSPALSWRSTGGILDVYIFL | |
| | GPEPKSVVQQYLDVVGYPFMPPYWGLGFHLCRWGYSSTAITRQVV | |
| | ENMTRAHFPLDVQWNDLDYMDSRRDFTFNKDGFRDFPAMVQELH | |
| | QGGRRYMMIVDPAISSSGPAGSYRPYDEGLRRGVFITNETGQPLIGK | |
| | VWPGSTAFPDFTNPTALAWWEDMVAEFHDQVPFDGMWIDMNEPS | |
| | NFIRGSEDGCPNNELENPPYVPGVVGGTLQAATICASSHQFLSTHYN | |
| | LHNLYGLTEAIASHRALVKARGTRPFVISRSTFAGHGRYAGHWTGD | |
| | VWSSWEQLASSVPEILQFNLLGVPLVGADVCGFLGNTSEELCVRWT | |
| | QLGAFYPFMRNHNSLLSLPQEPYSFSEPAQQAMRKALTLRYALLPH | |
| | LYTLFHQAHVAGETVARPLFLEFPKDSSTWTVDHQLLWGEALLITP | |
| | VLQAGKAEVTGYFPLGTWYDLQTVPVEALGSLPPPPAAPREPAIHSE | |
| | GQWVTLPAPLDTINVHLRAGYIIPLQGPGLTTTESRQQPMALAVALT | |
| | KGGEARGELFWDDGESLEVLERGAYTQVIFLARNNTIVNELVRVTS | |
| | EGAGLQLQKVTVLGVATAPQQVLSNGVPVSNFTYSPDTKVLDICVS | |
| | LLMGEQFLVSWC | |
| BiP-vIGF2- | MKLSLVAAMLLLLSAARASRTLCGGELVDTLQFVCGDRGFLFSRPA | 15 |
| 20-2GS- | SRVSRRSRGILEECCFRSCDLALLETYCATPARSEGGGGSGGGGSRP | |
| GAA | GPRDAQAHPGRPRAVPTQCDVPPNSRFDCAPDKAITQEQCEARGCC | |
| | YIPAKQGLQGAQMGQPWCFFPPSYPSYKLENLSSSEMGYTATLTRT | |
| | TPTFFPKDILTLRLDVMMETENRLHFTIKDPANRRYEVPLETPHVHS | |
| | RAPSPLYSVEFSEEPFGVIVRRQLDGRVLLNTTVAPLFFADQFLQLST | |
| | SLPSQYITGLAEHLSPLMLSTSWTRITLWNRDLAPTPGANLYGSHPF | |
| | YLALEDGGSAHGVFLLNSNAMDVVLQPSPALSWRSTGGILDVYIFL | |
| | GPEPKSVVQQYLDVVGYPFMPPYWGLGFHLCRWGYSSTAITRQVV | |
| | ENMTRAHFPLDVQWNDLDYMDSRRDFTFNKDGFRDFPAMVQELH | |
| | QGGRRYMMIVDPAISSSGPAGSYRPYDEGLRRGVFITNETGQPLIGK | |
| | VWPGSTAFPDFTNPTALAWWEDMVAEFHDQVPFDGMWIDMNEPS | |
| | NFIRGSEDGCPNNELENPPYVPGVVGGTLQAATICASSHQFLSTHYN | |
| | LHNLYGLTEAIASHRALVKARGTRPFVISRSTFAGHGRYAGHWTGD | |
| | VWSSWEQLASSVPEILQFNLLGVPLVGADVCGFLGNTSEELCVRWT | |
| | QLGAFYPFMRNHNSLLSLPQEPYSFSEPAQQAMRKALTLRYALLPH | |
| | LYTLFHQAHVAGETVARPLFLEFPKDSSTWTVDHQLLWGEALLITP | |
| | VLQAGKAEVTGYFPLGTWYDLQTVPVEALGSLPPPPAAPREPAIHSE | |
| | GQWVTLPAPLDTINVHLRAGYIIPLQGPGLTTTESRQQPMALAVALT | |
| | KGGEARGELFWDDGESLEVLERGAYTQVIFLARNNTIVNELVRVTS | |
| | EGAGLQLQKVTVLGVATAPQQVLSNGVPVSNFTYSPDTKVLDICVS | |
| | LLMGEQFLVSWC | |
| BiP-vIGF2- | MKLSLVAAMLLLLSAARASRTLCGGELVDTLQFVCGDRGFLFSRG | 16 |
| 22-2GS- | GGGSRGIVEECCFRSCDLALLETYCATPARSEGGGGSGGGGSRPGPR | |
| GAA | DAQAHPGRPRAVPTQCDVPPNSRFDCAPDKAITQEQCEARGCCYIP | |
| | AKQGLQGAQMGQPWCFFPPSYPSYKLENLSSSEMGYTATLTRTTPT | |
| | FFPKDILTLRLDVMMETENRLHFTIKDPANRRYEVPLETPHVHSRAP | |
| | SPLYSVEFSEEPFGVIVRRQLDGRVLLNTTVAPLFFADQFLQLSTSLP | |
| | SQYITGLAEHLSPLMLSTSWTRITLWNRDLAPTPGANLYGSHPFYLA | |
| | LEDGGSAHGVFLLNSNAMDVVLQPSPALSWRSTGGILDVYIFLGPEP | |
| | KSVVQQYLDVVGYPFMPPYWGLGFHLCRWGYSSTAITRQVVENM | |
| | TRAHFPLDVQWNDLDYMDSRRDFTFNKDGFRDFPAMVQELHQGG | |
| | RRYMMIVDPAISSSGPAGSYRPYDEGLRRGVFITNETGQPLIGKVWP | |
| | GSTAFPDFTNPTALAWWEDMVAEFHDQVPFDGMWIDMNEPSNFIR | |
| | GSEDGCPNNELENPPYVPGVVGGTLQAATICASSHQFLSTHYNLHN | |
| | LYGLTEAIASHRALVKARGTRPFVISRSTFAGHGRYAGHWTGDVWS | |
| | SWEQLASSVPEILQFNLLGVPLVGADVCGFLGNTSEELCVRWTQLG | |
| | AFYPFMRNHNSLLSLPQEPYSFSEPAQQAMRKALTLRYALLPHLYT | |
| | LFHQAHVAGETVARPLFLEFPKDSSTWTVDHQLLWGEALLITPVLQ | |
| | AGKAEVTGYFPLGTWYDLQTVPVEALGSLPPPPAAPREPAIHSEGQ | |
| | WVTLPAPLDTINVHLRAGYIIPLQGPGLTTTESRQQPMALAVALTKG | |
| | GEARGELFWDDGESLEVLERGAYTQVIFLARNNTIVNELVRVTSEG | |
| | AGLQLQKVTVLGVATAPQQVLSNGVPVSNFTYSPDTKVLDICVSLL | |
| | MGEQFLVSWC | |
| PPT1-3 | MKLSLVAAMLLLLSAARADPPAPLPLVIWHGMGDSCCNPLSMGAI | 17 |
| (BiP-PPT1) | KKMVEKKIPGIYVLSLEIGKTLMEDVENSFFLNVNSQVTTVCQALA | |
| | KDPKLQQGYNAMGFSQGGQFLRAVAQRCPSPPMINLISVGGQHQG | |
| | VFGLPRCPGESSHICDFIRKTLNAGAYSKVVQERLVQAEYWHDPIKE | |
| | DVYRNHSIFLADINQERGINESYKKNLMALKKFVMVKFLNDSIVDP | |
| | VDSEWFGFYRSGQAKETIPLQETSLYTQDRLGLKEMDNAGQLVFLA | |
| | TEGDHLQLSEEWFYAHIIPFLG | |
| PPT1-4 | MKLSLVAAMLLLLSAARASRTLCGGELVDTLQFVCGDRGFLFSRPA | 18 |
| (BiP-vIGF2- | SRVSRRSRGIVEECCFRSCDLALLETYCATPARSEGGGGSGGGGSRP | |
| PPT1) | RAVPTQDPPAPLPLVIWHGMGDSCCNPLSMGAIKKMVEKKIPGIYV | |
| | LSLEIGKTLMEDVENSFFLNVNSQVTTVCQALAKDPKLQQGYNAM | |
| | GFSQGGQFLRAVAQRCPSPPMINLISVGGQHQGVFGLPRCPGESSHI | |
| | CDFIRKTLNAGAYSKVVQERLVQAEYWHDPIKEDVYRNHSIFLADI | |
| | NQERGINESYKKNLMALKKFVMVKFLNDSIVDPVDSEWFGFYRSG | |
| | QAKETIPLQETSLYTQDRLGLKEMDNAGQLVFLATEGDHLQLSEEW | |
| | FYAHIIPFLG | |
| PPT1-5 (wt- | MASPGCLWLLAVALLPWTCASRALQHLDPPAPLPLVIWHGMGDSC | 19 |
| PPT1- | CNPLSMGAIKKMVEKKIPGIYVLSLEIGKTLMEDVENSFFLNVNSQV | |
| vIGF2) | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLGRPRAVPTQGGGGSGGG | |
| | GSRTLCGGELVDTLQFVCGDRGFLFSRPASRVSRRSRGIVEECCFRS | |
| | CDLALLETYCATPARSE | |
| PPT1-9 (wt- | MASPGCLWLLAVALLPWTCASRALQHLDPPAPLPLVIWHGMGDSC | 20 |
| PPT1) | CNPLSMGAIKKMVEKKIPGIYVLSLEIGKTLMEDVENSFFLNVNSQV | |
| | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLG | |
| PPT1-10 | MASPGCLWLLAVALLPWTCASRALQHLDPPAPLPLVIWHGMGDSC | 21 |
| (wt-PPT1- | CNPLSMGAIKKMVEKKIPGIYVLSLEIGKTLMEDVENSFFLNVNSQV | |
| vIGF2_2) | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLGRPRAVPTQGGSGSGSTS | |
| | SRTLCGGELVDTLQFVCGDRGFLFSRPASRVSRRSRGIVEECCFRSC | |
| | DLALLETYCATPARSE | |
| PPT1-11 | MKLSLVAAMLLLLSAARASRALQHLDPPAPLPLVIWHGMGDSCCN | 22 |
| (BiP- | PLSMGAIKKMVEKKIPGIYVLSLEIGKTLMEDVENSFFLNVNSQVTT | |
| PPT1_2) | VCQALAKDPKLQQGYNAMGFSQGGQFLRAVAQRCPSPPMINLISV | |
| | GGQHQGVFGLPRCPGESSHICDFIRKTLNAGAYSKVVQERLVQAEY | |
| | WHDPIKEDVYRNHSIFLADINQERGINESYKKNLMALKKFVMVKFL | |
| | NDSIVDPVDSEWFGFYRSGQAKETIPLQETSLYTQDRLGLKEMDNA | |
| | GQLVFLATEGDHLQLSEEWFYAHIIPFLG | |
| PPT1-12 | MKLSLVAAMLLLLSAARASRALQHLDPPAPLPLVIWHGMGDSCCN | 23 |
| (BiPaa- | PLSMGAIKKMVEKKIPGIYVLSLEIGKTLMEDVENSFFLNVNSQVTT | |
| PPT1_2) | VCQALAKDPKLQQGYNAMGFSQGGQFLRAVAQRCPSPPMINLISV | |
| | GGQHQGVFGLPRCPGESSHICDFIRKTLNAGAYSKVVQERLVQAEY | |
| | WHDPIKEDVYRNHSIFLADINQERGINESYKKNLMALKKFVMVKFL | |
| | NDSIVDPVDSEWFGFYRSGQAKETIPLQETSLYTQDRLGLKEMDNA | |
| | GQLVFLATEGDHLQLSEEWFYAHIIPFLG | |
| PPT1-13 | MKLSLVAAMLLLLSAARAAADPPAPLPLVIWHGMGDSCCNPLSMG | 24 |
| (BiPaa- | AIKKMVEKKIPGIYVLSLEIGKTLMEDVENSFFLNVNSQVTTVCQAL | |
| PPT1) | AKDPKLQQGYNAMGFSQGGQFLRAVAQRCPSPPMINLISVGGQHQ | |
| | GVFGLPRCPGESSHICDFIRKTLNAGAYSKVVQERLVQAEYWHDPI | |
| | KEDVYRNHSIFLADINQERGINESYKKNLMALKKFVMVKFLNDSIV | |
| | DPVDSEWFGFYRSGQAKETIPLQETSLYTQDRLGLKEMDNAGQLVF | |
| | LATEGDHLQLSEEWFYAHIIPFLG | |
| PPT1-14 | MKLSLVAAMLLLLSLVAAMLLLLSAARASRTLCGGELVDTLQFVC | 25 |
| (BiP1- | GDRGFLFSRPASRVSRRSRGIVEECCFRSCDLALLETYCATPARSEG | |
| vIGF2- | GGGSGGGGSRPRAVPTQDPPAPLPLVIWHGMGDSCCNPLSMGAIKK | |
| PPT1) | MVEKKIPGIYVLSLEIGKTLMEDVENSFFLNVNSQVTTVCQALAKDP | |
| | KLQQGYNAMGFSQGGQFLRAVAQRCPSPPMINLISVGGQHQGVFG | |
| | LPRCPGESSHICDFIRKTLNAGAYSKVVQERLVQAEYWHDPIKEDV | |
| | YRNHSIFLADINQERGINESYKKNLMALKKFVMVKFLNDSIVDPVD | |
| | SEWFGFYRSGQAKETIPLQETSLYTQDRLGLKEMDNAGQLVFLATE | |
| | GDHLQLSEEWFYAHIIPFLG | |
| PPT1-15 | MKLSLVAAMLLLLSLVAAMLLLLSAARAAASRTLCGGELVDTLQF | 26 |
| (BiPlaa- | VCGDRGFLFSRPASRVSRRSRGIVEECCFRSCDLALLETYCATPARS | |
| vIGF2- | EGGGGSGGGGSRPRAVPTQDPPAPLPLVIWHGMGDSCCNPLSMGAI | |
| PPT1) | KKMVEKKIPGIYVLSLEIGKTLMEDVENSFFLNVNSQVTTVCQALA | |
| | KDPKLQQGYNAMGFSQGGQFLRAVAQRCPSPPMINLISVGGQHQG | |
| | VFGLPRCPGESSHICDFIRKTLNAGAYSKVVQERLVQAEYWHDPIKE | |
| | DVYRNHSIFLADINQERGINESYKKNLMALKKFVMVKFLNDSIVDP | |
| | VDSEWFGFYRSGQAKETIPLQETSLYTQDRLGLKEMDNAGQLVFLA | |
| | TEGDHLQLSEEWFYAHIIPFLG | |
| PPT1-16 | MKLSLVAAMLLLLSLVAAMLLLLSAARAAASRALQHLDPPAPLPL | 27 |
| (BiP1aa- | VIWHGMGDSCCNPLSMGAIKKMVEKKIPGIYVLSLEIGKTLMEDVE | |
| PPT1_2) | NSFFLNVNSQVTTVCQALAKDPKLQQGYNAMGFSQGGQFLRAVAQ | |
| | RCPSPPMINLISVGGQHQGVFGLPRCPGESSHICDFIRKTLNAGAYSK | |
| | VVQERLVQAEYWHDPIKEDVYRNHSIFLADINQERGINESYKKNLM | |
| | ALKKFVMVKFLNDSIVDPVDSEWFGFYRSGQAKETIPLQETSLYTQ | |
| | DRLGLKEMDNAGQLVFLATEGDHLQLSEEWFYAHIIPFLG | |
| PPT1- | MASPGSLWLLAVALLPWTCASRALQHLDPPAPLPLVIWHGMGDSC | 28 |
| 17(wt- | CNPLSMGAIKKMVEKKIPGIYVLSLEIGKTLMEDVENSFFLNVNSQV | |
| PPT1-C6S) | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLG | |
| PPT1-18 | MKLSLVAAMLLLLWVALLLLSAARAAASRALQHLDPPAPLPLVIW | 29 |
| (BiP2aa- | HGMGDSCCNPLSMGAIKKMVEKKIPGIYVLSLEIGKTLMEDVENSF | |
| PPT1 | FLNVNSQVTTVCQALAKDPKLQQGYNAMGFSQGGQFLRAVAQRC | |
| | PSPPMINLISVGGQHQGVFGLPRCPGESSHICDFIRKTLNAGAYSKVV | |
| | QERLVQAEYWHDPIKEDVYRNHSIFLADINQERGINESYKKNLMAL | |
| | KKFVMVKFLNDSIVDPVDSEWFGFYRSGQAKETIPLQETSLYTQDR | |
| | LGLKEMDNAGQLVFLATEGDHLQLSEEWFYAHIIPFLG | |
| PPT1-19 | MGVKVLFALICIAVAEAAASRALQHLDPPAPLPLVIWHGMGDSCCN | 30 |
| (GaussiaAA- | PLSMGAIKKMVEKKIPGIYVLSLEIGKTLMEDVENSFFLNVNSQVTT | |
| PPT1_2) | VCQALAKDPKLQQGYNAMGFSQGGQFLRAVAQRCPSPPMINLISV | |
| | GGQHQGVFGLPRCPGESSHICDFIRKTLNAGAYSKVVQERLVQAEY | |
| | WHDPIKEDVYRNHSIFLADINQERGINESYKKNLMALKKFVMVKFL | |
| | NDSIVDPVDSEWFGFYRSGQAKETIPLQETSLYTQDRLGLKEMDNA | |
| | GQLVFLATEGDHLQLSEEWFYAHIIPFLG | |
| PPT1-20 | MGVKVLFALICIAVAEAAASRTLCGGELVDTLQFVCGDRGFLFSRP | 31 |
| (GaussiaAA- | ASRVSRRSRGIVEECCFRSCDLALLETYCATPARSEGGGGSGGGGSR | |
| vIGF2- | PRAVPTQDPPAPLPLVIWHGMGDSCCNPLSMGAIKKMVEKKIPGIY | |
| PPT1) | VLSLEIGKTLMEDVENSFFLNVNSQVTTVCQALAKDPKLQQGYNA | |
| | MGFSQGGQFLRAVAQRCPSPPMINLISVGGQHQGVFGLPRCPGESS | |
| | HICDFIRKTLNAGAYSKVVQERLVQAEYWHDPIKEDVYRNHSIFLA | |
| | DINQERGINESYKKNLMALKKFVMVKFLNDSIVDPVDSEWEGFYRS | |
| | GQAKETIPLQETSLYTQDRLGLKEMDNAGQLVFLATEGDHLQLSEE | |
| | WFYAHIIPFLG | |
| PPT1-21 | MLGLWGQRLPAAWVLLLLPFLPLLLLADPPAPLPLVIWHGMGDSC | 32 |
| (ppt2ss- | CNPLSMGAIKKMVEKKIPGIYVLSLEIGKTLMEDVENSFFLNVNSQV | |
| PPT1) | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLG | |
| PPT1-22 | MLGLWGQRLPAAWVLLLLPFLPLLLLASRALQHLDPPAPLPLVIWH | 33 |
| (ppt2ss- | GMGDSCCNPLSMGAIKKMVEKKIPGIYVLSLEIGKTLMEDVENSFFL | |
| PPT1_2) | NVNSQVTTVCQALAKDPKLQQGYNAMGFSQGGQFLRAVAQRCPSP | |
| | PMINLISVGGQHQGVFGLPRCPGESSHICDFIRKTLNAGAYSKVVQE | |
| | RLVQAEYWHDPIKEDVYRNHSIFLADINQERGINESYKKNLMALKK | |
| | FVMVKFLNDSIVDPVDSEWFGFYRSGQAKETIPLQETSLYTQDRLGL | |
| | KEMDNAGQLVFLATEGDHLQLSEEWFYAHIIPFLG | |
| PPT1-23 | MASPSCLWLLAVALLPWSCAARALGHLDPPAPLPLVIWHGMGDSC | 34 |
| (consensusSS- | CNPLSMGAIKKMVEKKIPGIYVLSLEIGKTLMEDVENSFFLNVNSQV | |
| PPT1) | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLG | |
| PPT1-24 | MASPSCLWLLAVALLPWSCAARALGHLDPPAPLPLVIWHGMGDSC | 35 |
| (consensus- | CNPLSMGAIKKMVEKKIPGIYVLSLEIGKTLMEDVENSFFLNVNSQV | |
| PPT1) | TTVCQILAKDPKLQQGYNAMGFSQGGQFLRAVAQRCPSPPMINLIS | |
| | VGGQHQGVFGLPRCPGESSHICDFIRKTLNAGAYSKAVQERLVQAE | |
| | YWHDPIKEDVYRNHSIFLADINQERGVNESYKKNLMALKKFVMVK | |
| | FLNDSIVDPVDSEWFGFYRSGQAKETIPLQETTLYTQDRLGLKEMD | |
| | KAGQLVFLATEGDHLQLSEEWFYAHIIPFLE | |
| PPT1-25 | MASPGCLWLLAVALLPWTCASRALQHLDPPAPLPLVIWHGMGDSC | 36 |
| (wt-PPT1 | CNPLSMGAIKKMVEKKIPGIYVLSLEIGKTLMEDVENSFFLNVNSQV | |
| L283C | TTVCQALAKDPKLQQGYNAMGFSQGGQFLRAVAQRCPSPPMINLIS | |
| H300C) | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFCATEGDHLQLSEEWFYACIIPFLG | |
| PPT1-26 | MASPGCLWLLAVALLPWTCASRALQHLDPPAPLPLVIWHGMGDSC | 37 |
| (wt-PPT1 | CNPLSMGAIKKMVEKKIPGIYVLSLEIGKTLMEDVENSFFLNVNSQV | |
| G113C | TTVCQALAKDPKLQQGYNAMCFSQGGQFCRAVAQRCPSPPMINLIS | |
| L121C) | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLG | |
| PPT1-27 | MASPGCLWLLAVALLPWTCASRALQHLDPPAPLPLVIWHGMGDSC | 38 |
| (wt-PPT1 | CNPLSMGAIKKMVEKKIPGIYVLSLEIGKTLMEDVENSFFLNVNSQV | |
| A171C | TTVCQALAKDPKLQQGYNAMGFSQGGQFLRAVAQRCPSPPMINLIS | |
| A183C) | VGGQHQGVFGLPRCPGESSHICDFIRKTLNAGCYSKVVQERLVQCE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLG | |
| PPT1-28 | MKLSLVAAMLLLLWVALLLLSAARAAASRALQHLDPPAPLPLVIW | 39 |
| (BiP2aa- | HGMGDSCCNPLSMGAIKKMVEKKIPGIYVLSLEIGKTLMEDVENSF | |
| PPT1) | FLNVNSQVTTVCQALAKDPKLQQGYNAMGFSQGGQFLRAVAQRC | |
| | PSPPMINLISVGGQHQGVFGLPRCPGESSHICDFIRKTLNAGAYSKVV | |
| | QERLVQAEYWHDPIKEDVYRNHSIFLADINQERGINESYKKNLMAL | |
| | KKFVMVKFLNDSIVDPVDSEWFGFYRSGQAKETIPLQETSLYTQDR | |
| | LGLKEMDNAGQLVFLATEGDHLQLSEEWFYAHIIPFLG | |
| PPT1-31 | MKLSLVAAMLLLLSLVAAMLLLLSAARASRTLCGGELVDTLQFVC | 40 |
| (BiP1- | GDRGFLFSRPASRVSRRSRGIVEECCFRSCDLALLETYCATPARSEG | |
| vIGF2-PPT1 | GGGSGGGGSRPRAVPTQDPPAPLPLVIWHGMGDSCCNPLSMGAIKK | |
| | MVEKKIPGIYVLSLEIGKTLMEDVENSFFLNVNSQVTTVCQALAKDP | |
| | KLQQGYNAMGFSQGGQFLRAVAQRCPSPPMINLISVGGQHQGVFG | |
| | LPRCPGESSHICDFIRKTLNAGAYSKVVQERLVQAEYWHDPIKEDV | |
| | YRNHSIFLADINQERGINESYKKNLMALKKFVMVKFLNDSIVDPVD | |
| | SEWFGFYRSGQAKETIPLQETSLYTQDRLGLKEMDNAGQLVFLATE | |
| | GDHLQLSEEWFYAHIIPFLG | |
| PPT1-32 | MASPGCLWLLAVALLPWTCASRALQHLDPPAPLPLVIWHGMGDSC | 41 |
| (wt-PPT1- | CNPLSMGAIKKMVEKKIPGIYVLSLEIGKTLMEDVENSFFLNVNSQV | |
| vIGF2-32) | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLGRPRAVPTQGGSGSGSTS | |
| | SSRTLCGGELVDTLQFVCGDRGFLFSRGGGGSRGILEECCFRECDLA | |
| | LLETYCATPARSE | |
| PPT1-33 | MASPGCLWLLAVALLPWTCASRALQHLDPPAPLPLVIWHGMGDSC | 42 |
| (wt-PPT1- | CNPLSMGAIKKMVEKKIPGIYVLSLEIGKTLMEDVENSFFLNVNSQV | |
| vIGF2-8Q) | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLGRPRAVPTQGGSGSGSTS | |
| | SSRTLCGGELVDTLQFVCGDRGFLFSRPASRVSRRSRGIVEECCFRE | |
| | CDLALLETYCATPARSE | |
| PPT1-34 | MASPGCLWLLAVALLPWTCASRALQHLDPPAPLPLVIWHGMGDSC | 43 |
| (wt-PPT1- | CNPLSMGAIKKMVEKKIPGIYVLSLEIGKTLMEDVENSFFLNVNSQV | |
| vIGF2-8Q) | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLGRPRAVPTQGGSGSGSTS | |
| | SSRTLCGGELVDTLQFVCGDRGFLFSRPASRVSRRSRGIVEECCFRE | |
| | CDLALLETYCATPARSE | |
| PPT1-35 | MASPGCLWLLAVALLPWTCASRALQHLDPPAPLPLVIWHGMGDSC | 44 |
| (wt-PPT1- | CNPLSMGAIKKMVEKKIPGIYVLSLEIGKTLMEDVENSFFLNVNSQV | |
| vIGF2-8Q) | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLGRPRAVPTQGGSGSGSTS | |
| | SSRTLCGGELVDTLQFVCGDRGFLFSRPASRVSRRSRGIVEECCFRE | |
| | CDLALLETYCATPARSE | |
| Human | SYSPEPDQRRTLPPGWVSLGRADPEEELSLTFALRQQNVERLSELVQ | 45 |
| TPP1 | AVSDPSSPQYGKYLTLENVADLVRPSPLTLHTVQKWLLAAGAQKC | |
| Propeptide | HSVITQDFLTCWLSIRQAELLLPGAEFHHYVGGPTETHVVRSPHPYQ | |
| | LPQALAPHVDFVGGLHRFPPTSSLRQRPEPQVTGTVG | |
| Human | LHLGVTPSVIRKRYNLTSQDVGSGTSNNSQACAQFLEQYFHDSDLA | 46 |
| TPP1 | QFMRLFGGNFAHQASVARVVGQQGRGRAGIEASLDVQYLMSAGA | |
| Mature | NISTWVYSSPGRHEGQEPFLQWLMLLSNESALPHVHTVSYGDDEDS | |
| Peptide | LSSAYIQRVNTELMKAAARGLTLLFASGDSGAGCWSVSGRHQFRPT | |
| | FPASSPYVTTVGGTSFQEPFLITNEIVDYISGGGFSNVFPRPSYQEEAV | |
| | TKFLSSSPHLPPSSYFNASGRAYPDVAALSDGYWVVSNRVPIPWVS | |
| | GTSASTPVFGGILSLINEHRILSGRPPLGFLNPRLYQQHGAGLFDVTR | |
| | GCHESCLDEEVEGQGFCSGPGWDPVTGWGTPNFPALLKTLLNP | |
| pSvelte001- | MGLQACLLGLFALILSGKCSRTLCGGELVDTLQFVCGDRGFLFSRPA | 47 |
| Native TPP1 | SRVSRRSRGIVEECCFRSCDLALLETYCATPARSEGGGGSGGGGSRP | |
| Signal | RAVPTQSYSPEPDQRRTLPPGWVSLGRADPEEELSLTFALRQQNVER | |
| Peptide- | LSELVQAVSDPSSPQYGKYLTLENVADLVRPSPLTLHTVQKWLLAA | |
| vIGF2-GS | GAQKCHSVITQDFLTCWLSIRQAELLLPGAEFHHYVGGPTETHVVR | |
| linker- | SPHPYQLPQALAPHVDFVGGLHRFPPTSSLRQRPEPQVTGTVGLHLG | |
| Lyso Cleave- | VTPSVIRKRYNLTSQDVGSGTSNNSQACAQFLEQYFHDSDLAQFMR | |
| TPP1 | LFGGNFAHQASVARVVGQQGRGRAGIEASLDVQYLMSAGANISTW | |
| propeptide- | VYSSPGRHEGQEPFLQWLMLLSNESALPHVHTVSYGDDEDSLSSAY | |
| TPP1 | IQRVNTELMKAAARGLTLLFASGDSGAGCWSVSGRHQFRPTFPASS | |
| mature | PYVTTVGGTSFQEPFLITNEIVDYISGGGFSNVFPRPSYQEEAVTKFL | |
| peptide | SSSPHLPPSSYFNASGRAYPDVAALSDGYWVVSNRVPIPWVSGTSAS | |
| | TPVFGGILSLINEHRILSGRPPLGFLNPRLYQQHGAGLFDVTRGCHES | |
| | CLDEEVEGQGFCSGPGWDPVTGWGTPNFPALLKTLLNPG | |
| Svelte057- | MGLQACLLGLFALILSGKCSRTLCGGELVDTLQFVCGDRGFLFSRPA | 48 |
| Native TPP1 | SRVSRRSRGIVEECCFRECDLALLETYCATPARSEGGGGSGGGGSRP | |
| Signal | RAVPTQASYSPEPDQRRTLPPGWVSLGRADPEEELSLTFALRQQNV | |
| Peptide- | ERLSELVQAVSDPSSPQYGKYLTLENVADLVRPSPLTLHTVQKWLL | |
| vIGF2v17 | AAGAQKCHSVITQDFLTCWLSIRQAELLLPGAEFHHYVGGPTETHV | |
| GS linker- | VRSPHPYQLPQALAPHVDFVGGLHRFPPTSSLRQRPEPQVTGTVGLH | |
| Lyso Cleave- | LGVTPSVIRKRYNLTSQDVGSGTSNNSQACAQFLEQYFHDSDLAQF | |
| TPP1 | MRLFGGNFAHQASVARVVGQQGRGRAGIEASLDVQYLMSAGANIS | |
| propeptide- | TWVYSSPGRHEGQEPFLQWLMLLSNESALPHVHTVSYGDDEDSLSS | |
| TPP1 | AYIQRVNTELMKAAARGLTLLFASGDSGAGCWSVSGRHQFRPTFPA | |
| mature | SSPYVTTVGGTSFQEPFLITNEIVDYISGGGFSNVFPRPSYQEEAVTKF | |
| peptide | LSSSPHLPPSSYFNASGRAYPDVAALSDGYWVVSNRVPIPWVSGTS | |
| | ASTPVFGGILSLINEHRILSGRPPLGFLNPRLYQQHGAGLFDVTRGCH | |
| | ESCLDEEVEGQGFCSGPGWDPVTGWGTPNFPALLKTLLNPG | |
| pSvelte059- | MGLQACLLGLFALILSGKCSRTLCGGELVDTLQFVCGDRGFLFSRG | 49 |
| Native | GGGSRGIVEECCFRSCDLALLETYCATPARSEGGGGSGGGGSRPRA | |
| TPP1 Signal | VPTQASYSPEPDQRRTLPPGWVSLGRADPEEELSLTFALRQQNVERL | |
| Peptide- | SELVQAVSDPSSPQYGKYLTLENVADLVRPSPLTLHTVQKWLLAAG | |
| vIGF2v22- | AQKCHSVITQDFLTCWLSIRQAELLLPGAEFHHYVGGPTETHVVRSP | |
| GS linker- | HPYQLPQALAPHVDFVGGLHRFPPTSSLRQRPEPQVTGTVGLHLGV | |
| Lyso Cleave- | TPSVIRKRYNLTSQDVGSGTSNNSQACAQFLEQYFHDSDLAQFMRL | |
| TPP1 | FGGNFAHQASVARVVGQQGRGRAGIEASLDVQYLMSAGANISTWV | |
| propeptide- | YSSPGRHEGQEPFLQWLMLLSNESALPHVHTVSYGDDEDSLSSAYI | |
| TPP1 | QRVNTELMKAAARGLTLLFASGDSGAGCWSVSGRHQFRPTFPASSP | |
| mature | YVTTVGGTSFQEPFLITNEIVDYISGGGFSNVFPRPSYQEEAVTKFLS | |
| peptide | SSPHLPPSSYFNASGRAYPDVAALSDGYWVVSNRVPIPWVSGTSAS | |
| | TPVFGGILSLINEHRILSGRPPLGFLNPRLYQQHGAGLFDVTRGCHES | |
| | CLDEEVEGQGFCSGPGWDPVTGWGTPNFPALLKTLLNPG | |
| pSvelte060- | MGLQACLLGLFALILSGKCSRTLCGGELVDTLQFVCGDRGFLFSRG | 50 |
| Native | GGGSRGILEECCFRSCDLALLETYCATPARSEGGGGSGGGGSRPRA | |
| TPP1 Signal | VPTQASYSPEPDQRRTLPPGWVSLGRADPEEELSLTFALRQQNVERL | |
| Peptide- | SELVQAVSDPSSPQYGKYLTLENVADLVRPSPLTLHTVQKWLLAAG | |
| vIGF2v24- | AQKCHSVITQDFLTCWLSIRQAELLLPGAEFHHYVGGPTETHVVRSP | |
| GS linker- | HPYQLPQALAPHVDFVGGLHRFPPTSSLRQRPEPQVTGTVGLHLGV | |
| Lyso Cleave- | TPSVIRKRYNLTSQDVGSGTSNNSQACAQFLEQYFHDSDLAQFMRL | |
| TPP1 | FGGNFAHQASVARVVGQQGRGRAGIEASLDVQYLMSAGANISTWV | |
| propeptide- | YSSPGRHEGQEPFLQWLMLLSNESALPHVHTVSYGDDEDSLSSAYI | |
| TPP1 | QRVNTELMKAAARGLTLLFASGDSGAGCWSVSGRHQFRPTFPASSP | |
| mature | YVTTVGGTSFQEPFLITNEIVDYISGGGFSNVFPRPSYQEEAVTKFLS | |
| peptide | SSPHLPPSSYFNASGRAYPDVAALSDGYWVVSNRVPIPWVSGTSAS | |
| | TPVFGGILSLINEHRILSGRPPLGFLNPRLYQQHGAGLFDVTRGCHES | |
| | CLDEEVEGQGFCSGPGWDPVTGWGTPNFPALLKTLLNPG | |
| pSvelte061- | MGLQACLLGLFALILSGKCSRTLCGGELVDVLQFVCGRRGFLFSRP | 51 |
| Native TPP1 | ASRVSRRSRGIVEECCFRDCDLALLETYCATPARSEGGGGSGGGGS | |
| Signal | RPRAVPTQASYSPEPDQRRTLPPGWVSLGRADPEEELSLTFALRQQN | |
| Peptide- | VERLSELVQAVSDPSSPQYGKYLTLENVADLVRPSPLTLHTVQKWL | |
| vIGF2v30- | LAAGAQKCHSVITQDFLTCWLSIRQAELLLPGAEFHHYVGGPTETH | |
| GS linker- | VVRSPHPYQLPQALAPHVDFVGGLHRFPPTSSLRQRPEPQVTGTVGL | |
| Lyso Cleave- | HLGVTPSVIRKRYNLTSQDVGSGTSNNSQACAQFLEQYFHDSDLAQ | |
| TPP1 | FMRLFGGNFAHQASVARVVGQQGRGRAGIEASLDVQYLMSAGANI | |
| propeptide- | STWVYSSPGRHEGQEPFLQWLMLLSNESALPHVHTVSYGDDEDSLS | |
| TPP1 | SAYIQRVNTELMKAAARGLTLLFASGDSGAGCWSVSGRHQFRPTFP | |
| mature | ASSPYVTTVGGTSFQEPFLITNEIVDYISGGGFSNVFPRPSYQEEAVT | |
| peptide | KFLSSSPHLPPSSYFNASGRAYPDVAALSDGYWVVSNRVPIPWVSG | |
| | TSASTPVFGGILSLINEHRILSGRPPLGFLNPRLYQQHGAGLFDVTRG | |
| | CHESCLDEEVEGQGFCSGPGWDPVTGWGTPNFPALLKTLLNPG* | |
| pSvelte062- | MGLQACLLGLFALILSGKCSRTLCGGELVDTLQFVCGDRGFLFSRG | 52 |
| Native TPP1 | GGGSRGILEECCFRDCDLALLETYCATPARSEGGGGSGGGGSRPRA | |
| Signal | VPTQASYSPEPDQRRTLPPGWVSLGRADPEEELSLTFALRQQNVERL | |
| Peptide | SELVQAVSDPSSPQYGKYLTLENVADLVRPSPLTLHTVQKWLLAAG | |
| vIGF2v31- | AQKCHSVITQDFLTCWLSIRQAELLLPGAEFHHYVGGPTETHVVRSP | |
| GS linker- | HPYQLPQALAPHVDFVGGLHRFPPTSSLRQRPEPQVTGTVGLHLGV | |
| Lyso Cleave- | TPSVIRKRYNLTSQDVGSGTSNNSQACAQFLEQYFHDSDLAQFMRL | |
| TPP1 | FGGNFAHQASVARVVGQQGRGRAGIEASLDVQYLMSAGANISTWV | |
| propeptide- | YSSPGRHEGQEPFLQWLMLLSNESALPHVHTVSYGDDEDSLSSAYI | |
| TPP1 | QRVNTELMKAAARGLTLLFASGDSGAGCWSVSGRHQFRPTFPASSP | |
| mature | YVTTVGGTSFQEPFLITNEIVDYISGGGFSNVFPRPSYQEEAVTKFLS | |
| peptide | SSPHLPPSSYFNASGRAYPDVAALSDGYWVVSNRVPIPWVSGTSAS | |
| | TPVFGGILSLINEHRILSGRPPLGFLNPRLYQQHGAGLFDVTRGCHES | |
| | CLDEEVEGQGFCSGPGWDPVTGWGTPNFPALLKTLLNPG | |
| pSvelte063- | MGLQACLLGLFALILSGKCSRTLCGGELVDTLQFVCGDRGFLFSRG | 53 |
| Native TPP1 | GGGSRGILEECCFRECDLALLETYCATPARSEGGGGSGGGGSRPRA | |
| Signal | VPTQASYSPEPDQRRTLPPGWVSLGRADPEEELSLTFALRQQNVERL | |
| Peptide- | SELVQAVSDPSSPQYGKYLTLENVADLVRPSPLTLHTVQKWLLAAG | |
| vIGF2v32- | AQKCHSVITQDFLTCWLSIRQAELLLPGAEFHHYVGGPTETHVVRSP | |
| GS linker- | HPYQLPQALAPHVDFVGGLHRFPPTSSLRQRPEPQVTGTVGLHLGV | |
| Lyso Cleave- | TPSVIRKRYNLTSQDVGSGTSNNSQACAQFLEQYFHDSDLAQFMRL | |
| TPP1 | FGGNFAHQASVARVVGQQGRGRAGIEASLDVQYLMSAGANISTWV | |
| propeptide- | YSSPGRHEGQEPFLQWLMLLSNESALPHVHTVSYGDDEDSLSSAYI | |
| TPP1 | QRVNTELMKAAARGLTLLFASGDSGAGCWSVSGRHQFRPTFPASSP | |
| mature | YVTTVGGTSFQEPFLITNEIVDYISGGGFSNVFPRPSYQEEAVTKFLS | |
| peptide | SSPHLPPSSYFNASGRAYPDVAALSDGYWVVSNRVPIPWVSGTSAS | |
| | TPVFGGILSLINEHRILSGRPPLGFLNPRLYQQHGAGLFDVTRGCHES | |
| | CLDEEVEGQGFCSGPGWDPVTGWGTPNFPALLKTLLNPG | |
| Wt- | MEAVAVAAAVGVLLLAGAGGAAGDEAREAAAVRALVARLLGPGP | 54 |
| NAGLU | AADFSVSVERALAAKPGLDTYSLGGGGAARVRVRGSTGVAAAAGL | |
| | HRYLRDFCGCHVAWSGSQLRLPRPLPAVPGELTEATPNRYRYYQN | |
| | VCTQSYSFVWWDWARWEREIDWMALNGINLALAWSGQEAIWQR | |
| | VYLALGLTQAEINEFFTGPAFLAWGRMGNLHTWDGPLPPSWHIKQL | |
| | YLQHRVLDQMRSFGMTPVLPAFAGHVPEAVTRVFPQVNVTKMGS | |
| | WGHFNCSYSCSFLLAPEDPIFPIIGSLFLRELIKEFGTDHIYGADTFNE | |
| | MQPPSSEPSYLAAATTAVYEAMTAVDTEAVWLLQGWLFQHQPQF | |
| | WGPAQIRAVLGAVPRGRLLVLDLFAESQPVYTRTASFQGQPFIWCM | |
| | LHNFGGNHGLFGALEAVNGGPEAARLFPNSTMVGTGMAPEGISQN | |
| | EVVYSLMAELGWRKDPVPDLAAWVTSFAARRYGVSHPDAGAAWR | |
| | LLLRSVYNCSGEACRGHNRSPLVRRPSLQMNTSIWYNRSDVFEAWR | |
| | LLLTSAPSLATSPAFRYDLLDLTRQAVQELVSLYYEEARSAYLSKEL | |
| | ASLLRAGGVLAYELLPALDEVLASDSRFLLGSWLEQARAAAVSEAE | |
| | ADFYEQNSRYQLTLWGPEGNILDYANKQLAGLVANYYTPRWRLFL | |
| | EALVDSVAQGIPFQQHQFDKNVFQLEQAFVLSKQRYPSQPRGDTVD | |
| | LAKKIFLKYYPRWVAGSW | |
| Wt | MEAVAVAAAVGVLLLAGAGGAAGDEAREAAAVRALVARLLGPGP | 55 |
| NAGLU- | AADFSVSVERALAAKPGLDTYSLGGGGAARVRVRGSTGVAAAAGL | |
| HPC4 | HRYLRDFCGCHVAWSGSQLRLPRPLPAVPGELTEATPNRYRYYQN | |
| | VCTQSYSFVWWDWARWEREIDWMALNGINLALAWSGQEAIWQR | |
| | VYLALGLTQAEINEFFTGPAFLAWGRMGNLHTWDGPLPPSWHIKQL | |
| | YLQHRVLDQMRSFGMTPVLPAFAGHVPEAVTRVFPQVNVTKMGS | |
| | WGHFNCSYSCSFLLAPEDPIFPIIGSLFLRELIKEFGTDHIYGADTENE | |
| | MQPPSSEPSYLAAATTAVYEAMTAVDTEAVWLLQGWLFQHQPQF | |
| | WGPAQIRAVLGAVPRGRLLVLDLFAESQPVYTRTASFQGQPFIWCM | |
| | LHNFGGNHGLFGALEAVNGGPEAARLFPNSTMVGTGMAPEGISQN | |
| | EVVYSLMAELGWRKDPVPDLAAWVTSFAARRYGVSHPDAGAAWR | |
| | LLLRSVYNCSGEACRGHNRSPLVRRPSLQMNTSIWYNRSDVFEAWR | |
| | LLLTSAPSLATSPAFRYDLLDLTRQAVQELVSLYYEEARSAYLSKEL | |
| | ASLLRAGGVLAYELLPALDEVLASDSRFLLGSWLEQARAAAVSEAE | |
| | ADFYEQNSRYQLTLWGPEGNILDYANKQLAGLVANYYTPRWRLFL | |
| | EALVDSVAQGIPFQQHQFDKNVFQLEQAFVLSKQRYPSQPRGDTVD | |
| | LAKKIFLKYYPRWVAGSWGLEVLFQGPEDQVDPRLIDGK | |
| vIGF2- | MEAVAVAAAVGVLLLAGAGGAAGDASRTLCGGELVDTLQFVCGD | 56 |
| NAGLU- | RGFLFSRPASRVSRRSRGIVEECCFRSCDLALLETYCATPARSEGGG | |
| HPC4 | GSGGGGSRPRAVPTQAEAREAAAVRALVARLLGPGPAADFSVSVE | |
| | RALAAKPGLDTYSLGGGGAARVRVRGSTGVAAAAGLHRYLRDFC | |
| | GCHVAWSGSQLRLPRPLPAVPGELTEATPNRYRYYQNVCTQSYSFV | |
| | WWDWARWEREIDWMALNGINLALAWSGQEAIWQRVYLALGLTQ | |
| | AEINEFFTGPAFLAWGRMGNLHTWDGPLPPSWHIKQLYLQHRVLD | |
| | QMRSFGMTPVLPAFAGHVPEAVTRVFPQVNVTKMGSWGHFNCSYS | |
| | CSFLLAPEDPIFPIIGSLFLRELIKEFGTDHIYGADTFNEMQPPSSEPSY | |
| | LAAATTAVYEAMTAVDTEAVWLLQGWLFQHQPQFWGPAQIRAVL | |
| | GAVPRGRLLVLDLFAESQPVYTRTASFQGQPFIWCMLHNFGGNHGL | |
| | FGALEAVNGGPEAARLFPNSTMVGTGMAPEGISQNEVVYSLMAEL | |
| | GWRKDPVPDLAAWVTSFAARRYGVSHPDAGAAWRLLLRSVYNCS | |
| | GEACRGHNRSPLVRRPSLQMNTSIWYNRSDVFEAWRLLLTSAPSLA | |
| | TSPAFRYDLLDLTRQAVQELVSLYYEEARSAYLSKELASLLRAGGV | |
| | LAYELLPALDEVLASDSRFLLGSWLEQARAAAVSEAEADFYEQNSR | |
| | YQLTLWGPEGNILDYANKQLAGLVANYYTPRWRLFLEALVDSVAQ | |
| | GIPFQQHQFDKNVFQLEQAFVLSKQRYPSQPRGDTVDLAKKIFLKY | |
| | YPRWVAGSWGLEVLFQGPEDQVDPRLIDGK | |
| vIGF2-17- | MEAVAVAAAVGVLLLAGAGGAAGDASRTLCGGELVDTLQFVCGD | 57 |
| NAGLU | RGFLFSRPASRVSRRSRGIVEECCFRECDLALLETYCATPARSEGGG | |
| | GSGGGGSRPRAVPTQAEAREAAAVRALVARLLGPGPAADFSVSVE | |
| | RALAAKPGLDTYSLGGGGAARVRVRGSTGVAAAAGLHRYLRDFC | |
| | GCHVAWSGSQLRLPRPLPAVPGELTEATPNRYRYYQNVCTQSYSFV | |
| | WWDWARWEREIDWMALNGINLALAWSGQEAIWQRVYLALGLTQ | |
| | AEINEFFTGPAFLAWGRMGNLHTWDGPLPPSWHIKQLYLQHRVLD | |
| | QMRSFGMTPVLPAFAGHVPEAVTRVFPQVNVTKMGSWGHFNCSYS | |
| | CSFLLAPEDPIFPIIGSLFLRELIKEFGTDHIYGADTFNEMQPPSSEPSY | |
| | LAAATTAVYEAMTAVDTEAVWLLQGWLFQHQPQFWGPAQIRAVL | |
| | GAVPRGRLLVLDLFAESQPVYTRTASFQGQPFIWCMLHNFGGNHGL | |
| | FGALEAVNGGPEAARLFPNSTMVGTGMAPEGISQNEVVYSLMAEL | |
| | GWRKDPVPDLAAWVTSFAARRYGVSHPDAGAAWRLLLRSVYNCS | |
| | GEACRGHNRSPLVRRPSLQMNTSIWYNRSDVFEAWRLLLTSAPSLA | |
| | TSPAFRYDLLDLTRQAVQELVSLYYEEARSAYLSKELASLLRAGGV | |
| | LAYELLPALDEVLASDSRFLLGSWLEQARAAAVSEAEADFYEQNSR | |
| | YQLTLWGPEGNILDYANKQLAGLVANYYTPRWRLFLEALVDSVAQ | |
| | GIPFQQHQFDKNVFQLEQAFVLSKQRYPSQPRGDTVDLAKKIFLKY | |
| | YPRWVAGSWGLEVLFQGPEDQVDPRLIDGK | |
| vIGF2-31- | MEAVAVAAAVGVLLLAGAGGAAGDASRTLCGGELVDTLQFVCGD | 58 |
| NAGLU- | RGFLFSRGGGGSRGILEECCFRDCDLALLETYCATPARSEGGGGSGG | |
| HPC4 | GGSRPRAVPTQAEAREAAAVRALVARLLGPGPAADFSVSVERALA | |
| | AKPGLDTYSLGGGGAARVRVRGSTGVAAAAGLHRYLRDFCGCHV | |
| | AWSGSQLRLPRPLPAVPGELTEATPNRYRYYQNVCTQSYSFVWWD | |
| | WARWEREIDWMALNGINLALAWSGQEAIWQRVYLALGLTQAEINE | |
| | FFTGPAFLAWGRMGNLHTWDGPLPPSWHIKQLYLQHRVLDQMRSF | |
| | GMTPVLPAFAGHVPEAVTRVFPQVNVTKMGSWGHFNCSYSCSFLL | |
| | APEDPIFPIIGSLFLRELIKEFGTDHIYGADTFNEMQPPSSEPSYLAAA | |
| | TTAVYEAMTAVDTEAVWLLQGWLFQHQPQFWGPAQIRAVLGAVP | |
| | RGRLLVLDLFAESQPVYTRTASFQGQPFIWCMLHNFGGNHGLFGAL | |
| | EAVNGGPEAARLFPNSTMVGTGMAPEGISQNEVVYSLMAELGWRK | |
| | DPVPDLAAWVTSFAARRYGVSHPDAGAAWRLLLRSVYNCSGEACR | |
| | GHNRSPLVRRPSLQMNTSIWYNRSDVFEAWRLLLTSAPSLATSPAFR | |
| | YDLLDLTRQAVQELVSLYYEEARSAYLSKELASLLRAGGVLAYELL | |
| | PALDEVLASDSRFLLGSWLEQARAAAVSEAEADFYEQNSRYQLTL | |
| | WGPEGNILDYANKQLAGLVANYYTPRWRLFLEALVDSVAQGIPFQ | |
| | QHQFDKNVFQLEQAFVLSKQRYPSQPRGDTVDLAKKIFLKYYPRW | |
| | VAGSWGLEVLFQGPEDQVDPRLIDGK | |
| vIGF2-32- | MEAVAVAAAVGVLLLAGAGGAAGDASRTLCGGELVDTLQFVCGD | 59 |
| NAGLU | RGFLFSRGGGGSRGILEECCFRECDLALLETYCATPARSEGGGGSGG | |
| | GGSRPRAVPTQAEAREAAAVRALVARLLGPGPAADFSVSVERALA | |
| | AKPGLDTYSLGGGGAARVRVRGSTGVAAAAGLHRYLRDFCGCHV | |
| | AWSGSQLRLPRPLPAVPGELTEATPNRYRYYQNVCTQSYSFVWWD | |
| | WARWEREIDWMALNGINLALAWSGQEAIWQRVYLALGLTQAEINE | |
| | FFTGPAFLAWGRMGNLHTWDGPLPPSWHIKQLYLQHRVLDQMRSF | |
| | GMTPVLPAFAGHVPEAVTRVFPQVNVTKMGSWGHFNCSYSCSFLL | |
| | APEDPIFPIIGSLFLRELIKEFGTDHIYGADTFNEMQPPSSEPSYLAAA | |
| | TTAVYEAMTAVDTEAVWLLQGWLFQHQPQFWGPAQIRAVLGAVP | |
| | RGRLLVLDLFAESQPVYTRTASFQGQPFIWCMLHNFGGNHGLFGAL | |
| | EAVNGGPEAARLFPNSTMVGTGMAPEGISQNEVVYSLMAELGWRK | |
| | DPVPDLAAWVTSFAARRYGVSHPDAGAAWRLLLRSVYNCSGEACR | |
| | GHNRSPLVRRPSLQMNTSIWYNRSDVFEAWRLLLTSAPSLATSPAFR | |
| | YDLLDLTRQAVQELVSLYYEEARSAYLSKELASLLRAGGVLAYELL | |
| | PALDEVLASDSRFLLGSWLEQARAAAVSEAEADFYEQNSRYQLTL | |
| | WGPEGNILDYANKQLAGLVANYYTPRWRLFLEALVDSVAQGIPFQ | |
| | QHQFDKNVFQLEQAFVLSKQRYPSQPRGDTVDLAKKIFLKYYPRW | |
| | VAGSWGLEVLFQGPEDQVDPRLIDGK | |
| PPT1-101 | MKLSLVAAMLLLLWVALLLLSAARAAASRTLCGGELVDTLQFVCG | 60 |
| | DRGFLFSRGGGGSRGILEECCFRDCDLALLETYCATPARSEGGGGSG | |
| | GGGSRPRAVPTQDPPAPLPLVIWHGMGDSCCNPLSMGAIKKMVEK | |
| | KIPGIYVLSLEIGKTLMEDVENSFFLNVNSQVTTVCQALAKDPKLQQ | |
| | GYNAMGFSQGGQFLRAVAQRCPSPPMINLISVGGQHQGVFGLPRCP | |
| | GESSHICDFIRKTLNAGAYSKVVQERLVQAEYWHDPIKEDVYRNHS | |
| | IFLADINQERGINESYKKNLMALKKFVMVKFLNDSIVDPVDSEWFG | |
| | FYRSGQAKETIPLQETSLYTQDRLGLKEMDNAGQLVFLATEGDHLQ | |
| | LSEEWFYAHIIPFLG | |
| PPT1-104 | MASPGSLWLLAVALLPWTCASRALQHLDPPAPLPLVIWHGMGDSC | 61 |
| | CNPLSMGAIKKMVEKKIPGIYVLSLEIGKTLMEDVENSFFLNVNSQV | |
| | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLGRPRAVPTQGGSGSGSTS | |
| | SSRTLCGGELVDTLQFVCGDRGFLFSRGGGGSRGILEECCFRECDLA | |
| | LLETYCATPARSE | |
| PPT1-112 | MASPGSLWLLAVALLPWTCASRALQHLAASRTLCGGELVDTLQFV | 62 |
| | CGDRGFLFSRGGGGSRGILEECCFRDCDLALLETYCATPARSEGGG | |
| | GSGGGGSRPRAVPTQDPPAPLPLVIWHGMGDSCCNPLSMGAIKKM | |
| | VEKKIPGIYVLSLEIGKTLMEDVENSFFLNVNSQVTTVCQALAKDPK | |
| | LQQGYNAMGFSQGGQFLRAVAQRCPSPPMINLISVGGQHQGVFGLP | |
| | RCPGESSHICDFIRKTLNAGAYSKVVQERLVQAEYWHDPIKEDVYR | |
| | NHSIFLADINQERGINESYKKNLMALKKFVMVKFLNDSIVDPVDSE | |
| | WFGFYRSGQAKETIPLQETSLYTQDRLGLKEMDNAGQLVFLATEGD | |
| | HLQLSEEWFYAHIIPFLG | |
| PPT1-114 | MASPGSLWLLAVALLPWTCASRALQHLAASRTLCGGELVDTLQFV | 63 |
| | CGDRGFLFSRGGGGSRGILEECCFRECDLALLETYCATPARSEGGGG | |
| | SGGGGSRPRAVPTQDPPAPLPLVIWHGMGDSCCNPLSMGAIKKMVE | |
| | KKIPGIYVLSLEIGKTLMEDVENSFFLNVNSQVTTVCQALAKDPKLQ | |
| | QGYNAMGFSQGGQFLRAVAQRCPSPPMINLISVGGQHQGVFGLPRC | |
| | PGESSHICDFIRKTLNAGAYSKVVQERLVQAEYWHDPIKEDVYRNH | |
| | SIFLADINQERGINESYKKNLMALKKFVMVKFLNDSIVDPVDSEWF | |
| | GFYRSGQAKETIPLQETSLYTQDRLGLKEMDNAGQLVFLATEGDHL | |
| | QLSEEWFYAHIIPFLG | |
| PPT1-115 | MASPGSLWLLAVALLPWTCASRALQHLAADPPAPLPLVIWHGMGD | 64 |
| | SCCNPLSMGAIKKMVEKKIPGIYVLSLEIGKTLMEDVENSFFLNVNS | |
| | QVTTVCQALAKDPKLQQGYNAMGFSQGGQFLRAVAQRCPSPPMIN | |
| | LISVGGQHQGVFGLPRCPGESSHICDFIRKTLNAGAYSKVVQERLVQ | |
| | AEYWHDPIKEDVYRNHSIFLADINQERGINESYKKNLMALKKFVMV | |
| | KFLNDSIVDPVDSEWFGFYRSGQAKETIPLQETSLYTQDRLGLKEM | |
| | DNAGQLVFLATEGDHLQLSEEWFYAHIIPFLGRPRAVPTQGGSGSGS | |
| | TSSSRTLCGGELVDTLQFVCGDRGFLFSRGGGGSRGILEECCFRECD | |
| | LALLETYCATPARSE | |
| PPT1-116 | MASPGSLWLLAVALLPWTCASRALQHLAADPPAPLPLVIWHGMGD | 65 |
| | SCCNPLSMGAIKKMVEKKIPGIYVLSLEIGKTLMEDVENSFFLNVNS | |
| | QVTTVCQALAKDPKLQQGYNAMGFSQGGQFLRAVAQRCPSPPMIN | |
| | LISVGGQHQGVFGLPRCPGESSHICDFIRKTLNAGAYSKVVQERLVQ | |
| | AEYWHDPIKEDVYRNHSIFLADINQERGINESYKKNLMALKKFVMV | |
| | KFLNDSIVDPVDSEWFGFYRSGQAKETIPLQETSLYTQDRLGLKEM | |
| | DNAGQLVFLATEGDHLQLSEEWFYAHIIPFLGRPRAVPTQGGGGSG | |
| | SGGGGSSRTLCGGELVDTLQFVCGDRGFLFSRGGGGSRGILEECCFR | |
| | ECDLALLETYCATPARSE | |
| PPT1-117 | MASPGSLWLLAVALLPWTCASRALQHLDPPAPLPLVIWHGMGDSC | 66 |
| | CNPLSMGAIKKMVEKKIPGIYVLSLEIGKTLMEDVENSFFLNVNSQV | |
| | TTVCQALAKDPKLQQGYNAMGFSQGGQFLRAVAQRCPSPPMINLIS | |
| | VGGQHQGVFGLPRCPGESSHICDFIRKTLNAGAYSKVVQERLVQAE | |
| | YWHDPIKEDVYRNHSIFLADINQERGINESYKKNLMALKKFVMVKF | |
| | LNDSIVDPVDSEWFGFYRSGQAKETIPLQETSLYTQDRLGLKEMDN | |
| | AGQLVFLATEGDHLQLSEEWFYAHIIPFLGRPRAVPTQGGGGSGSG | |
| | GGGSSRTLCGGELVDTLQFVCGDRGFLFSRGGGGSRGILEECCFREC | |
| | DLALLETYCATPARSE | |
| PPT1-118 | MASPGSLWLLAVALLPWTCASRALQHLAADPPAPLPLVIWHGMGD | 67 |
| | SCCNPLSMGAIKKMVEKKIPGIYVLSLEIGKTLMEDVENSFFLNVNS | |
| | QVTTVCQALAKDPKLQQGYNAMGFSQGGQFLRAVAQRCPSPPMIN | |
| | LISVGGQHQGVFGLPRCPGESSHICDFIRKTLNAGAYSKVVQERLVQ | |
| | AEYWHDPIKEDVYRNHSIFLADINQERGINESYKKNLMALKKFVMV | |
| | KFLNDSIVDPVDSEWFGFYRSGQAKETIPLQETSLYTQDRLGLKEM | |
| | DNAGQLVFLATEGDHLQLSEEWFYAHIIPFLGRPRAVPTQGGGGSG | |
| | GGGSSRTLCGGELVDTLQFVCGDRGFLFSRGGGGSRGILEECCFREC | |
| | DLALLETYCATPARSE | |

Components of fusion proteins provided herein are further described below.

Peptides that Bind CI-MPR (e.g., vIGF2 Peptides)

Provided herein are peptides that bind CI-MPR. Fusion proteins comprising such peptides and a therapeutic protein, when expressed from a gene therapy vector, target the therapeutic protein to the cells where it is needed, increase cellular uptake by such cells and target the therapeutic protein to a subcellular location (e.g., a lysosome). In some embodiments, the peptide is fused to the N-terminus of the therapeutic peptide. In some embodiments, the peptide is fused to the C-terminus of the therapeutic protein. In some embodiments, the peptide is a vIGF2 peptide. Some vIGF2 peptides maintain high affinity binding to CI-MPR while their affinity for IGF1 receptor, insulin receptor, and IGF binding proteins (IGFBP) is decreased or eliminated. Some vIGF2 peptides increase affinity of binding to CI-MPR. Thus, some variant IGF2 peptides are substantially more selective and have reduced safety risks compared to wt IGF2. vIGF2 peptides herein include those having the amino acid sequence of SEQ ID NO: 31, 120 and 121. Variant IGF2 peptides further include those with variant amino acids at positions 6, 26, 27, 31-38, 43, 48, 49, 50, 54, 55, or 65 compared to wt IGF2 (SEQ ID NO: 68). In some embodiments, the vIGF2 peptide has a sequence having one or more substitutions from the group consisting of E6R, F26S, Y27L, V43L, F48T, R49S, S50E, S50I, A54R, L55R, and K65R. In some embodiments, the vIGF2 peptide has a sequence having a substitution of E6R. In some embodiments, the vIGF2 peptide has a sequence having a substitution of F26S. In some embodiments, the vIGF2 peptide has a sequence having a substitution of Y27L. In some embodiments, the vIGF2 peptide has a sequence having a substitution of V43L. In some embodiments, the vIGF2 peptide has a sequence having a substitution of F48T. In some embodiments, the vIGF2 peptide has a sequence having a substitution of R49S. In some embodiments, the vIGF2 peptide has a sequence having a substitution of S50I. In some embodiments, the vIGF2 peptide has a sequence having a substitution of S50E. In some embodiments, the vIGF2 peptide having a sequence having a substitution of 550E has increased binding to the CI-MPR. In some embodiments, the vIGF2 peptide has a sequence having a substitution of A54R. In some embodiments, the vIGF2 peptide has a sequence having a substitution of L55R. In some embodiments, the vIGF2 peptide has a sequence having a substitution of K65R. In some embodiments, the vIGF2 peptide has a sequence having a substitution of E6R, F26S, Y27L, V43L, F48T, R49S, 5501, A54R, and L55R. In some embodiments, the vIGF2 peptide has an N-terminal deletion. In some embodiments, the vIGF2 peptide has an N-terminal deletion of one amino acid. In some embodiments, the vIGF2 peptide has an N-terminal deletion of two amino acids. In some embodiments, the vIGF2 peptide has an N-terminal deletion of three amino acids. In some embodiments, the vIGF2 peptide has an N-terminal deletion of four amino acids. In some embodiments, the vIGF2 peptide has an N-terminal deletion of four amino acids and a substitution of E6R, Y27L, and K65R. In some embodiments, the vIGF2 peptide has an N-terminal deletion of four amino acids and a substitution of E6R and Y27L. In some embodiments, the vIGF2 peptide has an N-terminal deletion of five amino acids. In some embodiments, the vIGF2 peptide has an N-terminal deletion of six amino acids. In some embodiments, the vIGF2 peptide has an N-terminal deletion of seven amino acids. In some embodiments, the vIGF2 peptide has an N-terminal deletion of seven amino acids and a substitution of Y27L and K65R. In some embodiments, Bmax for CIMPR binding by SEQ ID NO:83 is enhanced compared to SEQ ID NO:80.

TABLE 3
IGF2 Amino Acid Sequences (variant residues
are underlined)

| | | SEQ |
|---|---|---|
| | | ID |
| Peptide | Sequence | NO |
| Wildtype | AYRPSETLCGGELVDTLQFVCGDRGFYFSRP | 68 |
| | ASRVSRRSRGIVEECCFRSCDLALLETYCATP | |
| | AKSE | |
| F26S | AYRPSETLCGGELVDTLQFVCGDRGSYFSRP | 69 |
| | ASRVSRRSRGIVEECCFRSCDLALLETYCATP | |
| | AKSE | |
| Y27L | AYRPSETLCGGELVDTLQFVCGDRGFLFSRPA | 70 |
| | SRVSRRSRGIVEECCFRSCDLALLETYCATPA | |
| | KSE | |
| V43L | AYRPSETLCGGELVDTLQFVCGDRGFYFSRP | 71 |
| | ASRVSRRSRGILEECCFRSCDLALLETYCATP | |
| | AKSE | |
| F48T | AYRPSETLCGGELVDTLQFVCGDRGFYFSRP | 72 |
| | ASRVSRRSRGIVEECCTRSCDLALLETYCATP | |
| | AKSE | |
| R49S | AYRPSETLCGGELVDTLQFVCGDRGFYFSRP | 73 |
| | ASRVSRRSRGIVEECCFSSCDLALLETYCATP | |
| | AKSE | |
| S50I | AYRPSETLCGGELVDTLQFVCGDRGFYFSRP | 74 |
| | ASRVSRRSRGIVEECCFRICDLALLETYCATPA | |
| | KSE | |
| A54R | AYRPSETLCGGELVDTLQFVCGDRGFYFSRP | 75 |
| | ASRVSRRSRGIVEECCFRSCDLRLLETYCATP | |
| | AKSE | |
| L55R | AYRPSETLCGGELVDTLQFVCGDRGFYFSRP | 76 |
| | ASRVSRRSRGIVEECCFRSCDLARLETYCATP | |
| | AKSE | |
| F26S, Y27L, | AYRPSETLCGGELVDTLQFVCGDRGSLFSRPA | 77 |
| V43L, F48T, | SRVSRRSRGILEECCTSICDLRRLETYCATPAK | |
| R49S, S50I, | SE | |
| A54R, L55R | | |
| Δ1-6, Y27L, | TLCGGELVDTLQFVCGDRGFLFSRPASRVSRR | 78 |
| K65R | SRGIVEECCFRSCDLALLETYCATPARSE | |
| Δ1-7, Y27L, | LCGGELVDTLQFVCGDRGFLFSRPASRVSRRS | 79 |
| K65R | RGIVEECCFRSCDLALLETYCATPARSE | |
| Δ1-4, E6R, | SRTLCGGELVDTLQFVCGDRGFLFSRPASRVS | 80 |
| Y27L, K65R | RRSRGIVEECCFRSCDLALLETYCATPARSE | |
| Δ1-4, E6R, | SRTLCGGELVDTLQFVCGDRGFLFSRPASRVS | 81 |
| Y27L | RRSRGIVEECCFRSCDLALLETYCATPAKSE | |
| E6R | AYRPSRTLCGGELVDTLQFVCGDRGFYFSRP | 82 |
| | ASRVSRRSRGIVEECCFRSCDLALLETYCATP | |
| | AKSE | |
| Δ1-4, E6R, | SRTLCGGELVDTLQFVCGDRGFLFSRPASRVS | 83 |
| Y27L, S50E, | RRSRGIVEECCFRECDLALLETYCATPARSE | |
| K65R | | |
| Cleavable | GGGGSGGGGSRPRAVPTQ | 84 |
| IGF2 | | |
| variant-N | | |
| terminal | | |
| Cleavable | YIPAKQGLQGAQMGQPGGGGSGGGG | 85 |
| IGF2 | | |
| variant-C | | |
| terminal | | |
| Cleavable | RPRAVPTQGGSGSGSTSS | 86 |
| IGF2 | | |
| variant-C | | |
| terminal | | |
| Cleavable | SRTLCGGELVDTLQFVCGDRGFLFSRPASRVS | 87 |
| IGF2 | RRSRGIVEECCFRSCDLALLETYCATPARSEG | |
| variant-N | GGGSGGGGSRPRAVPTQ | |
| terminal | | |
| Cleavable | YIPAKQGLQGAQMGQPGGGGSGGGGSRTLC | 88 |
| IGF2 | GGELVDTLQFVCGDRGFLFSRPASRVSRRSRG | |
| variant-C | IVEECCFRSCDLALLETYCATPARSE | |
| terminal | | |
| Cleavable | RPRAVPTQGGSGSGSTSSSRTLCGGELVDTLQ | 89 |
| IGF2 | FVCGDRGFLFSRPASRVSRRSRGIVEECCFRE | |
| variant-C | CDLALLETYCATPARSE | |
| terminal | | |
| vIGF2-1 | SRTLCGGELVDTNQFVCGDRGFLFSRGASRV | 90 |
| (vIGF2_1_ | SRGSRGIVEWCCFRGCDLALLETYCATPMRG | |
| NGGWGMG) | E | |
| VIGF2-2 | SRTLCGGELVDTLQFVCGDRGFLFSRGASRV | 91 |
| (vIGF2_2_ | SRGSRGIVEWCCFRGCDLALLETYCATPMRG | |
| GGWGMG) | E | |
| vIGF2-3 | SRTLCGGELVDTNQFVCGDRGFLFSRGASRV | 92 |
| (vIGF2_3_ | SRGSRGIVEECCFRGCDLALLETYCATPMRG | |
| NGGGMG) | E | |
| vIGF2-4 | SRTLCGGELVDTLQFVCGDRGFLFSRPIVEEC | 93 |
| (vIGF2_4_ | CFRSCDLALLETYCATPARSE | |
| Δ32-41, | | |
| 53aa) | | |
| vIGF2-5 | SRTLCGGELVDTLQFVCGDRGFLFSRGIVEEC | 94 |
| (vIGF2 | CFRSCDLALLETYCATPARSE | |
| Δ30-39, | | |
| 53aa) | | |
| vIGF2-6 | SRTLCGGELVDTLQFVCGDRGFLFSRPAGIVE | 95 |
| (vIGF2 | ECCFRSCDLALLETYCATPARSE | |
| Δ33-40, | | |
| 55aa) | | |
| vIGF2-7 | SRTLCGGELDDTLQFVCGDRGALRSRGIDEE | 96 |
| (vIGF2 | CCFRSCDLALLETYCATPARSE | |
| Δ30- | | |
| 39/V14D/ | | |
| F28R/V4 | | |
| 3D/F26A) | | |
| vIGF2-8 | SRTLCGGELVDTLQFVCGRRGELFSRPASRVS | 97 |
| (vIGF2_8_ | RRSRGIVEECCFRECDLALLETYC ATPARSE | |
| REE) | | |
| vIGF2-9 | SRTLCGGELVDTLQFVCGRRGELFSRPAGIVE | 98 |
| (vIGF2_9_ | ECCFRECDLALLETYCATPARSE | |
| Δ30-39- | | |
| REE; | | |
| vIGF2) | | |
| vIGF2-10 | SRTLCGGELVDTLQFVCGRRGFLFSRPASRVS | 99 |
| (vIGF2_ | RRSRGIVEECCFRSCDLALLETYCATPARSE | |
| 1Q; | | |
| vIGF2 | | |
| D23R) | | |
| vIGF2-11 | SRTLCGGELVDTLQWVCGDRGFLFSRPASRV | 100 |
| (vIGF2_ | SRRSRGIVEECCFRSCDLALLETYCATPARSE | |
| 2Q; | | |
| vIGF2 | | |
| F19W) | | |
| vIGF2-12 | SRTLCGGELVDWLQFVCGDRGFLFSRPASRV | 101 |
| (vIGF2_ | SRRSRGIVEECCFRSCDLALLETYCATPARSE | |
| 3Q; | | |
| vIGF2 | | |
| T16W) | | |
| vIGF2-13 | SRTLCGGELVDTLQFVCGKRGFLFSRPASRVS | 102 |
| (vIGF2_ | RRSRGIVEECCFRSCDLALLETYCATPARSE | |
| 4Q; | | |
| vIGF2 | | |
| D23K) | | |
| vIGF2-14 | SRTLCGGELVDYLQFVCGDRGFLFSRPASRVS | 103 |
| (vIGF2_ | RRSRGIVEECCFRSCDLALLETYCATPARSE | |
| 5Q; | | |
| vIGF2 | | |
| T16Y) | | |
| vIGF2-15 | SRTLCGGELVDTLQFVCGDRGELFSRPASRVS | 104 |
| (vIGF2_ | RRSRGIVEECCFRSCDLALLETYCATPARSE | |
| 6Q; | | |
| vIGF2 | | |
| F26E) | | |
| vIGF2-16 | SRTLCGGELVDVLQFVCGDRGFLFSRPASRVS | 105 |
| (vIGF2_ | RRSRGIVEECCFRSCDLALLETYCATPARSE | |
| 7Q; | | |
| vIGF2 | | |
| T16V) | | |
| vIGF2-17 | SRTLCGGELVDTLQFVCGDRGFLFSRPASRVS | 106 |
| (vIGF2_ | RRSRGIVEECCFRECDLALLETYCATPARSE | |
| 8Q; | | |
| vIGF2 | | |
| S50E) | | |
| vIGF2-18 | SRTLCGGELVDTLQFVCGDRGFLFSRPASRVS | 107 |
| (vIGF2_ | RRSRGIVEECCFRDCDLALLETYCATPARSE | |
| 9Q; | | |
| vIGF2 | | |
| S50D) | | |
| vIGF2-19 | SRTLCGGELVDTLQFVCGDRGSLFSRPASRVS | 108 |
| (vIGF2 | RRSRGILEECCFRSCDLALLETYCATPARSE | |
| F26S | | |
| V43L) | | |
| vIGF2-20 | SRTLCGGELVDTLQFVCGDRGFLFSRPASRVS | 109 |
| (vIGF2 | RRSRGILEECCFRSCDLALLETYCATPARSE | |
| V43L) | | |
| vIGF2-21 | SRTLCGGELEDTLQFVCGDRGSLRSRPASRVS | 110 |
| (vIGF2_ | RRSRGIEEECCFRSCDLALLETYCATPARSE | |
| ESRE; | | |
| vIGF2 V14E | | |
| F26S F28R | | |
| V43E) | | |
| vIGF2-22 | SRTLCGGELVDTLQFVCGDRGFLFSRGGGGS | 111 |
| (vIGF2 | RGIVEECCFRSCDLALLETYCATPARSE | |
| Δ31- | | |
| 38GGGG) | | |
| vIGF2-23 | SRTLCGGELVDTLQFVCGDRGFLFSGGGSGIV | 112 |
| (vIGF2 | EECCFRSCDLALLETYCATPARSE | |
| Δ30- | | |
| 40GGGG) | | |
| vIGF2-24 | SRTLCGGELVDTLQFVCGDRGFLFSRGGGGS | 113 |
| (vIGF2 | RGILEECCFRSCDLALLETYCATPARSE | |
| Δ31- | | |
| 38GGGG | | |
| V43L) | | |
| vIGF2-25 | SRTACGGELVDTLQFVCGDRGFLFSRPASRVS | 114 |
| (vIGF2 | RRSRGIVEECCFRSCDLALLETYCATPARSE | |
| L8A) | | |
| vIGF2-26 | SQAACGGELVDTLQFVCGDRGFLFSRPASRV | 115 |
| (vIGF2 | SRRSRGIVEECCFRSCDLALLETYCATPARSE | |
| R6Q T7A | | |
| L8A) | | |
| vIGF2-27 | SRTLCGGELVDTLQFVCGDEGFLFSRPASEVS | 116 |
| (vIGF2 | EESRGIVEECCFRSCDLALLETYCATPARSE | |
| R24E R34E | | |
| R37E | | |
| R38E) | | |
| vIGF2-28 | SRTLCGGELVDTLQFVCGDEGFLFSRPASEVS | 117 |
| (vIGF2 | RRSRGIVEECCFRSCDLALLETYCATPARSE | |
| R24E | | |
| R34E) | | |
| vIGF2-29 | SRTLCGGELVDTLQFVCGRRGFLFSRPASRVS | 118 |
| (vIGF2 | RRSRGIVEECCFRDCDLALLETYCATPARSE | |
| D23R | | |
| S40D) | | |
| vIGF2-30 | SRTLCGGELVDVLQFVCGRRGFLFSRPASRVS | 119 |
| (vIGF2 | RRSRGIVEECCFRDCDLALLETYCATPARSE | |
| T16V D23R | | |
| S50D) | | |
| vIGF2-31 | SRTLCGGELVDTLQFVCGDRGFLFSRGGGGS | 120 |
| (vIGF2 | RGILEECCFRDCDLALLETYCATPARSE | |
| Δ31- | | |
| 38GGGG | | |
| V43L | | |
| S50D) | | |
| vIGF2-32 | SRTLCGGELVDTLQFVCGDRGFLFSRGGGGS | 121 |
| (vIGF2 | RGILEECCFRECDLALLETYCATPARSE | |
| Δ31- | | |
| 38GGGG | | |
| V43L | | |
| S50E) | | |
| vIGF2-33 | SRTLCGGELVDTLQFVCGDRGFLFRLPSRPVS | 122 |
| (vIGF2- | RHSHRRSRGIVEECCFQRCNLALLETYCATPA | |
| N1) | RSE | |
| vIGF2-34 | SRTLCGGELVDTLQFVCGDRGFLFRLPSRPVS | 123 |
| (vIGF2- | RHSHRRSRGILEECCFQECNLALLETYCATPA | |
| N1 | RSE | |
| V43L | | |
| S50E) | | |
| vIGF2-1 | SRTLCGGELVDTLQFVCGDRGFLFSRPASRVS | 124 |
| R38G | RGSRGIVEECCFRSCDLALLETYCATPARSE | |
| vIGF2-2 | SRTLCGGELVDTLQFVCGDRGFLFSRPASRVS | 125 |
| R38G, E45W | RGSRGIVEWCCFRSCDLALLETYCATPARSE | |
| vIGF2-3 | SRTLCGGELVDTLQFVCGDRGFLFSRPASRVS | 126 |
| R38G, E45W, | RGSRGIVEWCCFRGCDLALLETYCATPARSE | |
| S50G | | |
| vIGF2-4 | SRTLCGGELVDTLQFVCGDRGFLFSRGASRV | 127 |
| P31G, | SRGSRGIVEWCCFRGCDLALLETYCATPARS | |
| R38G, E45W, | E | |
| S50G | | |
| VIGF2-5 | SRTLCGGELVDTNQFVCGDRGFLFSRGASRV | 128 |
| L17N, P31G, | SRGSRGIVEWCCFRGCDLALLETYCATPARS | |
| R38G, E45W, | E | |
| S50G | | |
| vIGF2-6 | SRTLCGGELVDTNQFVCGDRGFLFSRGASRV | 129 |
| L17N, P31G, | SRGSRGIVEWCCFRGCDLALLETYCATPARG | |
| R38G, E45W, | E | |
| S50G, S66G | | |
| vIGF2-7 | SRTLCGGELVDTNQFVCGDRGFLFSRGASRV | 130 |
| L17N, P31G, | SRGSRGIVEWCCFRGCDLALLETYCATPMRG | |
| R38G, E45W, | E | |
| S50G, A64M, | | |
| S66G | | |
| vIGF2-8 | LRTLCGGELVDTNQFVCGDRGFLFSRGASRV | 131 |
| S5L, L17N, | SRGSRGIVEWCCFRGCDLALLETYCATPMRG | |
| P31G, R38G, | E | |
| E45W, S50G, | | |
| A64M, S66G | | |

TABLE 4
IGF2 DNA Coding Sequences

| | | SEQ |
|---|---|---|
| | | ID |
| Peptide | DNA Sequence | NO |
| Mature | GCTTACCGCCCCAGTGAGACCCTGTGCGGC | 132 |
| WT IGF2 | GGGGAGCTGGTGGACACCCTCCAGTTCGTC | |
| | TGTGGGGACCGCGGCTTCTACTTCAGCAGG | |
| | CCCGCAAGCCGTGTGAGCCGTCGCAGCCGT | |
| | GGCATCGTTGAGGAGTGCTGTTTCCGCAGC | |
| | TGTGACCTGGCCCTCCTGGAGACGTACTGT | |
| | GCTACCCCCGCCAAGTCCGAG | |
| vIGF2 | TCTAGAACACTGTGCGGAGGGGAGCTTGTA | 133 |
| Δ1-4, E6R, | GACACTCTTCAGTTCGTGTGTGGAGATCGC | |
| Y27L, K65R | GGGTTCCTCTTCTCTCGCCCCGCTTCCAGAG | |
| | TTTCACGGAGGTCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2 | TCTAGAACACTGTGCGGAGGGGAGCTTGTA | 134 |
| Δ1-4, E6R, | GACACTCTTCAGTTCGTGTGTGGAGATCGC | |
| Y27L, S50E, | GGGTTCCTCTTCTCTCGCCCCGCTTCCAGAG | |
| K65R | TTTCACGGAGGTCTAGGGGTATAGTAGAGG | |
| | AGTGTTGTTTCAGGGAGTGTGACTTGGCGC | |
| | TCCTCGAGACCTATTGCGCGACGCCAGCCA | |
| | GGTCCGAA | |
| vIGF2-1 | TCTAGAACACTGTGCGGAGGGGAGCTTGTA | 135 |
| (vIGF2_1_ | GACACTAACCAGTTCGTGTGTGGAGATCGC | |
| NGGWGMG) | GGGTTCCTCTTCTCTCGCGGCGCTTCCAGAG | |
| | TTTCACGGGGCTCTAGGGGTATAGTAGAGT | |
| | GGTGTTGTTTCAGGGGCTGTGACTTGGCGC | |
| | TCCTCGAGACCTATTGCGCGACGCCAATGA | |
| | GGGGCGAA | |
| vIGF2-2 | TCTAGAACACTGTGCGGAGGGGAGCTTGTA | 136 |
| (vIGF2_2_ | GACACTCTTCAGTTCGTGTGTGGAGATCGC | |
| GGWGMG) | GGGTTCCTCTTCTCTCGCGGCGCTTCCAGAG | |
| | TTTCACGGGGCTCTAGGGGTATAGTAGAGT | |
| | GGTGTTGTTTCAGGGGCTGTGACTTGGCGC | |
| | TCCTCGAGACCTATTGCGCGACGCCAATGA | |
| | GGGGCGAA | |
| vIGF2-3 | TCTAGAACACTGTGCGGAGGGGAGCTTGTA | 137 |
| (vIGF2_3_ | GACACTAACCAGTTCGTGTGTGGAGATCGC | |
| NGGGMG) | GGGTTCCTCTTCTCTCGCGGCGCTTCCAGAG | |
| | TTTCACGGGGCTCTAGGGGTATAGTAGAGG | |
| | AGTGTTGTTTCAGGGGCTGTGACTTGGCGC | |
| | TCCTCGAGACCTATTGCGCGACGCCAATGA | |
| | GGGGCGAA | |
| vIGF2-4 | TCTAGAACACTGTGCGGAGGGGAGCTTGTA | 138 |
| (vIGF2_4_ | GACACTCTTCAGTTCGTGTGTGGAGATCGC | |
| Δ32-41, | GGGTTCCTCTTCTCTCGCCCCATAGTAGAGG | |
| 53aa) | AGTGTTGTTTCAGGTCCTGTGACTTGGCGCT | |
| | CCTCGAGACCTATTGCGCGACGCCAGCCAG | |
| | GTCCGAA | |
| vIGF2-5 | TCTAGAACACTGTGCGGAGGGGAGCTTGTA | 139 |
| (vIGF2 | GACACTCTTCAGTTCGTGTGTGGAGATCGC | |
| Δ30-39, | GGGTTCCTCTTCTCTAGGGGTATAGTAGAG | |
| 53aa) | GAGTGTTGTTTCAGGTCCTGTGACTTGGCGC | |
| | TCCTCGAGACCTATTGCGCGACGCCAGCCA | |
| | GGTCCGAA | |
| vIGF2-6 | TCTAGAACACTGTGCGGAGGGGAGCTTGTA | 140 |
| (vIGF2 | GACACTCTTCAGTTCGTGTGTGGAGATCGC | |
| Δ33-40, | GGGTTCCTCTTCTCTCGCCCCGCTGGTATAG | |
| 55aa) | TAGAGGAGTGTTGTTTCAGGTCCTGTGACTT | |
| | GGCGCTCCTCGAGACCTATTGCGCGACGCC | |
| | AGCCAGGTCCGAA | |
| VIGF2-7 | TCTAGAACACTGTGCGGAGGGGAGCTTGAC | 141 |
| (vIGF2 | GACACTCTTCAGTTCGTGTGTGGAGATCGC | |
| Δ30- | GGGGCCCTCAGATCTAGGGGTATAGACGAG | |
| 39/V14D/ | GAGTGTTGTTTCAGGTCCTGTGACTTGGCGC | |
| F28R/V4 | TCCTCGAGACCTATTGCGCGACGCCAGCCA | |
| 3D/F26A) | GGTCCGAA | |
| vIGF2-8 | TCTAGAACACTGTGCGGAGGGGAGCTTGTA | 142 |
| (vIGF2_ | GACACTCTTCAGTTCGTGTGTGGAAGACGC | |
| 8_REE) | GGGGAGCTCTTCTCTCGCCCCGCTTCCAGA | |
| | GTTTCACGGAGGTCTAGGGGTATAGTAGAG | |
| | GAGTGTTGTTTCAGGGAGTGTGACTTGGCG | |
| | CTCCTCGAGACCTATTGCGCGACGCCAGCC | |
| | AGGTCCGAA | |
| vIGF2-9 | TCTAGAACACTGTGCGGAGGGGAGCTTGTA | 143 |
| (vIGF2_9_ | GACACTCTTCAGTTCGTGTGTGGAAGACGC | |
| Δ30-39- | GGGGAGCTCTTCTCTCGCCCCGCTGGTATA | |
| REE; | GTAGAGGAGTGTTGTTTCAGGGAGTGTGAC | |
| vIGF2 | TTGGCGCTCCTCGAGACCTATTGCGCGACG | |
| Homerun) | CCAGCCAGGTCCGAA | |
| vIGF2-10 | TCTAGAACACTGTGCGGAGGGGAGCTTGTA | 144 |
| (vIGF2_ | GACACTCTTCAGTTCGTGTGTGGACGTCGC | |
| 1Q; | GGGTTCCTCTTCTCTCGCCCCGCTTCCAGAG | |
| vIGF2 D23R) | TTTCACGGAGGTCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-11 | TCTAGAACACTGTGCGGAGGGGAGCTTGTA | 145 |
| (vIGF2_ | GACACTCTTCAGTGGGTGTGTGGAGATCGC | |
| 2Q; | GGGTTCCTCTTCTCTCGCCCCGCTTCCAGAG | |
| vIGF2 F19W) | TTTCACGGAGGTCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-12 | TCTAGAACACTGTGCGGAGGGGAGCTTGTA | 146 |
| (vIGF2_ | GACTGGCTTCAGTTCGTGTGTGGAGATCGC | |
| 3Q; | GGGTTCCTCTTCTCTCGCCCCGCTTCCAGAG | |
| vIGF2 T16W) | TTTCACGGAGGTCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-13 | TCTAGAACACTGTGCGGAGGGGAGCTTGTA | 147 |
| (vIGF2_ | GACACTCTTCAGTTCGTGTGTGGAAAGCGC | |
| 4Q; | GGGTTCCTCTTCTCTCGCCCCGCTTCCAGAG | |
| vIGF2 D23K) | TTTCACGGAGGTCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-14 | TCTAGAACACTGTGCGGAGGGGAGCTTGTA | 148 |
| (vIGF2_ | GACTATCTTCAGTTCGTGTGTGGAGATCGC | |
| 5Q; | GGGTTCCTCTTCTCTCGCCCCGCTTCCAGAG | |
| vIGF2 T16Y) | TTTCACGGAGGTCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-15 | TCTAGAACACTGTGCGGAGGGGAGCTTGTA | 149 |
| (vIGF2_ | GACACTCTTCAGTTCGTGTGTGGAGATCGC | |
| 6Q; | GGGGAGCTCTTCTCTCGCCCCGCTTCCAGA | |
| vIGF2 F26E) | GTTTCACGGAGGTCTAGGGGTATAGTAGAG | |
| | GAGTGTTGTTTCAGGTCCTGTGACTTGGCGC | |
| | TCCTCGAGACCTATTGCGCGACGCCAGCCA | |
| | GGTCCGAA | |
| vIGF2-16 | TCTAGAACACTGTGCGGAGGGGAGCTTGTA | 150 |
| (vIGF2_ | GACGTTCTTCAGTTCGTGTGTGGAGATCGC | |
| 7Q; | GGGTTCCTCTTCTCTCGCCCCGCTTCCAGAG | |
| vIGF2 T16V) | TTTCACGGAGGTCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-17 | TCTAGAACACTGTGCGGAGGGGAGCTTGTA | 151 |
| (vIGF2_ | GACACTCTTCAGTTCGTGTGTGGAGATCGC | |
| 8Q; | GGGTTCCTCTTCTCTCGCCCCGCTTCCAGAG | |
| vIGF2 S50E) | TTTCACGGAGGTCTAGGGGTATAGTAGAGG | |
| | AGTGTTGTTTCAGGGAGTGTGACTTGGCGC | |
| | TCCTCGAGACCTATTGCGCGACGCCAGCCA | |
| | GGTCCGAA | |
| vIGF2-18 | TCTAGAACACTGTGCGGAGGGGAGCTTGTA | 152 |
| (vIGF2_ | GACACTCTTCAGTTCGTGTGTGGAGATCGC | |
| 9Q; | GGGTTCCTCTTCTCTCGCCCCGCTTCCAGAG | |
| vIGF2 S50D) | TTTCACGGAGGTCTAGGGGTATAGTAGAGG | |
| | AGTGTTGTTTCAGGGACTGTGACTTGGCGC | |
| | TCCTCGAGACCTATTGCGCGACGCCAGCCA | |
| | GGTCCGAA | |
| vIGF2-19 | TCTAGAACACTGTGCGGAGGGGAGCTTGTA | 153 |
| (vIGF2 | GACACTCTTCAGTTCGTGTGTGGAGATCGC | |
| F26S V43L) | GGGAGCCTCTTCTCTCGCCCCGCTTCCAGA | |
| | GTTTCACGGAGGTCTAGGGGTATACTGGAG | |
| | GAGTGTTGTTTCAGGTCCTGTGACTTGGCGC | |
| | TCCTCGAGACCTATTGCGCGACGCCAGCCA | |
| | GGTCCGAA | |
| vIGF2-20 | TCTAGAACACTGTGCGGAGGGGAGCTTGTA | 154 |
| (vIGF2 | GACACTCTTCAGTTCGTGTGTGGAGATCGC | |
| V43L) | GGGTTCCTCTTCTCTCGCCCCGCTTCCAGAG | |
| | TTTCACGGAGGTCTAGGGGTATACTGGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-21 | TCTAGAACACTGTGCGGAGGGGAGCTTGAG | 155 |
| (vIGF2_ | GACACTCTTCAGTTCGTGTGTGGAGATCGC | |
| ESRE; | GGGAGCCTCAGATCTCGCCCCGCTTCCAGA | |
| vIGF2 V14E | GTTTCACGGAGGTCTAGGGGTATAGAGGAG | |
| F26S F28R | GAGTGTTGTTTCAGGTCCTGTGACTTGGCGC | |
| V43E) | TCCTCGAGACCTATTGCGCGACGCCAGCCA | |
| | GGTCCGAA | |
| vIGF2-22 | TCTAGAACACTGTGCGGAGGGGAGCTTGTA | 156 |
| (vIGF2 | GACACTCTTCAGTTCGTGTGTGGAGATCGC | |
| Δ31- | GGGTTCCTCTTCTCTCGCGGAGGTGGAGGT | |
| 38GGGG) | TCTAGGGGTATAGTAGAGGAGTGTTGTTTC | |
| | AGGTCCTGTGACTTGGCGCTCCTCGAGACC | |
| | TATTGCGCGACGCCAGCCAGGTCCGAA | |
| vIGF2-23 | TCTAGAACACTGTGCGGAGGGGAGCTTGTA | 157 |
| (vIGF2 | GACACTCTTCAGTTCGTGTGTGGAGATCGC | |
| Δ30- | GGGTTCCTCTTCTCTGGTGGAGGTTCTGGTA | |
| 40GGGG) | TAGTAGAGGAGTGTTGTTTCAGGTCCTGTG | |
| | ACTTGGCGCTCCTCGAGACCTATTGCGCGA | |
| | CGCCAGCCAGGTCCGAA | |
| vIGF2-24 | TCTAGAACACTGTGCGGAGGGGAGCTTGTA | 158 |
| (vIGF2 | GACACTCTTCAGTTCGTGTGTGGAGATCGC | |
| Δ31- | GGGTTCCTCTTCTCTCGCGGAGGTGGAGGT | |
| 38GGGG | TCTAGGGGTATACTGGAGGAGTGTTGTTTC | |
| V43L) | AGGTCCTGTGACTTGGCGCTCCTCGAGACC | |
| | TATTGCGCGACGCCAGCCAGGTCCGAA | |
| vIGF2-25 | TCTCAGGCCGCGTGCGGAGGGGAGCTTGTA | 159 |
| (vIGF2 | GACACTCTTCAGTTCGTGTGTGGAGATCGC | |
| L8A) | GGGTTCCTCTTCTCTCGCCCCGCTTCCAGAG | |
| | TTTCACGGAGGTCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-26 | TCTCAGGCCGCGTGCGGAGGGGAGCTTGTA | 160 |
| (vIGF2 | GACACTCTTCAGTTCGTGTGTGGAGATCGC | |
| R6Q T7A | GGGTTCCTCTTCTCTCGCCCCGCTTCCAGAG | |
| L8A) | TTTCACGGAGGTCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-27 | TCTAGAACACTGTGCGGAGGGGAGCTTGTA | 161 |
| (vIGF2 | GACACTCTTCAGTTCGTGTGTGGAGATGAG | |
| R24E R34E | GGGTTCCTCTTCTCTCGCCCCGCTTCCGAGG | |
| R37E R38E) | TTTCAGAGGAATCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-28 | TCTAGAACACTGTGCGGAGGGGAGCTTGTA | 162 |
| (vIGF2 | GACACTCTTCAGTTCGTGTGTGGAGATGAG | |
| R24E R34E) | GGGTTCCTCTTCTCTCGCCCCGCTTCCGAGG | |
| | TTTCACGGAGGTCTAGGGGTATAGTAGAGG | |
| | AGTGTTGTTTCAGGTCCTGTGACTTGGCGCT | |
| | CCTCGAGACCTATTGCGCGACGCCAGCCAG | |
| | GTCCGAA | |
| vIGF2-29 | TCTAGAACACTGTGCGGAGGGGAGCTTGTA | 163 |
| (vIGF2 | GACACTCTTCAGTTCGTGTGTGGAAGACGC | |
| D23R S40D) | GGGTTCCTCTTCTCTCGCCCCGCTTCCAGAG | |
| | TTTCACGGAGGTCTAGGGGTATAGTAGAGG | |
| | AGTGTTGTTTCAGGGACTGTGACTTGGCGC | |
| | TCCTCGAGACCTATTGCGCGACGCCAGCCA | |
| | GGTCCGAA | |
| vIGF2-30 | TCTAGAACACTGTGCGGAGGGGAGCTTGTA | 164 |
| (vIGF2 | GACGTGCTTCAGTTCGTGTGTGGAAGACGC | |
| T16V D23R | GGGTTCCTCTTCTCTCGCCCCGCTTCCAGAG | |
| S50D) | TTTCACGGAGGTCTAGGGGTATAGTAGAGG | |
| | AGTGTTGTTTCAGGGACTGTGACTTGGCGC | |
| | TCCTCGAGACCTATTGCGCGACGCCAGCCA | |
| | GGTCCGAA | |
| vIGF2-31 | TCTAGAACACTGTGCGGAGGGGAGCTTGTA | 165 |
| (vIGF2 | GACACTCTTCAGTTCGTGTGTGGAGATCGC | |
| Δ31- | GGGTTCCTCTTCTCTCGCGGAGGTGGAGGT | |
| 38GGGG | TCTAGGGGTATACTGGAGGAGTGTTGTTTC | |
| V43L S50D) | AGGGACTGTGACTTGGCGCTCCTCGAGACC | |
| | TATTGCGCGACGCCAGCCAGGTCCGAA | |
| vIGF2-32 | TCTAGAACACTGTGCGGAGGGGAGCTTGTA | 166 |
| (vIGF2 | GACACTCTTCAGTTCGTGTGTGGAGATCGC | |
| Δ31- | GGGTTCCTCTTCTCTCGCGGAGGTGGAGGT | |
| 38GGGG | TCTAGGGGTATACTGGAGGAGTGTTGTTTC | |
| V43L S50E) | AGGGAGTGTGACTTGGCGCTCCTCGAGACC | |
| | TATTGCGCGACGCCAGCCAGGTCCGAA | |
| vIGF2-33 | TCTAGAACACTGTGCGGAGGGGAGCTTGTA | 167 |
| (vIGF2- | GACACTCTTCAGTTCGTGTGTGGAGATCGC | |
| N1) | GGGTTCTTGTTTCGATTGCCGTCCAGGCCCG | |
| | TGTCCCGGCACAGTCACCGCAGGTCAAGGG | |
| | GGATAGTTGAAGAATGTTGCTTTCAGAGGT | |
| | GTAATTTGGCGCTCCTCGAGACCTATTGCG | |
| | CGACGCCAGCCAGGTCCGAA | |
| vIGF2-34 | TCTAGAACACTGTGCGGAGGGGAGCTTGTA | 168 |
| (vIGF2- | GACACTCTTCAGTTCGTGTGTGGAGATCGC | |
| N1 | GGGTTCTTGTTTCGATTGCCGTCCAGGCCCG | |
| V43L S50E) | TGTCCCGGCACAGTCACCGCAGGTCAAGGG | |
| | GGATACTGGAAGAATGTTGCTTTCAGGAGT | |
| | GTAATTTGGCGCTCCTCGAGACCTATTGCG | |
| | CGACGCCAGCCAGGTCCGAA | |

Internal Ribosomal Entry Sequences

Provided herein are gene therapy constructs useful in treating a disorder further comprising an internal ribosome entry sequence (IRES) for increasing gene expression by bypassing the bottleneck of translation initiation. Suitable internal ribosomal entry sequences for optimizing expression for gene therapy include but are not limited to a cricket paralysis virus (CrPV) IRES, a picornavirus IRES, an Aphthovirus IRES, a Kaposi's sarcoma-associated herpesvirus IRES, a Hepatitis A IRES, a Hepatitis C IRES, a Pestivirus IRES, a Cripavirus IRES, a Rhopalosiphum padi virus IRES, a Merek's disease virus IRES, and other suitable IRES sequences. In some embodiments, the gene therapy construct comprises a CrPV IRES. In some embodiments, the CrPV IRES has a nucleic acid sequence of AAAAATGTGATCTTGCTTGTAAATACAATTTTGAGAGGTTAATAAATTACAAGTAG TGCTATTTTTGTATTTAGGTTAGCTATTTAGCTTTACGTTCCAGGATGCCTAGTGGC AGCCCCACAATATCCAGGAAGCCCTCTCTGCGGTTTTTCAGATTAGGTAGTCGAAA AACCTAAGAAATTTACCTGCT (SEQ ID NO: 191). In some embodiments, the CrPV IRES sequence is at least 90%, at least 91%, at least 92%, at least 93%, at least 94%, at least 95%, at least 96%, at least 97%, at least 98%, or at least 99% identical to SEQ ID NO: 191.

Signal Peptides

Gene therapy constructs provided herein, in some embodiments, further comprise a signal peptide, which improves secretion of the therapeutic protein from the cell transduced with the gene therapy construct. The signal peptide in some embodiments improves protein processing of therapeutic proteins and facilitates translocation of the nascent polypeptide-ribosome complex to the ER and ensuring proper co-translational and post-translational modifications. In some embodiments, the signal peptide is located (i) in between the translation initiation sequence and the therapeutic protein or (ii) a downstream position of the therapeutic protein. Signal peptides useful in gene therapy constructs include but are not limited to binding immunoglobulin protein (BiP) signal peptide from the family of HSP70 proteins (e.g., HSPA5, heat shock protein family A member 5) and Gaussia signal peptides, and variants thereof. These signal peptides have ultrahigh affinity to the signal recognition particle. Examples of BiP and Gaussia amino acid sequences are provided in Table 5 below. In some embodiments, the signal peptide has an amino acid sequence that is at least 90, 95, 96, 97, 98 or 99% identical to a sequence selected from the group consisting of SEQ ID NOs: 169-180. In some embodiments, the signal peptide differs from a sequence selected from the group consisting of SEQ ID NOs: 169-180 by 5 or fewer, 4 or fewer, 3 or fewer, 2 or fewer, or 1 amino acid. In some embodiments, the native signal peptide, referred to interchangeably herein as the “endogenous signal peptide” of a lysosomal protein is used.

TABLE 5
Signal Peptide Sequences

| | | SEQ |
|---|---|---|
| Signal | | ID |
| Peptide | Amino Acid Sequence | NO: |
| Native human | MKLSLVAAMLLLLSAARA | 169 |
| BiP | | |
| Modified | MKLSLVAAMLLLLSLVAAMLLLLSAARA | 170 |
| BiP-1 | | |
| Modified | MKLSLVAAMLLLLWVALLLLSAARA | 171 |
| BiP-2 | | |
| Modified | MKLSLVAAMLLLLSLVALLLLSAARA | 172 |
| BiP-3 | | |
| Modified | MKLSLVAAMLLLLALVALLLLSAARA | 173 |
| BiP-4 | | |
| Gaussia | MGVKVLFALICIAVAEA | 174 |
| Native PPT1 | MASPGCLWLLAVALLPWTCASRALQHL | 175 |
| Signal | | |
| Peptide | | |
| (eSP) | | |
| Native PPT1 | MASPGCLWLLAVALLPWTCASRALQHLAA | 176 |
| Signal | | |
| Peptide | | |
| (eSP AA) | | |
| Native PPT1 | MASPGSLWLLAVALLPWTCASRALQHL | 177 |
| Signal | | |
| Peptide C6S | | |
| (eSP C6S) | | |
| Native PPT1 | MASPGSLWLLAVALLPWTCASRALQHLAA | 178 |
| Signal | | |
| Peptide C6S | | |
| (eSP C6S AA) | | |
| Native TPP1 | MGLQACLLGLFALILSGKC | 179 |
| Signal | | |
| Peptide | | |
| Native NAGLU | MEAVAVAAAVGVLLLAGAGGAAGD | 180 |
| Signal | | |
| Peptide | | |

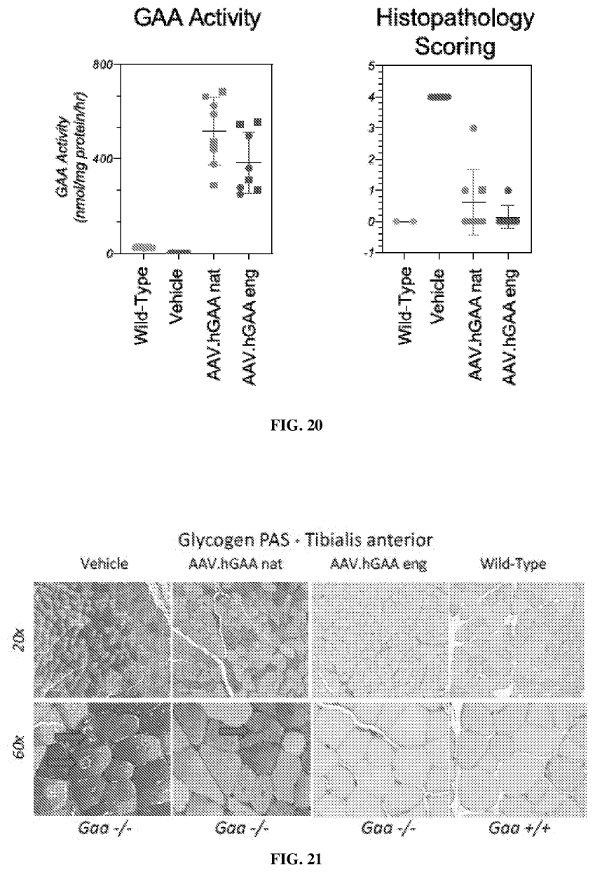

The BiP signal peptide-signal recognition particle (SRP) interaction facilitates translocation to the ER. This interaction is illustrated in FIG. 20.

The Gaussia signal peptide is derived from the luciferase from Gaussia princeps and directs increased protein synthesis and secretion of therapeutic proteins fused to this signal peptide. In some embodiments, the Gaussia signal peptide has an amino acid sequence that is at least 90, 95, 96, 97, 98 or 99% identical to SEQ ID NO: 174. In some embodiments, the signal peptide differs from SEQ ID NO: 174 by 5 or fewer, 4 or fewer, 3 or fewer, 2 or fewer, or 1 amino acid.

Linker

Gene therapy constructs provided herein, in some embodiments, comprise a linker between the targeting peptide and the therapeutic protein. Such linkers, in some embodiments, maintain correct spacing and mitigate steric clash between the vIGF2 peptide and the therapeutic protein. Linkers, in some embodiments, comprise repeated glycine residues, repeated glycine-serine residues, and combinations thereof. In some embodiments, the linker consists of 5-20 amino acids, 5-15 amino acids, 5-10 amino acids, 8-12 amino acids, or about 5, 6, 7, 8, 9, 10, 11, 12 or 13 amino acids. Suitable linkers for gene therapy and enzyme replacement therapy constructs herein include but are not limited to those provided in Table 6 below.

TABLE 6
Linker Sequences

| GS Linkers | Sequence | SEQ ID NO: |
|---|---|---|
| | GGGGSGGGG | 181 |
| | GGGGS | 182 |
| | GGGSGGGGS | 183 |
| | GGGGSGGGS | 184 |
| | GGSGSGSTS | 185 |
| | GGGGSGGGGS | 186 |
| | GGGGSGSGGGGS | 187 |
| Lysosomal | RPRAVPTQA | 188 |
| cleavage | | |
| linker | | |

Translation Initiation Sequence

Gene therapy constructs provided herein comprise a nucleic acid having a translation initiation sequence, such as a Kozak sequence which aids in initiation of translation of the mRNA. Kozak sequences contemplated herein have a consensus sequence of (gcc)RccATGG where a lowercase letter denotes the most common base at the position and the base varies, uppercase letters indicate highly conserved bases that only vary rarely change. R indicates that a purine (adenine or guanine) is always observed at that position. The sequence in parentheses (gcc) is of uncertain significance. In some embodiments, the Kozak sequence comprises the sequence AX₁X₂ATGA, wherein each of X₁ and X₂ is any nucleotide. In some embodiments, X₁ comprises A. In some embodiments, X₂ comprises G. In some embodiments, the Kozak sequence comprises a nucleic acid sequence at least 85% identical to AAGATGA. In some embodiments, the Kozak sequence differs from the sequence of AAGATGA by one or two nucleotides. In some embodiments, Kozak sequences provided herein have a sequence of AAGATGA. In some embodiments the Kozak sequence comprises a nucleic acid sequence at least 85% identical to GCAAGATG. In some embodiments the Kozak sequence differs from the sequence of GCAAGATG by one or two nucleotides. In some embodiments, the Kozak sequence comprises GCAAGATG. In some embodiments the Kozak sequence comprises a nucleic acid sequence at least 85% identical to CACCATG. In some embodiments the Kozak sequence differs from the sequence of CACCATG by one or two nucleotides. In some embodiments, the Kozak sequence comprises CACCATG.

Therapeutic Protein

Gene therapy constructs provided herein comprise a nucleic acid encoding a therapeutic protein for treating a genetic disorder due to a genetic defect in an individual resulting in an absent or defective protein. The therapeutic protein expressed from the gene therapy construct replaces the absent or defective protein. Therapeutic proteins, therefore, are chosen based on the genetic defect in need of treatment in an individual. In some embodiments, the therapeutic protein is a structural protein. In some embodiments, the therapeutic protein is an enzyme. In some embodiments, the therapeutic protein is a regulatory protein. In some embodiments, the therapeutic protein is a receptor. In some embodiments, the therapeutic protein is a peptide hormone. In some embodiments, the therapeutic protein is a cytokine or a chemokine.

In some embodiments, gene therapy constructs herein encode an enzyme, such as an enzyme having a genetic defect in an individual with a lysosomal storage disorder. In some embodiments, gene therapy constructs encode a lysosomal enzyme, such as a glycosidase, a protease, or a sulfatase. In some embodiments, enzymes encoded by gene therapy constructs provided herein include but are not limited to α-D-mannosidase; N-aspartyl-β-glucosaminidase; β-galactosidase; ceramidase; fucosidase; galactocerebrosidase; arylsulfatase A; N-acetylglucosamine-1-phosphotransferase; iduronate sulfatase; N-acetylglucosaminidase; acetyl-CoA:α-glucosaminide acetyltransferase; N-acetylglucosamine 6-sulfatase; β-glucuronidase; hyaluronidase; sialidase; sulfatase; sphingomyelinase; acid β-mannosidase; cathepsin K; 3-hexosaminidase A; β-hexosaminidase B; α-N-acetylgalactosaminidase; sialin; hexosaminidase A; beta-glucosidase; α-iduronidase; α-galactosidase A; β-glucocerebrosidase; lysosomal acid lipase; glycosaminoglycan alpha-L-iduronohydrolase; iduronate-2-sulfatase; N-acetylgalactosamine-6-sulfatase; glycosaminoglycan N-acetylgalactosamine 4-sulfatase; alpha-glucosidase; heparan sulfamidase; gp-91 subunit of NADPH oxidase; adenosine deaminase; cyclin dependent kinase like 5; and palmitoyl protein thioesterase 1. In some embodiments, enzymes encoded by gene therapy constructs provided herein comprise alpha-glucosidase. In some embodiments, the therapeutic protein is associated with a genetic disorder selected from the group consisting of cystic fibrosis, alpha- and beta-thalassemias, sickle cell anemia, Marfan syndrome, fragile X syndrome, Huntington's disease, hemochromatosis, Congenital Deafness (nonsyndromic), Tay-Sachs, Familial hypercholesterolemia, Duchenne muscular dystrophy, Stargardt disease, Usher syndrome, choroideremia, achromatopsia, X-linked retinoschisis, hemophilia, Wiskott-Aldrich syndrome, X-linked chronic granulomatous disease, aromatic L-amino acid decarboxylase deficiency, recessive dystrophic epidermolysis bullosa, alpha 1 antitrypsin deficiency, Hutchinson-Gilford progeria syndrome (HGPS), Noonan syndrome, X-linked severe combined immunodeficiency (X-SCID).

Gene Therapy Vector Examples

Gene Therapy Vectors and Compositions

Provided herein are gene therapy vectors in which a nucleic acid, such as a DNA, encoding a therapeutic fusion protein, such as a vIGF2 fusion, optionally having a signal peptide. The gene therapy vector optionally comprises an internal ribosomal entry sequence. Vectors derived from retroviruses such as the lentivirus are suitable tools to achieve long-term gene transfer since they allow long-term, stable integration of a transgene and its propagation in daughter cells. Lentiviral and adeno-associated viral vectors have the added advantage over vectors derived from onco-retroviruses such as murine leukemia viruses in that they are capable of transducing non-proliferating cells, such as hepatocytes and neurons. They also have the added advantage of low immunogenicity.

Exemplary gene therapy vectors herein encode therapeutic proteins and therapeutic fusion proteins comprising a vIGF2 peptide. Nucleic acids encoding exemplary fusion protein amino acid sequences are provided in Table 7 below.

TABLE 7
DNA Sequences

| | | SEQ |
|---|---|---|
| | | ID |
| Construct | DNA Sequence | NO |
| Kozak- | GCAAGATGGGAGTGAGGCACCCGCCCTGCTCCCACCGGCTCCTG | 189 |
| hGAA | GCCGTCTGCGCCCTCGTGTCCTTGGCAACCGCTGCACTCCTGGGG | |
| (Natural | CACATCCTACTCCATGATTTCCTGCTGGTTCCCCGAGAGCTGAGT | |
| GAA) | GGCTCCTCCCCAGTCCTGGAGGAGACTCACCCAGCTCACCAGCA | |
| | GGGAGCCAGTAGACCAGGGCCCCGGGATGCCCAGGCACACCCC | |
| | GGCCGTCCCAGAGCAGTGCCCACACAGTGCGACGTCCCCCCCAA | |
| | CAGCCGCTTCGATTGCGCCCCTGACAAGGCCATCACCCAGGAAC | |
| | AGTGCGAGGCCCGCGGCTGTTGCTACATCCCTGCAAAGCAGGGG | |
| | CTGCAGGGAGCCCAGATGGGGCAGCCCTGGTGCTTCTTCCCACC | |
| | CAGCTACCCCAGCTACAAGCTGGAGAACCTGAGCTCCTCTGAAA | |
| | TGGGCTACACGGCCACCCTGACCCGTACCACCCCCACCTTCTTCC | |
| | CCAAGGACATCCTGACCCTGCGGCTGGACGTGATGATGGAGACT | |
| | GAGAACCGCCTCCACTTCACGATCAAAGATCCAGCTAACAGGCG | |
| | CTACGAGGTGCCCTTGGAGACCCCGCATGTCCACAGCCGGGCAC | |
| | CGTCCCCACTCTACAGCGTGGAGTTCTCCGAGGAGCCCTTCGGG | |
| | GTGATCGTGCGCCGGCAGCTGGACGGCCGCGTGCTGCTGAACAC | |
| | GACGGTGGCGCCCCTGTTCTTTGCGGACCAGTTCCTTCAGCTGTC | |
| | CACCTCGCTGCCCTCGCAGTATATCACAGGCCTCGCCGAGCACCT | |
| | CAGTCCCCTGATGCTCAGCACCAGCTGGACCAGGATCACCCTGT | |
| | GGAACCGGGACCTTGCGCCCACGCCCGGTGCGAACCTCTACGGG | |
| | TCTCACCCTTTCTACCTGGCGCTGGAGGACGGCGGGTCGGCACA | |
| | CGGGGTGTTCCTGCTAAACAGCAATGCCATGGATGTGGTCCTGC | |
| | AGCCGAGCCCTGCCCTTAGCTGGAGGTCGACAGGTGGGATCCTG | |
| | GATGTCTACATCTTCCTGGGCCCAGAGCCCAAGAGCGTGGTGCA | |
| | GCAGTACCTGGACGTTGTGGGATACCCGTTCATGCCGCCATACTG | |
| | GGGCCTGGGCTTCCACCTGTGCCGCTGGGGCTACTCCTCCACCGC | |
| | TATCACCCGCCAGGTGGTGGAGAACATGACCAGGGCCCACTTCC | |
| | CCCTGGACGTCCAGTGGAACGACCTGGACTACATGGACTCCCGG | |
| | AGGGACTTCACGTTCAACAAGGATGGCTTCCGGGACTTCCCGGC | |
| | CATGGTGCAGGAGCTGCACCAGGGCGGCCGGCGCTACATGATGA | |
| | TCGTGGATCCTGCCATCAGCAGCTCGGGCCCTGCCGGGAGCTAC | |
| | AGGCCCTACGACGAGGGTCTGCGGAGGGGGGTTTTCATCACCAA | |
| | CGAGACCGGCCAGCCGCTGATTGGGAAGGTATGGCCCGGGTCCA | |
| | CTGCCTTCCCCGACTTCACCAACCCCACAGCCCTGGCCTGGTGGG | |
| | AGGACATGGTGGCTGAGTTCCATGACCAGGTGCCCTTCGACGGC | |
| | ATGTGGATTGACATGAACGAGCCTTCCAACTTCATCAGGGGCTCT | |
| | GAGGACGGCTGCCCCAACAATGAGCTGGAGAACCCACCCTACGT | |
| | GCCTGGGGTGGTTGGGGGGACCCTCCAGGCGGCCACCATCTGTG | |
| | CCTCCAGCCACCAGTTTCTCTCCACACACTACAACCTGCACAACC | |
| | TCTACGGCCTGACCGAAGCCATCGCCTCCCACAGGGCGCTGGTG | |
| | AAGGCTCGGGGGACACGCCCATTTGTGATCTCCCGCTCGACCTTT | |
| | GCTGGCCACGGCCGATACGCCGGCCACTGGACGGGGGACGTGTG | |
| | GAGCTCCTGGGAGCAGCTCGCCTCCTCCGTGCCAGAAATCCTGC | |
| | AGTTTAACCTGCTGGGGGTGCCTCTGGTCGGGGCCGACGTCTGC | |
| | GGCTTCCTGGGCAACACCTCAGAGGAGCTGTGTGTGCGCTGGAC | |
| | CCAGCTGGGGGCCTTCTACCCCTTCATGCGGAACCACAACAGCC | |
| | TGCTCAGTCTGCCCCAGGAGCCGTACAGCTTCAGCGAGCCGGCC | |
| | CAGCAGGCCATGAGGAAGGCCCTCACCCTGCGCTACGCACTCCT | |
| | CCCCCACCTCTACACACTGTTCCACCAGGCCCACGTCGCGGGGG | |
| | AGACCGTGGCCCGGCCCCTCTTCCTGGAGTTCCCCAAGGACTCTA | |
| | GCACCTGGACTGTGGACCACCAGCTCCTGTGGGGGGAGGCCCTG | |
| | CTCATCACCCCAGTGCTCCAGGCCGGGAAGGCCGAAGTGACTGG | |
| | CTACTTCCCCTTGGGCACATGGTACGACCTGCAGACGGTGCCAGT | |
| | AGAGGCCCTTGGCAGCCTCCCACCCCCACCTGCAGCTCCCCGTG | |
| | AGCCAGCCATCCACAGCGAGGGGCAGTGGGTGACGCTGCCGGCC | |
| | CCCCTGGACACCATCAACGTCCACCTCCGGGCTGGGTACATCATC | |
| | CCCCTGCAGGGCCCTGGCCTCACAACCACAGAGTCCCGCCAGCA | |
| | GCCCATGGCCCTGGCTGTGGCCCTGACCAAGGGTGGGGAGGCCC | |
| | GAGGGGAGCTTTTCTGGGACGATGGAGAGAGCCTGGAAGTGCTG | |
| | GAGCGAGGGGCCTACACACAGGTCATCTTCCTGGCCAGGAATAA | |
| | CACGATCGTGAATGAGCTGGTACGTGTGACCAGTGAGGGAGCTG | |
| | GCCTGCAGCTGCAGAAGGTGACTGTCCTGGGCGTGGCCACGGCG | |
| | CCCCAGCAGGTCCTCTCCAACGGTGTCCCTGTCTCCAACTTCACC | |
| | TACAGCCCCGACACCAAGGTCCTGGACATCTGTGTCTCGCTGTTG | |
| | ATGGGAGAGCAGTTTCTCGTCAGCTGGTGTTAG | |
| Kozak BiP- | GCAAGATGAAGCTCTCCCTGGTGGCCGCGATGCTGCTGCTGCTC | 190 |
| vIGF2-GAA | AGCGCGGCGCGGGCCTCTAGAACACTGTGCGGAGGGGAGCTTGT | |
| (“Engineered | AGACACTCTTCAGTTCGTGTGTGGAGATCGCGGGTTCCTCTTCTC | |
| hGAA”) | TCGCCCCGCTTCCAGAGTTTCACGGAGGTCTAGGGGTATAGTAG | |
| | AGGAGTGTTGTTTCAGGTCCTGTGACTTGGCGCTCCTCGAGACCT | |
| | ATTGCGCGACGCCAGCCAGGTCCGAAGGGGGCGGTGGCTCAGGT | |
| | GGTGGAGGTAGCAGACCAGGGCCCCGGGATGCCCAGGCACACC | |
| | CCGGCCGTCCCAGAGCAGTGCCCACACAGTGCGACGTCCCCCCC | |
| | AACAGCCGCTTCGATTGCGCCCCTGACAAGGCCATCACCCAGGA | |
| | ACAGTGCGAGGCCCGCGGCTGTTGCTACATCCCTGCAAAGCAGG | |
| | GGCTGCAGGGAGCCCAGATGGGGCAGCCCTGGTGCTTCTTCCCA | |
| | CCCAGCTACCCCAGCTACAAGCTGGAGAACCTGAGCTCCTCTGA | |
| | AATGGGCTACACGGCCACCCTGACCCGTACCACCCCCACCTTCTT | |
| | CCCCAAGGACATCCTGACCCTGCGGCTGGACGTGATGATGGAGA | |
| | CTGAGAACCGCCTCCACTTCACGATCAAAGATCCAGCTAACAGG | |
| | CGCTACGAGGTGCCCTTGGAGACCCCGCATGTCCACAGCCGGGC | |
| | ACCGTCCCCACTCTACAGCGTGGAGTTCTCCGAGGAGCCCTTCGG | |
| | GGTGATCGTGCGCCGGCAGCTGGACGGCCGCGTGCTGCTGAACA | |
| | CGACGGTGGCGCCCCTGTTCTTTGCGGACCAGTTCCTTCAGCTGT | |
| | CCACCTCGCTGCCCTCGCAGTATATCACCGGCCTCGCCGAGCACC | |
| | TCAGTCCCCTGATGCTCAGCACCAGCTGGACCAGGATCACCCTGT | |
| | GGAACCGGGACCTTGCGCCCACGCCCGGTGCGAACCTCTACGGG | |
| | TCTCACCCTTTCTACCTGGCGCTGGAGGACGGCGGGTCGGCACA | |
| | CGGGGTGTTCCTGCTAAACAGCAATGCCATGGATGTGGTCCTGC | |
| | AGCCGAGCCCTGCCCTTAGCTGGAGGTCGACAGGTGGGATCCTG | |
| | GATGTCTACATCTTCCTGGGCCCAGAGCCCAAGAGCGTGGTGCA | |
| | GCAGTACCTGGACGTTGTGGGATACCCGTTCATGCCGCCATACTG | |
| | GGGCCTGGGCTTCCACCTGTGCCGCTGGGGCTACTCCTCCACCGC | |
| | TATCACCCGCCAGGTGGTGGAGAACATGACCAGGGCCCACTTCC | |
| | CCCTGGACGTCCAGTGGAACGACCTGGACTACATGGACTCCCGG | |
| | AGGGACTTCACGTTCAACAAGGATGGCTTCCGGGACTTCCCGGC | |
| | CATGGTGCAGGAGCTGCACCAGGGCGGCCGGCGCTACATGATGA | |
| | TCGTGGATCCTGCCATCAGCAGCTCGGGCCCTGCCGGGAGCTAC | |
| | AGGCCCTACGACGAGGGTCTGCGGAGGGGGGTTTTCATCACCAA | |
| | CGAGACCGGCCAGCCGCTGATTGGGAAGGTATGGCCCGGGTCCA | |
| | CTGCCTTCCCCGACTTCACCAACCCCACAGCCCTGGCCTGGTGGG | |
| | AGGACATGGTGGCTGAGTTCCATGACCAGGTGCCCTTCGACGGC | |
| | ATGTGGATTGACATGAACGAGCCTTCCAACTTCATCAGGGGCTCT | |
| | GAGGACGGCTGCCCCAACAATGAGCTGGAGAACCCACCCTACGT | |
| | GCCTGGGGTGGTTGGGGGGACCCTCCAGGCGGCCACCATCTGTG | |
| | CCTCCAGCCACCAGTTTCTCTCCACACACTACAACCTGCACAACC | |
| | TCTACGGCCTGACCGAAGCCATCGCCTCCCACAGGGCGCTGGTG | |
| | AAGGCTCGGGGGACACGCCCATTTGTGATCTCCCGCTCGACCTTT | |
| | GCTGGCCACGGCCGATACGCCGGCCACTGGACGGGGGACGTGTG | |
| | GAGCTCCTGGGAGCAGCTCGCCTCCTCCGTGCCAGAAATCCTGC | |
| | AGTTTAACCTGCTGGGGGTGCCTCTGGTCGGGGCCGACGTCTGC | |
| | GGCTTCCTGGGCAACACCTCAGAGGAGCTGTGTGTGCGCTGGAC | |
| | CCAGCTGGGGGCCTTCTACCCCTTCATGCGGAACCACAACAGCC | |
| | TGCTCAGTCTGCCCCAGGAGCCGTACAGCTTCAGCGAGCCGGCC | |
| | CAGCAGGCCATGAGGAAGGCCCTCACCCTGCGCTACGCACTCCT | |
| | CCCCCACCTCTACACACTGTTCCACCAGGCCCACGTCGCGGGGG | |
| | AGACCGTGGCCCGGCCCCTCTTCCTGGAGTTCCCCAAGGACTCTA | |
| | GCACCTGGACTGTGGACCACCAGCTCCTGTGGGGGGAGGCCCTG | |
| | CTCATCACCCCAGTGCTCCAGGCCGGGAAGGCCGAAGTGACTGG | |
| | CTACTTCCCCTTGGGCACATGGTACGACCTGCAGACGGTGCCAGT | |
| | AGAGGCCCTTGGCAGCCTCCCACCCCCACCTGCAGCTCCCCGTG | |
| | AGCCAGCCATCCACAGCGAGGGGCAGTGGGTGACGCTGCCGGCC | |
| | CCCCTGGACACCATCAACGTCCACCTCCGGGCTGGGTACATCATC | |
| | CCCCTGCAGGGCCCTGGCCTCACAACCACAGAGTCCCGCCAGCA | |
| | GCCCATGGCCCTGGCTGTGGCCCTGACCAAGGGTGGGGAGGCCC | |
| | GAGGGGAGCTGTTCTGGGACGATGGAGAGAGCCTGGAAGTGCTG | |
| | GAGCGAGGGGCCTACACACAGGTCATCTTCCTGGCCAGGAATAA | |
| | CACGATCGTGAATGAGCTGGTACGTGTGACCAGTGAGGGAGCTG | |
| | GCCTGCAGCTGCAGAAGGTGACTGTCCTGGGCGTGGCCACGGCG | |
| | CCCCAGCAGGTCCTCTCCAACGGTGTCCCTGTCTCCAACTTCACC | |
| | TACAGCCCCGACACCAAGGTCCTGGACATCTGTGTCTCGCTGTTG | |
| | ATGGGAGAGCAGTTTCTCGTCAGCTGGTGTTAG | |
| Cricket | AAAAATGTGATCTTGCTTGTAAATACAATTTTGAGAGGTTAATAAATTA | 191 |
| Paralysis | CAAGTAGTGCTATTTTTGTATTTAGGTTAGCTATTTAGCTTTACGTTCC | |
| Virus IRES | AGGATGCCTAGTGGCAGCCCCACAATATCCAGGAAGCCCTCTCTGCG | |
| (underlined)- | GTTTTTCAGATTAGGTAGTCGAAAAACCTAAGAAATTTACCTGCTATG | |
| BiP-vIGF2- | AAGCTCTCCCTGGTGGCCGCGATGCTGCTGCTGCTCAGCGCGGC | |
| GAA | GCGGGCCTCTAGAACACTGTGCGGAGGGGAGCTTGTAGACACTC | |
| | TTCAGTTCGTGTGTGGAGATCGCGGGTTCCTCTTCTCTCGCCCCG | |
| | CTTCCAGAGTTTCACGGAGGTCTAGGGGTATAGTAGAGGAGTGT | |
| | TGTTTCAGGTCCTGTGACTTGGCGCTCCTCGAGACCTATTGCGCG | |
| | ACGCCAGCCAGGTCCGAAGGGGGCGGTGGCTCAGGTGGTGGAG | |
| | GTAGCAGACCAGGGCCCCGGGATGCCCAGGCACACCCCGGCCGT | |
| | CCCAGAGCAGTGCCCACACAGTGCGACGTCCCCCCCAACAGCCG | |
| | CTTCGATTGCGCCCCTGACAAGGCCATCACCCAGGAACAGTGCG | |
| | AGGCCCGCGGCTGTTGCTACATCCCTGCAAAGCAGGGGCTGCAG | |
| | GGAGCCCAGATGGGGCAGCCCTGGTGCTTCTTCCCACCCAGCTA | |
| | CCCCAGCTACAAGCTGGAGAACCTGAGCTCCTCTGAAATGGGCT | |
| | ACACGGCCACCCTGACCCGTACCACCCCCACCTTCTTCCCCAAGG | |
| | ACATCCTGACCCTGCGGCTGGACGTGATGATGGAGACTGAGAAC | |
| | CGCCTCCACTTCACGATCAAAGATCCAGCTAACAGGCGCTACGA | |
| | GGTGCCCTTGGAGACCCCGCATGTCCACAGCCGGGCACCGTCCC | |
| | CACTCTACAGCGTGGAGTTCTCCGAGGAGCCCTTCGGGGTGATC | |
| | GTGCGCCGGCAGCTGGACGGCCGCGTGCTGCTGAACACGACGGT | |
| | GGCGCCCCTGTTCTTTGCGGACCAGTTCCTTCAGCTGTCCACCTC | |
| | GCTGCCCTCGCAGTATATCACAGGCCTCGCCGAGCACCTCAGTCC | |
| | CCTGATGCTCAGCACCAGCTGGACCAGGATCACCCTGTGGAACC | |
| | GGGACCTTGCGCCCACGCCCGGTGCGAACCTCTACGGGTCTCAC | |
| | CCTTTCTACCTGGCGCTGGAGGACGGCGGGTCGGCACACGGGGT | |
| | GTTCCTGCTAAACAGCAATGCCATGGATGTGGTCCTGCAGCCGA | |
| | GCCCTGCCCTTAGCTGGAGGTCGACAGGTGGGATCCTGGATGTC | |
| | TACATCTTCCTGGGCCCAGAGCCCAAGAGCGTGGTGCAGCAGTA | |
| | CCTGGACGTTGTGGGATACCCGTTCATGCCGCCATACTGGGGCCT | |
| | GGGCTTCCACCTGTGCCGCTGGGGCTACTCCTCCACCGCTATCAC | |
| | CCGCCAGGTGGTGGAGAACATGACCAGGGCCCACTTCCCCCTGG | |
| | ACGTCCAGTGGAACGACCTGGACTACATGGACTCCCGGAGGGAC | |
| | TTCACGTTCAACAAGGATGGCTTCCGGGACTTCCCGGCCATGGTG | |
| | CAGGAGCTGCACCAGGGCGGCCGGCGCTACATGATGATCGTGGA | |
| | TCCTGCCATCAGCAGCTCGGGCCCTGCCGGGAGCTACAGGCCCT | |
| | ACGACGAGGGTCTGCGGAGGGGGGTTTTCATCACCAACGAGACC | |
| | GGCCAGCCGCTGATTGGGAAGGTATGGCCCGGGTCCACTGCCTT | |
| | CCCCGACTTCACCAACCCCACAGCCCTGGCCTGGTGGGAGGACA | |
| | TGGTGGCTGAGTTCCATGACCAGGTGCCCTTCGACGGCATGTGG | |
| | ATTGACATGAACGAGCCTTCCAACTTCATCAGGGGCTCTGAGGA | |
| | CGGCTGCCCCAACAATGAGCTGGAGAACCCACCCTACGTGCCTG | |
| | GGGTGGTTGGGGGGACCCTCCAGGCGGCCACCATCTGTGCCTCC | |
| | AGCCACCAGTTTCTCTCCACACACTACAACCTGCACAACCTCTAC | |
| | GGCCTGACCGAAGCCATCGCCTCCCACAGGGCGCTGGTGAAGGC | |
| | TCGGGGGACACGCCCATTTGTGATCTCCCGCTCGACCTTTGCTGG | |
| | CCACGGCCGATACGCCGGCCACTGGACGGGGGACGTGTGGAGCT | |
| | CCTGGGAGCAGCTCGCCTCCTCCGTGCCAGAAATCCTGCAGTTTA | |
| | ACCTGCTGGGGGTGCCTCTGGTCGGGGCCGACGTCTGCGGCTTCC | |
| | TGGGCAACACCTCAGAGGAGCTGTGTGTGCGCTGGACCCAGCTG | |
| | GGGGCCTTCTACCCCTTCATGCGGAACCACAACAGCCTGCTCAGT | |
| | CTGCCCCAGGAGCCGTACAGCTTCAGCGAGCCGGCCCAGCAGGC | |
| | CATGAGGAAGGCCCTCACCCTGCGCTACGCACTCCTCCCCCACCT | |
| | CTACACACTGTTCCACCAGGCCCACGTCGCGGGGGAGACCGTGG | |
| | CCCGGCCCCTCTTCCTGGAGTTCCCCAAGGACTCTAGCACCTGGA | |
| | CTGTGGACCACCAGCTCCTGTGGGGGGAGGCCCTGCTCATCACC | |
| | CCAGTGCTCCAGGCCGGGAAGGCCGAAGTGACTGGCTACTTCCC | |
| | CTTGGGCACATGGTACGACCTGCAGACGGTGCCAGTAGAGGCCC | |
| | TTGGCAGCCTCCCACCCCCACCTGCAGCTCCCCGTGAGCCAGCCA | |
| | TCCACAGCGAGGGGCAGTGGGTGACGCTGCCGGCCCCCCTGGAC | |
| | ACCATCAACGTCCACCTCCGGGCTGGGTACATCATCCCCCTGCAG | |
| | GGCCCTGGCCTCACAACCACAGAGTCCCGCCAGCAGCCCATGGC | |
| | CCTGGCTGTGGCCCTGACCAAGGGTGGGGAGGCCCGAGGGGAGC | |
| | TGTTCTGGGACGATGGAGAGAGCCTGGAAGTGCTGGAGCGAGGG | |
| | GCCTACACACAGGTCATCTTCCTGGCCAGGAATAACACGATCGT | |
| | GAATGAGCTGGTACGTGTGACCAGTGAGGGAGCTGGCCTGCAGC | |
| | TGCAGAAGGTGACTGTCCTGGGCGTGGCCACGGCGCCCCAGCAG | |
| | GTCCTCTCCAACGGTGTCCCTGTCTCCAACTTCACCTACAGCCCC | |
| | GACACCAAGGTCCTGGACATCTGTGTCTCGCTGTTGATGGGAGA | |
| | GCAGTTTCTCGTCAGCTGGTGTTAG | |
| wt-PPT1 | ATGGCATCACCGGGTTGCCTCTGGTTGTTGGCCGTTGCGTTGCTT | 192 |
| IDT codon | CCGTGGACATGTGCATCAAGAGCTCTTCAACATCTGGATCCCCCA | |
| optimized | GCTCCCCTGCCGCTCGTAATCTGGCACGGGATGGGGGATTCATGT | |
| | TGTAACCCGTTGTCAATGGGCGCGATAAAAAAGATGGTTGAAAA | |
| | GAAGATTCCAGGCATCTACGTTCTGTCCCTGGAAATCGGTAAGA | |
| | CACTGATGGAAGACGTGGAGAACTCCTTCTTTCTCAACGTCAATA | |
| | GTCAGGTCACTACCGTCTGTCAAGCATTGGCAAAGGACCCTAAA | |
| | CTTCAGCAGGGGTACAATGCGATGGGGTTTAGCCAGGGCGGACA | |
| | GTTTCTTAGAGCCGTCGCACAGCGCTGTCCATCTCCCCCGATGAT | |
| | TAACCTTATATCTGTCGGGGGACAACACCAGGGTGTTTTTGGTCT | |
| | TCCTCGCTGTCCTGGTGAAAGCTCCCACATCTGTGATTTCATACG | |
| | CAAAACGTTGAACGCAGGAGCTTATAGTAAAGTCGTCCAAGAAC | |
| | GGCTTGTTCAAGCGGAGTATTGGCATGACCCAATAAAAGAAGAC | |
| | GTTTATAGGAATCACTCTATCTTCTTGGCCGATATCAACCAAGAA | |
| | CGCGGAATCAACGAAAGCTACAAAAAGAATCTTATGGCTCTCAA | |
| | GAAATTTGTTATGGTGAAATTCCTTAATGACTCTATAGTAGATCC | |
| | TGTCGATTCAGAATGGTTCGGGTTCTACAGGTCTGGCCAGGCGA | |
| | AGGAGACTATTCCCCTCCAAGAAACGTCTCTCTATACACAAGAC | |
| | AGACTCGGACTGAAAGAGATGGATAATGCGGGCCAGTTGGTCTT | |
| | CTTGGCTACGGAAGGCGATCATCTCCAACTCTCCGAAGAGTGGT | |
| | TCTATGCCCATATAATCCCGTTCCTGGGCTAA | |
| PPT1-2 (wt- | ATGGCATCCCCCGGATGTTTGTGGCTGCTGGCGGTTGCGCTTCTG | 193 |
| vIGF2- | CCATGGACGTGCGCCTCCCGAGCCCTCCAACACCTGTCCAGGAC | |
| PPT1; | ACTTTGCGGCGGAGAGTTGGTCGATACGCTTCAATTCGTGTGTGG | |
| Codon | GGATAGAGGCTTCCTTTTTTCTCGGCCCGCTAGCCGCGTGTCCCG | |
| optimized | AAGGTCCCGGGGTATCGTTGAGGAATGCTGTTTCCGGTCCTGCG | |
| by IDT | ATCTTGCACTGTTGGAGACATACTGTGCTACGCCTGCGAGAAGC | |
| codon | GAGGGTGGAGGGGGTTCTGGAGGTGGAGGGAGCCGGCCTCGGG | |
| optimization | CGGTTCCCACCCAGGATCCTCCAGCTCCTCTGCCTCTGGTCATCT | |
| tool) | GGCATGGGATGGGGGACTCATGTTGTAACCCGCTGAGTATGGGG | |
| | GCAATTAAAAAAATGGTTGAAAAGAAAATTCCAGGTATTTATGT | |
| | CCTCTCTCTTGAAATCGGTAAGACACTTATGGAGGATGTGGAAA | |
| | ACTCCTTTTTCCTTAATGTCAATTCTCAGGTCACAACAGTTTGTCA | |
| | GGCTCTGGCGAAGGATCCTAAGCTGCAGCAAGGCTACAACGCCA | |
| | TGGGTTTTTCCCAGGGAGGCCAATTTCTCAGAGCGGTAGCTCAGC | |
| | GATGTCCATCACCACCGATGATAAATCTGATCAGTGTCGGCGGA | |
| | CAACACCAGGGAGTTTTCGGGCTGCCCAGGTGTCCGGGGGAATC | |
| | TAGTCACATATGTGACTTCATTCGCAAGACCCTTAACGCCGGCGC | |
| | TTACTCAAAGGTGGTTCAAGAACGGCTTGTGCAGGCTGAATACT | |
| | GGCACGATCCCATCAAGGAAGATGTATATAGGAACCACAGTATC | |
| | TTTCTGGCAGACATAAATCAGGAAAGGGGTATTAACGAAAGCTA | |
| | CAAGAAAAATCTCATGGCCCTGAAGAAATTTGTAATGGTTAAGT | |
| | TTTTGAACGATTCTATAGTAGATCCTGTTGACTCCGAGTGGTTCG | |
| | GGTTCTATCGATCTGGTCAAGCCAAGGAGACGATTCCGCTTCAG | |
| | GAAACTTCACTGTACACACAGGATCGGCTGGGACTCAAGGAGAT | |
| | GGACAATGCGGGCCAGTTGGTGTTTCTGGCTACAGAGGGAGACC | |
| | ATCTCCAGTTGAGTGAAGAATGGTTCTATGCACATATTATCCCAT | |
| | TCCTCGGCTAA | |
| PPT1-29 | ATGAAGCTCTCCCTGGTGGCCGCGATGCTGCTGCTGCTCTGGGTG | 194 |
| (BiP2aa- | GCACTGCTGCTGCTCAGCGCGGCGAGGGCCGCCGCGAGTCGCAC | |
| vIGF2- | GTTGTGTGGAGGTGAACTCGTCGACACCCTTCAGTTCGTATGTGG | |
| PPT1; | AGATCGCGGTTTCCTCTTCTCACGCCCAGCTTCCAGAGTTTCCCG | |
| native | AAGATCACGAGGAATAGTTGAGGAGTGCTGTTTTCGGTCTTGTG | |
| human | ATCTGGCTCTCCTCGAGACTTATTGTGCTACGCCGGCCCGCTCTG | |
| sequence) | AAGGAGGTGGTGGCAGTGGAGGAGGAGGGAGTCGGCCTAGGGC | |
| | AGTCCCAACCCAGGACCCGCCGGCGCCGCTGCCGTTGGTGATCT | |
| | GGCATGGGATGGGAGACAGCTGTTGCAATCCCTTAAGCATGGGT | |
| | GCTATTAAAAAAATGGTGGAGAAGAAAATACCTGGAATTTACGT | |
| | CTTATCTTTAGAGATTGGGAAGACCCTGATGGAGGACGTGGAGA | |
| | ACAGCTTCTTCTTGAATGTCAATTCCCAAGTAACAACAGTGTGTC | |
| | AGGCACTTGCTAAGGATCCTAAATTGCAGCAAGGCTACAATGCT | |
| | ATGGGATTCTCCCAGGGAGGCCAATTTCTGAGGGCAGTGGCTCA | |
| | GAGATGCCCTTCACCTCCCATGATCAATCTGATCTCGGTTGGGGG | |
| | ACAACATCAAGGTGTTTTTGGACTCCCTCGATGCCCAGGAGAGA | |
| | GCTCTCACATCTGTGACTTCATCCGAAAAACACTGAATGCTGGG | |
| | GCGTACTCCAAAGTTGTTCAGGAACGCCTCGTGCAAGCCGAATA | |
| | CTGGCATGACCCCATAAAGGAGGATGTGTATCGCAACCACAGCA | |
| | TCTTCTTGGCAGATATAAATCAGGAGCGGGGTATCAATGAGTCC | |
| | TACAAGAAAAACCTGATGGCCCTGAAGAAGTTTGTGATGGTGAA | |
| | ATTCCTCAATGATTCCATTGTGGACCCTGTAGATTCGGAGTGGTT | |
| | TGGATTTTACAGAAGTGGCCAAGCCAAGGAAACCATTCCCTTAC | |
| | AGGAGACCTCCCTGTACACACAGGACCGCCTGGGGCTAAAGGAA | |
| | ATGGACAATGCAGGACAGCTAGTGTTTCTGGCTACAGAAGGGGA | |
| | CCATCTTCAGTTGTCTGAAGAATGGTTTTATGCCCACATCATACC | |
| | ATTCCTTGGATGA | |
| PPT1 | ATGGCGTCGCCCGGCTGCCTGTGGCTCTTGGCTGTGGCTCTCCTG | 195 |
| engineered | CCATGGACCTGCGCTTCTCGGGCGCTGCAGCATCTGGACCCGCC | |
| | GGCGCCGCTGCCGTTGGTGATCTGGCATGGGATGGGAGACAGCT | |
| | GTTGCAATCCCTTAAGCATGGGTGCTATTAAAAAAATGGTGGAG | |
| | AAGAAAATACCTGGAATTTACGTCTTATCTTTAGAGATTGGGAA | |
| | GACCCTGATGGAGGACGTGGAGAACAGCTTCTTCTTGAATGTCA | |
| | ATTCCCAAGTAACAACAGTGTGTCAGGCACTTGCTAAGGATCCT | |
| | AAATTGCAGCAAGGCTACAATGCTATGGGATTCTCCCAGGGAGG | |
| | CCAATTTCTGAGGGCAGTGGCTCAGAGATGCCCTTCACCTCCCAT | |
| | GATCAATCTGATCTCGGTTGGGGGACAACATCAAGGTGTTTTTGG | |
| | ACTCCCTCGATGCCCAGGAGAGAGCTCTCACATCTGTGACTTCAT | |
| | CCGAAAAACACTGAATGCTGGGGCGTACTCCAAAGTTGTTCAGG | |
| | AACGCCTCGTGCAAGCCGAATACTGGCATGACCCCATAAAGGAG | |
| | GATGTGTATCGCAACCACAGCATCTTCTTGGCAGATATAAATCA | |
| | GGAGCGGGGTATCAATGAGTCCTACAAGAAAAACCTGATGGCCC | |
| | TGAAGAAGTTTGTGATGGTGAAATTCCTCAATGATTCCATTGTGG | |
| | ACCCTGTAGATTCGGAGTGGTTTGGATTTTACAGAAGTGGCCAA | |
| | GCCAAGGAAACCATTCCCTTACAGGAGACCTCCCTGTACACACA | |
| | GGACCGCCTGGGGCTAAAGGAAATGGACAATGCAGGACAGCTA | |
| | GTGTTTCTGGCTACAGAAGGGGACCATCTTCAGTTGTCTGAAGA | |
| | ATGGTTTTATGCCCACATCATACCATTCCTTGGAAGACCTAGAGC | |
| | AGTGCCTACGCAGGGAGGGAGTGGGAGTGGATCCACTTCATCCT | |
| | CTAGAACACTGTGCGGAGGGGAGCTTGTAGACACTCTTCAGTTC | |
| | GTGTGTGGAGATCGCGGGTTCCTCTTCTCTCGCCCCGCTTCCAGA | |
| | GTTTCACGGAGGTCTAGGGGTATAGTAGAGGAGTGTTGTTTCAG | |
| | GGAGTGTGACTTGGCGCTCCTCGAGACCTATTGCGCGACGCCAG | |
| | CCAGGTCCGAATGA | |
| TPP1 | ATGGGACTCCAAGCCTGCCTCCTAGGGCTCTTTGCCCTCATCCTC | 196 |
| wildtype | TCTGGCAAATGCAGTTACAGCCCGGAGCCCGACCAGCGGAGGAC | |
| | GCTGCCCCCAGGCTGGGTGTCCCTGGGCCGTGCGGACCCTGAGG | |
| | AAGAGCTGAGTCTCACCTTTGCCCTGAGACAGCAGAATGTGGAA | |
| | AGACTCTCGGAGCTGGTGCAGGCTGTGTCGGATCCCAGCTCTCCT | |
| | CAATACGGAAAATACCTGACCCTAGAGAATGTGGCTGATCTGGT | |
| | GAGGCCATCCCCACTGACCCTCCACACGGTGCAAAAATGGCTCT | |
| | TGGCAGCCGGAGCCCAGAAGTGCCATTCTGTGATCACACAGGAC | |
| | TTTCTGACTTGCTGGCTGAGCATCCGACAAGCAGAGCTGCTGCTC | |
| | CCTGGGGCTGAGTTTCATCACTATGTGGGAGGACCTACGGAAAC | |
| | CCATGTTGTAAGGTCCCCACATCCCTACCAGCTTCCACAGGCCTT | |
| | GGCCCCCCATGTGGACTTTGTGGGGGGACTGCACCGTTTTCCCCC | |
| | AACATCATCCCTGAGGCAACGTCCTGAGCCGCAGGTGACAGGGA | |
| | CTGTAGGCCTGCATCTGGGGGTAACCCCCTCTGTGATCCGTAAGC | |
| | GATACAACTTGACCTCACAAGACGTGGGCTCTGGCACCAGCAAT | |
| | AACAGCCAAGCCTGTGCCCAGTTCCTGGAGCAGTATTTCCATGA | |
| | CTCAGACCTGGCTCAGTTCATGCGCCTCTTCGGTGGCAACTTTGC | |
| | ACATCAGGCATCAGTAGCCCGTGTGGTTGGACAACAGGGCCGGG | |
| | GCCGGGCCGGGATTGAGGCCAGTCTAGATGTGCAGTACCTGATG | |
| | AGTGCTGGTGCCAACATCTCCACCTGGGTCTACAGTAGCCCTGGC | |
| | CGGCATGAGGGACAGGAGCCCTTCCTGCAGTGGCTCATGCTGCT | |
| | CAGTAATGAGTCAGCCCTGCCACATGTGCATACTGTGAGCTATG | |
| | GAGATGATGAGGACTCCCTCAGCAGCGCCTACATCCAGCGGGTC | |
| | AACACTGAGCTCATGAAGGCTGCCGCTCGGGGTCTCACCCTGCT | |
| | CTTCGCCTCAGGTGACAGTGGGGCCGGGTGTTGGTCTGTCTCTGG | |
| | AAGACACCAGTTCCGCCCTACCTTCCCTGCCTCCAGCCCCTATGT | |
| | CACCACAGTGGGAGGCACATCCTTCCAGGAACCTTTCCTCATCAC | |
| | AAATGAAATTGTTGACTATATCAGTGGTGGTGGCTTCAGCAATGT | |
| | GTTCCCACGGCCTTCATACCAGGAGGAAGCTGTAACGAAGTTCC | |
| | TGAGCTCTAGCCCCCACCTGCCACCATCCAGTTACTTCAATGCCA | |
| | GTGGCCGTGCCTACCCAGATGTGGCTGCACTTTCTGATGGCTACT | |
| | GGGTGGTCAGCAACAGAGTGCCCATTCCATGGGTGTCCGGAACC | |
| | TCGGCCTCTACTCCAGTGTTTGGGGGGATCCTATCCTTGATCAAT | |
| | GAGCACAGGATCCTTAGTGGCCGCCCCCCTCTTGGCTTTCTCAAC | |
| | CCAAGGCTCTACCAGCAGCATGGGGCAGGACTCTTTGATGTAAC | |
| | CCGTGGCTGCCATGAGTCCTGTCTGGATGAAGAGGTAGAGGGCC | |
| | AGGGTTTCTGCTCTGGTCCTGGCTGGGATCCTGTAACAGGCTGGG | |
| | GAACACCCAACTTCCCAGCTTTGCTGAAGACTCTACTCAACCCCT | |
| | GA | |
| TPP1 | ATGGGACTCCAAGCCTGCCTCCTAGGGCTCTTTGCCCTCATCCTC | 197 |
| engineered | TCTGGCAAATGCTCTAGAACACTGTGCGGAGGGGAGCTTGTAGA | |
| | CACTCTTCAGTTCGTGTGTGGAGATCGCGGGTTCCTCTTCTCTCG | |
| | CCCCGCTTCCAGAGTTTCACGGAGGTCTAGGGGTATAGTAGAGG | |
| | AGTGTTGTTTCAGGTCCTGTGACTTGGCGCTCCTCGAGACCTATT | |
| | GCGCGACGCCAGCCAGGTCCGAAGGAGGTGGTGGCAGTGGAGG | |
| | AGGAGGGAGTAGACCTAGAGCAGTGCCTACGCAGAGTTACAGCC | |
| | CGGAGCCCGACCAGCGGAGGACGCTGCCCCCAGGCTGGGTGTCC | |
| | CTGGGCCGTGCGGACCCTGAGGAAGAGCTGAGTCTCACCTTTGC | |
| | CCTGAGACAGCAGAATGTGGAAAGACTCTCGGAGCTGGTGCAGG | |
| | CTGTGTCGGATCCCAGCTCTCCTCAATACGGAAAATACCTGACCC | |
| | TAGAGAATGTGGCTGATCTGGTGAGGCCATCCCCACTGACCCTC | |
| | CACACGGTGCAAAAATGGCTCTTGGCAGCCGGAGCCCAGAAGTG | |
| | CCATTCTGTGATCACACAGGACTTTCTGACTTGCTGGCTGAGCAT | |
| | CCGACAAGCAGAGCTGCTGCTCCCTGGGGCTGAGTTTCATCACT | |
| | ATGTGGGAGGACCTACGGAAACCCATGTTGTAAGGTCCCCACAT | |
| | CCCTACCAGCTTCCACAGGCCTTGGCCCCCCATGTGGACTTTGTG | |
| | GGGGGACTGCACCGTTTTCCCCCAACATCATCCCTGAGGCAACG | |
| | TCCTGAGCCGCAGGTGACAGGGACTGTAGGCCTGCATCTGGGGG | |
| | TAACCCCCTCTGTGATCCGTAAGCGATACAACTTGACCTCACAAG | |
| | ACGTGGGCTCTGGCACCAGCAATAACAGCCAAGCCTGTGCCCAG | |
| | TTCCTGGAGCAGTATTTCCATGACTCAGACCTGGCTCAGTTCATG | |
| | CGCCTCTTCGGTGGCAACTTTGCACATCAGGCATCAGTAGCCCGT | |
| | GTGGTTGGACAACAGGGCCGGGGCCGGGCCGGGATTGAGGCCA | |
| | GTCTAGATGTGCAGTACCTGATGAGTGCTGGTGCCAACATCTCCA | |
| | CCTGGGTCTACAGTAGCCCTGGCCGGCATGAGGGACAGGAGCCC | |
| | TTCCTGCAGTGGCTCATGCTGCTCAGTAATGAGTCAGCCCTGCCA | |
| | CATGTGCATACTGTGAGCTATGGAGATGATGAGGACTCCCTCAG | |
| | CAGCGCCTACATCCAGCGGGTCAACACTGAGCTCATGAAGGCTG | |
| | CCGCTCGGGGTCTCACCCTGCTCTTCGCCTCAGGTGACAGTGGGG | |
| | CCGGGTGTTGGTCTGTCTCTGGAAGACACCAGTTCCGCCCTACCT | |
| | TCCCTGCCTCCAGCCCCTATGTCACCACAGTGGGAGGCACATCCT | |
| | TCCAGGAACCTTTCCTCATCACAAATGAAATTGTTGACTATATCA | |
| | GTGGTGGTGGCTTCAGCAATGTGTTCCCACGGCCTTCATACCAGG | |
| | AGGAAGCTGTAACGAAGTTCCTGAGCTCTAGCCCCCACCTGCCA | |
| | CCATCCAGTTACTTCAATGCCAGTGGCCGTGCCTACCCAGATGTG | |
| | GCTGCACTTTCTGATGGCTACTGGGTGGTCAGCAACAGAGTGCC | |
| | CATTCCATGGGTGTCCGGAACCTCGGCCTCTACTCCAGTGTTTGG | |
| | GGGGATCCTATCCTTGATCAATGAGCACAGGATCCTTAGTGGCC | |
| | GCCCCCCTCTTGGCTTTCTCAACCCAAGGCTCTACCAGCAGCATG | |
| | GGGCAGGACTCTTTGATGTAACCCGTGGCTGCCATGAGTCCTGTC | |
| | TGGATGAAGAGGTAGAGGGCCAGGGTTTCTGCTCTGGTCCTGGC | |
| | TGGGATCCTGTAACAGGCTGGGGAACACCCAACTTCCCAGCTTT | |
| | GCTGAAGACTCTACTCAACCCCTGA | |
| AGA | ATGGCGCGGAAGTCGAACTTGCCTGTGCTTCTCGTGCCGTTTCTG | 198 |
| wildtype | CTCTGCCAGGCCCTAGTGCGCTGCTCCAGCCCTCTGCCCCTGGTC | |
| | GTCAACACTTGGCCCTTTAAGAATGCAACCGAAGCAGCGTGGAG | |
| | GGCATTAGCATCTGGAGGCTCTGCCCTGGATGCAGTGGAGAGCG | |
| | GCTGTGCCATGTGTGAGAGAGAGCAGTGTGACGGCTCTGTAGGC | |
| | TTTGGAGGAAGTCCTGATGAACTTGGAGAAACCACACTAGATGC | |
| | CATGATCATGGATGGCACTACTATGGATGTAGGAGCAGTAGGAG | |
| | ATCTCAGACGAATTAAAAATGCTATTGGTGTGGCACGGAAAGTA | |
| | CTGGAACATACAACACACACACTTTTAGTAGGAGAGTCAGCCAC | |
| | CACATTTGCTCAAAGTATGGGGTTTATCAATGAAGACTTATCTAC | |
| | CACTGCTTCTCAAGCTCTTCATTCAGATTGGCTTGCTCGGAATTG | |
| | CCAGCCAAATTATTGGAGGAATGTTATACCAGATCCCTCAAAAT | |
| | ACTGCGGACCCTACAAACCACCTGGTATCTTAAAGCAGGATATT | |
| | CCTATCCATAAAGAAACAGAAGATGATCGTGGTCATGACACTAT | |
| | TGGCATGGTTGTAATCCATAAGACAGGACATATTGCTGCTGGTA | |
| | CATCTACAAATGGTATAAAATTCAAAATACATGGCCGTGTAGGA | |
| | GACTCACCAATACCTGGAGCTGGAGCCTATGCTGACGATACTGC | |
| | AGGGGCAGCCGCAGCCACTGGGAATGGTGATATATTGATGCGCT | |
| | TCCTGCCAAGCTACCAAGCTGTAGAATACATGAGAAGAGGAGAA | |
| | GATCCAACCATAGCTTGCCAAAAAGTGATTTCAAGAATCCAGAA | |
| | GCATTTTCCAGAATTCTTTGGGGCTGTTATATGTGCCAATGTGAC | |
| | TGGAAGTTACGGTGCTGCTTGCAATAAACTTTCAACATTTACTCA | |
| | GTTTAGTTTCATGGTTTATAATTCCGAAAAAAATCAGCCAACTGA | |
| | GGAAAAAGTGGACTGCATCTAA | |
| AGA | ATGGCGCGGAAGTCGAACTTGCCTGTGCTTCTCGTGCCGTTTCTG | 199 |
| engineered | CTCTGCCAGGCCCTAGTGCGCTGCTCTAGAACACTGTGCGGAGG | |
| (N-terminal | GGAGCTTGTAGACACTCTTCAGTTCGTGTGTGGAGATCGCGGGTT | |
| fusion) | CCTCTTCTCTCGCCCCGCTTCCAGAGTTTCACGGAGGTCTAGGGG | |
| | TATAGTAGAGGAGTGTTGTTTCAGGTCCTGTGACTTGGCGCTCCT | |
| | CGAGACCTATTGCGCGACGCCAGCCAGGTCCGAAGGAGGTGGTG | |
| | GCAGTGGAGGAGGAGGGAGTAGACCTAGAGCAGTGCCTACGCA | |
| | GTCCAGCCCTCTGCCCCTGGTCGTCAACACTTGGCCCTTTAAGAA | |
| | TGCAACCGAAGCAGCGTGGAGGGCATTAGCATCTGGAGGCTCTG | |
| | CCCTGGATGCAGTGGAGAGCGGCTGTGCCATGTGTGAGAGAGAG | |
| | CAGTGTGACGGCTCTGTAGGCTTTGGAGGAAGTCCTGATGAACT | |
| | TGGAGAAACCACACTAGATGCCATGATCATGGATGGCACTACTA | |
| | TGGATGTAGGAGCAGTAGGAGATCTCAGACGAATTAAAAATGCT | |
| | ATTGGTGTGGCACGGAAAGTACTGGAACATACAACACACACACT | |
| | TTTAGTAGGAGAGTCAGCCACCACATTTGCTCAAAGTATGGGGT | |
| | TTATCAATGAAGACTTATCTACCACTGCTTCTCAAGCTCTTCATT | |
| | CAGATTGGCTTGCTCGGAATTGCCAGCCAAATTATTGGAGGAAT | |
| | GTTATACCAGATCCCTCAAAATACTGCGGACCCTACAAACCACC | |
| | TGGTATCTTAAAGCAGGATATTCCTATCCATAAAGAAACAGAAG | |
| | ATGATCGTGGTCATGACACTATTGGCATGGTTGTAATCCATAAGA | |
| | CAGGACATATTGCTGCTGGTACATCTACAAATGGTATAAAATTC | |
| | AAAATACATGGCCGTGTAGGAGACTCACCAATACCTGGAGCTGG | |
| | AGCCTATGCTGACGATACTGCAGGGGCAGCCGCAGCCACTGGGA | |
| | ATGGTGATATATTGATGCGCTTCCTGCCAAGCTACCAAGCTGTAG | |
| | AATACATGAGAAGAGGAGAAGATCCAACCATAGCTTGCCAAAA | |
| | AGTGATTTCAAGAATCCAGAAGCATTTTCCAGAATTCTTTGGGGC | |
| | TGTTATATGTGCCAATGTGACTGGAAGTTACGGTGCTGCTTGCAA | |
| | TAAACTTTCAACATTTACTCAGTTTAGTTTCATGGTTTATAATTCC | |
| | GAAAAAAATCAGCCAACTGAGGAAAAAGTGGACTGCATCTAA | |
| GLA | ATGAAGCTCTCCCTGGTGGCCGCGATGCTGCTGCTGCTCAGCGCG | 200 |
| wildtype | GCGCGGGCCCTGGACAATGGATTGGCAAGGACGCCTACCATGGG | |
| | CTGGCTGCACTGGGAGCGCTTCATGTGCAACCTTGACTGCCAGG | |
| | AAGAGCCAGATTCCTGCATCAGTGAGAAGCTCTTCATGGAGATG | |
| | GCAGAGCTCATGGTCTCAGAAGGCTGGAAGGATGCAGGTTATGA | |
| | GTACCTCTGCATTGATGACTGTTGGATGGCTCCCCAAAGAGATTC | |
| | AGAAGGCAGACTTCAGGCAGACCCTCAGCGCTTTCCTCATGGGA | |
| | TTCGCCAGCTAGCTAATTATGTTCACAGCAAAGGACTGAAGCTA | |
| | GGGATTTATGCAGATGTTGGAAATAAAACCTGCGCAGGCTTCCC | |
| | TGGGAGTTTTGGATACTACGACATTGATGCCCAGACCTTTGCTGA | |
| | CTGGGGAGTAGATCTGCTAAAATTTGATGGTTGTTACTGTGACAG | |
| | TTTGGAAAATTTGGCAGATGGTTATAAGCACATGTCCTTGGCCCT | |
| | GAATAGGACTGGCAGAAGCATTGTGTACTCCTGTGAGTGGCCTC | |
| | TTTATATGTGGCCCTTTCAAAAGCCCAATTATACAGAAATCCGAC | |
| | AGTACTGCAATCACTGGCGAAATTTTGCTGACATTGATGATTCCT | |
| | GGAAAAGTATAAAGAGTATCTTGGACTGGACATCTTTTAACCAG | |
| | GAGAGAATTGTTGATGTTGCTGGACCAGGGGGTTGGAATGACCC | |
| | AGATATGTTAGTGATTGGCAACTTTGGCCTCAGCTGGAATCAGC | |
| | AAGTAACTCAGATGGCCCTCTGGGCTATCATGGCTGCTCCTTTAT | |
| | TCATGTCTAATGACCTCCGACACATCAGCCCTCAAGCCAAAGCTC | |
| | TCCTTCAGGATAAGGACGTAATTGCCATCAATCAGGACCCCTTG | |
| | GGCAAGCAAGGGTACCAGCTTAGACAGGGAGACAACTTTGAAGT | |
| | GTGGGAACGACCTCTCTCAGGCTTAGCCTGGGCTGTAGCTATGAT | |
| | AAACCGGCAGGAGATTGGTGGACCTCGCTCTTATACCATCGCAG | |
| | TTGCTTCCCTGGGTAAAGGAGTGGCCTGTAATCCTGCCTGCTTCA | |
| | TCACACAGCTCCTCCCTGTGAAAAGGAAGCTAGGGTTCTATGAA | |
| | TGGACTTCAAGGTTAAGAAGTCACATAAATCCCACAGGCACTGT | |
| | TTTGCTTCAGCTAGAAAATACAATGCAGATGTCATTAAAAGACTT | |
| | ACTTTAA | |
| GLA | ATGAAGCTCTCCCTGGTGGCCGCGATGCTGCTGCTGCTCAGCGCG | 201 |
| engineered | GCGCGGGCCCTGGACAATGGATTGGCAAGGACGCCTACCATGGG | |
| | CTGGCTGCACTGGGAGCGCTTCATGTGCAACCTTGACTGCCAGG | |
| | AAGAGCCAGATTCCTGCATCAGTGAGAAGCTCTTCATGGAGATG | |
| | GCAGAGCTCATGGTCTCAGAAGGCTGGAAGGATGCAGGTTATGA | |
| | GTACCTCTGCATTGATGACTGTTGGATGGCTCCCCAAAGAGATTC | |
| | AGAAGGCAGACTTCAGGCAGACCCTCAGCGCTTTCCTCATGGGA | |
| | TTCGCCAGCTAGCTAATTATGTTCACAGCAAAGGACTGAAGCTA | |
| | GGGATTTATGCAGATGTTGGAAATAAAACCTGCGCAGGCTTCCC | |
| | TGGGAGTTTTGGATACTACGACATTGATGCCCAGACCTTTGCTGA | |
| | CTGGGGAGTAGATCTGCTAAAATTTGATGGTTGTTACTGTGACAG | |
| | TTTGGAAAATTTGGCAGATGGTTATAAGCACATGTCCTTGGCCCT | |
| | GAATAGGACTGGCAGAAGCATTGTGTACTCCTGTGAGTGGCCTC | |
| | TTTATATGTGGCCCTTTCAAAAGCCCAATTATACAGAAATCCGAC | |
| | AGTACTGCAATCACTGGCGAAATTTTGCTGACATTGATGATTCCT | |
| | GGAAAAGTATAAAGAGTATCTTGGACTGGACATCTTTTAACCAG | |
| | GAGAGAATTGTTGATGTTGCTGGACCAGGGGGTTGGAATGACCC | |
| | AGATATGTTAGTGATTGGCAACTTTGGCCTCAGCTGGAATCAGC | |
| | AAGTAACTCAGATGGCCCTCTGGGCTATCATGGCTGCTCCTTTAT | |
| | TCATGTCTAATGACCTCCGACACATCAGCCCTCAAGCCAAAGCTC | |
| | TCCTTCAGGATAAGGACGTAATTGCCATCAATCAGGACCCCTTG | |
| | GGCAAGCAAGGGTACCAGCTTAGACAGGGAGACAACTTTGAAGT | |
| | GTGGGAACGACCTCTCTCAGGCTTAGCCTGGGCTGTAGCTATGAT | |
| | AAACCGGCAGGAGATTGGTGGACCTCGCTCTTATACCATCGCAG | |
| | TTGCTTCCCTGGGTAAAGGAGTGGCCTGTAATCCTGCCTGCTTCA | |
| | TCACACAGCTCCTCCCTGTGAAAAGGAAGCTAGGGTTCTATGAA | |
| | TGGACTTCAAGGTTAAGAAGTCACATAAATCCCACAGGCACTGT | |
| | TTTGCTTCAGCTAGAAAATACAATGCAGATGTCATTAAAAGACTT | |
| | ACTTTACATCCCTGCAAAGCAGGGGCTGCAGGGAGCCCAGATGG | |
| | GGCAGCCCGGGGGCGGTGGCTCAGGTGGTGGAGGTTCAAGAAC | |
| | ACTGTGCGGAGGGGAGCTTGTAGACACTCTTCAGTTCGTGTGTG | |
| | GAGATCGCGGGTTCCTCTTCTCTCGCCCCGCTTCCAGAGTTTCAC | |
| | GGAGGTCTAGGGGTATAGTAGAGGAGTGTTGTTTCAGGTCCTGT | |
| | GACTTGGCGCTCCTCGAGACCTATTGCGCGACGCCAGCCAGGTC | |
| | CGAATAA | |
| BiP-vIGF2- | ATGAAGCTCTCCCTGGTGGCCGCGATGCTGCTGCTGCTCAGCGCG | 202 |
| 17-2GS- | GCGCGGGCCTCTAGAACACTGTGCGGAGGGGAGCTTGTAGACAC | |
| GAA | TCTTCAGTTCGTGTGTGGAGATCGCGGGTTCCTCTTCTCTCGCCC | |
| | CGCTTCCAGAGTTTCACGGAGGTCTAGGGGTATAGTAGAGGAGT | |
| | GTTGTTTCAGGGAGTGTGACTTGGCGCTCCTCGAGACCTATTGCG | |
| | CGACGCCAGCCAGGTCCGAAGGGGGCGGTGGCTCAGGTGGTGG | |
| | AGGTAGCAGACCAGGGCCCCGGGATGCCCAGGCACACCCCGGCC | |
| | GTCCCAGAGCAGTGCCCACACAGTGCGACGTCCCCCCCAACAGC | |
| | CGCTTCGATTGCGCCCCTGACAAGGCCATCACCCAGGAACAGTG | |
| | CGAGGCCCGCGGCTGTTGCTACATCCCTGCAAAGCAGGGGCTGC | |
| | AGGGAGCCCAGATGGGGCAGCCCTGGTGCTTCTTCCCACCCAGC | |
| | TACCCCAGCTACAAGCTGGAGAACCTGAGCTCCTCTGAAATGGG | |
| | CTACACGGCCACCCTGACCCGTACCACCCCCACCTTCTTCCCCAA | |
| | GGACATCCTGACCCTGCGGCTGGACGTGATGATGGAGACTGAGA | |
| | ACCGCCTCCACTTCACGATCAAAGATCCAGCTAACAGGCGCTAC | |
| | GAGGTGCCCTTGGAGACCCCGCATGTCCACAGCCGGGCACCGTC | |
| | CCCACTCTACAGCGTGGAGTTCTCCGAGGAGCCCTTCGGGGTGA | |
| | TCGTGCGCCGGCAGCTGGACGGCCGCGTGCTGCTGAACACGACG | |
| | GTGGCGCCCCTGTTCTTTGCGGACCAGTTCCTTCAGCTGTCCACC | |
| | TCGCTGCCCTCGCAGTATATCACAGGCCTCGCCGAGCACCTCAGT | |
| | CCCCTGATGCTCAGCACCAGCTGGACCAGGATCACCCTGTGGAA | |
| | CCGGGACCTTGCGCCCACGCCCGGTGCGAACCTCTACGGGTCTC | |
| | ACCCTTTCTACCTGGCGCTGGAGGACGGCGGGTCGGCACACGGG | |
| | GTGTTCCTGCTAAACAGCAATGCCATGGATGTGGTCCTGCAGCC | |
| | GAGCCCTGCCCTTAGCTGGAGGTCGACAGGTGGGATCCTGGATG | |
| | TCTACATCTTCCTGGGCCCAGAGCCCAAGAGCGTGGTGCAGCAG | |
| | TACCTGGACGTTGTGGGATACCCGTTCATGCCGCCATACTGGGGC | |
| | CTGGGCTTCCACCTGTGCCGCTGGGGCTACTCCTCCACCGCTATC | |
| | ACCCGCCAGGTGGTGGAGAACATGACCAGGGCCCACTTCCCCCT | |
| | GGACGTCCAGTGGAACGACCTGGACTACATGGACTCCCGGAGGG | |
| | ACTTCACGTTCAACAAGGATGGCTTCCGGGACTTCCCGGCCATG | |
| | GTGCAGGAGCTGCACCAGGGCGGCCGGCGCTACATGATGATCGT | |
| | GGATCCTGCCATCAGCAGCTCGGGCCCTGCCGGGAGCTACAGGC | |
| | CCTACGACGAGGGTCTGCGGAGGGGGGTTTTCATCACCAACGAG | |
| | ACCGGCCAGCCGCTGATTGGGAAGGTATGGCCCGGGTCCACTGC | |
| | CTTCCCCGACTTCACCAACCCCACAGCCCTGGCCTGGTGGGAGG | |
| | ACATGGTGGCTGAGTTCCATGACCAGGTGCCCTTCGACGGCATG | |
| | TGGATTGACATGAACGAGCCTTCCAACTTCATCAGGGGCTCTGA | |
| | GGACGGCTGCCCCAACAATGAGCTGGAGAACCCACCCTACGTGC | |
| | CTGGGGTGGTTGGGGGGACCCTCCAGGCGGCCACCATCTGTGCC | |
| | TCCAGCCACCAGTTTCTCTCCACACACTACAACCTGCACAACCTC | |
| | TACGGCCTGACCGAAGCCATCGCCTCCCACAGGGCGCTGGTGAA | |
| | GGCTCGGGGGACACGCCCATTTGTGATCTCCCGCTCGACCTTTGC | |
| | TGGCCACGGCCGATACGCCGGCCACTGGACGGGGGACGTGTGGA | |
| | GCTCCTGGGAGCAGCTCGCCTCCTCCGTGCCAGAAATCCTGCAGT | |
| | TTAACCTGCTGGGGGTGCCTCTGGTCGGGGCCGACGTCTGCGGCT | |
| | TCCTGGGCAACACCTCAGAGGAGCTGTGTGTGCGCTGGACCCAG | |
| | CTGGGGGCCTTCTACCCCTTCATGCGGAACCACAACAGCCTGCTC | |
| | AGTCTGCCCCAGGAGCCGTACAGCTTCAGCGAGCCGGCCCAGCA | |
| | GGCCATGAGGAAGGCCCTCACCCTGCGCTACGCACTCCTCCCCC | |
| | ACCTCTACACACTGTTCCACCAGGCCCACGTCGCGGGGGAGACC | |
| | GTGGCCCGGCCCCTCTTCCTGGAGTTCCCCAAGGACTCTAGCACC | |
| | TGGACTGTGGACCACCAGCTCCTGTGGGGGGAGGCCCTGCTCAT | |
| | CACCCCAGTGCTCCAGGCCGGGAAGGCCGAAGTGACTGGCTACT | |
| | TCCCCTTGGGCACATGGTACGACCTGCAGACGGTGCCAGTAGAG | |
| | GCCCTTGGCAGCCTCCCACCCCCACCTGCAGCTCCCCGTGAGCCA | |
| | GCCATCCACAGCGAGGGGCAGTGGGTGACGCTGCCGGCCCCCCT | |
| | GGACACCATCAACGTCCACCTCCGGGCTGGGTACATCATCCCCCT | |
| | GCAGGGCCCTGGCCTCACAACCACAGAGTCCCGCCAGCAGCCCA | |
| | TGGCCCTGGCTGTGGCCCTGACCAAGGGTGGGGAGGCCCGAGGG | |
| | GAGCTGTTCTGGGACGATGGAGAGAGCCTGGAAGTGCTGGAGCG | |
| | AGGGGCCTACACACAGGTCATCTTCCTGGCCAGGAATAACACGA | |
| | TCGTGAATGAGCTGGTACGTGTGACCAGTGAGGGAGCTGGCCTG | |
| | CAGCTGCAGAAGGTGACTGTCCTGGGCGTGGCCACGGCGCCCCA | |
| | GCAGGTCCTCTCCAACGGTGTCCCTGTCTCCAACTTCACCTACAG | |
| | CCCCGACACCAAGGTCCTGGACATCTGTGTCTCGCTGTTGATGGG | |
| | AGAGCAGTTTCTCGTCAGCTGGTGTTAG | |
| BiP-vIGF2- | ATGAAGCTCTCCCTGGTGGCCGCGATGCTGCTGCTGCTCAGCGCG | 203 |
| 20-2GS- | GCGCGGGCCTCTAGAACACTGTGCGGAGGGGAGCTTGTAGACAC | |
| GAA | TCTTCAGTTCGTGTGTGGAGATCGCGGGTTCCTCTTCTCTCGCCC | |
| | CGCTTCCAGAGTTTCACGGAGGTCTAGGGGTATACTGGAGGAGT | |
| | GTTGTTTCAGGTCCTGTGACTTGGCGCTCCTCGAGACCTATTGCG | |
| | CGACGCCAGCCAGGTCCGAAGGGGGCGGTGGCTCAGGTGGTGG | |
| | AGGTAGCAGACCAGGGCCCCGGGATGCCCAGGCACACCCCGGCC | |
| | GTCCCAGAGCAGTGCCCACACAGTGCGACGTCCCCCCCAACAGC | |
| | CGCTTCGATTGCGCCCCTGACAAGGCCATCACCCAGGAACAGTG | |
| | CGAGGCCCGCGGCTGTTGCTACATCCCTGCAAAGCAGGGGCTGC | |
| | AGGGAGCCCAGATGGGGCAGCCCTGGTGCTTCTTCCCACCCAGC | |
| | TACCCCAGCTACAAGCTGGAGAACCTGAGCTCCTCTGAAATGGG | |
| | CTACACGGCCACCCTGACCCGTACCACCCCCACCTTCTTCCCCAA | |
| | GGACATCCTGACCCTGCGGCTGGACGTGATGATGGAGACTGAGA | |
| | ACCGCCTCCACTTCACGATCAAAGATCCAGCTAACAGGCGCTAC | |
| | GAGGTGCCCTTGGAGACCCCGCATGTCCACAGCCGGGCACCGTC | |
| | CCCACTCTACAGCGTGGAGTTCTCCGAGGAGCCCTTCGGGGTGA | |
| | TCGTGCGCCGGCAGCTGGACGGCCGCGTGCTGCTGAACACGACG | |
| | GTGGCGCCCCTGTTCTTTGCGGACCAGTTCCTTCAGCTGTCCACC | |
| | TCGCTGCCCTCGCAGTATATCACAGGCCTCGCCGAGCACCTCAGT | |
| | CCCCTGATGCTCAGCACCAGCTGGACCAGGATCACCCTGTGGAA | |
| | CCGGGACCTTGCGCCCACGCCCGGTGCGAACCTCTACGGGTCTC | |
| | ACCCTTTCTACCTGGCGCTGGAGGACGGCGGGTCGGCACACGGG | |
| | GTGTTCCTGCTAAACAGCAATGCCATGGATGTGGTCCTGCAGCC | |
| | GAGCCCTGCCCTTAGCTGGAGGTCGACAGGTGGGATCCTGGATG | |
| | TCTACATCTTCCTGGGCCCAGAGCCCAAGAGCGTGGTGCAGCAG | |
| | TACCTGGACGTTGTGGGATACCCGTTCATGCCGCCATACTGGGGC | |
| | CTGGGCTTCCACCTGTGCCGCTGGGGCTACTCCTCCACCGCTATC | |
| | ACCCGCCAGGTGGTGGAGAACATGACCAGGGCCCACTTCCCCCT | |
| | GGACGTCCAGTGGAACGACCTGGACTACATGGACTCCCGGAGGG | |
| | ACTTCACGTTCAACAAGGATGGCTTCCGGGACTTCCCGGCCATG | |
| | GTGCAGGAGCTGCACCAGGGCGGCCGGCGCTACATGATGATCGT | |
| | GGATCCTGCCATCAGCAGCTCGGGCCCTGCCGGGAGCTACAGGC | |
| | CCTACGACGAGGGTCTGCGGAGGGGGGTTTTCATCACCAACGAG | |
| | ACCGGCCAGCCGCTGATTGGGAAGGTATGGCCCGGGTCCACTGC | |
| | CTTCCCCGACTTCACCAACCCCACAGCCCTGGCCTGGTGGGAGG | |
| | ACATGGTGGCTGAGTTCCATGACCAGGTGCCCTTCGACGGCATG | |
| | TGGATTGACATGAACGAGCCTTCCAACTTCATCAGGGGCTCTGA | |
| | GGACGGCTGCCCCAACAATGAGCTGGAGAACCCACCCTACGTGC | |
| | CTGGGGTGGTTGGGGGGACCCTCCAGGCGGCCACCATCTGTGCC | |
| | TCCAGCCACCAGTTTCTCTCCACACACTACAACCTGCACAACCTC | |
| | TACGGCCTGACCGAAGCCATCGCCTCCCACAGGGCGCTGGTGAA | |
| | GGCTCGGGGGACACGCCCATTTGTGATCTCCCGCTCGACCTTTGC | |
| | TGGCCACGGCCGATACGCCGGCCACTGGACGGGGGACGTGTGGA | |
| | GCTCCTGGGAGCAGCTCGCCTCCTCCGTGCCAGAAATCCTGCAGT | |
| | TTAACCTGCTGGGGGTGCCTCTGGTCGGGGCCGACGTCTGCGGCT | |
| | TCCTGGGCAACACCTCAGAGGAGCTGTGTGTGCGCTGGACCCAG | |
| | CTGGGGGCCTTCTACCCCTTCATGCGGAACCACAACAGCCTGCTC | |
| | AGTCTGCCCCAGGAGCCGTACAGCTTCAGCGAGCCGGCCCAGCA | |
| | GGCCATGAGGAAGGCCCTCACCCTGCGCTACGCACTCCTCCCCC | |
| | ACCTCTACACACTGTTCCACCAGGCCCACGTCGCGGGGGAGACC | |
| | GTGGCCCGGCCCCTCTTCCTGGAGTTCCCCAAGGACTCTAGCACC | |
| | TGGACTGTGGACCACCAGCTCCTGTGGGGGGAGGCCCTGCTCAT | |
| | CACCCCAGTGCTCCAGGCCGGGAAGGCCGAAGTGACTGGCTACT | |
| | TCCCCTTGGGCACATGGTACGACCTGCAGACGGTGCCAGTAGAG | |
| | GCCCTTGGCAGCCTCCCACCCCCACCTGCAGCTCCCCGTGAGCCA | |
| | GCCATCCACAGCGAGGGGCAGTGGGTGACGCTGCCGGCCCCCCT | |
| | GGACACCATCAACGTCCACCTCCGGGCTGGGTACATCATCCCCCT | |
| | GCAGGGCCCTGGCCTCACAACCACAGAGTCCCGCCAGCAGCCCA | |
| | TGGCCCTGGCTGTGGCCCTGACCAAGGGTGGGGAGGCCCGAGGG | |
| | GAGCTGTTCTGGGACGATGGAGAGAGCCTGGAAGTGCTGGAGCG | |
| | AGGGGCCTACACACAGGTCATCTTCCTGGCCAGGAATAACACGA | |
| | TCGTGAATGAGCTGGTACGTGTGACCAGTGAGGGAGCTGGCCTG | |
| | CAGCTGCAGAAGGTGACTGTCCTGGGCGTGGCCACGGCGCCCCA | |
| | GCAGGTCCTCTCCAACGGTGTCCCTGTCTCCAACTTCACCTACAG | |
| | CCCCGACACCAAGGTCCTGGACATCTGTGTCTCGCTGTTGATGGG | |
| | AGAGCAGTTTCTCGTCAGCTGGTGTTAG | |
| BiP-vIGF2- | ATGAAGCTCTCCCTGGTGGCCGCGATGCTGCTGCTGCTCAGCGCG | 204 |
| 22-2GS- | GCGCGGGCCTCTAGAACACTGTGCGGAGGGGAGCTTGTAGACAC | |
| GAA | TCTTCAGTTCGTGTGTGGAGATCGCGGGTTCCTCTTCTCTCGCGG | |
| | AGGTGGAGGTTCTAGGGGTATAGTAGAGGAGTGTTGTTTCAGGT | |
| | CCTGTGACTTGGCGCTCCTCGAGACCTATTGCGCGACGCCAGCCA | |
| | GGTCCGAAGGGGGCGGTGGCTCAGGTGGTGGAGGTAGCAGACC | |
| | AGGGCCCCGGGATGCCCAGGCACACCCCGGCCGTCCCAGAGCAG | |
| | TGCCCACACAGTGCGACGTCCCCCCCAACAGCCGCTTCGATTGC | |
| | GCCCCTGACAAGGCCATCACCCAGGAACAGTGCGAGGCCCGCGG | |
| | CTGTTGCTACATCCCTGCAAAGCAGGGGCTGCAGGGAGCCCAGA | |
| | TGGGGCAGCCCTGGTGCTTCTTCCCACCCAGCTACCCCAGCTACA | |
| | AGCTGGAGAACCTGAGCTCCTCTGAAATGGGCTACACGGCCACC | |
| | CTGACCCGTACCACCCCCACCTTCTTCCCCAAGGACATCCTGACC | |
| | CTGCGGCTGGACGTGATGATGGAGACTGAGAACCGCCTCCACTT | |
| | CACGATCAAAGATCCAGCTAACAGGCGCTACGAGGTGCCCTTGG | |
| | AGACCCCGCATGTCCACAGCCGGGCACCGTCCCCACTCTACAGC | |
| | GTGGAGTTCTCCGAGGAGCCCTTCGGGGTGATCGTGCGCCGGCA | |
| | GCTGGACGGCCGCGTGCTGCTGAACACGACGGTGGCGCCCCTGT | |
| | TCTTTGCGGACCAGTTCCTTCAGCTGTCCACCTCGCTGCCCTCGC | |
| | AGTATATCACAGGCCTCGCCGAGCACCTCAGTCCCCTGATGCTCA | |
| | GCACCAGCTGGACCAGGATCACCCTGTGGAACCGGGACCTTGCG | |
| | CCCACGCCCGGTGCGAACCTCTACGGGTCTCACCCTTTCTACCTG | |
| | GCGCTGGAGGACGGCGGGTCGGCACACGGGGTGTTCCTGCTAAA | |
| | CAGCAATGCCATGGATGTGGTCCTGCAGCCGAGCCCTGCCCTTA | |
| | GCTGGAGGTCGACAGGTGGGATCCTGGATGTCTACATCTTCCTG | |
| | GGCCCAGAGCCCAAGAGCGTGGTGCAGCAGTACCTGGACGTTGT | |
| | GGGATACCCGTTCATGCCGCCATACTGGGGCCTGGGCTTCCACCT | |
| | GTGCCGCTGGGGCTACTCCTCCACCGCTATCACCCGCCAGGTGGT | |
| | GGAGAACATGACCAGGGCCCACTTCCCCCTGGACGTCCAGTGGA | |
| | ACGACCTGGACTACATGGACTCCCGGAGGGACTTCACGTTCAAC | |
| | AAGGATGGCTTCCGGGACTTCCCGGCCATGGTGCAGGAGCTGCA | |
| | CCAGGGCGGCCGGCGCTACATGATGATCGTGGATCCTGCCATCA | |
| | GCAGCTCGGGCCCTGCCGGGAGCTACAGGCCCTACGACGAGGGT | |
| | CTGCGGAGGGGGGTTTTCATCACCAACGAGACCGGCCAGCCGCT | |
| | GATTGGGAAGGTATGGCCCGGGTCCACTGCCTTCCCCGACTTCAC | |
| | CAACCCCACAGCCCTGGCCTGGTGGGAGGACATGGTGGCTGAGT | |
| | TCCATGACCAGGTGCCCTTCGACGGCATGTGGATTGACATGAAC | |
| | GAGCCTTCCAACTTCATCAGGGGCTCTGAGGACGGCTGCCCCAA | |
| | CAATGAGCTGGAGAACCCACCCTACGTGCCTGGGGTGGTTGGGG | |
| | GGACCCTCCAGGCGGCCACCATCTGTGCCTCCAGCCACCAGTTTC | |
| | TCTCCACACACTACAACCTGCACAACCTCTACGGCCTGACCGAA | |
| | GCCATCGCCTCCCACAGGGCGCTGGTGAAGGCTCGGGGGACACG | |
| | CCCATTTGTGATCTCCCGCTCGACCTTTGCTGGCCACGGCCGATA | |
| | CGCCGGCCACTGGACGGGGGACGTGTGGAGCTCCTGGGAGCAGC | |
| | TCGCCTCCTCCGTGCCAGAAATCCTGCAGTTTAACCTGCTGGGGG | |
| | TGCCTCTGGTCGGGGCCGACGTCTGCGGCTTCCTGGGCAACACCT | |
| | CAGAGGAGCTGTGTGTGCGCTGGACCCAGCTGGGGGCCTTCTAC | |
| | CCCTTCATGCGGAACCACAACAGCCTGCTCAGTCTGCCCCAGGA | |
| | GCCGTACAGCTTCAGCGAGCCGGCCCAGCAGGCCATGAGGAAGG | |
| | CCCTCACCCTGCGCTACGCACTCCTCCCCCACCTCTACACACTGT | |
| | TCCACCAGGCCCACGTCGCGGGGGAGACCGTGGCCCGGCCCCTC | |
| | TTCCTGGAGTTCCCCAAGGACTCTAGCACCTGGACTGTGGACCAC | |
| | CAGCTCCTGTGGGGGGAGGCCCTGCTCATCACCCCAGTGCTCCA | |
| | GGCCGGGAAGGCCGAAGTGACTGGCTACTTCCCCTTGGGCACAT | |
| | GGTACGACCTGCAGACGGTGCCAGTAGAGGCCCTTGGCAGCCTC | |
| | CCACCCCCACCTGCAGCTCCCCGTGAGCCAGCCATCCACAGCGA | |
| | GGGGCAGTGGGTGACGCTGCCGGCCCCCCTGGACACCATCAACG | |
| | TCCACCTCCGGGCTGGGTACATCATCCCCCTGCAGGGCCCTGGCC | |
| | TCACAACCACAGAGTCCCGCCAGCAGCCCATGGCCCTGGCTGTG | |
| | GCCCTGACCAAGGGTGGGGAGGCCCGAGGGGAGCTGTTCTGGGA | |
| | CGATGGAGAGAGCCTGGAAGTGCTGGAGCGAGGGGCCTACACA | |
| | CAGGTCATCTTCCTGGCCAGGAATAACACGATCGTGAATGAGCT | |
| | GGTACGTGTGACCAGTGAGGGAGCTGGCCTGCAGCTGCAGAAGG | |
| | TGACTGTCCTGGGCGTGGCCACGGCGCCCCAGCAGGTCCTCTCC | |
| | AACGGTGTCCCTGTCTCCAACTTCACCTACAGCCCCGACACCAAG | |
| | GTCCTGGACATCTGTGTCTCGCTGTTGATGGGAGAGCAGTTTCTC | |
| | GTCAGCTGGTGTTAG | |
| PPT1-3 | ATGAAACTGTCTCTGGTTGCAGCAATGCTCTTGCTGTTGAGTGCG | 205 |
| (BiP-PPT1; | GCCCGCGCGGATCCACCTGCTCCCCTGCCCCTCGTTATATGGCAT | |
| Codon | GGCATGGGAGATTCCTGTTGTAATCCCCTCAGCATGGGGGCCAT | |
| optimized | CAAAAAAATGGTGGAAAAAAAAATACCTGGCATATATGTACTCT | |
| IDT) | CACTTGAAATCGGTAAGACCCTTATGGAAGACGTCGAAAATTCC | |
| | TTCTTTTTGAACGTGAACTCACAAGTTACGACCGTCTGTCAAGCT | |
| | CTCGCGAAAGACCCTAAGCTCCAGCAAGGTTATAATGCAATGGG | |
| | CTTCTCACAGGGAGGTCAGTTCTTGCGAGCGGTAGCCCAGAGGT | |
| | GTCCGTCTCCGCCAATGATCAACTTGATCTCAGTGGGGGGTCAGC | |
| | ACCAAGGCGTTTTTGGACTCCCTAGATGCCCTGGAGAGAGCTCTC | |
| | ACATTTGCGATTTTATACGGAAGACGCTGAATGCCGGCGCGTATT | |
| | CAAAGGTCGTTCAAGAGCGACTCGTCCAGGCTGAATACTGGCAC | |
| | GATCCGATTAAGGAAGACGTGTATCGAAACCATTCTATCTTTCTT | |
| | GCCGACATTAACCAGGAGCGAGGGATCAACGAAAGTTATAAAA | |
| | AAAACCTGATGGCACTCAAGAAATTTGTAATGGTTAAATTCCTG | |
| | AACGATTCAATAGTTGATCCGGTGGATTCCGAGTGGTTCGGCTTC | |
| | TACCGGTCCGGTCAGGCCAAGGAAACAATCCCATTGCAAGAAAC | |
| | CAGTCTCTATACTCAGGACCGCCTGGGTCTGAAAGAAATGGACA | |
| | ACGCTGGCCAACTTGTTTTTCTGGCAACGGAGGGTGATCACTTGC | |
| | AGCTCTCTGAAGAATGGTTTTACGCACACATCATTCCTTTCCTTG | |
| | GTTAA | |
| PPT1-4 | ATGAAGTTGTCCCTCGTAGCTGCAATGTTGCTGCTCCTCAGTGCA | 206 |
| (BiP-vIGF2- | GCGCGGGCAAGTCGCACGTTGTGTGGAGGTGAACTCGTCGACAC | |
| PPT1; | CCTTCAGTTCGTATGTGGAGATCGCGGTTTCCTCTTCTCACGCCC | |
| Codon | AGCTTCCAGAGTTTCCCGAAGATCACGAGGAATAGTTGAGGAGT | |
| optimized | GCTGTTTTCGGTCTTGTGATCTGGCTCTCCTCGAGACTTATTGTGC | |
| IDT) | TACGCCGGCCCGCTCTGAAGGAGGTGGTGGCAGTGGAGGAGGA | |
| | GGGAGTCGGCCTAGGGCAGTCCCAACCCAGGATCCCCCAGCACC | |
| | CCTCCCCCTGGTAATTTGGCATGGAATGGGTGATTCCTGCTGTAA | |
| | CCCACTCTCAATGGGGGCAATTAAGAAAATGGTAGAGAAAAAG | |
| | ATCCCTGGCATTTATGTTCTGTCACTCGAAATCGGTAAAACGCTC | |
| | ATGGAGGACGTAGAAAACAGCTTTTTTCTGAATGTTAATTCACA | |
| | GGTTACCACGGTCTGCCAAGCATTGGCAAAGGACCCGAAATTGC | |
| | AACAAGGCTATAACGCGATGGGGTTCAGCCAAGGCGGGCAGTTT | |
| | CTTCGAGCTGTGGCTCAGCGCTGCCCTTCCCCACCGATGATAAAT | |
| | TTGATTAGCGTAGGGGGACAACATCAAGGGGTTTTCGGTTTGCC | |
| | AAGGTGTCCTGGCGAATCTTCACATATTTGCGACTTTATACGGAA | |
| | GACCTTGAATGCGGGGGCGTATAGTAAAGTCGTCCAGGAACGGC | |
| | TTGTCCAAGCTGAATACTGGCACGATCCCATCAAAGAAGATGTC | |
| | TATCGGAATCACAGCATTTTTCTCGCCGACATAAACCAAGAACG | |
| | CGGAATTAATGAGTCATACAAGAAGAACTTGATGGCACTTAAAA | |
| | AATTTGTGATGGTTAAGTTTTTGAATGATAGTATCGTAGATCCCG | |
| | TAGATAGTGAATGGTTTGGTTTCTATCGATCCGGACAGGCTAAA | |
| | GAAACGATACCATTGCAGGAAACCTCTTTGTATACTCAAGATAG | |
| | GTTGGGCCTCAAGGAGATGGATAATGCGGGGCAACTTGTCTTCC | |
| | TCGCGACTGAGGGTGACCACCTCCAGCTCAGCGAGGAATGGTTT | |
| | TACGCCCACATCATTCCTTTCCTTGGTTAA | |
| PPT1-5 (wt- | ATGGCAAGTCCAGGGTGTCTTTGGTTGCTCGCGGTTGCCTTGCTC | 207 |
| PPT1- | CCTTGGACGTGCGCGTCCCGAGCCCTTCAACACCTCGATCCACCA | |
| vIGF2; | GCCCCGCTTCCTCTCGTGATATGGCACGGCATGGGCGACAGTTGC | |
| Codon | TGCAATCCCTTGTCTATGGGCGCAATTAAAAAGATGGTGGAAAA | |
| optimized | GAAAATCCCTGGTATCTACGTTTTGAGCCTCGAAATTGGGAAAA | |
| IDT) | CGCTCATGGAGGATGTCGAGAACAGCTTCTTTCTTAACGTCAATT | |
| | CCCAAGTTACCACGGTTTGTCAAGCCTTGGCGAAAGATCCCAAG | |
| | CTTCAGCAAGGGTATAACGCTATGGGATTTAGCCAGGGCGGACA | |
| | GTTCCTGAGGGCGGTAGCACAGAGGTGTCCTAGTCCACCAATGA | |
| | TAAATCTCATCTCAGTCGGGGGCCAGCACCAGGGCGTCTTCGGG | |
| | CTTCCTCGATGCCCCGGCGAATCCAGCCACATATGTGACTTCATT | |
| | AGAAAAACTTTGAATGCAGGGGCCTACAGTAAAGTGGTTCAAGA | |
| | ACGCCTGGTACAAGCAGAGTATTGGCATGACCCGATTAAGGAAG | |
| | ATGTCTACAGAAATCACTCTATTTTTTTGGCGGACATCAATCAGG | |
| | AACGAGGCATTAACGAGTCTTACAAGAAGAACCTGATGGCGCTG | |
| | AAAAAGTTCGTCATGGTCAAGTTCTTGAATGACTCCATTGTCGAT | |
| | CCTGTAGACAGCGAGTGGTTTGGCTTCTACAGGTCTGGTCAAGC | |
| | AAAGGAGACAATACCACTTCAGGAAACCAGTCTCTATACACAAG | |
| | ACAGACTGGGTTTGAAGGAAATGGACAATGCAGGCCAACTGGTA | |
| | TTCCTGGCTACAGAGGGAGATCATCTTCAACTGAGCGAAGAGTG | |
| | GTTTTATGCCCACATAATCCCCTTTCTGGGAAGACCTAGAGCAGT | |
| | GCCTACGCAGGGTGGTGGTGGCTCTGGAGGAGGAGGCTCCAGGA | |
| | CTCTGTGTGGGGGCGAGCTGGTGGACACCTTGCAATTCGTGTGTG | |
| | GCGACCGAGGATTTCTGTTCAGTCGACCTGCCTCAAGAGTAAGC | |
| | CGGAGGAGTCGGGGGATCGTTGAAGAATGCTGTTTCCGGAGCTG | |
| | CGACTTGGCGTTGCTCGAGACTTATTGTGCCACACCTGCAAGGA | |
| | GTGAGTGA | |
| PPT1-9 (wt- | ATGGCGTCGCCCGGCTGCCTGTGGCTCTTGGCTGTGGCTCTCCTG | 208 |
| PPT1; | CCATGGACCTGCGCTTCTCGGGCGCTGCAGCATCTGGACCCGCC | |
| native | GGCGCCGCTGCCGTTGGTGATCTGGCATGGGATGGGAGACAGCT | |
| human | GTTGCAATCCCTTAAGCATGGGTGCTATTAAAAAAATGGTGGAG | |
| sequence) | AAGAAAATACCTGGAATTTACGTCTTATCTTTAGAGATTGGGAA | |
| | GACCCTGATGGAGGACGTGGAGAACAGCTTCTTCTTGAATGTCA | |
| | ATTCCCAAGTAACAACAGTGTGTCAGGCACTTGCTAAGGATCCT | |
| | AAATTGCAGCAAGGCTACAATGCTATGGGATTCTCCCAGGGAGG | |
| | CCAATTTCTGAGGGCAGTGGCTCAGAGATGCCCTTCACCTCCCAT | |
| | GATCAATCTGATCTCGGTTGGGGGACAACATCAAGGTGTTTTTGG | |
| | ACTCCCTCGATGCCCAGGAGAGAGCTCTCACATCTGTGACTTCAT | |
| | CCGAAAAACACTGAATGCTGGGGCGTACTCCAAAGTTGTTCAGG | |
| | AACGCCTCGTGCAAGCCGAATACTGGCATGACCCCATAAAGGAG | |
| | GATGTGTATCGCAACCACAGCATCTTCTTGGCAGATATAAATCA | |
| | GGAGCGGGGTATCAATGAGTCCTACAAGAAAAACCTGATGGCCC | |
| | TGAAGAAGTTTGTGATGGTGAAATTCCTCAATGATTCCATTGTGG | |
| | ACCCTGTAGATTCGGAGTGGTTTGGATTTTACAGAAGTGGCCAA | |
| | GCCAAGGAAACCATTCCCTTACAGGAGACCTCCCTGTACACACA | |
| | GGACCGCCTGGGGCTAAAGGAAATGGACAATGCAGGACAGCTA | |
| | GTGTTTCTGGCTACAGAAGGGGACCATCTTCAGTTGTCTGAAGA | |
| | ATGGTTTTATGCCCACATCATACCATTCCTTGGATGA | |
| PPT1-10 | ATGGCATCACCGGGTTGCCTCTGGTTGTTGGCCGTTGCGTTGCTT | 209 |
| (wt-PPT1- | CCGTGGACATGTGCATCAAGAGCTCTTCAACATCTGGATCCCCCA | |
| vIGF2_2; | GCTCCCCTGCCGCTCGTAATCTGGCACGGGATGGGGGATTCATGT | |
| Codon | TGTAACCCGTTGTCAATGGGCGCGATAAAAAAGATGGTTGAAAA | |
| optimized | GAAGATTCCAGGCATCTACGTTCTGTCCCTGGAAATCGGTAAGA | |
| IDT) | CACTGATGGAAGACGTGGAGAACTCCTTCTTTCTCAACGTCAATA | |
| | GTCAGGTCACTACCGTCTGTCAAGCATTGGCAAAGGACCCTAAA | |
| | CTTCAGCAGGGGTACAATGCGATGGGGTTTAGCCAGGGCGGACA | |
| | GTTTCTTAGAGCCGTCGCACAGCGCTGTCCATCTCCCCCGATGAT | |
| | TAACCTTATATCTGTCGGGGGACAACACCAGGGTGTTTTTGGTCT | |
| | TCCTCGCTGTCCTGGTGAAAGCTCCCACATCTGTGATTTCATACG | |
| | CAAAACGTTGAACGCAGGAGCTTATAGTAAAGTCGTCCAAGAAC | |
| | GGCTTGTTCAAGCGGAGTATTGGCATGACCCAATAAAAGAAGAC | |
| | GTTTATAGGAATCACTCTATCTTCTTGGCCGATATCAACCAAGAA | |
| | CGCGGAATCAACGAAAGCTACAAAAAGAATCTTATGGCTCTCAA | |
| | GAAATTTGTTATGGTGAAATTCCTTAATGACTCTATAGTAGATCC | |
| | TGTCGATTCAGAATGGTTCGGGTTCTACAGGTCTGGCCAGGCGA | |
| | AGGAGACTATTCCCCTCCAAGAAACGTCTCTCTATACACAAGAC | |
| | AGACTCGGACTGAAAGAGATGGATAATGCGGGCCAGTTGGTCTT | |
| | CTTGGCTACGGAAGGCGATCATCTCCAACTCTCCGAAGAGTGGT | |
| | TCTATGCCCATATAATCCCGTTCCTGGGCAGACCTAGAGCAGTGC | |
| | CTACGCAGGGAGGGAGTGGGAGTGGATCCACTTCATCCAGGACT | |
| | CTGTGTGGGGGCGAGCTGGTGGACACCTTGCAATTCGTGTGTGG | |
| | CGACCGAGGATTTCTGTTCAGTCGACCTGCCTCAAGAGTAAGCC | |
| | GGAGGAGTCGGGGGATCGTTGAAGAATGCTGTTTCCGGAGCTGC | |
| | GACTTGGCGTTGCTCGAGACTTATTGTGCCACACCTGCAAGGAGT | |
| | GAATGA | |
| PPT1-11 | ATGAAGCTCTCCCTGGTGGCCGCGATGCTGCTGCTGCTCAGCGCG | 210 |
| (BiP- | GCGCGGGCCTCAAGAGCTCTTCAACATCTGGATCCCCCAGCTCCC | |
| PPT1_2; | CTGCCGCTCGTAATCTGGCACGGGATGGGGGATTCATGTTGTAA | |
| Codon | CCCGTTGTCAATGGGCGCGATAAAAAAGATGGTTGAAAAGAAGA | |
| optimized | TTCCAGGCATCTACGTTCTGTCCCTGGAAATCGGTAAGACACTGA | |
| IDT) | TGGAAGACGTGGAGAACTCCTTCTTTCTCAACGTCAATAGTCAG | |
| | GTCACTACCGTCTGTCAAGCATTGGCAAAGGACCCTAAACTTCA | |
| | GCAGGGGTACAATGCGATGGGGTTTAGCCAGGGCGGACAGTTTC | |
| | TTAGAGCCGTCGCACAGCGCTGTCCATCTCCCCCGATGATTAACC | |
| | TTATATCTGTCGGGGGACAACACCAGGGTGTTTTTGGTCTTCCTC | |
| | GCTGTCCTGGTGAAAGCTCCCACATCTGTGATTTCATACGCAAAA | |
| | CGTTGAACGCAGGAGCTTATAGTAAAGTCGTCCAAGAACGGCTT | |
| | GTTCAAGCGGAGTATTGGCATGACCCAATAAAAGAAGACGTTTA | |
| | TAGGAATCACTCTATCTTCTTGGCCGATATCAACCAAGAACGCG | |
| | GAATCAACGAAAGCTACAAAAAGAATCTTATGGCTCTCAAGAAA | |
| | TTTGTTATGGTGAAATTCCTTAATGACTCTATAGTAGATCCTGTC | |
| | GATTCAGAATGGTTCGGGTTCTACAGGTCTGGCCAGGCGAAGGA | |
| | GACTATTCCCCTCCAAGAAACGTCTCTCTATACACAAGACAGACT | |
| | CGGACTGAAAGAGATGGATAATGCGGGCCAGTTGGTCTTCTTGG | |
| | CTACGGAAGGCGATCATCTCCAACTCTCCGAAGAGTGGTTCTAT | |
| | GCCCATATAATCCCGTTCCTGGGCTAA | |
| PPT1-12 | ATGAAGCTCTCCCTGGTGGCCGCGATGCTGCTGCTGCTCAGCGCG | 211 |
| (BiPaa- | GCGCGGGCCTCAAGAGCTCTTCAACATCTGGATCCCCCAGCTCCC | |
| PPT1_2; | CTGCCGCTCGTAATCTGGCACGGGATGGGGGATTCATGTTGTAA | |
| Codon | CCCGTTGTCAATGGGCGCGATAAAAAAGATGGTTGAAAAGAAGA | |
| optimized | TTCCAGGCATCTACGTTCTGTCCCTGGAAATCGGTAAGACACTGA | |
| IDT) | TGGAAGACGTGGAGAACTCCTTCTTTCTCAACGTCAATAGTCAG | |
| | GTCACTACCGTCTGTCAAGCATTGGCAAAGGACCCTAAACTTCA | |
| | GCAGGGGTACAATGCGATGGGGTTTAGCCAGGGCGGACAGTTTC | |
| | TTAGAGCCGTCGCACAGCGCTGTCCATCTCCCCCGATGATTAACC | |
| | TTATATCTGTCGGGGGACAACACCAGGGTGTTTTTGGTCTTCCTC | |
| | GCTGTCCTGGTGAAAGCTCCCACATCTGTGATTTCATACGCAAAA | |
| | CGTTGAACGCAGGAGCTTATAGTAAAGTCGTCCAAGAACGGCTT | |
| | GTTCAAGCGGAGTATTGGCATGACCCAATAAAAGAAGACGTTTA | |
| | TAGGAATCACTCTATCTTCTTGGCCGATATCAACCAAGAACGCG | |
| | GAATCAACGAAAGCTACAAAAAGAATCTTATGGCTCTCAAGAAA | |
| | TTTGTTATGGTGAAATTCCTTAATGACTCTATAGTAGATCCTGTC | |
| | GATTCAGAATGGTTCGGGTTCTACAGGTCTGGCCAGGCGAAGGA | |
| | GACTATTCCCCTCCAAGAAACGTCTCTCTATACACAAGACAGACT | |
| | CGGACTGAAAGAGATGGATAATGCGGGCCAGTTGGTCTTCTTGG | |
| | CTACGGAAGGCGATCATCTCCAACTCTCCGAAGAGTGGTTCTAT | |
| | GCCCATATAATCCCGTTCCTGGGCTAA | |
| PPT1-13 | ATGAAACTGTCTCTGGTTGCAGCAATGCTCTTGCTGTTGAGTGCG | 212 |
| (BiPaa- | GCCCGCGCGGCGGCCGATCCACCTGCTCCCCTGCCCCTCGTTATA | |
| PPT1; | TGGCATGGCATGGGAGATTCCTGTTGTAATCCCCTCAGCATGGG | |
| Codon | GGCCATCAAAAAAATGGTGGAAAAAAAAATACCTGGCATATATG | |
| optimized | TACTCTCACTTGAAATCGGTAAGACCCTTATGGAAGACGTCGAA | |
| IDT) | AATTCCTTCTTTTTGAACGTGAACTCACAAGTTACGACCGTCTGT | |
| | CAAGCTCTCGCGAAAGACCCTAAGCTCCAGCAAGGTTATAATGC | |
| | AATGGGCTTCTCACAGGGAGGTCAGTTCTTGCGAGCGGTAGCCC | |
| | AGAGGTGTCCGTCTCCGCCAATGATCAACTTGATCTCAGTGGGG | |
| | GGTCAGCACCAAGGCGTTTTTGGACTCCCTAGATGCCCTGGAGA | |
| | GAGCTCTCACATTTGCGATTTTATACGGAAGACGCTGAATGCCG | |
| | GCGCGTATTCAAAGGTCGTTCAAGAGCGACTCGTCCAGGCTGAA | |
| | TACTGGCACGATCCGATTAAGGAAGACGTGTATCGAAACCATTC | |
| | TATCTTTCTTGCCGACATTAACCAGGAGCGAGGGATCAACGAAA | |
| | GTTATAAAAAAAACCTGATGGCACTCAAGAAATTTGTAATGGTT | |
| | AAATTCCTGAACGATTCAATAGTTGATCCGGTGGATTCCGAGTG | |
| | GTTCGGCTTCTACCGGTCCGGTCAGGCCAAGGAAACAATCCCAT | |
| | TGCAAGAAACCAGTCTCTATACTCAGGACCGCCTGGGTCTGAAA | |
| | GAAATGGACAACGCTGGCCAACTTGTTTTTCTGGCAACGGAGGG | |
| | TGATCACTTGCAGCTCTCTGAAGAATGGTTTTACGCACACATCAT | |
| | TCCTTTCCTTGGTTAA | |
| PPT1-14 | ATGAAGCTCAGTCTCGTGGCAGCTATGCTCCTCCTGCTGTCCCTG | 213 |
| (BiP1- | GTTGCGGCAATGTTGCTCTTGCTGAGCGCCGCGAGAGCAAGTCG | |
| vIGF2- | CACGTTGTGTGGAGGTGAACTCGTCGACACCCTTCAGTTCGTATG | |
| PPT1; | TGGAGATCGCGGTTTCCTCTTCTCACGCCCAGCTTCCAGAGTTTC | |
| Codon | CCGAAGATCACGAGGAATAGTTGAGGAGTGCTGTTTTCGGTCTT | |
| optimized | GTGATCTGGCTCTCCTCGAGACTTATTGTGCTACGCCGGCCCGCT | |
| IDT) | CTGAAGGAGGTGGTGGCAGTGGAGGAGGAGGGAGTCGGCCTAG | |
| | GGCAGTCCCAACCCAGGATCCCCCAGCACCCCTCCCCCTGGTAA | |
| | TTTGGCATGGAATGGGTGATTCCTGCTGTAACCCACTCTCAATGG | |
| | GGGCAATTAAGAAAATGGTAGAGAAAAAGATCCCTGGCATTTAT | |
| | GTTCTGTCACTCGAAATCGGTAAAACGCTCATGGAGGACGTAGA | |
| | AAACAGCTTTTTTCTGAATGTTAATTCACAGGTTACCACGGTCTG | |
| | CCAAGCATTGGCAAAGGACCCGAAATTGCAACAAGGCTATAACG | |
| | CGATGGGGTTCAGCCAAGGCGGGCAGTTTCTTCGAGCTGTGGCT | |
| | CAGCGCTGCCCTTCCCCACCGATGATAAATTTGATTAGCGTAGGG | |
| | GGACAACATCAAGGGGTTTTCGGTTTGCCAAGGTGTCCTGGCGA | |
| | ATCTTCACATATTTGCGACTTTATACGGAAGACCTTGAATGCGGG | |
| | GGCGTATAGTAAAGTCGTCCAGGAACGGCTTGTCCAAGCTGAAT | |
| | ACTGGCACGATCCCATCAAAGAAGATGTCTATCGGAATCACAGC | |
| | ATTTTTCTCGCCGACATAAACCAAGAACGCGGAATTAATGAGTC | |
| | ATACAAGAAGAACTTGATGGCACTTAAAAAATTTGTGATGGTTA | |
| | AGTTTTTGAATGATAGTATCGTAGATCCCGTAGATAGTGAATGGT | |
| | TTGGTTTCTATCGATCCGGACAGGCTAAAGAAACGATACCATTG | |
| | CAGGAAACCTCTTTGTATACTCAAGATAGGTTGGGCCTCAAGGA | |
| | GATGGATAATGCGGGGCAACTTGTCTTCCTCGCGACTGAGGGTG | |
| | ACCACCTCCAGCTCAGCGAGGAATGGTTTTACGCCCACATCATTC | |
| | CTTTCCTTGGTTAA | |
| PPT1-15 | ATGAAGCTCAGTCTCGTGGCAGCTATGCTCCTCCTGCTGTCCCTG | 214 |
| (BiP1aa- | GTTGCGGCAATGTTGCTCTTGCTGAGCGCCGCGAGAGCAGCAGC | |
| vIGF2- | TAGTCGCACGTTGTGTGGAGGTGAACTCGTCGACACCCTTCAGTT | |
| PPT1; | CGTATGTGGAGATCGCGGTTTCCTCTTCTCACGCCCAGCTTCCAG | |
| Codon | AGTTTCCCGAAGATCACGAGGAATAGTTGAGGAGTGCTGTTTTC | |
| optimized | GGTCTTGTGATCTGGCTCTCCTCGAGACTTATTGTGCTACGCCGG | |
| IDT) | CCCGCTCTGAAGGAGGTGGTGGCAGTGGAGGAGGAGGGAGTCG | |
| | GCCTAGGGCAGTCCCAACCCAGGATCCCCCAGCACCCCTCCCCC | |
| | TGGTAATTTGGCATGGAATGGGTGATTCCTGCTGTAACCCACTCT | |
| | CAATGGGGGCAATTAAGAAAATGGTAGAGAAAAAGATCCCTGG | |
| | CATTTATGTTCTGTCACTCGAAATCGGTAAAACGCTCATGGAGGA | |
| | CGTAGAAAACAGCTTTTTTCTGAATGTTAATTCACAGGTTACCAC | |
| | GGTCTGCCAAGCATTGGCAAAGGACCCGAAATTGCAACAAGGCT | |
| | ATAACGCGATGGGGTTCAGCCAAGGCGGGCAGTTTCTTCGAGCT | |
| | GTGGCTCAGCGCTGCCCTTCCCCACCGATGATAAATTTGATTAGC | |
| | GTAGGGGGACAACATCAAGGGGTTTTCGGTTTGCCAAGGTGTCC | |
| | TGGCGAATCTTCACATATTTGCGACTTTATACGGAAGACCTTGAA | |
| | TGCGGGGGCGTATAGTAAAGTCGTCCAGGAACGGCTTGTCCAAG | |
| | CTGAATACTGGCACGATCCCATCAAAGAAGATGTCTATCGGAAT | |
| | CACAGCATTTTTCTCGCCGACATAAACCAAGAACGCGGAATTAA | |
| | TGAGTCATACAAGAAGAACTTGATGGCACTTAAAAAATTTGTGA | |
| | TGGTTAAGTTTTTGAATGATAGTATCGTAGATCCCGTAGATAGTG | |
| | AATGGTTTGGTTTCTATCGATCCGGACAGGCTAAAGAAACGATA | |
| | CCATTGCAGGAAACCTCTTTGTATACTCAAGATAGGTTGGGCCTC | |
| | AAGGAGATGGATAATGCGGGGCAACTTGTCTTCCTCGCGACTGA | |
| | GGGTGACCACCTCCAGCTCAGCGAGGAATGGTTTTACGCCCACA | |
| | TCATTCCTTTCCTTGGTTAA | |
| PPT1-16 | ATGAAGCTCAGTCTCGTGGCAGCTATGCTCCTCCTGCTGTCCCTG | 215 |
| (BiP1aa- | GTTGCGGCAATGTTGCTCTTGCTGAGCGCCGCGAGAGCAGCCGC | |
| PPT1_2; | GTCAAGAGCTCTTCAACATCTGGATCCCCCAGCTCCCCTGCCGCT | |
| Codon | CGTAATCTGGCACGGGATGGGGGATTCATGTTGTAACCCGTTGTC | |
| optimized | AATGGGCGCGATAAAAAAGATGGTTGAAAAGAAGATTCCAGGC | |
| IDT) | ATCTACGTTCTGTCCCTGGAAATCGGTAAGACACTGATGGAAGA | |
| | CGTGGAGAACTCCTTCTTTCTCAACGTCAATAGTCAGGTCACTAC | |
| | CGTCTGTCAAGCATTGGCAAAGGACCCTAAACTTCAGCAGGGGT | |
| | ACAATGCGATGGGGTTTAGCCAGGGCGGACAGTTTCTTAGAGCC | |
| | GTCGCACAGCGCTGTCCATCTCCCCCGATGATTAACCTTATATCT | |
| | GTCGGGGGACAACACCAGGGTGTTTTTGGTCTTCCTCGCTGTCCT | |
| | GGTGAAAGCTCCCACATCTGTGATTTCATACGCAAAACGTTGAA | |
| | CGCAGGAGCTTATAGTAAAGTCGTCCAAGAACGGCTTGTTCAAG | |
| | CGGAGTATTGGCATGACCCAATAAAAGAAGACGTTTATAGGAAT | |
| | CACTCTATCTTCTTGGCCGATATCAACCAAGAACGCGGAATCAA | |
| | CGAAAGCTACAAAAAGAATCTTATGGCTCTCAAGAAATTTGTTA | |
| | TGGTGAAATTCCTTAATGACTCTATAGTAGATCCTGTCGATTCAG | |
| | AATGGTTCGGGTTCTACAGGTCTGGCCAGGCGAAGGAGACTATT | |
| | CCCCTCCAAGAAACGTCTCTCTATACACAAGACAGACTCGGACT | |
| | GAAAGAGATGGATAATGCGGGCCAGTTGGTCTTCTTGGCTACGG | |
| | AAGGCGATCATCTCCAACTCTCCGAAGAGTGGTTCTATGCCCATA | |
| | TAATCCCGTTCCTGGGCTAA | |
| PPT1-17 | ATGGCGTCGCCCGGCAGCCTGTGGCTCTTGGCTGTGGCTCTCCTG | 216 |
| (wt-PPT1- | CCATGGACCTGCGCTTCTCGGGCGCTGCAGCATCTGGACCCGCC | |
| C6S; natural | GGCGCCGCTGCCGTTGGTGATCTGGCATGGGATGGGAGACAGCT | |
| human | GTTGCAATCCCTTAAGCATGGGTGCTATTAAAAAAATGGTGGAG | |
| sequence) | AAGAAAATACCTGGAATTTACGTCTTATCTTTAGAGATTGGGAA | |
| | GACCCTGATGGAGGACGTGGAGAACAGCTTCTTCTTGAATGTCA | |
| | ATTCCCAAGTAACAACAGTGTGTCAGGCACTTGCTAAGGATCCT | |
| | AAATTGCAGCAAGGCTACAATGCTATGGGATTCTCCCAGGGAGG | |
| | CCAATTTCTGAGGGCAGTGGCTCAGAGATGCCCTTCACCTCCCAT | |
| | GATCAATCTGATCTCGGTTGGGGGACAACATCAAGGTGTTTTTGG | |
| | ACTCCCTCGATGCCCAGGAGAGAGCTCTCACATCTGTGACTTCAT | |
| | CCGAAAAACACTGAATGCTGGGGCGTACTCCAAAGTTGTTCAGG | |
| | AACGCCTCGTGCAAGCCGAATACTGGCATGACCCCATAAAGGAG | |
| | GATGTGTATCGCAACCACAGCATCTTCTTGGCAGATATAAATCA | |
| | GGAGCGGGGTATCAATGAGTCCTACAAGAAAAACCTGATGGCCC | |
| | TGAAGAAGTTTGTGATGGTGAAATTCCTCAATGATTCCATTGTGG | |
| | ACCCTGTAGATTCGGAGTGGTTTGGATTTTACAGAAGTGGCCAA | |
| | GCCAAGGAAACCATTCCCTTACAGGAGACCTCCCTGTACACACA | |
| | GGACCGCCTGGGGCTAAAGGAAATGGACAATGCAGGACAGCTA | |
| | GTGTTTCTGGCTACAGAAGGGGACCATCTTCAGTTGTCTGAAGA | |
| | ATGGTTTTATGCCCACATCATACCATTCCTTGGATGA | |
| PPT1-18 | ATGAAGCTCTCCCTGGTGGCCGCGATGCTGCTGCTGCTCTGGGTG | 217 |
| (BiP2aa- | GCACTGCTGCTGCTCAGCGCGGCGAGGGCCGCCGCGTCAAGAGC | |
| PPT1; | TCTTCAACATCTGGATCCCCCAGCTCCCCTGCCGCTCGTAATCTG | |
| Codon | GCACGGGATGGGGGATTCATGTTGTAACCCGTTGTCAATGGGCG | |
| optimized | CGATAAAAAAGATGGTTGAAAAGAAGATTCCAGGCATCTACGTT | |
| IDT) | CTGTCCCTGGAAATCGGTAAGACACTGATGGAAGACGTGGAGAA | |
| | CTCCTTCTTTCTCAACGTCAATAGTCAGGTCACTACCGTCTGTCA | |
| | AGCATTGGCAAAGGACCCTAAACTTCAGCAGGGGTACAATGCGA | |
| | TGGGGTTTAGCCAGGGCGGACAGTTTCTTAGAGCCGTCGCACAG | |
| | CGCTGTCCATCTCCCCCGATGATTAACCTTATATCTGTCGGGGGA | |
| | CAACACCAGGGTGTTTTTGGTCTTCCTCGCTGTCCTGGTGAAAGC | |
| | TCCCACATCTGTGATTTCATACGCAAAACGTTGAACGCAGGAGC | |
| | TTATAGTAAAGTCGTCCAAGAACGGCTTGTTCAAGCGGAGTATT | |
| | GGCATGACCCAATAAAAGAAGACGTTTATAGGAATCACTCTATC | |
| | TTCTTGGCCGATATCAACCAAGAACGCGGAATCAACGAAAGCTA | |
| | CAAAAAGAATCTTATGGCTCTCAAGAAATTTGTTATGGTGAAATT | |
| | CCTTAATGACTCTATAGTAGATCCTGTCGATTCAGAATGGTTCGG | |
| | GTTCTACAGGTCTGGCCAGGCGAAGGAGACTATTCCCCTCCAAG | |
| | AAACGTCTCTCTATACACAAGACAGACTCGGACTGAAAGAGATG | |
| | GATAATGCGGGCCAGTTGGTCTTCTTGGCTACGGAAGGCGATCA | |
| | TCTCCAACTCTCCGAAGAGTGGTTCTATGCCCATATAATCCCGTT | |
| | CCTGGGCTAA | |
| PPT1-19 | ATGGGTGTAAAGGTGTTGTTCGCTCTTATCTGCATTGCCGTTGCA | 218 |
| (GaussiaAA- | GAAGCTGCCGCGTCAAGAGCTCTTCAACATCTGGATCCCCCAGC | |
| PPT1_2; | TCCCCTGCCGCTCGTAATCTGGCACGGGATGGGGGATTCATGTTG | |
| Codon | TAACCCGTTGTCAATGGGCGCGATAAAAAAGATGGTTGAAAAGA | |
| optimized | AGATTCCAGGCATCTACGTTCTGTCCCTGGAAATCGGTAAGACA | |
| IDT) | CTGATGGAAGACGTGGAGAACTCCTTCTTTCTCAACGTCAATAGT | |
| | CAGGTCACTACCGTCTGTCAAGCATTGGCAAAGGACCCTAAACT | |
| | TCAGCAGGGGTACAATGCGATGGGGTTTAGCCAGGGCGGACAGT | |
| | TTCTTAGAGCCGTCGCACAGCGCTGTCCATCTCCCCCGATGATTA | |
| | ACCTTATATCTGTCGGGGGACAACACCAGGGTGTTTTTGGTCTTC | |
| | CTCGCTGTCCTGGTGAAAGCTCCCACATCTGTGATTTCATACGCA | |
| | AAACGTTGAACGCAGGAGCTTATAGTAAAGTCGTCCAAGAACGG | |
| | CTTGTTCAAGCGGAGTATTGGCATGACCCAATAAAAGAAGACGT | |
| | TTATAGGAATCACTCTATCTTCTTGGCCGATATCAACCAAGAACG | |
| | CGGAATCAACGAAAGCTACAAAAAGAATCTTATGGCTCTCAAGA | |
| | AATTTGTTATGGTGAAATTCCTTAATGACTCTATAGTAGATCCTG | |
| | TCGATTCAGAATGGTTCGGGTTCTACAGGTCTGGCCAGGCGAAG | |
| | GAGACTATTCCCCTCCAAGAAACGTCTCTCTATACACAAGACAG | |
| | ACTCGGACTGAAAGAGATGGATAATGCGGGCCAGTTGGTCTTCT | |
| | TGGCTACGGAAGGCGATCATCTCCAACTCTCCGAAGAGTGGTTC | |
| | TATGCCCATATAATCCCGTTCCTGGGCTAA | |
| PPT1-20 | ATGGGTGTAAAGGTGTTGTTCGCTCTTATCTGCATTGCCGTTGCA | 219 |
| (GaussiaAA- | GAAGCTGCAGCTAGTCGCACGTTGTGTGGAGGTGAACTCGTCGA | |
| VIGF2- | CACCCTTCAGTTCGTATGTGGAGATCGCGGTTTCCTCTTCTCACG | |
| PPT1; | CCCAGCTTCCAGAGTTTCCCGAAGATCACGAGGAATAGTTGAGG | |
| Codon | AGTGCTGTTTTCGGTCTTGTGATCTGGCTCTCCTCGAGACTTATTG | |
| optimized | TGCTACGCCGGCCCGCTCTGAAGGAGGTGGTGGCAGTGGAGGAG | |
| IDT) | GAGGGAGTCGGCCTAGGGCAGTCCCAACCCAGGATCCCCCAGCA | |
| | CCCCTCCCCCTGGTAATTTGGCATGGAATGGGTGATTCCTGCTGT | |
| | AACCCACTCTCAATGGGGGCAATTAAGAAAATGGTAGAGAAAA | |
| | AGATCCCTGGCATTTATGTTCTGTCACTCGAAATCGGTAAAACGC | |
| | TCATGGAGGACGTAGAAAACAGCTTTTTTCTGAATGTTAATTCAC | |
| | AGGTTACCACGGTCTGCCAAGCATTGGCAAAGGACCCGAAATTG | |
| | CAACAAGGCTATAACGCGATGGGGTTCAGCCAAGGCGGGCAGTT | |
| | TCTTCGAGCTGTGGCTCAGCGCTGCCCTTCCCCACCGATGATAAA | |
| | TTTGATTAGCGTAGGGGGACAACATCAAGGGGTTTTCGGTTTGCC | |
| | AAGGTGTCCTGGCGAATCTTCACATATTTGCGACTTTATACGGAA | |
| | GACCTTGAATGCGGGGGCGTATAGTAAAGTCGTCCAGGAACGGC | |
| | TTGTCCAAGCTGAATACTGGCACGATCCCATCAAAGAAGATGTC | |
| | TATCGGAATCACAGCATTTTTCTCGCCGACATAAACCAAGAACG | |
| | CGGAATTAATGAGTCATACAAGAAGAACTTGATGGCACTTAAAA | |
| | AATTTGTGATGGTTAAGTTTTTGAATGATAGTATCGTAGATCCCG | |
| | TAGATAGTGAATGGTTTGGTTTCTATCGATCCGGACAGGCTAAA | |
| | GAAACGATACCATTGCAGGAAACCTCTTTGTATACTCAAGATAG | |
| | GTTGGGCCTCAAGGAGATGGATAATGCGGGGCAACTTGTCTTCC | |
| | TCGCGACTGAGGGTGACCACCTCCAGCTCAGCGAGGAATGGTTT | |
| | TACGCCCACATCATTCCTTTCCTTGGTTAA | |
| PPT1-21 | ATGCTGGGGCTCTGGGGGCAGCGGCTCCCCGCGGCGTGGGTCCT | 220 |
| (ppt2ss- | GCTTCTGTTGCCTTTCCTGCCGCTGCTGCTGCTTGCAGATCCCCCA | |
| PPT1; | GCTCCCCTGCCGCTCGTAATCTGGCACGGGATGGGGGATTCATGT | |
| Codon | TGTAACCCGTTGTCAATGGGCGCGATAAAAAAGATGGTTGAAAA | |
| optimized | GAAGATTCCAGGCATCTACGTTCTGTCCCTGGAAATCGGTAAGA | |
| IDT) | CACTGATGGAAGACGTGGAGAACTCCTTCTTTCTCAACGTCAATA | |
| | GTCAGGTCACTACCGTCTGTCAAGCATTGGCAAAGGACCCTAAA | |
| | CTTCAGCAGGGGTACAATGCGATGGGGTTTAGCCAGGGCGGACA | |
| | GTTTCTTAGAGCCGTCGCACAGCGCTGTCCATCTCCCCCGATGAT | |
| | TAACCTTATATCTGTCGGGGGACAACACCAGGGTGTTTTTGGTCT | |
| | TCCTCGCTGTCCTGGTGAAAGCTCCCACATCTGTGATTTCATACG | |
| | CAAAACGTTGAACGCAGGAGCTTATAGTAAAGTCGTCCAAGAAC | |
| | GGCTTGTTCAAGCGGAGTATTGGCATGACCCAATAAAAGAAGAC | |
| | GTTTATAGGAATCACTCTATCTTCTTGGCCGATATCAACCAAGAA | |
| | CGCGGAATCAACGAAAGCTACAAAAAGAATCTTATGGCTCTCAA | |
| | GAAATTTGTTATGGTGAAATTCCTTAATGACTCTATAGTAGATCC | |
| | TGTCGATTCAGAATGGTTCGGGTTCTACAGGTCTGGCCAGGCGA | |
| | AGGAGACTATTCCCCTCCAAGAAACGTCTCTCTATACACAAGAC | |
| | AGACTCGGACTGAAAGAGATGGATAATGCGGGCCAGTTGGTCTT | |
| | CTTGGCTACGGAAGGCGATCATCTCCAACTCTCCGAAGAGTGGT | |
| | TCTATGCCCATATAATCCCGTTCCTGGGCTAA | |
| PPT1-22 | ATGCTGGGGCTCTGGGGGCAGCGGCTCCCCGCGGCGTGGGTCCT | 221 |
| (ppt2ss- | GCTTCTGTTGCCTTTCCTGCCGCTGCTGCTGCTTGCATCAAGAGC | |
| PPT1_2; | TCTTCAACATCTGGATCCCCCAGCTCCCCTGCCGCTCGTAATCTG | |
| Codon | GCACGGGATGGGGGATTCATGTTGTAACCCGTTGTCAATGGGCG | |
| optimized | CGATAAAAAAGATGGTTGAAAAGAAGATTCCAGGCATCTACGTT | |
| IDT) | CTGTCCCTGGAAATCGGTAAGACACTGATGGAAGACGTGGAGAA | |
| | CTCCTTCTTTCTCAACGTCAATAGTCAGGTCACTACCGTCTGTCA | |
| | AGCATTGGCAAAGGACCCTAAACTTCAGCAGGGGTACAATGCGA | |
| | TGGGGTTTAGCCAGGGCGGACAGTTTCTTAGAGCCGTCGCACAG | |
| | CGCTGTCCATCTCCCCCGATGATTAACCTTATATCTGTCGGGGGA | |
| | CAACACCAGGGTGTTTTTGGTCTTCCTCGCTGTCCTGGTGAAAGC | |
| | TCCCACATCTGTGATTTCATACGCAAAACGTTGAACGCAGGAGC | |
| | TTATAGTAAAGTCGTCCAAGAACGGCTTGTTCAAGCGGAGTATT | |
| | GGCATGACCCAATAAAAGAAGACGTTTATAGGAATCACTCTATC | |
| | TTCTTGGCCGATATCAACCAAGAACGCGGAATCAACGAAAGCTA | |
| | CAAAAAGAATCTTATGGCTCTCAAGAAATTTGTTATGGTGAAATT | |
| | CCTTAATGACTCTATAGTAGATCCTGTCGATTCAGAATGGTTCGG | |
| | GTTCTACAGGTCTGGCCAGGCGAAGGAGACTATTCCCCTCCAAG | |
| | AAACGTCTCTCTATACACAAGACAGACTCGGACTGAAAGAGATG | |
| | GATAATGCGGGCCAGTTGGTCTTCTTGGCTACGGAAGGCGATCA | |
| | TCTCCAACTCTCCGAAGAGTGGTTCTATGCCCATATAATCCCGTT | |
| | CCTGGGCTAA | |
| PPT1-23 | ATGGCAAGTCCTTCCTGTCTTTGGCTGCTGGCTGTTGCCTTGCTTC | 222 |
| (concensusS | CTTGGTCTTGTGCGGCGCGGGCACTCGGCCATTTGGACCCACCAG | |
| S-PPT1; | CCCCACTGCCCTTGGTTATATGGCATGGAATGGGAGATAGTTGCT | |
| Codon | GTAATCCACTGAGCATGGGAGCCATAAAGAAAATGGTTGAGAAA | |
| optimized | AAAATACCGGGAATATATGTTCTGAGCCTGGAGATAGGTAAGAC | |
| IDT) | ACTCATGGAAGACGTTGAAAACTCATTTTTTTTGAACGTGAATAG | |
| | TCAAGTCACAACGGTCTGTCAAGCTCTGGCTAAAGATCCTAAGTT | |
| | GCAACAGGGTTACAATGCGATGGGATTTAGTCAAGGTGGACAGT | |
| | TCCTGCGGGCCGTCGCACAGAGGTGCCCGAGTCCGCCAATGATA | |
| | AATCTCATTTCAGTAGGCGGACAACATCAGGGCGTGTTCGGTCTT | |
| | CCTCGCTGCCCGGGTGAGTCTTCTCACATTTGCGATTTCATACGC | |
| | AAAACACTTAACGCGGGGGCTTACTCCAAGGTAGTTCAAGAAAG | |
| | GCTCGTGCAGGCCGAATACTGGCATGATCCAATCAAAGAAGACG | |
| | TCTATAGAAATCACTCTATATTCTTGGCCGACATCAACCAAGAGC | |
| | GAGGTATAAATGAAAGTTACAAGAAAAACCTCATGGCTCTTAAA | |
| | AAATTTGTTATGGTAAAATTTCTTAATGACTCTATCGTTGACCCG | |
| | GTCGATAGTGAGTGGTTTGGGTTTTATAGGAGCGGACAGGCCAA | |
| | AGAGACAATTCCGTTGCAGGAGACAAGTTTGTACACGCAGGATA | |
| | GGCTTGGTCTTAAGGAGATGGACAACGCGGGCCAACTTGTATTT | |
| | TTGGCTACTGAAGGTGATCACCTCCAATTGTCTGAAGAGTGGTTT | |
| | TATGCGCATATTATTCCTTTCCTCGGCTAA | |
| PPT1-24 | ATGGCAAGCCCTTCCTGCCTCTGGTTGCTTGCTGTTGCTTTGCTTC | 223 |
| (consensus- | CTTGGTCTTGTGCTGCAAGAGCACTTGGCCACCTTGATCCTCCTG | |
| PPT1; | CACCTCTCCCGCTCGTTATATGGCACGGCATGGGGGATAGCTGTT | |
| Codon | GTAATCCACTGTCAATGGGGGCTATTAAGAAAATGGTGGAGAAG | |
| optimized | AAAATTCCGGGAATTTATGTGCTCTCCCTGGAGATAGGCAAAAC | |
| IDT) | GCTTATGGAAGACGTGGAGAACAGTTTTTTTCTTAACGTAAATTC | |
| | ACAGGTTACCACCGTCTGTCAAATTTTGGCCAAAGATCCCAAACT | |
| | GCAACAAGGGTATAACGCTATGGGCTTCAGTCAAGGGGGTCAAT | |
| | TTTTGAGGGCGGTTGCGCAACGCTGCCCTAGTCCGCCCATGATAA | |
| | ACTTGATCAGTGTTGGGGGACAGCACCAGGGAGTATTTGGTCTG | |
| | CCGAGGTGTCCAGGCGAGTCTTCACACATCTGTGACTTTATTCGC | |
| | AAGACCTTGAACGCGGGCGCTTATTCCAAGGCTGTGCAGGAAAG | |
| | GCTTGTGCAAGCGGAATATTGGCACGATCCTATAAAGGAAGATG | |
| | TGTATCGCAACCACTCTATCTTCCTGGCGGATATCAATCAAGAAC | |
| | GAGGAGTCAATGAGTCCTACAAGAAAAATCTGATGGCGCTTAAA | |
| | AAGTTCGTAATGGTCAAGTTCCTGAATGACAGCATAGTAGATCC | |
| | GGTGGATTCTGAATGGTTCGGATTCTACCGGTCAGGACAGGCCA | |
| | AGGAGACAATCCCCCTTCAAGAGACGACCCTGTACACACAAGAT | |
| | AGATTGGGACTGAAAGAAATGGATAAGGCCGGTCAATTGGTCTT | |
| | CTTGGCCACAGAAGGGGACCATCTCCAACTGAGTGAAGAATGGT | |
| | TTTATGCACATATAATTCCCTTCCTGGAGTAA | |
| PPT1-25 | ATGGCATCACCGGGTTGCCTCTGGTTGTTGGCCGTTGCGTTGCTT | 224 |
| (wt-PPT1 | CCGTGGACATGTGCATCAAGAGCTCTTCAACATCTGGATCCCCCA | |
| L283C | GCTCCCCTGCCGCTCGTAATCTGGCACGGGATGGGGGATTCATGT | |
| H300C; | TGTAACCCGTTGTCAATGGGCGCGATAAAAAAGATGGTTGAAAA | |
| Codon | GAAGATTCCAGGCATCTACGTTCTGTCCCTGGAAATCGGTAAGA | |
| optimized | CACTGATGGAAGACGTGGAGAACTCCTTCTTTCTCAACGTCAATA | |
| IDT) | GTCAGGTCACTACCGTCTGTCAAGCATTGGCAAAGGACCCTAAA | |
| | CTTCAGCAGGGGTACAATGCGATGGGGTTTAGCCAGGGCGGACA | |
| | GTTTCTTAGAGCCGTCGCACAGCGCTGTCCATCTCCCCCGATGAT | |
| | TAACCTTATATCTGTCGGGGGACAACACCAGGGTGTTTTTGGTCT | |
| | TCCTCGCTGTCCTGGTGAAAGCTCCCACATCTGTGATTTCATACG | |
| | CAAAACGTTGAACGCAGGAGCTTATAGTAAAGTCGTCCAAGAAC | |
| | GGCTTGTTCAAGCGGAGTATTGGCATGACCCAATAAAAGAAGAC | |
| | GTTTATAGGAATCACTCTATCTTCTTGGCCGATATCAACCAAGAA | |
| | CGCGGAATCAACGAAAGCTACAAAAAGAATCTTATGGCTCTCAA | |
| | GAAATTTGTTATGGTGAAATTCCTTAATGACTCTATAGTAGATCC | |
| | TGTCGATTCAGAATGGTTCGGGTTCTACAGGTCTGGCCAGGCGA | |
| | AGGAGACTATTCCCCTCCAAGAAACGTCTCTCTATACACAAGAC | |
| | AGACTCGGACTGAAAGAGATGGATAATGCGGGCCAGTTGGTCTT | |
| | CTGCGCTACGGAAGGCGATCATCTCCAACTCTCCGAAGAGTGGT | |
| | TCTATGCCTGCATAATCCCGTTCCTGGGCTAA | |
| PPT1-26 | ATGGCATCACCGGGTTGCCTCTGGTTGTTGGCCGTTGCGTTGCTT | 225 |
| (wt-PPT1 | CCGTGGACATGTGCATCAAGAGCTCTTCAACATCTGGATCCCCCA | |
| G113C | GCTCCCCTGCCGCTCGTAATCTGGCACGGGATGGGGGATTCATGT | |
| L121C; | TGTAACCCGTTGTCAATGGGCGCGATAAAAAAGATGGTTGAAAA | |
| Codon | GAAGATTCCAGGCATCTACGTTCTGTCCCTGGAAATCGGTAAGA | |
| optimized | CACTGATGGAAGACGTGGAGAACTCCTTCTTTCTCAACGTCAATA | |
| IDT) | GTCAGGTCACTACCGTCTGTCAAGCATTGGCAAAGGACCCTAAA | |
| | CTTCAGCAGGGGTACAATGCGATGTGCTTTAGCCAGGGCGGACA | |
| | GTTTTGCAGAGCCGTCGCACAGCGCTGTCCATCTCCCCCGATGAT | |
| | TAACCTTATATCTGTCGGGGGACAACACCAGGGTGTTTTTGGTCT | |
| | TCCTCGCTGTCCTGGTGAAAGCTCCCACATCTGTGATTTCATACG | |
| | CAAAACGTTGAACGCAGGAGCTTATAGTAAAGTCGTCCAAGAAC | |
| | GGCTTGTTCAAGCGGAGTATTGGCATGACCCAATAAAAGAAGAC | |
| | GTTTATAGGAATCACTCTATCTTCTTGGCCGATATCAACCAAGAA | |
| | CGCGGAATCAACGAAAGCTACAAAAAGAATCTTATGGCTCTCAA | |
| | GAAATTTGTTATGGTGAAATTCCTTAATGACTCTATAGTAGATCC | |
| | TGTCGATTCAGAATGGTTCGGGTTCTACAGGTCTGGCCAGGCGA | |
| | AGGAGACTATTCCCCTCCAAGAAACGTCTCTCTATACACAAGAC | |
| | AGACTCGGACTGAAAGAGATGGATAATGCGGGCCAGTTGGTCTT | |
| | CTTGGCTACGGAAGGCGATCATCTCCAACTCTCCGAAGAGTGGT | |
| | TCTATGCCCATATAATCCCGTTCCTGGGCTAA | |
| PPT1-27 | ATGGCATCACCGGGTTGCCTCTGGTTGTTGGCCGTTGCGTTGCTT | 226 |
| (wt-PPT1 | CCGTGGACATGTGCATCAAGAGCTCTTCAACATCTGGATCCCCCA | |
| A171C | GCTCCCCTGCCGCTCGTAATCTGGCACGGGATGGGGGATTCATGT | |
| A183C; | TGTAACCCGTTGTCAATGGGCGCGATAAAAAAGATGGTTGAAAA | |
| Codon | GAAGATTCCAGGCATCTACGTTCTGTCCCTGGAAATCGGTAAGA | |
| optimized | CACTGATGGAAGACGTGGAGAACTCCTTCTTTCTCAACGTCAATA | |
| IDT) | GTCAGGTCACTACCGTCTGTCAAGCATTGGCAAAGGACCCTAAA | |
| | CTTCAGCAGGGGTACAATGCGATGGGGTTTAGCCAGGGCGGACA | |
| | GTTTCTTAGAGCCGTCGCACAGCGCTGTCCATCTCCCCCGATGAT | |
| | TAACCTTATATCTGTCGGGGGACAACACCAGGGTGTTTTTGGTCT | |
| | TCCTCGCTGTCCTGGTGAAAGCTCCCACATCTGTGATTTCATACG | |
| | CAAAACGTTGAACGCAGGATGCTATAGTAAAGTCGTCCAAGAAC | |
| | GGCTTGTTCAATGCGAGTATTGGCATGACCCAATAAAAGAAGAC | |
| | GTTTATAGGAATCACTCTATCTTCTTGGCCGATATCAACCAAGAA | |
| | CGCGGAATCAACGAAAGCTACAAAAAGAATCTTATGGCTCTCAA | |
| | GAAATTTGTTATGGTGAAATTCCTTAATGACTCTATAGTAGATCC | |
| | TGTCGATTCAGAATGGTTCGGGTTCTACAGGTCTGGCCAGGCGA | |
| | AGGAGACTATTCCCCTCCAAGAAACGTCTCTCTATACACAAGAC | |
| | AGACTCGGACTGAAAGAGATGGATAATGCGGGCCAGTTGGTCTT | |
| | CTTGGCTACGGAAGGCGATCATCTCCAACTCTCCGAAGAGTGGT | |
| | TCTATGCCCATATAATCCCGTTCCTGGGCTAA | |
| PPT1-28 | ATGAAACTTAGTCTCGTCGCAGCAATGTTGCTTCTCCTGTGGGTT | 227 |
| (BiP2aa- | GCCCTCCTGTTGCTCAGCGCAGCTAGGGCTGCTGCGTCTCGGGCG | |
| PPT1; | CTGCAGCATCTGGACCCGCCGGCGCCGCTGCCGTTGGTGATCTG | |
| native | GCATGGGATGGGAGACAGCTGTTGCAATCCCTTAAGCATGGGTG | |
| human | CTATTAAAAAAATGGTGGAGAAGAAAATACCTGGAATTTACGTC | |
| sequence) | TTATCTTTAGAGATTGGGAAGACCCTGATGGAGGACGTGGAGAA | |
| | CAGCTTCTTCTTGAATGTCAATTCCCAAGTAACAACAGTGTGTCA | |
| | GGCACTTGCTAAGGATCCTAAATTGCAGCAAGGCTACAATGCTA | |
| | TGGGATTCTCCCAGGGAGGCCAATTTCTGAGGGCAGTGGCTCAG | |
| | AGATGCCCTTCACCTCCCATGATCAATCTGATCTCGGTTGGGGGA | |
| | CAACATCAAGGTGTTTTTGGACTCCCTCGATGCCCAGGAGAGAG | |
| | CTCTCACATCTGTGACTTCATCCGAAAAACACTGAATGCTGGGGC | |
| | GTACTCCAAAGTTGTTCAGGAACGCCTCGTGCAAGCCGAATACT | |
| | GGCATGACCCCATAAAGGAGGATGTGTATCGCAACCACAGCATC | |
| | TTCTTGGCAGATATAAATCAGGAGCGGGGTATCAATGAGTCCTA | |
| | CAAGAAAAACCTGATGGCCCTGAAGAAGTTTGTGATGGTGAAAT | |
| | TCCTCAATGATTCCATTGTGGACCCTGTAGATTCGGAGTGGTTTG | |
| | GATTTTACAGAAGTGGCCAAGCCAAGGAAACCATTCCCTTACAG | |
| | GAGACCTCCCTGTACACACAGGACCGCCTGGGGCTAAAGGAAAT | |
| | GGACAATGCAGGACAGCTAGTGTTTCTGGCTACAGAAGGGGACC | |
| | ATCTTCAGTIGTCTGAAGAATGGTTTTATGCCCACATCATACCAT | |
| | TCCTTGGATGA | |
| PPT1-101 | ATGAAGCTCTCCCTGGTGGCCGCGATGCTGCTGCTGCTCTGGGTG | 228 |
| | GCACTGCTGCTGCTCAGCGCGGCGAGGGCCGCCGCGTCTAGAAC | |
| | ACTGTGCGGAGGGGAGCTTGTAGACACTCTTCAGTTCGTGTGTG | |
| | GAGATCGCGGGTTCCTCTTCTCTCGCGGAGGTGGAGGTTCTAGG | |
| | GGTATACTGGAGGAGTGTTGTTTCAGGGACTGTGACTTGGCGCTC | |
| | CTCGAGACCTATTGCGCGACGCCAGCCAGGTCCGAAGGAGGTGG | |
| | TGGCAGTGGAGGAGGAGGGAGTCGGCCTAGGGCAGTCCCAACC | |
| | CAGGACCCGCCGGCGCCGCTGCCGTTGGTGATCTGGCATGGGAT | |
| | GGGAGACAGCTGTTGCAATCCCTTAAGCATGGGTGCTATTAAAA | |
| | AAATGGTGGAGAAGAAAATACCTGGAATTTACGTCTTATCTTTA | |
| | GAGATTGGGAAGACCCTGATGGAGGACGTGGAGAACAGCTTCTT | |
| | CTTGAATGTCAATTCCCAAGTAACAACAGTGTGTCAGGCACTTGC | |
| | TAAGGATCCTAAATTGCAGCAAGGCTACAATGCTATGGGATTCT | |
| | CCCAGGGAGGCCAATTTCTGAGGGCAGTGGCTCAGAGATGCCCT | |
| | TCACCTCCCATGATCAATCTGATCTCGGTTGGGGGACAACATCAA | |
| | GGTGTTTTTGGACTCCCTCGATGCCCAGGAGAGAGCTCTCACATC | |
| | TGTGACTTCATCCGAAAAACACTGAATGCTGGGGCGTACTCCAA | |
| | AGTTGTTCAGGAACGCCTCGTGCAAGCCGAATACTGGCATGACC | |
| | CCATAAAGGAGGATGTGTATCGCAACCACAGCATCTTCTTGGCA | |
| | GATATAAATCAGGAGCGGGGTATCAATGAGTCCTACAAGAAAAA | |
| | CCTGATGGCCCTGAAGAAGTTTGTGATGGTGAAATTCCTCAATG | |
| | ATTCCATTGTGGACCCTGTAGATTCGGAGTGGTTTGGATTTTACA | |
| | GAAGTGGCCAAGCCAAGGAAACCATTCCCTTACAGGAGACCTCC | |
| | CTGTACACACAGGACCGCCTGGGGCTAAAGGAAATGGACAATGC | |
| | AGGACAGCTAGTGTTTCTGGCTACAGAAGGGGACCATCTTCAGT | |
| | TGTCTGAAGAATGGTTTTATGCCCACATCATACCATTCCTTGGAT | |
| | GA | |
| PPT1-31 | ATGAAGCTCAGTCTCGTGGCAGCTATGCTCCTCCTGCTGTCCCTG | 229 |
| (BiP1- | GTTGCGGCAATGTTGCTCTTGCTGAGCGCCGCGAGAGCAAGTCG | |
| vIGF2- | CACGTTGTGTGGAGGTGAACTCGTCGACACCCTTCAGTTCGTATG | |
| PPT1; | TGGAGATCGCGGTTTCCTCTTCTCACGCCCAGCTTCCAGAGTTTC | |
| native | CCGAAGATCACGAGGAATAGTTGAGGAGTGCTGTTTTCGGTCTT | |
| human | GTGATCTGGCTCTCCTCGAGACTTATTGTGCTACGCCGGCCCGCT | |
| sequence) | CTGAAGGAGGTGGTGGCAGTGGAGGAGGAGGGAGTCGGCCTAG | |
| | GGCAGTCCCAACCCAGGACCCGCCGGCGCCGCTGCCGTTGGTGA | |
| | TCTGGCATGGGATGGGAGACAGCTGTTGCAATCCCTTAAGCATG | |
| | GGTGCTATTAAAAAAATGGTGGAGAAGAAAATACCTGGAATTTA | |
| | CGTCTTATCTTTAGAGATTGGGAAGACCCTGATGGAGGACGTGG | |
| | AGAACAGCTTCTTCTTGAATGTCAATTCCCAAGTAACAACAGTGT | |
| | GTCAGGCACTTGCTAAGGATCCTAAATTGCAGCAAGGCTACAAT | |
| | GCTATGGGATTCTCCCAGGGAGGCCAATTTCTGAGGGCAGTGGC | |
| | TCAGAGATGCCCTTCACCTCCCATGATCAATCTGATCTCGGTTGG | |
| | GGGACAACATCAAGGTGTTTTTGGACTCCCTCGATGCCCAGGAG | |
| | AGAGCTCTCACATCTGTGACTTCATCCGAAAAACACTGAATGCT | |
| | GGGGCGTACTCCAAAGTTGTTCAGGAACGCCTCGTGCAAGCCGA | |
| | ATACTGGCATGACCCCATAAAGGAGGATGTGTATCGCAACCACA | |
| | GCATCTTCTTGGCAGATATAAATCAGGAGCGGGGTATCAATGAG | |
| | TCCTACAAGAAAAACCTGATGGCCCTGAAGAAGTTTGTGATGGT | |
| | GAAATTCCTCAATGATTCCATTGTGGACCCTGTAGATTCGGAGTG | |
| | GTTTGGATTTTACAGAAGTGGCCAAGCCAAGGAAACCATTCCCT | |
| | TACAGGAGACCTCCCTGTACACACAGGACCGCCTGGGGCTAAAG | |
| | GAAATGGACAATGCAGGACAGCTAGTGTTTCTGGCTACAGAAGG | |
| | GGACCATCTTCAGTTGTCTGAAGAATGGTTTTATGCCCACATCAT | |
| | ACCATTCCTTGGATGA | |
| PPT1-32 | ATGGCGTCGCCCGGCTGCCTGTGGCTCTTGGCTGTGGCTCTCCTG | 230 |
| (wt-PPT1- | CCATGGACCTGCGCTTCTCGGGCGCTGCAGCATCTGGACCCGCC | |
| vIGF2-32; | GGCGCCGCTGCCGTTGGTGATCTGGCATGGGATGGGAGACAGCT | |
| native | GTTGCAATCCCTTAAGCATGGGTGCTATTAAAAAAATGGTGGAG | |
| human | AAGAAAATACCTGGAATTTACGTCTTATCTTTAGAGATTGGGAA | |
| sequence) | GACCCTGATGGAGGACGTGGAGAACAGCTTCTTCTTGAATGTCA | |
| | ATTCCCAAGTAACAACAGTGTGTCAGGCACTTGCTAAGGATCCT | |
| | AAATTGCAGCAAGGCTACAATGCTATGGGATTCTCCCAGGGAGG | |
| | CCAATTTCTGAGGGCAGTGGCTCAGAGATGCCCTTCACCTCCCAT | |
| | GATCAATCTGATCTCGGTTGGGGGACAACATCAAGGTGTTTTTGG | |
| | ACTCCCTCGATGCCCAGGAGAGAGCTCTCACATCTGTGACTTCAT | |
| | CCGAAAAACACTGAATGCTGGGGCGTACTCCAAAGTTGTTCAGG | |
| | AACGCCTCGTGCAAGCCGAATACTGGCATGACCCCATAAAGGAG | |
| | GATGTGTATCGCAACCACAGCATCTTCTTGGCAGATATAAATCA | |
| | GGAGCGGGGTATCAATGAGTCCTACAAGAAAAACCTGATGGCCC | |
| | TGAAGAAGTTTGTGATGGTGAAATTCCTCAATGATTCCATTGTGG | |
| | ACCCTGTAGATTCGGAGTGGTTTGGATTTTACAGAAGTGGCCAA | |
| | GCCAAGGAAACCATTCCCTTACAGGAGACCTCCCTGTACACACA | |
| | GGACCGCCTGGGGCTAAAGGAAATGGACAATGCAGGACAGCTA | |
| | GTGTTTCTGGCTACAGAAGGGGACCATCTTCAGTTGTCTGAAGA | |
| | ATGGTTTTATGCCCACATCATACCATTCCTTGGAAGACCTAGAGC | |
| | AGTGCCTACGCAGGGAGGGAGTGGGAGTGGATCCACTTCATCCT | |
| | CTAGAACACTGTGCGGAGGGGAGCTTGTAGACACTCTTCAGTTC | |
| | GTGTGTGGAGATCGCGGGTTCCTCTTCTCTCGCGGAGGTGGAGGT | |
| | TCTAGGGGTATACTGGAGGAGTGTTGTTTCAGGGAGTGTGACTT | |
| | GGCGCTCCTCGAGACCTATTGCGCGACGCCAGCCAGGTCCGAAT | |
| | GA | |
| PPT1-33 | ATGGCGTCGCCCGGCTGCCTGTGGCTCTTGGCTGTGGCTCTCCTG | 231 |
| (wt-PPT1- | CCATGGACCTGCGCTTCTCGGGCGCTGCAGCATCTGGACCCGCC | |
| vIGF2-8Q; | GGCGCCGCTGCCGTTGGTGATCTGGCATGGGATGGGAGACAGCT | |
| native | GTTGCAATCCCTTAAGCATGGGTGCTATTAAAAAAATGGTGGAG | |
| human | AAGAAAATACCTGGAATTTACGTCTTATCTTTAGAGATTGGGAA | |
| sequence) | GACCCTGATGGAGGACGTGGAGAACAGCTTCTTCTTGAATGTCA | |
| | ATTCCCAAGTAACAACAGTGTGTCAGGCACTTGCTAAGGATCCT | |
| | AAATTGCAGCAAGGCTACAATGCTATGGGATTCTCCCAGGGAGG | |
| | CCAATTTCTGAGGGCAGTGGCTCAGAGATGCCCTTCACCTCCCAT | |
| | GATCAATCTGATCTCGGTTGGGGGACAACATCAAGGTGTTTTTGG | |
| | ACTCCCTCGATGCCCAGGAGAGAGCTCTCACATCTGTGACTTCAT | |
| | CCGAAAAACACTGAATGCTGGGGCGTACTCCAAAGTTGTTCAGG | |
| | AACGCCTCGTGCAAGCCGAATACTGGCATGACCCCATAAAGGAG | |
| | GATGTGTATCGCAACCACAGCATCTTCTTGGCAGATATAAATCA | |
| | GGAGCGGGGTATCAATGAGTCCTACAAGAAAAACCTGATGGCCC | |
| | TGAAGAAGTTTGTGATGGTGAAATTCCTCAATGATTCCATTGTGG | |
| | ACCCTGTAGATTCGGAGTGGTTTGGATTTTACAGAAGTGGCCAA | |
| | GCCAAGGAAACCATTCCCTTACAGGAGACCTCCCTGTACACACA | |
| | GGACCGCCTGGGGCTAAAGGAAATGGACAATGCAGGACAGCTA | |
| | GTGTTTCTGGCTACAGAAGGGGACCATCTTCAGTTGTCTGAAGA | |
| | ATGGTTTTATGCCCACATCATACCATTCCTTGGAAGACCTAGAGC | |
| | AGTGCCTACGCAGGGAGGGAGTGGGAGTGGATCCACTTCATCCT | |
| | CTAGAACACTGTGCGGAGGGGAGCTTGTAGACACTCTTCAGTTC | |
| | GTGTGTGGAGATCGCGGGTTCCTCTTCTCTCGCCCCGCTTCCAGA | |
| | GTTTCACGGAGGTCTAGGGGTATAGTAGAGGAGTGTTGTTTCAG | |
| | GGAGTGTGACTTGGCGCTCCTCGAGACCTATTGCGCGACGCCAG | |
| | CCAGGTCCGAATGA | |
| PPT1-34 | ATGGCCTCCCCAGGCTGCTTATGGTTGCTGGCCGTAGCACTTTTA | 232 |
| (wt-PPT1- | CCATGGACATGTGCTAGTCGAGCTTTACAACACTTAGACCCGCC | |
| vIGF2-8Q; | AGCGCCTCTTCCTTTAGTTATCTGGCACGGCATGGGCGACTCGTG | |
| Codon | TTGTAACCCGCTCAGTATGGGTGCCATAAAGAAGATGGTGGAGA | |
| optimized | AGAAAATTCCCGGAATCTATGTGCTTAGCCTCGAAATCGGCAAA | |
| GENEius) | ACACTTATGGAGGACGTAGAGAACTCATTCTTCCTGAATGTAAA | |
| | TAGCCAAGTCACCACGGTATGTCAAGCTCTAGCGAAGGACCCTA | |
| | AACTCCAGCAGGGGTATAACGCAATGGGATTTTCTCAGGGCGGC | |
| | CAGTTTCTGCGTGCTGTCGCACAGCGTTGCCCTTCTCCGCCTATG | |
| | ATAAACTTAATTTCCGTAGGAGGGCAACACCAAGGGGTATTCGG | |
| | CTTACCGAGGTGTCCAGGCGAATCTTCACATATATGCGACTTCAT | |
| | CCGAAAGACCCTTAATGCCGGGGCCTATTCCAAGGTGGTACAGG | |
| | AACGGTTGGTGCAAGCTGAGTATTGGCACGACCCTATAAAGGAA | |
| | GATGTGTATCGGAATCACTCAATCTTTCTTGCGGATATAAATCAA | |
| | GAGCGCGGCATTAACGAGAGCTACAAGAAGAACCTCATGGCTCT | |
| | TAAGAAATTCGTCATGGTCAAATTCCTCAACGACAGTATAGTTG | |
| | ATCCCGTCGATTCGGAGTGGTTTGGATTCTACCGCTCTGGGCAAG | |
| | CCAAAGAGACCATACCACTACAGGAAACATCGCTATATACCCAA | |
| | GATCGCTTGGGTTTGAAAGAAATGGATAACGCCGGTCAGCTTGT | |
| | GTTCTTAGCGACAGAGGGTGATCATCTCCAGCTGTCGGAAGAAT | |
| | GGTTCTATGCCCACATAATACCTTTCCTTGGACGACCCCGTGCGG | |
| | TCCCAACGCAGGGTGGATCAGGTAGCGGCTCAACTAGTTCCAGC | |
| | CGTACGTTGTGCGGCGGAGAACTAGTAGACACTCTTCAATTCGTT | |
| | TGTGGGGATCGGGGCTTCCTCTTCAGCAGGCCAGCGTCACGCGT | |
| | GTCGCGTCGGAGCCGAGGTATAGTGGAAGAATGCTGCTTCCGCG | |
| | AATGTGATCTAGCACTCCTTGAAACCTACTGCGCGACGCCTGCCC | |
| | GAAGTGAATGA | |
| PPT1-35 | ATGGCTTCCCCTGGCTGCCTGTGGCTGCTCGCTGTGGCCCTCCTG | 233 |
| (wt-PPT1- | CCCTGGACCTGTGCTTCTCGGGCCCTTCAGCATCTGGACCCTCCA | |
| vIGF2-8Q; | GCCCCCCTCCCCTTGGTCATCTGGCACGGCATGGGCGACAGCTGC | |
| Codon | TGCAACCCTCTGTCCATGGGGGCCATCAAGAAAATGGTTGAGAA | |
| optimized | GAAGATCCCAGGCATCTACGTGCTGAGCCTGGAAATTGGCAAGA | |
| COOL) | CACTGATGGAGGATGTGGAAAACAGCTTCTTCCTGAATGTGAAC | |
| | TCCCAGGTGACCACCGTGTGCCAGGCTCTGGCCAAAGATCCCAA | |
| | GCTGCAGCAGGGCTACAATGCCATGGGATTCAGCCAGGGGGGCC | |
| | AGTTTCTGCGGGCTGTTGCCCAGAGGTGCCCCAGCCCCCCCATGA | |
| | TCAATCTCATCTCTGTGGGCGGGCAGCACCAGGGTGTGTTTGGCC | |
| | TGCCTCGCTGCCCTGGAGAAAGCAGCCACATTTGTGATTTCATCA | |
| | GGAAGACCTTAAATGCTGGAGCCTACAGCAAGGTGGTCCAGGAA | |
| | AGGCTGGTGCAGGCAGAGTACTGGCATGACCCCATCAAAGAGGA | |
| | CGTGTACAGAAACCACAGCATCTTCCTGGCTGACATCAACCAGG | |
| | AGAGAGGAATTAATGAGAGCTACAAGAAGAACCTCATGGCCTTG | |
| | AAAAAGTTTGTGATGGTGAAGTTCTTGAATGACTCCATCGTGGAT | |
| | CCTGTGGACAGTGAATGGTTTGGGTTCTACCGCTCTGGACAGGCC | |
| | AAGGAAACCATCCCCCTGCAAGAAACATCCCTGTACACCCAGGA | |
| | CCGCCTGGGGCTGAAGGAGATGGACAACGCCGGCCAACTGGTCT | |
| | TCCTTGCCACAGAAGGAGACCACCTGCAGCTGTCTGAGGAGTGG | |
| | TTCTATGCCCACATCATCCCCTTCCTGGGCCGGCCCAGGGCCGTG | |
| | CCCACACAGGGAGGCAGTGGCAGCGGCTCCACCAGCTCCAGCAG | |
| | GACCCTGTGTGGCGGCGAGCTGGTTGACACCCTCCAGTTCGTGTG | |
| | TGGGGACAGAGGCTTCCTCTTCTCCAGGCCCGCCAGCCGGGTGA | |
| | GCCGCCGCTCCCGGGGCATTGTGGAGGAATGTTGCTTCCGGGAG | |
| | TGTGACCTGGCCCTGCTGGAGACCTACTGTGCCACCCCTGCCCGG | |
| | AGTGAGTGA | |
| PPT1-101 | ATGAAGCTCTCCCTGGTGGCCGCGATGCTGCTGCTGCTCTGGGTG | 234 |
| (sequence | GCACTGCTGCTGCTCAGCGCGGCGAGGGCCGCCGCGTCTAGAAC | |
| encoding | ACTGTGCGGAGGGGAGCTTGTAGACACTCTTCAGTTCGTGTGTG | |
| signal | GAGATCGCGGGTTCCTCTTCTCTCGCGGAGGTGGAGGTTCTAGG | |
| peptide | GGTATACTGGAGGAGTGTTGTTTCAGGGACTGTGACTTGGCGCTC | |
| underlined) | CTCGAGACCTATTGCGCGACGCCAGCCAGGTCCGAAGGAGGTGG | |
| | TGGCAGTGGAGGAGGAGGGAGTCGGCCTAGGGCAGTCCCAACC | |
| | CAGGACCCGCCGGCGCCGCTGCCGTTGGTGATCTGGCATGGGAT | |
| | GGGAGACAGCTGTTGCAATCCCTTAAGCATGGGTGCTATTAAAA | |
| | AAATGGTGGAGAAGAAAATACCTGGAATTTACGTCTTATCTTTA | |
| | GAGATTGGGAAGACCCTGATGGAGGACGTGGAGAACAGCTTCTT | |
| | CTTGAATGTCAATTCCCAAGTAACAACAGTGTGTCAGGCACTTGC | |
| | TAAGGATCCTAAATTGCAGCAAGGCTACAATGCTATGGGATTCT | |
| | CCCAGGGAGGCCAATTTCTGAGGGCAGTGGCTCAGAGATGCCCT | |
| | TCACCTCCCATGATCAATCTGATCTCGGTTGGGGGACAACATCAA | |
| | GGTGTTTTTGGACTCCCTCGATGCCCAGGAGAGAGCTCTCACATC | |
| | TGTGACTTCATCCGAAAAACACTGAATGCTGGGGCGTACTCCAA | |
| | AGTTGTTCAGGAACGCCTCGTGCAAGCCGAATACTGGCATGACC | |
| | CCATAAAGGAGGATGTGTATCGCAACCACAGCATCTTCTTGGCA | |
| | GATATAAATCAGGAGCGGGGTATCAATGAGTCCTACAAGAAAAA | |
| | CCTGATGGCCCTGAAGAAGTTTGTGATGGTGAAATTCCTCAATG | |
| | ATTCCATTGTGGACCCTGTAGATTCGGAGTGGTTTGGATTTTACA | |
| | GAAGTGGCCAAGCCAAGGAAACCATTCCCTTACAGGAGACCTCC | |
| | CTGTACACACAGGACCGCCTGGGGCTAAAGGAAATGGACAATGC | |
| | AGGACAGCTAGTGTTTCTGGCTACAGAAGGGGACCATCTTCAGT | |
| | TGTCTGAAGAATGGTTTTATGCCCACATCATACCATTCCTTGGAT | |
| | GA | |
| PPT1-104 | ATGGCGTCGCCCGGCAGCCTGTGGCTCTTGGCTGTGGCTCTCCTG | 235 |
| (sequence | CCATGGACCTGCGCTTCTCGGGCGCTGCAGCATCTGGACCCGCC | |
| encoding | GGCGCCGCTGCCGTTGGTGATCTGGCATGGGATGGGAGACAGCT | |
| signal | GTTGCAATCCCTTAAGCATGGGTGCTATTAAAAAAATGGTGGAG | |
| peptide | AAGAAAATACCTGGAATTTACGTCTTATCTTTAGAGATTGGGAA | |
| underlined) | GACCCTGATGGAGGACGTGGAGAACAGCTTCTTCTTGAATGTCA | |
| | ATTCCCAAGTAACAACAGTGTGTCAGGCACTTGCTAAGGATCCT | |
| | AAATTGCAGCAAGGCTACAATGCTATGGGATTCTCCCAGGGAGG | |
| | CCAATTTCTGAGGGCAGTGGCTCAGAGATGCCCTTCACCTCCCAT | |
| | GATCAATCTGATCTCGGTTGGGGGACAACATCAAGGTGTTTTTGG | |
| | ACTCCCTCGATGCCCAGGAGAGAGCTCTCACATCTGTGACTTCAT | |
| | CCGAAAAACACTGAATGCTGGGGCGTACTCCAAAGTTGTTCAGG | |
| | AACGCCTCGTGCAAGCCGAATACTGGCATGACCCCATAAAGGAG | |
| | GATGTGTATCGCAACCACAGCATCTTCTTGGCAGATATAAATCA | |
| | GGAGCGGGGTATCAATGAGTCCTACAAGAAAAACCTGATGGCCC | |
| | TGAAGAAGTTTGTGATGGTGAAATTCCTCAATGATTCCATTGTGG | |
| | ACCCTGTAGATTCGGAGTGGTTTGGATTTTACAGAAGTGGCCAA | |
| | GCCAAGGAAACCATTCCCTTACAGGAGACCTCCCTGTACACACA | |
| | GGACCGCCTGGGGCTAAAGGAAATGGACAATGCAGGACAGCTA | |
| | GTGTTTCTGGCTACAGAAGGGGACCATCTTCAGTTGTCTGAAGA | |
| | ATGGTTTTATGCCCACATCATACCATTCCTTGGAAGACCTAGAGC | |
| | AGTGCCTACGCAGGGAGGGAGTGGGAGTGGATCCACTTCATCCT | |
| | CTAGAACACTGTGCGGAGGGGAGCTTGTAGACACTCTTCAGTTC | |
| | GTGTGTGGAGATCGCGGGTTCCTCTTCTCTCGCGGAGGTGGAGGT | |
| | TCTAGGGGTATACTGGAGGAGTGTTGTTTCAGGGAGTGTGACTT | |
| | GGCGCTCCTCGAGACCTATTGCGCGACGCCAGCCAGGTCCGAAT | |
| | GA | |
| PPT-112 | ATGGCGTCGCCCGGCAGCCTGTGGCTCTTGGCTGTGGCTCTCCTG | 236 |
| (sequence | CCATGGACCTGCGCTTCTCGGGCGCTGCAGCATCTGGCCGCGTCT | |
| encoding | AGAACACTGTGCGGAGGGGAGCTTGTAGACACTCTTCAGTTCGT | |
| signal | GTGTGGAGATCGCGGGTTCCTCTTCTCTCGCGGAGGTGGAGGTTC | |
| peptide | TAGGGGTATACTGGAGGAGTGTTGTTTCAGGGACTGTGACTTGG | |
| underlined) | CGCTCCTCGAGACCTATTGCGCGACGCCAGCCAGGTCCGAAGGA | |
| | GGTGGTGGCAGTGGAGGAGGAGGGAGTCGGCCTAGGGCAGTCC | |
| | CAACCCAGGACCCGCCGGCGCCGCTGCCGTTGGTGATCTGGCAT | |
| | GGGATGGGAGACAGCTGTTGCAATCCCTTAAGCATGGGTGCTAT | |
| | TAAAAAAATGGTGGAGAAGAAAATACCTGGAATTTACGTCTTAT | |
| | CTTTAGAGATTGGGAAGACCCTGATGGAGGACGTGGAGAACAGC | |
| | TTCTTCTTGAATGTCAATTCCCAAGTAACAACAGTGTGTCAGGCA | |
| | CTTGCTAAGGATCCTAAATTGCAGCAAGGCTACAATGCTATGGG | |
| | ATTCTCCCAGGGAGGCCAATTTCTGAGGGCAGTGGCTCAGAGAT | |
| | GCCCTTCACCTCCCATGATCAATCTGATCTCGGTTGGGGGACAAC | |
| | ATCAAGGTGTTTTTGGACTCCCTCGATGCCCAGGAGAGAGCTCTC | |
| | ACATCTGTGACTTCATCCGAAAAACACTGAATGCTGGGGCGTAC | |
| | TCCAAAGTTGTTCAGGAACGCCTCGTGCAAGCCGAATACTGGCA | |
| | TGACCCCATAAAGGAGGATGTGTATCGCAACCACAGCATCTTCT | |
| | TGGCAGATATAAATCAGGAGCGGGGTATCAATGAGTCCTACAAG | |
| | AAAAACCTGATGGCCCTGAAGAAGTTTGTGATGGTGAAATTCCT | |
| | CAATGATTCCATTGTGGACCCTGTAGATTCGGAGTGGTTTGGATT | |
| | TTACAGAAGTGGCCAAGCCAAGGAAACCATTCCCTTACAGGAGA | |
| | CCTCCCTGTACACACAGGACCGCCTGGGGCTAAAGGAAATGGAC | |
| | AATGCAGGACAGCTAGTGTTTCTGGCTACAGAAGGGGACCATCT | |
| | TCAGTTGTCTGAAGAATGGTTTTATGCCCACATCATACCATTCCT | |
| | TGGATGA | |
| PPT-114 | ATGGCGTCGCCCGGCAGCCTGTGGCTCTTGGCTGTGGCTCTCCTG | 237 |
| (sequence | CCATGGACCTGCGCTTCTCGGGCGCTGCAGCATCTGGCCGCGTCT | |
| encoding | AGAACACTGTGCGGAGGGGAGCTTGTAGACACTCTTCAGTTCGT | |
| signal | GTGTGGAGATCGCGGGTTCCTCTTCTCTCGCGGAGGTGGAGGTTC | |
| peptide | TAGGGGTATACTGGAGGAGTGTTGTTTCAGGGAGTGTGACTTGG | |
| underlined) | CGCTCCTCGAGACCTATTGCGCGACGCCAGCCAGGTCCGAAGGA | |
| | GGTGGTGGCAGTGGAGGAGGAGGGAGTCGGCCTAGGGCAGTCC | |
| | CAACCCAGGACCCGCCGGCGCCGCTGCCGTTGGTGATCTGGCAT | |
| | GGGATGGGAGACAGCTGTTGCAATCCCTTAAGCATGGGTGCTAT | |
| | TAAAAAAATGGTGGAGAAGAAAATACCTGGAATTTACGTCTTAT | |
| | CTTTAGAGATTGGGAAGACCCTGATGGAGGACGTGGAGAACAGC | |
| | TTCTTCTTGAATGTCAATTCCCAAGTAACAACAGTGTGTCAGGCA | |
| | CTTGCTAAGGATCCTAAATTGCAGCAAGGCTACAATGCTATGGG | |
| | ATTCTCCCAGGGAGGCCAATTTCTGAGGGCAGTGGCTCAGAGAT | |
| | GCCCTTCACCTCCCATGATCAATCTGATCTCGGTTGGGGGACAAC | |
| | ATCAAGGTGTTTTTGGACTCCCTCGATGCCCAGGAGAGAGCTCTC | |
| | ACATCTGTGACTTCATCCGAAAAACACTGAATGCTGGGGCGTAC | |
| | TCCAAAGTTGTTCAGGAACGCCTCGTGCAAGCCGAATACTGGCA | |
| | TGACCCCATAAAGGAGGATGTGTATCGCAACCACAGCATCTTCT | |
| | TGGCAGATATAAATCAGGAGCGGGGTATCAATGAGTCCTACAAG | |
| | AAAAACCTGATGGCCCTGAAGAAGTTTGTGATGGTGAAATTCCT | |
| | CAATGATTCCATTGTGGACCCTGTAGATTCGGAGTGGTTTGGATT | |
| | TTACAGAAGTGGCCAAGCCAAGGAAACCATTCCCTTACAGGAGA | |
| | CCTCCCTGTACACACAGGACCGCCTGGGGCTAAAGGAAATGGAC | |
| | AATGCAGGACAGCTAGTGTTTCTGGCTACAGAAGGGGACCATCT | |
| | TCAGTTGTCTGAAGAATGGTTTTATGCCCACATCATACCATTCCT | |
| | TGGATGA | |
| PPT1-115 | ATGGCGTCGCCCGGCAGCCTGTGGCTCTTGGCTGTGGCTCTCCTG | 238 |
| (sequence | CCATGGACCTGCGCTTCTCGGGCGCTGCAGCATCTGGCTGCCGAC | |
| encoding | CCGCCGGCGCCGCTGCCGTTGGTGATCTGGCATGGGATGGGAGA | |
| signal | CAGCTGTTGCAATCCCTTAAGCATGGGTGCTATTAAAAAAATGG | |
| peptide | TGGAGAAGAAAATACCTGGAATTTACGTCTTATCTTTAGAGATTG | |
| underlined) | GGAAGACCCTGATGGAGGACGTGGAGAACAGCTTCTTCTTGAAT | |
| | GTCAATTCCCAAGTAACAACAGTGTGTCAGGCACTTGCTAAGGA | |
| | TCCTAAATTGCAGCAAGGCTACAATGCTATGGGATTCTCCCAGG | |
| | GAGGCCAATTTCTGAGGGCAGTGGCTCAGAGATGCCCTTCACCT | |
| | CCCATGATCAATCTGATCTCGGTTGGGGGACAACATCAAGGTGT | |
| | TTTTGGACTCCCTCGATGCCCAGGAGAGAGCTCTCACATCTGTGA | |
| | CTTCATCCGAAAAACACTGAATGCTGGGGCGTACTCCAAAGTTG | |
| | TTCAGGAACGCCTCGTGCAAGCCGAATACTGGCATGACCCCATA | |
| | AAGGAGGATGTGTATCGCAACCACAGCATCTTCTTGGCAGATAT | |
| | AAATCAGGAGCGGGGTATCAATGAGTCCTACAAGAAAAACCTGA | |
| | TGGCCCTGAAGAAGTTTGTGATGGTGAAATTCCTCAATGATTCCA | |
| | TTGTGGACCCTGTAGATTCGGAGTGGTTTGGATTTTACAGAAGTG | |
| | GCCAAGCCAAGGAAACCATTCCCTTACAGGAGACCTCCCTGTAC | |
| | ACACAGGACCGCCTGGGGCTAAAGGAAATGGACAATGCAGGAC | |
| | AGCTAGTGTTTCTGGCTACAGAAGGGGACCATCTTCAGTTGTCTG | |
| | AAGAATGGTTTTATGCCCACATCATACCATTCCTTGGAAGACCTA | |
| | GAGCAGTGCCTACGCAGGGAGGGAGTGGGAGTGGATCCACTTCA | |
| | TCCTCTAGAACACTGTGCGGAGGGGAGCTTGTAGACACTCTTCA | |
| | GTTCGTGTGTGGAGATCGCGGGTTCCTCTTCTCTCGCGGAGGTGG | |
| | AGGTTCTAGGGGTATACTGGAGGAGTGTTGTTTCAGGGAGTGTG | |
| | ACTTGGCGCTCCTCGAGACCTATTGCGCGACGCCAGCCAGGTCC | |
| | GAATGA | |
| PPT-116 | ATGGCGTCGCCCGGCAGCCTGTGGCTCTTGGCTGTGGCTCTCCTG | 239 |
| (sequence | CCATGGACCTGCGCTTCTCGGGCGCTGCAGCATCTGGCTGCCGAC | |
| encoding | CCGCCGGCGCCGCTGCCGTTGGTGATCTGGCATGGGATGGGAGA | |
| signal | CAGCTGTTGCAATCCCTTAAGCATGGGTGCTATTAAAAAAATGG | |
| peptide | TGGAGAAGAAAATACCTGGAATTTACGTCTTATCTTTAGAGATTG | |
| underlined) | GGAAGACCCTGATGGAGGACGTGGAGAACAGCTTCTTCTTGAAT | |
| | GTCAATTCCCAAGTAACAACAGTGTGTCAGGCACTTGCTAAGGA | |
| | TCCTAAATTGCAGCAAGGCTACAATGCTATGGGATTCTCCCAGG | |
| | GAGGCCAATTTCTGAGGGCAGTGGCTCAGAGATGCCCTTCACCT | |
| | CCCATGATCAATCTGATCTCGGTTGGGGGACAACATCAAGGTGT | |
| | TTTTGGACTCCCTCGATGCCCAGGAGAGAGCTCTCACATCTGTGA | |
| | CTTCATCCGAAAAACACTGAATGCTGGGGCGTACTCCAAAGTTG | |
| | TTCAGGAACGCCTCGTGCAAGCCGAATACTGGCATGACCCCATA | |
| | AAGGAGGATGTGTATCGCAACCACAGCATCTTCTTGGCAGATAT | |
| | AAATCAGGAGCGGGGTATCAATGAGTCCTACAAGAAAAACCTGA | |
| | TGGCCCTGAAGAAGTTTGTGATGGTGAAATTCCTCAATGATTCCA | |
| | TTGTGGACCCTGTAGATTCGGAGTGGTTTGGATTTTACAGAAGTG | |
| | GCCAAGCCAAGGAAACCATTCCCTTACAGGAGACCTCCCTGTAC | |
| | ACACAGGACCGCCTGGGGCTAAAGGAAATGGACAATGCAGGAC | |
| | AGCTAGTGTTTCTGGCTACAGAAGGGGACCATCTTCAGTTGTCTG | |
| | AAGAATGGTTTTATGCCCACATCATACCATTCCTTGGAAGACCTA | |
| | GAGCAGTGCCTACGCAGGGAGGGGGTGGCAGTGGCAGTGGAGG | |
| | CGGCGGTTCCTCTAGAACACTGTGCGGAGGGGAGCTTGTAGACA | |
| | CTCTTCAGTTCGTGTGTGGAGATCGCGGGTTCCTCTTCTCTCGCG | |
| | GAGGTGGAGGTTCTAGGGGTATACTGGAGGAGTGTTGTTTCAGG | |
| | GAGTGTGACTTGGCGCTCCTCGAGACCTATTGCGCGACGCCAGC | |
| | CAGGTCCGAATGA | |
| PPT1-117 | ATGGCGTCGCCCGGCAGCCTGTGGCTCTTGGCTGTGGCTCTCCTG | 240 |
| (sequence | CCATGGACCTGCGCTTCTCGGGCGCTGCAGCATCTGGACCCGCC | |
| encoding | GGCGCCGCTGCCGTTGGTGATCTGGCATGGGATGGGAGACAGCT | |
| signal | GTTGCAATCCCTTAAGCATGGGTGCTATTAAAAAAATGGTGGAG | |
| peptide | AAGAAAATACCTGGAATTTACGTCTTATCTTTAGAGATTGGGAA | |
| underlined) | GACCCTGATGGAGGACGTGGAGAACAGCTTCTTCTTGAATGTCA | |
| | ATTCCCAAGTAACAACAGTGTGTCAGGCACTTGCTAAGGATCCT | |
| | AAATTGCAGCAAGGCTACAATGCTATGGGATTCTCCCAGGGAGG | |
| | CCAATTTCTGAGGGCAGTGGCTCAGAGATGCCCTTCACCTCCCAT | |
| | GATCAATCTGATCTCGGTTGGGGGACAACATCAAGGTGTTTTTGG | |
| | ACTCCCTCGATGCCCAGGAGAGAGCTCTCACATCTGTGACTTCAT | |
| | CCGAAAAACACTGAATGCTGGGGCGTACTCCAAAGTTGTTCAGG | |
| | AACGCCTCGTGCAAGCCGAATACTGGCATGACCCCATAAAGGAG | |
| | GATGTGTATCGCAACCACAGCATCTTCTTGGCAGATATAAATCA | |
| | GGAGCGGGGTATCAATGAGTCCTACAAGAAAAACCTGATGGCCC | |
| | TGAAGAAGTTTGTGATGGTGAAATTCCTCAATGATTCCATTGTGG | |
| | ACCCTGTAGATTCGGAGTGGTTTGGATTTTACAGAAGTGGCCAA | |
| | GCCAAGGAAACCATTCCCTTACAGGAGACCTCCCTGTACACACA | |
| | GGACCGCCTGGGGCTAAAGGAAATGGACAATGCAGGACAGCTA | |
| | GTGTTTCTGGCTACAGAAGGGGACCATCTTCAGTTGTCTGAAGA | |
| | ATGGTTTTATGCCCACATCATACCATTCCTTGGAAGACCTAGAGC | |
| | AGTGCCTACGCAGGGAGGGGGTGGCAGTGGCAGTGGAGGCGGC | |
| | GGTTCCTCTAGAACACTGTGCGGAGGGGAGCTTGTAGACACTCT | |
| | TCAGTTCGTGTGTGGAGATCGCGGGTTCCTCTTCTCTCGCGGAGG | |
| | TGGAGGTTCTAGGGGTATACTGGAGGAGTGTTGTTTCAGGGAGT | |
| | GTGACTTGGCGCTCCTCGAGACCTATTGCGCGACGCCAGCCAGG | |
| | TCCGAATGA | |
| PPT-118 | ATGGCGTCGCCCGGCAGCCTGTGGCTCTTGGCTGTGGCTCTCCTG | 241 |
| (sequence | CCATGGACCTGCGCTTCTCGGGCGCTGCAGCATCTGGCTGCCGAC | |
| encoding | CCGCCGGCGCCGCTGCCGTTGGTGATCTGGCATGGGATGGGAGA | |
| signal | CAGCTGTTGCAATCCCTTAAGCATGGGTGCTATTAAAAAAATGG | |
| peptide | TGGAGAAGAAAATACCTGGAATTTACGTCTTATCTTTAGAGATTG | |
| underlined) | GGAAGACCCTGATGGAGGACGTGGAGAACAGCTTCTTCTTGAAT | |
| | GTCAATTCCCAAGTAACAACAGTGTGTCAGGCACTTGCTAAGGA | |
| | TCCTAAATTGCAGCAAGGCTACAATGCTATGGGATTCTCCCAGG | |
| | GAGGCCAATTTCTGAGGGCAGTGGCTCAGAGATGCCCTTCACCT | |
| | CCCATGATCAATCTGATCTCGGTTGGGGGACAACATCAAGGTGT | |
| | TTTTGGACTCCCTCGATGCCCAGGAGAGAGCTCTCACATCTGTGA | |
| | CTTCATCCGAAAAACACTGAATGCTGGGGCGTACTCCAAAGTTG | |
| | TTCAGGAACGCCTCGTGCAAGCCGAATACTGGCATGACCCCATA | |
| | AAGGAGGATGTGTATCGCAACCACAGCATCTTCTTGGCAGATAT | |
| | AAATCAGGAGCGGGGTATCAATGAGTCCTACAAGAAAAACCTGA | |
| | TGGCCCTGAAGAAGTTTGTGATGGTGAAATTCCTCAATGATTCCA | |
| | TTGTGGACCCTGTAGATTCGGAGTGGTTTGGATTTTACAGAAGTG | |
| | GCCAAGCCAAGGAAACCATTCCCTTACAGGAGACCTCCCTGTAC | |
| | ACACAGGACCGCCTGGGGCTAAAGGAAATGGACAATGCAGGAC | |
| | AGCTAGTGTTTCTGGCTACAGAAGGGGACCATCTTCAGTTGTCTG | |
| | AAGAATGGTTTTATGCCCACATCATACCATTCCTTGGAAGACCTA | |
| | GAGCAGTGCCTACGCAGGGAGGGGGTGGCAGTGGAGGCGGCGG | |
| | TTCCTCTAGAACACTGTGCGGAGGGGAGCTTGTAGACACTCTTCA | |
| | GTTCGTGTGTGGAGATCGCGGGTTCCTCTTCTCTCGCGGAGGTGG | |
| | AGGTTCTAGGGGTATACTGGAGGAGTGTTGTTTCAGGGAGTGTG | |
| | ACTTGGCGCTCCTCGAGACCTATTGCGCGACGCCAGCCAGGTCC | |
| | GAATGA | |
| BIP2AA | ATGAAGCTCTCCCTGGTGGCCGCGATGCTGCTGCTGCTCTGGGTG | 242 |
| | GCACTGCTGCTGCTCAGCGCGGCGAGGGCCGCCGCG | |
| eSP C6S | ATGGCGTCGCCCGGCAGCCTGTGGCTCTTGGCTGTGGCTCTCCTG | 243 |
| | CCATGGACCTGCGCTTCTCGGGCGCTGCAGCATCTG | |
| eSP C6S | ATGGCGTCGCCCGGCAGCCTGTGGCTCTTGGCTGTGGCTCTCCTG | 244 |
| AA (used in | CCATGGACCTGCGCTTCTCGGGCGCTGCAGCATCTGGCCGCG | |
| PPT1-112 | | |
| and PPT1- | | |
| 114) | | |
| eSP C6S | ATGGCGTCGCCCGGCAGCCTGTGGCTCTTGGCTGTGGCTCTCCTG | 245 |
| AA (used in | CCATGGACCTGCGCTTCTCGGGCGCTGCAGCATCTGGCTGCC | |
| PPT1-115, | | |
| PPT1-116, | | |
| and PPT1- | | |
| 118)- | | |
| different | | |
| codon | | |
| usage for | | |
| AA portion | | |
| WT | ATGgaggccgtggccgtggccgccgccgtgggcgtgctgctgctggccgg | 246 |
| NAGLU- | cgccggcggcgccgccggcgacgaggcccgggaggccgccgccgtgcggg | |
| HPC4 | ccctggtggcccggctgctgggccccggccccgccgccgacttcagcgtt | |
| (sequence | agcgtggagcgggccctggccgccaagcccggcctggacacctacagcct | |
| encoding | gggcggcggcggcgccgcccgggtgcgggtgcggggcagcaccggcgtgg | |
| HPC4 | ccgccgccgccggcctgcaccggtatctgcgggacttctgcggctgccac | |
| capitalized) | gtggcctggagcggcagccagctgcggctgccccggcccctgcccgccgt | |
| | gcccggcgagctgaccgaggccacccccaaccggtatcggtactaccaga | |
| | acgtgtgcacccagagctacagcttcgtgtggtgggactgggcccggtgg | |
| | gagcgggagatcgactggatggccctgaacggcatcaacctggccctggc | |
| | ctggagcggccaggaggccatctggcagcgggtgtacctggccctgggcc | |
| | tgacccaggccgagatcaacgagttcttcaccggccccgccttcctggcc | |
| | tggggccggatgggcaacctgcacacctgggacggccccctgcctccaag | |
| | ctggcacatcaagcagctgtacctgcagcaccgggtgctggaccagatgc | |
| | ggagcttcggcatgacccccgtgctgcccgccttcgccggccacgtgccc | |
| | gaggccgtgacccgggtgttcccccaagttaacgtgaccaagatgggcag | |
| | ctggggccacttcaactgcagctacagctgcagcttcctgctggcccccg | |
| | aggaccccatcttccccatcatcggcagcctgttcctgcgggagctgatc | |
| | aaggagttcggcaccgaccacatctacggcgccgacaccttcaacgagat | |
| | gcagcctccaagcagcgagcccagctacctggccgccgccaccaccgccg | |
| | tgtacgaggccatgaccgccgtggacaccgaggccgtgtggctgctgcag | |
| | ggctggctgttccagcaccagccccagttctggggacctgcccagatccg | |
| | ggccgtgctgggcgccgtgcctagaggacggctgctggtgctggacctgt | |
| | tcgccgagagccagcccgtgtacacccggaccgccagcttccagggccag | |
| | cccttcatctggtgcatgctgcacaacttcggcggcaaccacggcctgtt | |
| | cggcgccctggaggccgtgaacggcggccccgaggccgcccggctgttcc | |
| | ccaacagcaccatggtgggcaccggcatggcccccgagggcatcagccag | |
| | aacgaggtggtgtacagcctgatggccgagctgggctggcggaaggaccc | |
| | cgtgcccgacctggccgcctgggtgaccagcttcgccgcccggcggtacg | |
| | gcgtgagccaccccgacgccggcgccgcctggcggctgctgctgcggagc | |
| | gtgtacaactgcagcggcgaggcctgccggggccacaaccggagccccct | |
| | ggtgcggcggcccagcctgcagatgaacaccagcatctggtacaaccgga | |
| | gcgacgtgttcgaggcctggcggctgctgctgaccagcgcccccagcctg | |
| | gccaccagccccgccttcagatacgacctgctggacctgacccggcaggc | |
| | cgtgcaggagctggtgagcctgtactacgaggaggcccggagcgcctacc | |
| | tgagcaaggagctggccagcctgctgcgggccggcggcgtgctggcctac | |
| | gagctgctgcccgccctggacgaggtgctggccagcgacagccggttcct | |
| | gctgggcagctggctggagcaggcccgggccgccgccgtgagcgaggccg | |
| | aggccgacttctacgagcagaacagccggtatcagctgaccctgtgggga | |
| | cctgagggcaacatcctggactacgccaacaagcagctggccggcctggt | |
| | ggccaactactacaccccaaggtggcggctgttcctggaggccctggtgg | |
| | acagcgtggcccagggcatccccttccagcagcaccagttcgacaagaac | |
| | gtgttccagctggagcaggccttcgtgctgagcaagcagcggtatcccag | |
| | ccagcctagaggagacaccgtggacctggccaagaagatcttcctgaagt | |
| | actacccccggtgggtggccggcagctggggaCTTGAGGTACTGTTCCAA | |
| | GGGCCCGAGGACCAGGTAGACCCACGACTCATTGATGGAAAATAG | |
| vIGF2- | ATGGAGGCTGTGGCTGTGGCAGCTGCGGTGGGGGTCCTTCTCCT | 247 |
| NAGLU- | GGCCGGGGCCGGGGGCGCGGCAGGCGACGcTTCTAGGACGTTGT | |
| HPC4 | GTGGTGGGGAACTTGTCGACACACTGCAGTTTGTCTGCGGCGAC | |
| | CGAGGATTTCTTTTTTCCAGGCCTGCCTCAAGAGTATCTAGGAGG | |
| | TCCCGCGGTATTGTTGAAGAGTGCTGTTTTAGGTCATGCGACCTT | |
| | GCGTTGTTGGAGACATATTGTGCTACCCCTGCACGCTCTGAAGGT | |
| | GGAGGTGGTTCAGGTGGTGGAGGTTCCAGGCCAAGGGCGGTCCC | |
| | TACTCAGGCCgaggcccgggaggccgccgccgtgcgggccctggtggccc | |
| | ggctgctgggccccggccccgccgccgacttcagcgttagcgtggagcgg | |
| | gccctggccgccaagcccggcctggacacctacagcctgggcggcggcgg | |
| | cgccgcccgggtgcgggtgcggggcagcaccggcgtggccgccgccgccg | |
| | gcctgcaccggtatctgcgggacttctgcggctgccacgtggcctggagc | |
| | ggcagccagctgcggctgccccggcccctgcccgccgtgcccggcgagct | |
| | gaccgaggccacccccaaccggtatcggtactaccagaacgtgtgcaccc | |
| | agagctacagcttcgtgtggtgggactgggcccggtgggagcgggagatc | |
| | gactggatggccctgaacggcatcaacctggccctggcctggagcggcca | |
| | ggaggccatctggcagcgggtgtacctggccctgggcctgacccaggccg | |
| | agatcaacgagttcttcaccggccccgccttcctggcctggggccggatg | |
| | ggcaacctgcacacctgggacggccccctgcctccaagctggcacatcaa | |
| | gcagctgtacctgcagcaccgggtgctggaccagatgcggagcttcggca | |
| | tgacccccgtgctgcccgccttcgccggccacgtgcccgaggccgtgacc | |
| | cgggtgttcccccaagttaacgtgaccaagatgggcagctggggccactt | |
| | caactgcagctacagctgcagcttcctgctggcccccgaggaccccatct | |
| | tccccatcatcggcagcctgttcctgcgggagctgatcaaggagttcggc | |
| | accgaccacatctacggcgccgacaccttcaacgagatgcagcctccaag | |
| | cagcgagcccagctacctggccgccgccaccaccgccgtgtacgaggcca | |
| | tgaccgccgtggacaccgaggccgtgtggctgctgcagggctggctgttc | |
| | cagcaccagccccagttctggggacctgcccagatccgggccgtgctggg | |
| | cgccgtgcctagaggacggctgctggtgctggacctgttcgccgagagcc | |
| | agcccgtgtacacccggaccgccagcttccagggccagcccttcatctgg | |
| | tgcatgctgcacaacttcggcggcaaccacggcctgttcggcgccctgga | |
| | ggccgtgaacggggccccgaggccgcccggctgttccccaacagcaccat | |
| | ggtgggcaccggcatggcccccgagggcatcagccagaacgaggtggtgt | |
| | acagcctgatggccgagctgggctggcggaaggaccccgtgcccgacctg | |
| | gccgcctgggtgaccagcttcgccgcccggcggtacggcgtgagccaccc | |
| | cgacgccggcgccgcctggcggctgctgctgcggagcgtgtacaactgca | |
| | gcggcgaggcctgccggggccacaaccggagccccctggtgcggcggccc | |
| | agcctgcagatgaacaccagcatctggtacaaccggagcgacgtgttcga | |
| | ggcctggcggctgctgctgaccagcgcccccagcctggccaccagccccg | |
| | ccttcagatacgacctgctggacctgacccggcaggccgtgcaggagctg | |
| | gtgagcctgtactacgaggaggcccggagcgcctacctgagcaaggagct | |
| | ggccagcctgctgcgggccggcggcgtgctggcctacgagctgctgcccg | |
| | ccctggacgaggtgctggccagcgacagccggttcctgctgggcagctgg | |
| | ctggagcaggcccgggccgccgccgtgagcgaggccgaggccgacttcta | |
| | cgagcagaacagccggtatcagctgaccctgtggggacctgagggcaaca | |
| | tcctggactacgccaacaagcagctggccggcctggtggccaactactac | |
| | accccaaggtggcggctgttcctggaggccctggtggacagcgtggccca | |
| | gggcatccccttccagcagcaccagttcgacaagaacgtgttccagctgg | |
| | agcaggccttcgtgctgagcaagcagcggtatcccagccagcctagagga | |
| | gacaccgtggacctggccaagaagatcttcctgaagtactacccccggtg | |
| | ggtggccggcagctggggaCTTGAGGTACTGTTCCAAGGGCCCGAGGACC | |
| | AGGTAGACCCACGACTCATTGATGGAAAATAG | |
| vIGF2-17- | ATGGAGGCTGTGGCTGTGGCAGCTGCGGTGGGGGTCCTTCTCCT | 248 |
| NAGLU- | GGCCGGGGCCGGGGGCGCGGCAGGCGACGcTagcagaacactttgtggcg | |
| HPC4 | gagagctggtggacaccctgcagtttgtgtgtggcgacagaggcttcctg | |
| | ttcagcagacctgcatccagagttagcaggcggtccagaggaatcgtgga | |
| | agagtgctgcttcagaGAAtgcgatctggccctgctggaaacctactgtg | |
| | ccacaccagccagatctgaaGGTGGAGGTGGTTCAGGTGGTGGAGGTTCC | |
| | AGGCCAAGGGCGGTCCCTACTCAGGCCgaggcccgggaggccgccgccgt | |
| | gcgggccctggtggcccggctgctgggccccggccccgccgccgacttca | |
| | gcgttagcgtggagcgggccctggccgccaagcccggcctggacacctac | |
| | agcctgggcggcggcggcgccgcccgggtgcgggtgcggggcagcaccgg | |
| | cgtggccgccgccgccggcctgcaccggtatctgcgggacttctgcggct | |
| | gccacgtggcctggagcggcagccagctgcggctgccccggcccctgccc | |
| | gccgtgcccggcgagctgaccgaggccacccccaaccggtatcggtacta | |
| | ccagaacgtgtgcacccagagctacagcttcgtgtggtgggactgggccc | |
| | ggtgggagcgggagatcgactggatggccctgaacggcatcaacctggcc | |
| | ctggcctggagcggccaggaggccatctggcagcgggtgtacctggccct | |
| | gggcctgacccaggccgagatcaacgagttcttcaccggccccgccttcc | |
| | tggcctggggccggatgggcaacctgcacacctgggacggccccctgcct | |
| | ccaagctggcacatcaagcagctgtacctgcagcaccgggtgctggacca | |
| | gatgcggagcttcggcatgacccccgtgctgcccgccttcgccggccacg | |
| | tgcccgaggccgtgacccgggtgttcccccaagttaacgtgaccaagatg | |
| | ggcagctggggccacttcaactgcagctacagctgcagcttcctgctggc | |
| | ccccgaggaccccatcttccccatcatcggcagcctgttcctgcgggagc | |
| | tgatcaaggagttcggcaccgaccacatctacggcgccgacaccttcaac | |
| | gagatgcagcctccaagcagcgagcccagctacctggccgccgccaccac | |
| | cgccgtgtacgaggccatgaccgccgtggacaccgaggccgtgtggctgc | |
| | tgcagggctggctgttccagcaccagccccagttctggggacctgcccag | |
| | atccgggccgtgctgggcgccgtgcctagaggacggctgctggtgctgga | |
| | cctgttcgccgagagccagcccgtgtacacccggaccgccagcttccagg | |
| | gccagcccttcatctggtgcatgctgcacaacttcggcggcaaccacggc | |
| | ctgttcggcgccctggaggccgtgaacggcggccccgaggccgcccggct | |
| | gttccccaacagcaccatggtgggcaccggcatggcccccgagggcatca | |
| | gccagaacgaggtggtgtacagcctgatggccgagctgggctggcggaag | |
| | gaccccgtgcccgacctggccgcctgggtgaccagcttcgccgcccggcg | |
| | gtacggcgtgagccaccccgacgccggcgccgcctggcggctgctgctgc | |
| | ggagcgtgtacaactgcagcggcgaggcctgccggggccacaaccggagc | |
| | cccctggtgcggcggcccagcctgcagatgaacaccagcatctggtacaa | |
| | ccggagcgacgtgttcgaggcctggcggctgctgctgaccagcgccccca | |
| | gcctggccaccagccccgccttcagatacgacctgctggacctgacccgg | |
| | caggccgtgcaggagctggtgagcctgtactacgaggaggcccggagcgc | |
| | ctacctgagcaaggagctggccagcctgctgcgggccggcggcgtgctgg | |
| | cctacgagctgctgcccgccctggacgaggtgctggccagcgacagccgg | |
| | ttcctgctgggcagctggctggagcaggcccgggccgccgccgtgagcga | |
| | ggccgaggccgacttctacgagcagaacagccggtatcagctgaccctgt | |
| | ggggacctgagggcaacatcctggactacgccaacaagcagctggccggc | |
| | ctggtggccaactactacaccccaaggtggcggctgttcctggaggccct | |
| | ggtggacagcgtggcccagggcatccccttccagcagcaccagttcgaca | |
| | agaacgtgttccagctggagcaggccttcgtgctgagcaagcagcggtat | |
| | cccagccagcctagaggagacaccgtggacctggccaagaagatcttcct | |
| | gaagtactacccccggtgggtggccggcagctggggaCTTGAGGTACTGT | |
| | TCCAAGGGCCCGAGGACCAGGTAGACCCACGACTCATTGATGGAAAATAG | |
| vIGF2-31- | ATGGAGGCTGTGGCTGTGGCAGCTGCGGTGGGGGTCCTTCTCCT | 249 |
| NAGLU- | GGCCGGGGCCGGGGGCGCGGCAGGCGACGcTagcagaacactttgtggcg | |
| HPC4 | gagagctggtggacaccctgcagtttgtgtgtggcgacagaggcttcctg | |
| | ttcagcagaGGTGGAGGTGGAtctagaggaatcCTGgaagagtgctgctt | |
| | cagaGATtgcgatctggccctgctggaaacctactgtgccacaccagcca | |
| | gatctgaaGGTGGAGGTGGTTCAGGTGGTGGAGGTTCCAGGCCAAGGGCG | |
| | GTCCCTACTCAGGCCgaggcccgggaggccgccgccgtgcgggccctggt | |
| | ggcccggctgctgggccccggccccgccgccgacttcagcgttagcgtgg | |
| | agcgggccctggccgccaagcccggcctggacacctacagcctgggcggc | |
| | ggcggcgccgcccgggtgcgggtgcggggcagcaccggcgtggccgccgc | |
| | cgccggcctgcaccggtatctgcgggacttctgcggctgccacgtggcct | |
| | ggagcggcagccagctgcggctgccccggcccctgcccgccgtgcccggc | |
| | gagctgaccgaggccacccccaaccggtatcggtactaccagaacgtgtg | |
| | cacccagagctacagcttcgtgtggtgggactgggcccggtgggagcggg | |
| | agatcgactggatggccctgaacggcatcaacctggccctggcctggagc | |
| | ggccaggaggccatctggcagcgggtgtacctggccctgggcctgaccca | |
| | ggccgagatcaacgagttcttcaccggccccgccttcctggcctggggcc | |
| | ggatgggcaacctgcacacctgggacggccccctgcctccaagctggcac | |
| | atcaagcagctgtacctgcagcaccgggtgctggaccagatgcggagctt | |
| | cggcatgacccccgtgctgcccgccttcgccggccacgtgcccgaggccg | |
| | tgacccgggtgttcccccaagttaacgtgaccaagatgggcagctggggc | |
| | cacttcaactgcagctacagctgcagcttcctgctggcccccgaggaccc | |
| | catcttccccatcatcggcagcctgttcctgcgggagctgatcaaggagt | |
| | tcggcaccgaccacatctacggcgccgacaccttcaacgagatgcagcct | |
| | ccaagcagcgagcccagctacctggccgccgccaccaccgccgtgtacga | |
| | ggccatgaccgccgtggacaccgaggccgtgtggctgctgcagggctggc | |
| | tgttccagcaccagccccagttctggggacctgcccagatccgggccgtg | |
| | ctgggcgccgtgcctagaggacggctgctggtgctggacctgttcgccga | |
| | gagccagcccgtgtacacccggaccgccagcttccagggccagcccttca | |
| | tctggtgcatgctgcacaacttcggcggcaaccacggcctgttcggcgcc | |
| | ctggaggccgtgaacggcggccccgaggccgcccggctgttccccaacag | |
| | caccatggtgggcaccggcatggcccccgagggcatcagccagaacgagg | |
| | tggtgtacagcctgatggccgagctgggctggcggaaggaccccgtgccc | |
| | gacctggccgcctgggtgaccagcttcgccgcccggcggtacggcgtgag | |
| | ccaccccgacgccggcgccgcctggcggctgctgctgcggagcgtgtaca | |
| | actgcagcggcgaggcctgccggggccacaaccggagccccctggtgcgg | |
| | cggcccagcctgcagatgaacaccagcatctggtacaaccggagcgacgt | |
| | gttcgaggcctggcggctgctgctgaccagcgcccccagcctggccacca | |
| | gccccgccttcagatacgacctgctggacctgacccggcaggccgtgcag | |
| | gagctggtgagcctgtactacgaggaggcccggagcgcctacctgagcaa | |
| | ggagctggccagcctgctgcgggccggcggcgtgctggcctacgagctgc | |
| | tgcccgccctggacgaggtgctggccagcgacagccggttcctgctgggc | |
| | agctggctggagcaggcccgggccgccgccgtgagcgaggccgaggccga | |
| | cttctacgagcagaacagccggtatcagctgaccctgtggggacctgagg | |
| | gcaacatcctggactacgccaacaagcagctggccggcctggtggccaac | |
| | tactacaccccaaggtggcggctgttcctggaggccctggtggacagcgt | |
| | ggcccagggcatccccttccagcagcaccagttcgacaagaacgtgttcc | |
| | agctggagcaggccttcgtgctgagcaagcagcggtatcccagccagcct | |
| | agaggagacaccgtggacctggccaagaagatcttcctgaagtactaccc | |
| | ccggtgggtggccggcagctggggaCTTGAGGTACTGTTCCAAGGGCCCG | |
| | AGGACCAGGTAGACCCACGACTCATTGATGGAAAATAG | |
| vIGF2-32- | ATGGAGGCTGTGGCTGTGGCAGCTGCGGTGGGGGTCCTTCTCCT | 250 |
| NAGLU- | GGCCGGGGCCGGGGGCGCGGCAGGCGACGcTagcagaacactttgtggcg | |
| HPC4 | gagagctggtggacaccctgcagtttgtgtgtggcgacagaggcttcctg | |
| | ttcagcagaGGTGGAGGTGGAtctagaggaatcCTGgaagagtgctgctt | |
| | cagaGAAtgcgatctggccctgctggaaacctactgtgccacaccagcca | |
| | gatctgaaGGTGGAGGTGGTTCAGGTGGTGGAGGTTCCAGGCCAAGGGCG | |
| | GTCCCTACTCAGGCCgaggcccgggaggccgccgccgtgcgggccctggt | |
| | ggcccggctgctgggccccggccccgccgccgacttcagcgttagcgtgg | |
| | agcgggccctggccgccaagcccggcctggacacctacagcctgggcggc | |
| | ggcggcgccgcccgggtgcgggtgcggggcagcaccggcgtggccgccgc | |
| | cgccggcctgcaccggtatctgcgggacttctgcggctgccacgtggcct | |
| | ggagcggcagccagctgcggctgccccggcccctgcccgccgtgcccggc | |
| | gagctgaccgaggccacccccaaccggtatcggtactaccagaacgtgtg | |
| | cacccagagctacagcttcgtgtggtgggactgggcccggtgggagcggg | |
| | agatcgactggatggccctgaacggcatcaacctggccctggcctggagc | |
| | ggccaggaggccatctggcagcgggtgtacctggccctgggcctgaccca | |
| | ggccgagatcaacgagttcttcaccggccccgccttcctggcctggggcc | |
| | ggatgggcaacctgcacacctgggacggccccctgcctccaagctggcac | |
| | atcaagcagctgtacctgcagcaccgggtgctggaccagatgcggagctt | |
| | cggcatgacccccgtgctgcccgccttcgccggccacgtgcccgaggccg | |
| | tgacccgggtgttcccccaagttaacgtgaccaagatgggcagctggggc | |
| | cacttcaactgcagctacagctgcagcttcctgctggcccccgaggaccc | |
| | catcttccccatcatcggcagcctgttcctgcgggagctgatcaaggagt | |
| | tcggcaccgaccacatctacggcgccgacaccttcaacgagatgcagcct | |
| | ccaagcagcgagcccagctacctggccgccgccaccaccgccgtgtacga | |
| | ggccatgaccgccgtggacaccgaggccgtgtggctgctgcagggctggc | |
| | tgttccagcaccagccccagttctggggacctgcccagatccgggccgtg | |
| | ctgggcgccgtgcctagaggacggctgctggtgctggacctgttcgccga | |
| | gagccagcccgtgtacacccggaccgccagcttccagggccagcccttca | |
| | tctggtgcatgctgcacaacttcggcggcaaccacggcctgttcggcgcc | |
| | ctggaggccgtgaacggcggccccgaggccgcccggctgttccccaacag | |
| | caccatggtgggcaccggcatggcccccgagggcatcagccagaacgagg | |
| | tggtgtacagcctgatggccgagctgggctggcggaaggaccccgtgccc | |
| | gacctggccgcctgggtgaccagcttcgccgcccggcggtacggcgtgag | |
| | ccaccccgacgccggcgccgcctggcggctgctgctgcggagcgtgtaca | |
| | actgcagcggcgaggcctgccggggccacaaccggagccccctggtgcgg | |
| | cggcccagcctgcagatgaacaccagcatctggtacaaccggagcgacgt | |
| | gttcgaggcctggcggctgctgctgaccagcgcccccagcctggccacca | |
| | gccccgccttcagatacgacctgctggacctgacccggcaggccgtgcag | |
| | gagctggtgagcctgtactacgaggaggcccggagcgcctacctgagcaa | |
| | ggagctggccagcctgctgcgggccggcggcgtgctggcctacgagctgc | |
| | tgcccgccctggacgaggtgctggccagcgacagccggttcctgctgggc | |
| | agctggctggagcaggcccgggccgccgccgtgagcgaggccgaggccga | |
| | cttctacgagcagaacagccggtatcagctgaccctgtggggacctgagg | |
| | gcaacatcctggactacgccaacaagcagctggccggcctggtggccaac | |
| | tactacaccccaaggtggcggctgttcctggaggccctggtggacagcgt | |
| | ggcccagggcatccccttccagcagcaccagttcgacaagaacgtgttcc | |
| | agctggagcaggccttcgtgctgagcaagcagcggtatcccagccagcct | |
| | agaggagacaccgtggacctggccaagaagatcttcctgaagtactaccc | |
| | ccggtgggtggccggcagctggggaCTTGAGGTACTGTTCCAAGGGCCCG | |
| | AGGACCAGGTAGACCCACGACTCATTGATGGAAAATAG | |

In some embodiments, the vector comprising the nucleic acid encoding the desired therapeutic fusion protein, such as a vIGF2 fusion or a signal peptide fusion, optionally having an internal ribosomal entry sequence, provided herein is an adeno-associated viral vector (A5/35).

In some embodiments, the nucleic acid encoding the therapeutic fusion protein, such as a vIGF2 fusion, optionally has an internal ribosomal entry sequence and can be cloned into various types of vectors. For example, in some embodiments, the nucleic acid is cloned into a vector including, but not limited to a plasmid, a phagemid, a phage derivative, an animal virus, and a cosmid. Vectors of interest include expression vectors, replication vectors, probe generation vectors, and sequencing vectors.

Further, the expression vector encoding the therapeutic fusion protein, such as a vIGF2 fusion or a signal peptide fusion, optionally having an internal ribosomal entry sequence, in some embodiments, is provided to a cell in the form of a viral vector. Viral vector technology is described, e.g., in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

Also provided herein are compositions and systems for gene transfer. A number of virally based systems have been developed for gene transfer into mammalian cells. For example, retroviruses provide a convenient platform for gene delivery systems. A selected gene, in some embodiments, is inserted into a vector and packaged in retroviral particles using suitable techniques. The recombinant virus is then isolated and delivered to cells of the subject either in vivo or ex vivo. A number of retroviral systems are suitable for gene therapy. In some embodiments, adenovirus vectors are used. A number of adenovirus vectors are suitable for gene therapy. In some embodiments, adeno-associated virus vectors are used. A number of adeno-associated viruses are suitable for gene therapy. In one embodiment, lentivirus vectors are used.

Gene therapy constructs provided herein comprise a vector (or gene therapy expression vector) into which the gene of interest is cloned or otherwise which includes the gene of interest in a manner such that the nucleotide sequences of the vector allow for the expression (constitutive or otherwise regulated in some manner) of the gene of interest. The vector constructs provided herein include any suitable gene expression vector that is capable of being delivered to a tissue of interest and which will provide for the expression of the gene of interest in the selected tissue of interest.

In some embodiments, the vector is an adeno-associated virus (AAV) vector because of the capacity of AAV vectors to cross the blood-brain barrier and transduction of neuronal tissue. In methods provided herein, AAV of any serotype is contemplated to be used. The serotype of the viral vector used in certain embodiments is selected from the group consisting of an AAV1 vector, an AAV2 vector, an AAV3 vector, an AAV4 vector, an AAV5 vector, an AAV6 vector, an AAV7 vector, an AAV8 vector, an AAV9 vector, an AAVrhS vector, an AAVrh10 vector, an AAVrh33 vector, an AAVrh34 vector, an AAVrh74 vector, an AAV Anc80 vector, an AAVPHP.B vector, an AAVhu68 vector, an AAV-DJ vector, and others suitable for gene therapy.

AAV vectors are DNA parvoviruses that are nonpathogenic for mammals. Briefly, AAV-based vectors have the rep and cap viral genes that account for 96% of the viral genome removed, leaving the two flanking 145 base pair inverted terminal repeats (ITRs) which are used to initiate viral DNA replication, packaging, and integration.

Further embodiments include use of other serotype capsids to create an AAV1 vector, an AAV2 vector, an AAV3 vector, an AAV4 vector, an AAV5 vector, an AAV6 vector, an AAV7 vector, an AAV8 vector, an AAV9 vector, an AAVrhS vector, an AAVrh10 vector, an AAVrh33 vector, an AAVrh34 vector, an AAVrh74 vector, an AAV Anc80 vector, an AAVPHP.B vector, an AAV-DJ vector, and others suitable for gene therapy. Optionally, the AAV viral capsid is AAV2/9, AAV9, AAVrhS, AAVrh10, AAVAnc80, or AAV PHP.B.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements is often increased to 50 bp apart before activity begins to decline. Depending on the promoter, it appears that individual elements function either cooperatively or independently to activate transcription.

An example of a promoter that is capable of expressing a therapeutic fusion protein, such as a vIGF2 fusion or a signal peptide fusion, optionally having an internal ribosomal entry sequence, transgene in a mammalian T-cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences are sometimes also used, including, but not limited to the chicken β actin promoter, the P546 promoter, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, gene therapy vectors are not contemplated to be limited to the use of constitutive promoters. Inducible promoters are also contemplated here. An inducible promoter provides a molecular switch capable of turning on expression of the polynucleotide sequence to which it is operatively linked when such expression is desired, and turning off expression when expression is not desired. Examples of inducible promoters include, but are not limited to a metallothionine promoter, a glucocorticoid promoter, a progesterone promoter, and a tetracycline-regulated promoter.

In order to assess the expression of a therapeutic fusion protein, such as a vIGF fusion or a signal peptide fusion, optionally having an internal ribosomal entry sequence, or portions thereof, the expression vector to be introduced into a cell often contains either a selectable marker gene or a reporter gene or both to facilitate identification and selection of expressing cells from the population of cells sought to be transfected or infected through viral vectors. In other aspects, the selectable marker is often carried on a separate piece of DNA and used in a co-transfection procedure. Both selectable markers and reporter genes are sometimes flanked with appropriate regulatory sequences to enable expression in the host cells. Useful selectable markers include, for example, antibiotic-resistance genes, such as neo and the like.

Methods and compositions for introducing and expressing genes into a cell are suitable for methods herein. In the context of an expression vector, the vector is readily introduced into a host cell, e.g., mammalian, bacterial, yeast, or insect cell by any method in the art. For example, the expression vector is transferred into a host cell by physical, chemical, or biological means.

Physical methods and compositions for introducing a polynucleotide into a host cell include calcium phosphate precipitation, lipofection, particle bombardment, microinjection, gene gun, electroporation, and the like. Methods for producing cells comprising vectors and/or exogenous nucleic acids are suitable for methods herein (see, e.g., Sambrook et al., 2012, Molecular Cloning: A Laboratory Manual, volumes 1-4, Cold Spring Harbor Press, NY). One method for the introduction of a polynucleotide into a host cell is calcium phosphate transfection.

Chemical means and compositions for introducing a polynucleotide into a host cell include colloidal dispersion systems, such as macromolecule complexes, nanocapsules, microspheres, beads, and lipid-based systems including oil-in-water emulsions, micelles, mixed micelles, nucleic acid-lipid particles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. For example, in some embodiments, dimyristyl phosphatidylcholine (“DMPC”) is obtained from Sigma, St. Louis, Mo.; in some embodiments, dicetyl phosphate (“DCP”) is obtained from K & K Laboratories (Plainview, N.Y.); cholesterol (“Choi”), in some embodiments, is obtained from Calbiochem-Behring; dimyristyl phosphatidylglycerol (“DMPG”) and other lipids are often obtained from Avanti Polar Lipids, Inc. (Birmingham, Ala.). Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids, in some embodiments, assume a micellar structure or merely exist as non-uniform aggregates of lipid molecules. Also contemplated are lipofectamine-nucleic acid complexes.

Regardless of the method used to introduce exogenous nucleic acids into a host cell or otherwise expose a cell to the therapeutic fusion protein, such as a vIGF2 fusion or a signal peptide fusion, optionally having an internal ribosomal entry sequence, provided herein, in order to confirm the presence of the recombinant DNA sequence in the host cell, a variety of assays are contemplated to be performed. Such assays include, for example, “molecular biological” assays suitable for methods herein, such as Southern and Northern blotting, RT-PCR and PCR; “biochemical” assays, such as detecting the presence or absence of a particular peptide, e.g., by immunological means (ELISAs and western blots) or by assays described herein to identify agents falling within the scope herein.

The present disclosure further provides a vector comprising a therapeutic fusion protein, such as a vIGF2 fusion or a signal peptide fusion, optionally having an internal ribosomal entry sequence, encoding nucleic acid molecule. In one aspect, a therapeutic fusion protein vector is capable of being directly transduced into a cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector can be used to express the vIGF2-therapeutic fusion protein construct in mammalian cells. In one aspect, the mammalian cell is a human cell.

Uses and Methods of Treatment

Also provided herein are methods of treating genetic disorders using gene therapy comprising administering to an individual a nucleic acid encoding a therapeutic fusion protein (such as a vIGF2 fusion or a signal peptide fusion or a signal peptide-vIGF2 fusion), optionally having an internal ribosomal entry sequence, disclosed herein. Genetic disorders suitable for treatment using methods herein comprise disorders in an individual caused by one or more mutations in the genome causing lack of expression or expression of a dysfunctional protein by the mutant gene.

Further provided herein are pharmaceutical compositions comprising a gene therapy vector, such as a gene therapy vector comprising a nucleic acid encoding a therapeutic fusion protein (such as a vIGF2 fusion or a signal peptide fusion or a signal peptide-vIGF2 fusion), optionally having an internal ribosomal entry sequence, disclosed herein and a pharmaceutically acceptable carrier or excipient for use in preparation of a medicament for treatment of a genetic disorder.

In some embodiments, genetic disorders suitable for treatment using methods provided herein are lysosomal storage disorder. In some embodiments, lysosomal storage disorders are treated herein using gene therapy to deliver missing or defective enzymes to the patient. In some embodiments, methods herein deliver an enzyme fused to a vIGF2 or fused to a signal peptide to the patient in order to deliver the enzyme to the cell where it is needed. In some embodiments, the lysosomal storage disorder is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, and Schindler disease type II. In some embodiments, the lysosomal storage disorder is selected from the group consisting of activator deficiency, GM2-gangliosidosis; GM2-gangliosidosis, AB variant; alpha-mannosidosis (type 2, moderate form; type 3, neonatal, severe); beta-mannosidosis; aspartylglucosaminuria; lysosomal acid lipase deficiency; cystinosis (late-onset juvenile or adolescent nephropathic type; infantile nephropathic); Chanarin-Dorfman syndrome; neutral lipid storage disease with myopathy; NLSDM; Danon disease; Fabry disease; Fabry disease type II, late-onset; Farber disease; Farber lipogranulomatosis; fucosidosis; galactosialidosis (combined neuraminidase & beta-galactosidase deficiency); Gaucher disease; type II Gaucher disease; type III Gaucher disease; type IIIC Gaucher disease; Gaucher disease, atypical, due to saposin C deficiency; GM1-gangliosidosis (late-infantile/juvenile GM1-gangliosidosis; adult/chronic GM1-gangliosidosis); Globoid cell leukodystrophy, Krabbe disease (Late infantile onset; Juvenile Onset; Adult Onset); Krabbe disease, atypical, due to saposin A deficiency; Metachromatic Leukodystrophy (juvenile; adult); partial cerebroside sulfate deficiency; pseudoarylsulfatase A deficiency; metachromatic leukodystrophy due to saposin B deficiency; Mucopolysaccharidoses disorders: MPS I, Hurler syndrome; MPS I, Hurler-Scheie syndrome; MPS I, Scheie syndrome; MPS II, Hunter syndrome; MPS II, Hunter syndrome; Sanfilippo syndrome Type A/MPS IIIA; Sanfilippo syndrome Type B/MPS IIIB; Sanfilippo syndrome Type C/MPS IIIC; Sanfilippo syndrome Type D/MPS IIID; Morquio syndrome, type A/MPS IVA; Morquio syndrome, type B/MPS IVB; MPS IX hyaluronidase deficiency; MPS VI Maroteaux-Lamy syndrome; MPS VII Sly syndrome; mucolipidosis I, sialidosis type II; I-cell disease, Leroy disease, mucolipidosis II; Pseudo-Hurler polydystrophy/mucolipidosis type III; mucolipidosis IIIC/ML III GAMMA; mucolipidosis type IV; multiple sulfatase deficiency; Niemann-Pick disease (type B; type C1/chronic neuronopathic form; type C2; type D/Nova Scotian type); Neuronal Ceroid Lipofuscinoses: CLN6 disease—Atypical Late Infantile, Late-Onset variant, Early Juvenile; Batten-Spielmeyer-Vogt/Juvenile NCL/CLN3 disease; Finnish Variant Late Infantile CLN5; Jansky-Bielschowsky disease/Late infantile CLN2/TPP1 Disease; Kufs/Adult-onset NCL/CLN4 disease (type B); Northern Epilepsy/variant late infantile CLN8; Santavuori-Haltia/Infantile CLN1/PPT disease; Pompe disease (glycogen storage disease type II); late-onset Pompe disease; Pycnodysostosis; Sandhoff disease/GM2 gangliosidosis; Sandhoff disease/GM2 gangliosidosis; Sandhoff disease/GM2 Gangliosidosis; Schindler disease (type III/intermediate, variable); Kanzaki disease; Salla disease; infantile free sialic acid storage disease (ISSD); spinal muscular atrophy with progressive myoclonic epilepsy (SMAPME); Tay-Sachs disease/GM2 gangliosidosis; juvenile-onset Tay-Sachs disease; late-onset Tay-Sachs disease; Christianson syndrome; Lowe oculocerebrorenal syndrome; Charcot-Marie-Tooth type 4J, CMT4J; Yunis-Varon syndrome; bilateral temporooccipital polymicrogyria (BTOP); X-linked hypercalciuric nephrolithiasis, Dent-1; and Dent disease 2. In some embodiments, the therapeutic protein is associated with a lysosomal storage disorder and the therapeutic protein is selected from the group consisting of GM2-activator protein; α-mannosidase; MAN2B1; lysosomal ß-mannosidase; glycosylasparaginase; lysosomal acid lipase; cystinosin; CTNS; PNPLA2; lysosome-associated membrane protein-2; α-galactosidase A; GLA; acid ceramidase; α-L-fucosidase; protective protein/cathepsin A; acid ß-glucosidase; GBA; PSAP; β-galactosidase-1; GLB1; galactosylceramide β-galactosidase; GALC; PSAP; arylsulfatase A; ARSA; α-L-iduronidase; iduronate 2-sulfatase; heparan N-sulfatase; N-α-acetylglucosaminidase; heparan acetyl CoA: α-glucosaminide acetyltransferase; N-acetylglucosamine 6-sulfatase; galactosamine-6-sulfate sulfatase; ß-galactosidase; hyaluronidase; arylsulfatase B; ß-glucuronidase; neuraminidase; NEU1; gamma subunit of N-acetylglucosamine-1-phosphotransferase; mucolipin-1; sulfatase-modifying factor-1; acid sphingomyelinase; SMPD1; NPC1; and NPC2.

In some embodiments, treatment via methods herein delivers a gene encoding a therapeutic protein to a cell in need of the therapeutic protein. In some embodiments, the treatment delivers the gene to all somatic cells in the individual. In some embodiments, the treatment replaces the defective gene in the targeted cells. In some embodiments, cells treated ex vivo to express the therapeutic protein are delivered to the individual.

Gene therapy for disorders disclosed herein provides superior treatment outcomes to conventional treatments, including enzyme replacement therapy, because it does not require long infusion treatments.

### Definitions

As used herein “ex vivo gene therapy” refers to methods where patient cells are genetically modified outside the subject, for example to express a therapeutic gene. Cells with the new genetic information are then returned to the subject from whom they were derived.

As used herein “in vivo gene therapy” refers to methods where a vector carrying the therapeutic gene(s) is directly administered to the subject.

As used herein “fusion protein” and “therapeutic fusion protein” are used interchangeably herein and refer to a therapeutic protein having at least one additional protein, peptide, or polypeptide, linked to it. In some instances, fusion proteins are a single protein molecule containing two or more proteins or fragments thereof, covalently linked via peptide bond within their respective peptide chains, without chemical linkers. In some embodiments, the fusion protein comprises a therapeutic protein and a signal peptide, a peptide that increases endocytosis of the fusion protein, or both. In some embodiments, the peptide that increases endocytosis is a peptide that binds CI-MPR.

As used herein “vector”, or “gene therapy vector”, used interchangeably herein, refers to gene therapy delivery vehicles, or carriers, that deliver therapeutic genes to cells. A gene therapy vector is any vector suitable for use in gene therapy, e.g., any vector suitable for the therapeutic delivery of nucleic acid polymers (encoding a polypeptide or a variant thereof) into target cells (e.g., sensory neurons) of a patient. In some embodiments, the gene therapy vector delivers the nucleic acid encoding a therapeutic protein or therapeutic fusion protein to a cell where the therapeutic protein or fusion is expressed and secreted from the cell. The vector may be of any type, for example it may be a plasmid vector or a minicircle DNA. Typically, the vector is a viral vector. These include both genetically disabled viruses such as adenovirus and nonviral vectors such as liposomes. The viral vector may for example be derived from an adeno-associated virus (AAV), a retrovirus, a lentivirus, a herpes simplex virus, or an adenovirus. AAV derived vectors. The vector may comprise an AAV genome or a derivative thereof.

“Construct” as used herein refers to a nucleic acid molecule or sequence that encodes a therapeutic protein or fusion protein and optionally comprises additional sequences such as a translation initiation sequence or IRES sequence.

As used herein “plasmid” refers to circular, double-stranded unit of DNA that replicates within a cell independently of the chromosomal DNA.

As used herein “promoter” refers to a site on DNA to which the enzyme RNA polymerase binds and initiates the transcription of DNA into RNA.

As used herein “somatic therapy” refers to methods where the manipulation of gene expression in cells that will be corrective to the patient but not inherited by the next generation. Somatic cells include all the non-reproductive cells in the human body

As used herein “somatic cells” refers to all body cells except the reproductive cells.

As used herein “tropism” refers to preference of a vector, such as a virus for a certain cell or tissue type. Various factors determine the ability of a vector to infect a particular cell. Viruses, for example, must bind to specific cell surface receptors to enter a cell. Viruses are typically unable to infect a cell if it does not express the necessary receptors.

The term “transduction” is used to refer to the administration/delivery of the nucleic acid encoding the therapeutic protein to a target cell either in vivo or in vitro, via a replication-deficient rAAV of the disclosure resulting in expression of a functional polypeptide by the recipient cell. Transduction of cells with a gene therapy vector such as a rAAV of the disclosure results in sustained expression of polypeptide or RNA encoded by the rAAV. The present disclosure thus provides methods of administering/delivering to a subject a gene therapy vector such as an rAAV encoding a therapeutic protein by an intrathecal, intraretinal, intraocular, intravitreous, intracerebroventricular, intraparechymal, or intravenous route, or any combination thereof. “Intrathecal” delivery refers to delivery into the space under the arachnoid membrane of the brain or spinal cord. In some embodiments, intrathecal administration is via intracisternal administration. The present disclosure also provides methods of administering/delivering cells that have been transduced ex vivo with a gene therapy vector such as an rAAV vector encoding a therapeutic protein by an intrathecal, intraretinal, intraocular, intravitreous, intracerebroventricular, intraparechymal, or intravenous route, or any combination thereof.

The terms “recipient”, “individual”, “subject”, “host”, and “patient”, are used interchangeably herein and in some cases, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human.

As used herein, the terms “treatment,” “treating,” “ameliorating a symptom,” and the like, in some cases, refer to administering an agent, or carrying out a procedure, for the purposes of obtaining a therapeutic effect, including inhibiting, attenuating, reducing, preventing or altering at least one aspect or marker of a disorder, in a statistically significant manner or in a clinically significant manner. The term “ameliorate” or “treat” does not state or imply a cure for the underlying condition. “Treatment,” or “to ameliorate” (and like) as used herein, may include treating a mammal, particularly in a human, and includes: (a) preventing the disorder or a symptom of a disorder from occurring in a subject which may be predisposed to the disorder but has not yet been diagnosed as having it (e.g., including disorders that may be associated with or caused by a primary disorder; (b) inhibiting the disorder, i.e., arresting its development; (c) relieving the disorder, i.e., causing regression of the disorder; and (d) improving at least one symptom of the disorder. Treating may refer to any indicia of success in the treatment or amelioration or prevention of a disorder, including any objective or subjective parameter such as abatement; remission; diminishing of symptoms or making the disorder condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with the disorder. The term “therapeutic effect” refers to the reduction, elimination, or prevention of the disorder, symptoms of the disorder, or side effects of the disorder in the subject.

The term “affinity” refers to the strength of binding between a molecule and its binding partner or receptor.

As used herein, the phrase “high affinity” refers to, for example, a therapeutic fusion containing such a peptide that binds CI-MPR which has an affinity to CI-MPR that is about 100 to 1,000 times or 500 to 1,000 times higher than that of the therapeutic protein without the peptide. In some embodiments, the affinity is at least 100, at least 500, or at least 1000 times higher than without the peptide. For example, where the therapeutic protein and CI-MPR are combined in relatively equal concentration, the peptide of high affinity will bind to the available CI-MPR so as to shift the equilibrium toward high concentration of the resulting complex.

“Secretion” as used herein refers to the release of a protein from a cell into, for example, the bloodstream to be carried to a tissue of interest or a site of action of the therapeutic protein. When a gene therapy product is secreted into the interstitial space of an organ, secretion can allow for cross-correction of neighboring cells.

“Delivery” as used herein means drug delivery. In some embodiments, the process of delivery means transporting a drug substance (e.g., therapeutic protein or fusion protein produced from a cell transduced with a gene therapy vector) from outside of a cell (e.g., blood, tissue, or interstitial space) into a target cell for therapeutic activity of the drug substance.

“Engineering” or “protein engineering” as used here in refers to the manipulation of the structures of a protein by providing appropriate a nucleic acid sequence that encodes for the protein as to produce desired properties, or the synthesis of the protein with particular structures.

A “therapeutically effective amount” in some cases means the amount that, when administered to a subject for treating a disorder, is sufficient to effect treatment for that disorder.

As used herein, the term “about” a number refers to a range spanning that from 10% less than that number through 10% more than that number, and including values within the range such as the number itself.

As used herein, the term “comprising” an element or elements of a claim refers to those elements but does not preclude the inclusion of an additional element or elements.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Binding of Variant IGF2 Peptide to CI-MPR Receptor

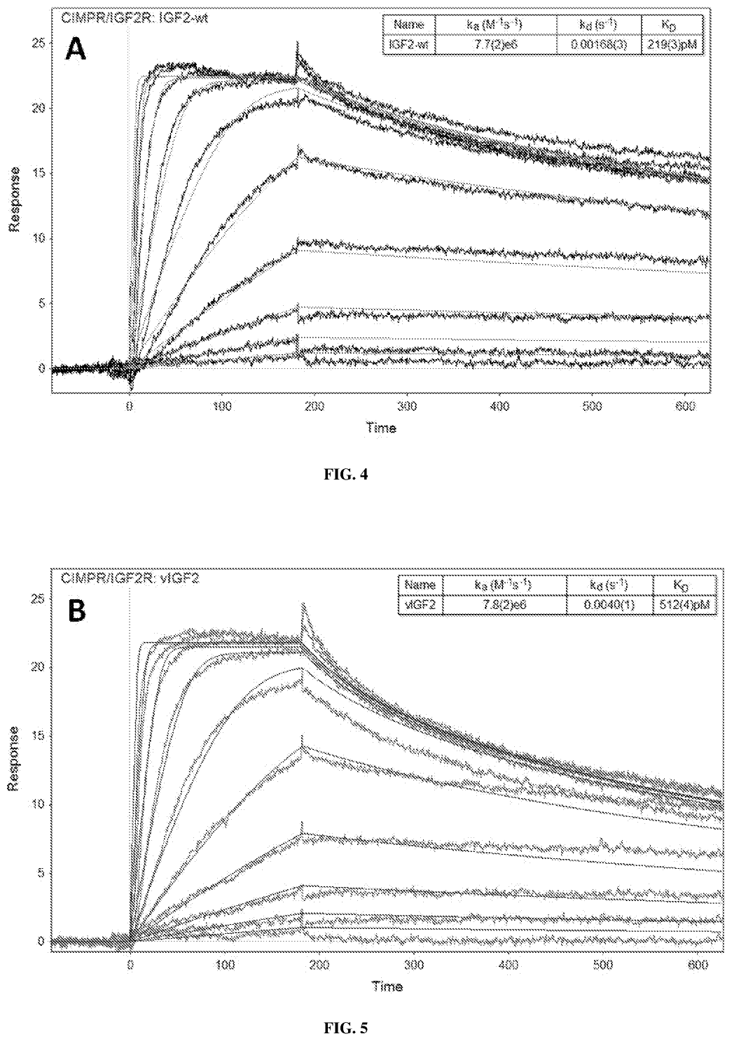

Surface plasmon resonance (SPR) experiments were conducted using Biacore to measure binding of wildtype and variant IGF2 (vIGF2) to the CI-MPR receptor. The wildtype, human mature IGF2 peptide (wt IGF2) has the sequence set forth in SEQ ID NO: 68. The vIGF2 sequence differs from wt IGF2 in that it lacks residues 1-4 and contains the following mutations: E6R, Y27L, and K65R. It has the amino acid sequence: SRTLCGGELVDTLQFVCGDRGFLFSRPASRVSRRSRGIVEECCFRSCDLALLETYCATP ARSE (SEQ ID NO: 80). vIGF2 also has an N-terminal linker with the sequence GGGGSGGGG (SEQ ID NO: 181). The combined sequence is GGGGSGGGGSRTLCGGELVDTLQFVCGDRGFLFSRPASRVSRRSRGIVEECCFRSCDL ALLETYCATPARSE. FIG. 4 shows that as expected, the wildtype IGF2 peptide binds to the CI-MPR receptor with high affinity (0.2 nM). FIG. 5 shows that the variant IGF2 peptide (vIGF2) also binds to the CI-MPR receptor with high affinity (0.5 nM). These data indicate that vIGF2 peptide has high affinity for the intended CI-MPR receptor for targeting therapeutics to lysosomes.

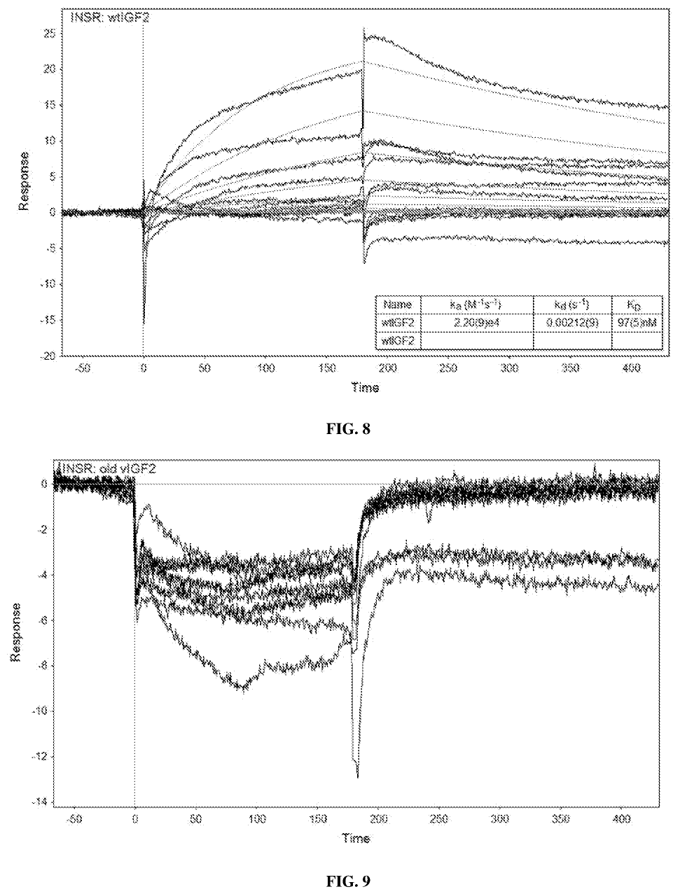

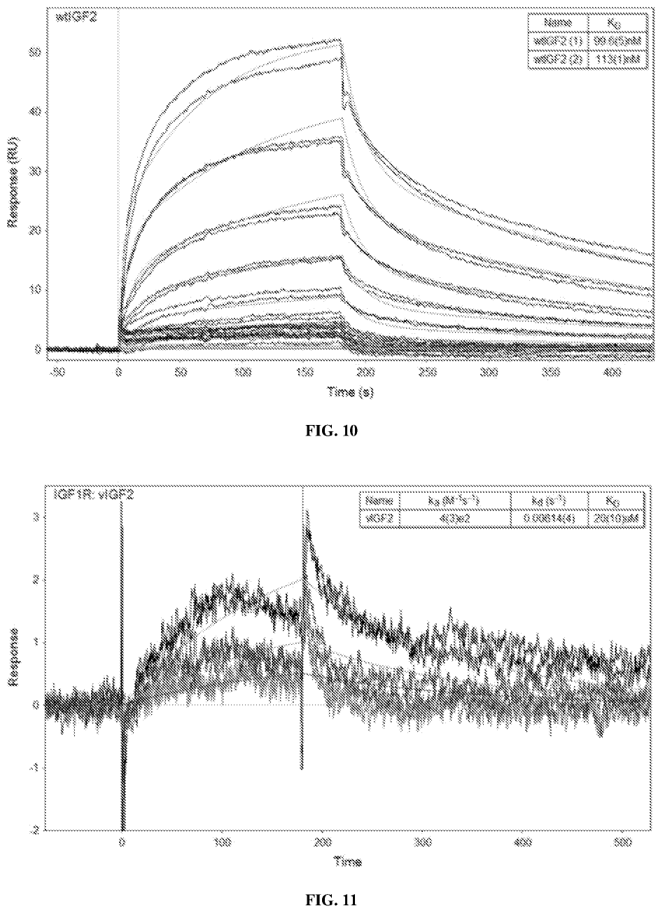

SPR was utilized to measure peptide binding to the Insulin Receptor to assess potential side effects. Insulin binds the Insulin Receptor with high affinity (˜8 nM; data not shown). Wildtype IGF2 and a vIGF2 were tested, where the vIGF2 had the sequence SRTLCGGELVDTLQFVCGDRGFLFSRPASRVSRRSRGIVEECCFRSCDLALLETYCATP ARSE (SEQ ID NO: 80) having an N-terminal linker with a sequence GGGGSGGGG (SEQ ID NO: 181). FIG. 8 shows that wildtype IGF2 also binds the Insulin Receptor with relatively high affinity (˜100 nM). IGF2 peptide from Biomarin/Zystor IGF2-GAA fusion protein (BMN-701) also binds the Insulin Receptor with high affinity and was shown to cause hypoglycemia in clinical trials. FIG. 9 shows no measurable binding of vIGF2 peptide to the insulin receptor. These data show that vIGF2 peptide confers a superior safety profile compared with wt IGF2 peptide fusions.

The same SPR binding analysis was utilized to characterize vIGF2 peptide interaction with the IGF1 Receptor. FIG. 10 shows that the wildtype IGF2 peptide binds IGF1 receptor with relatively high affinity (˜100 nM). FIG. 11 shows no measurable binding of vIGF2 peptide to the IGF1 Receptor, showing an improved safety profile compared to wt IGF2.

TABLE 8
SPR Affinity Results

| Receptor | wt IGF2 Kd (nM) | vIGF2 Kd (nM) |
|---|---|---|
| CI-MPR | 0.2 | 0.5 |
| Insulin Receptor | 100 | No Binding Detected |
| IGF1 Receptor | 100 | No Binding Detected |

### Example 2: vIGF2 Converts Low Affinity Ligand to High Affinity ERT for CI-MPR

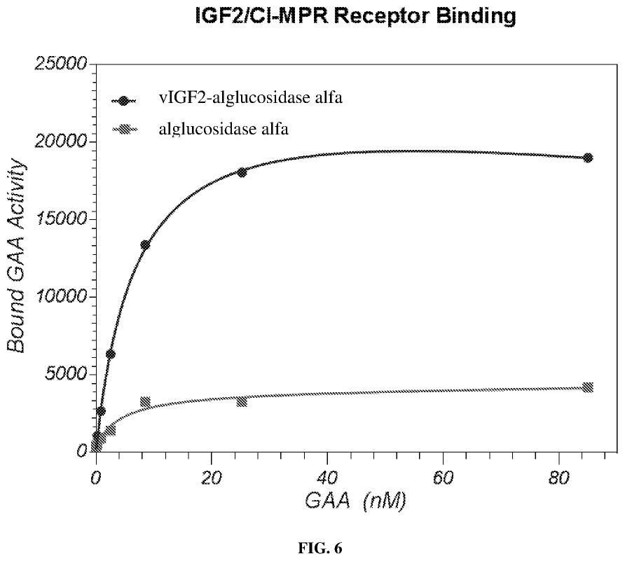

The vIGF2 peptide (SEQ ID NO: 80) with an N-terminal linker (SEQ ID NO: 181) was chemically coupled to alglucosidase-alfa, designated here as vIGF2-alglucosidase-alfa, to determine whether the vIGF2 peptide could improve affinity for CI-MPR. As shown in FIG. 6, binding affinities of alglucosidase-alfa and vIGF2-alglucosidase-alfa were directly compared using CI-MPR plate binding assays in 96-well plates coated with CI-MPR. Unbound enzyme was washed away prior to measuring bound enzyme activity. Varying concentrations of both enzyme preparations were used with or without free WT IGF2 peptide. vIGF2 substantially improved the affinity for CI-MPR. Further, binding of vIGF2-alglucosidase-alfa was blocked by free WT IGF2 indicating that binding was IGF2-dependent. (Data not shown.) Coupling of vIGF2 peptide did not impair GAA enzyme activity.

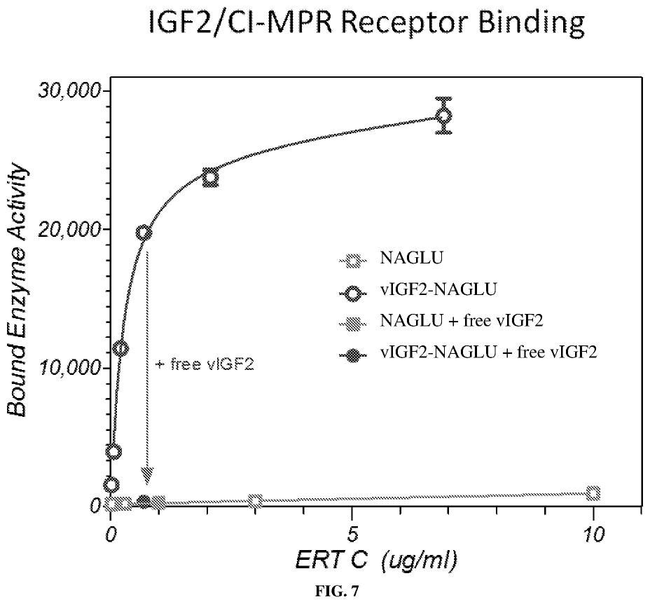

The vIGF2 was coupled to recombinant human N-acetyl-α-D-glucosaminidase (rhNAGLU). RrhNAGLU, a lysosomal enzyme lacking M6P, to determine whether peptide can convert a non-ligand to high affinity ligand for CI-MPR. In this experiment, rhNAGLU and vIGF2-rhNAGLU were directly compared using CI-MPR plate binding assays, utilizing CI-MPR-coated plates. Unbound enzyme was washed away prior to measuring bound enzyme activity. Varying concentrations of both enzyme preparations were used with or without free vIGF2 peptide. As shown in FIG. 7, vIGF2-rhNAGLU has significantly higher affinity for CI-MPR than rhNAGLU lacking vIGF2. Further, vIGF2-rhNAGLU binding was blocked by free vIGF2 peptide indicating that receptor binding was specific for IGF2 peptide. These results show that vIGF2 peptide can be utilized to improve drug targeting to lysosomes.

### Example 3: Myoblast Uptake of vIGF2-GAA Fusion Proteins

vIGF2-GAA fusion proteins (same sequences as in Examples 1-2) were administered and L6 myoblast uptake of the enzyme was measured. FIG. 6 shows superior uptake of the vIGF2-rhGAA compared to rhGAA and M6P-GAA. Therefore, vIGF2 is effective at targeting GAA to the cells.

### Example 4: Constructs for ERT Delivered by Gene Therapy

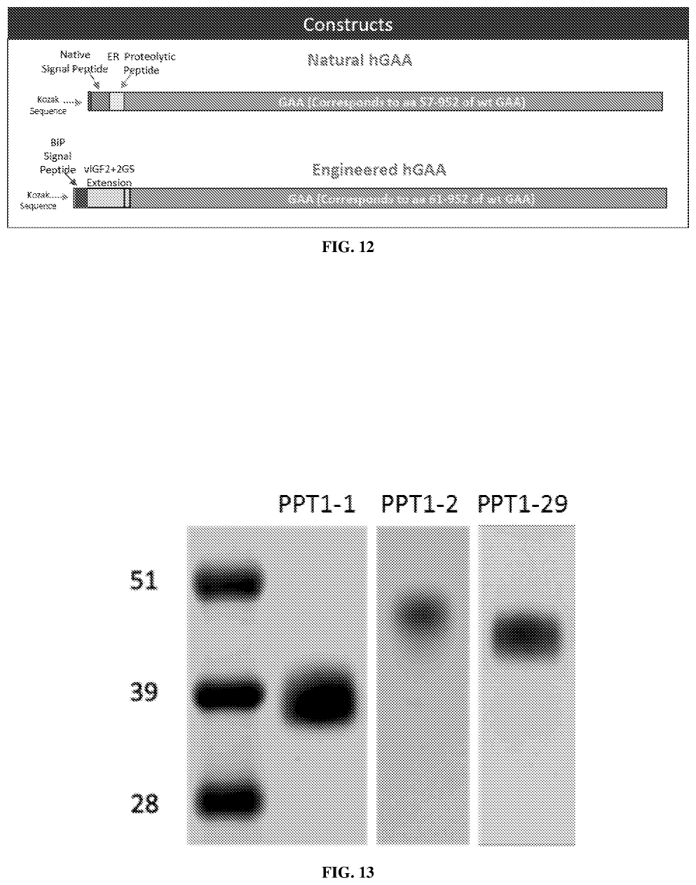

Two different constructs are illustrated in FIG. 12. In the top panel is a construct which contains a Kozak sequence and a nucleic acid encoding a recombinant human GAA with the native signal peptide encoding “natural hGAA” (SEQ ID NO: 189). In the middle panel is the construct Kozak-BiP-vIGF2-2GS-GAA, encoding “engineered hGAA” (SEQ ID NO: 190). This construct is characterized by a Kozak sequence, a nucleic acid encoding BiP signal peptide, a nucleic acid encoding the vIGF2 peptide having the sequence set forth in SEQ ID NO: 80, and a nucleic acid encoding a 2GS linker (SEQ ID NO:181) followed by a nucleic acid encoding a recombinant human GAA (SEQ ID NO:1) with the N-terminal 60 amino acids removed to prevent premature processing and removal of the vIGF2. The amino acid sequence of “engineered hGAA” is set forth in SEQ ID NO:2.

### Example 5: Enhanced Secretion of Gene Therapy Constructs

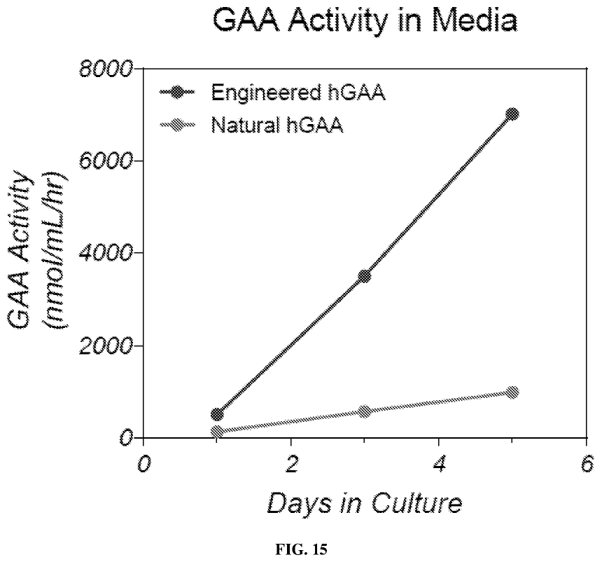

Engineered hGAA has greater secretion and is able to interact with a cell surface receptor appropriate for cellular uptake and lysosomal targeting CHO expressing engineered hGAA, described in more detail below, or natural hGAA were cultured and conditioned media was collected for measurement of GAA activity. FIG. 15 shows the relative activity of engineered and natural hGAA showing that engineered hGAA has increased activity compared to natural hGAA, indicative of more efficient secretion of engineered hGAA.

### Example 6: Analysis of PPT1 in Conditioned Media

Cloning of PPT1 Constructs

PPT1 constructs were cloned into the pcDNA3.1 expression vector (ThermoFisher cat#V79020), which contains a CMV promoter. The tested constructs included PPT1-1 (WT-PPT1) (SEQ ID NO: 4); PPT1-2 (WT-vIGF2-PPT1) (SEQ ID NO: 5); PPT1-29 (BiP2aa-vIGF2-PPT1) (SEQ ID NO: 6).

PPT1 Secretion & Binding

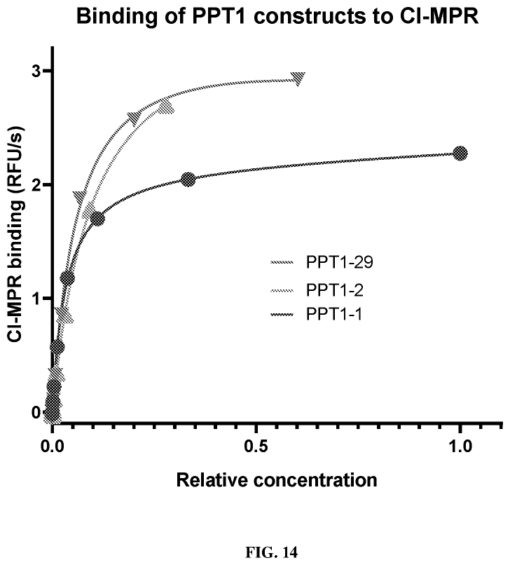

The PPT1 constructs were transiently expressed in HEK293T cells for 3 days and the PPT1 secreted into the media. Secreted PPT1 was quantified by Western Blotting, and assayed for CI-MPR binding using established methods. Secreted PPT1 is shown in FIG. 13. CI-MPR binding is shown in FIG. 14.

### Example 7: Testing Gene Therapy Vectors in an Animal Model of Pompe Disease

Pompe Gene Therapy: Preclinical Proof of Concept Study Design

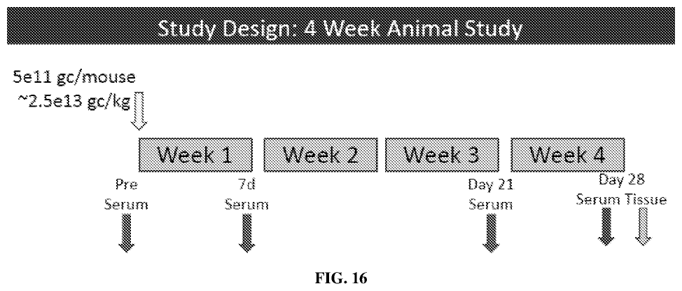

A preclinical study was conducted in GAA knockout (GAA KO) mice using a high dose for initial comparison of constructs. The constructs are shown in FIG. 12. Mice were treated with vehicle or one of two constructs, Natural—hGAA or Engineered—hGAA. Mice were administered Sell gc/mouse (approximately 2.5e13 gc/kg). GAA knockout mice were used at age 2 months. Normal (wildtype) mice were used as a control. The study design is outlined in FIG. 16.

Pompe Gene Therapy: Plasma

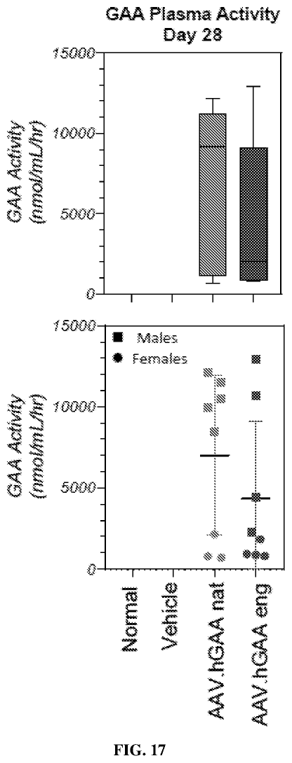

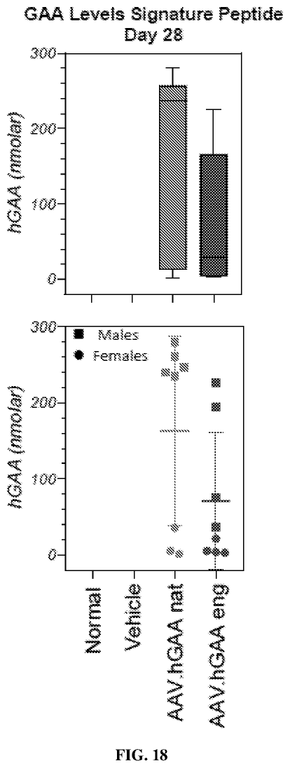

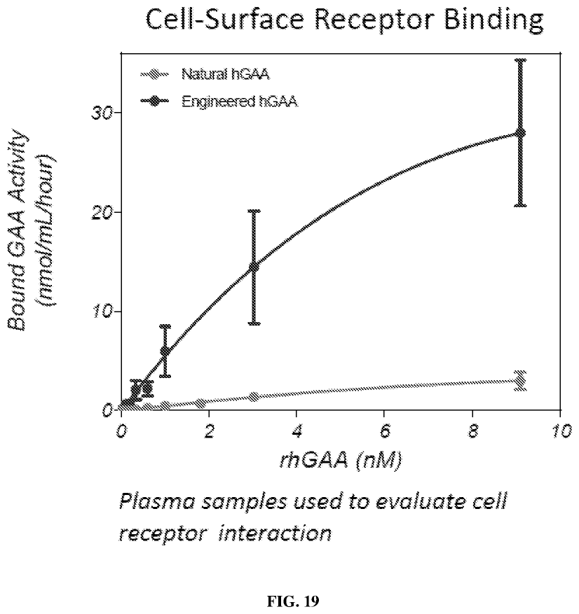

Plasma was collected from wild type (normal) mice or GAA KO mice treated with vehicle or a gene therapy vector as indicated and GAA activity and cell surface binding was measured. Data are summarized in FIG. 17, FIG. 27, and FIG. 19. Similar high GAA levels were seen in mice treated with gene therapy vectors (FIG. 17, FIG. 18). However, greater cell targeting receptor binding was observed with the engineered construct (FIG. 19).

Pompe Gene Therapy: Quadriceps

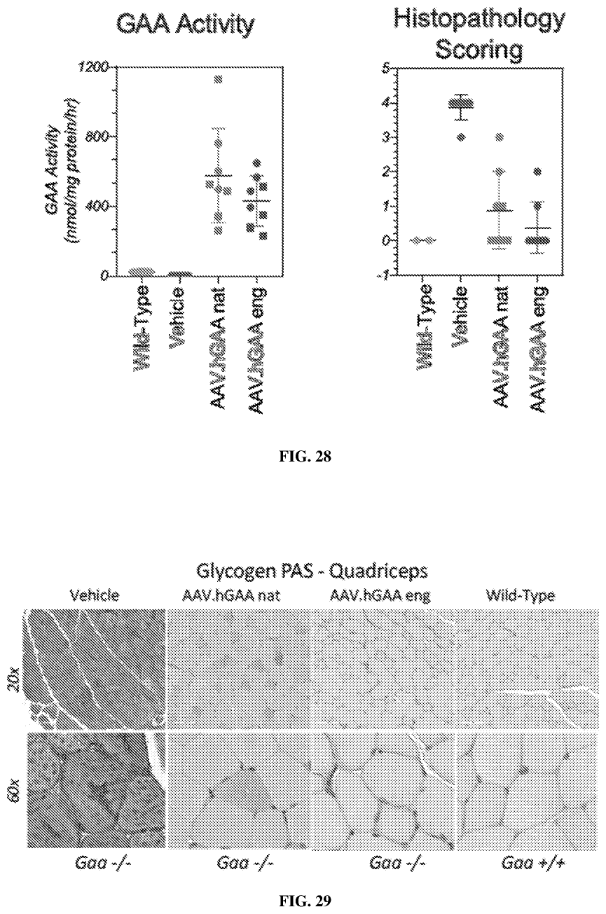

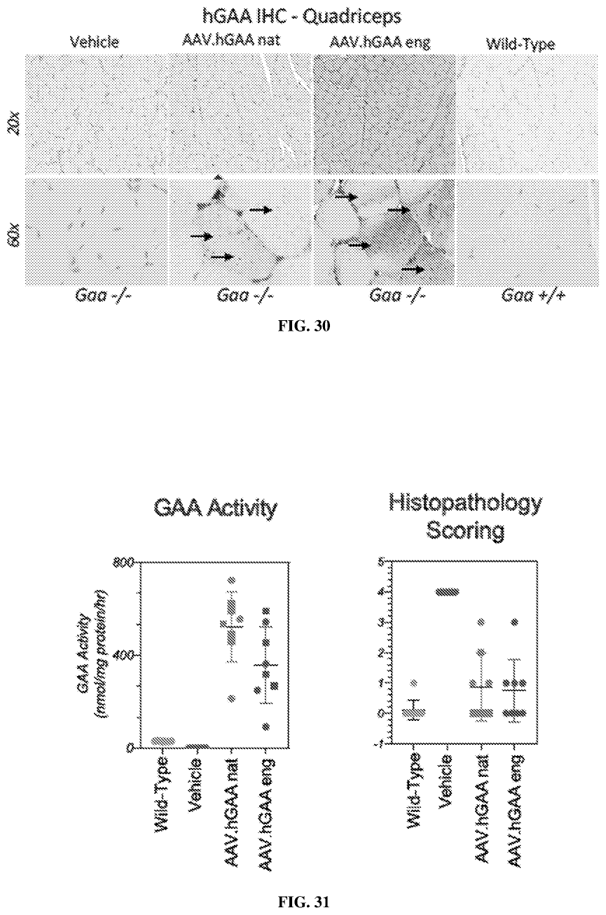

GAA activity, and glycogen storage/cytoplasmic vacuolization were assessed in normal (wild type) mice and treated GAA KO mice (FIG. 28). GAA activity in the quadriceps was about 20-fold higher than wild type. Glycogen PAS (FIG. 29) and immunohistochemistry (FIG. 30) were also assessed. Immunohistochemistry showed greater lysosomal targeting of engineered hGAA compared to wild type. Glycogen reduction was more consistent for engineered hGAA by PAS staining.

Pompe Gene Therapy: Triceps

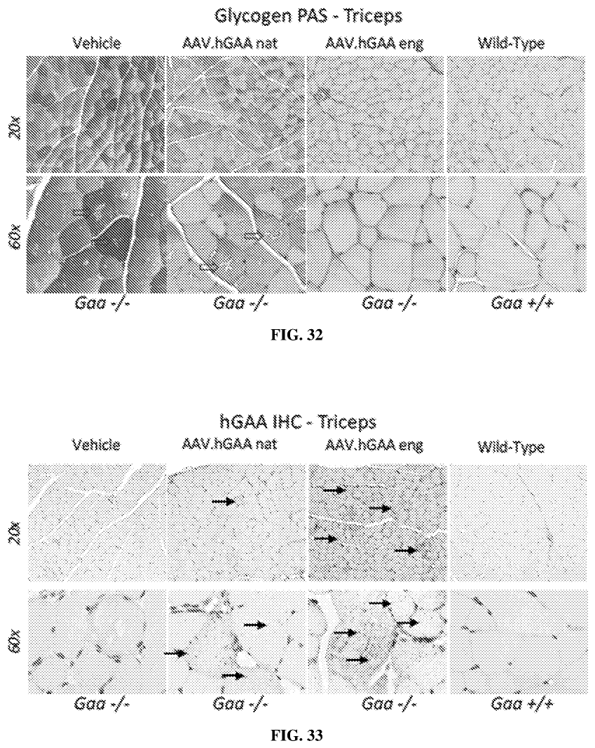

GAA activity, and glycogen storage/cytoplasmic vacuolization were assessed in normal (wild type) mice and in treated GAA KO mice (FIG. 31). GAA activity was about 10-15-fold higher than wild type. Immunohistochemistry and glycogen PAS were also assessed (FIG. 32 and FIG. 33). Immunohistochemistry illustrated greater lysosomal targeting of engineered hGAA compared to wildtype GAA. Glycogen reduction was more consistent for engineered hGAA as measured by PAS staining.

Pompe Gene Therapy: Tibialis Anterior (TA)

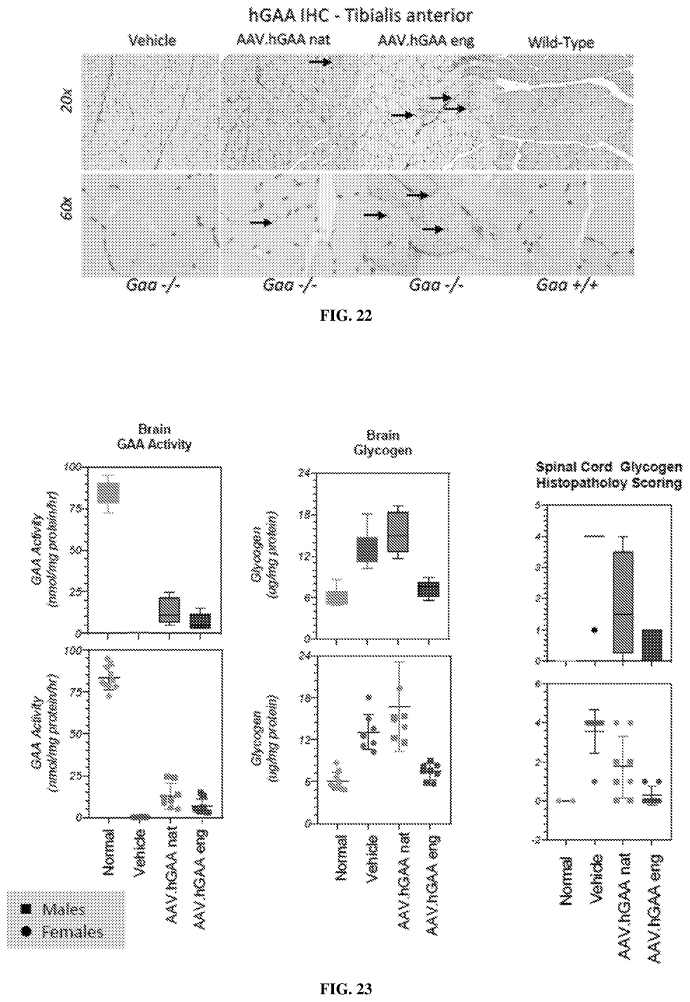

GAA activity, and glycogen storage/cytoplasmic vacuolization were assessed in normal (wild type) and treated GAA KO mice (FIG. 20). GAA activity in the TA was about 15-20-fold higher than wild type. Immunohistochemistry and glycogen PAS were also assessed (FIG. 21 and FIG. 22). Immunohistochemistry illustrated greater lysosomal targeting of engineered hGAA compared to wildtype GAA. Glycogen levels were close to wildtype levels. Glycogen reduction was more consistent for engineered hGAA by PAS staining.

Pompe Gene Therapy: Brain and Spinal Cord

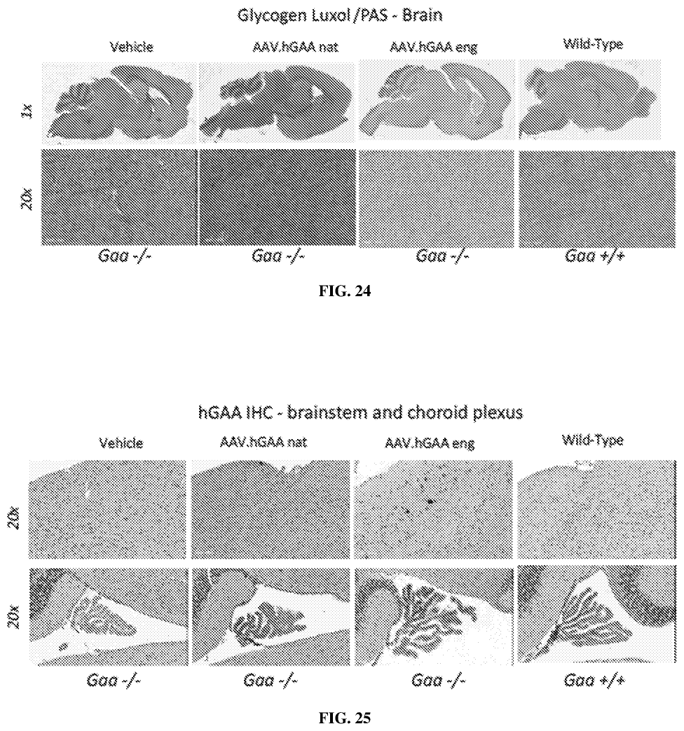

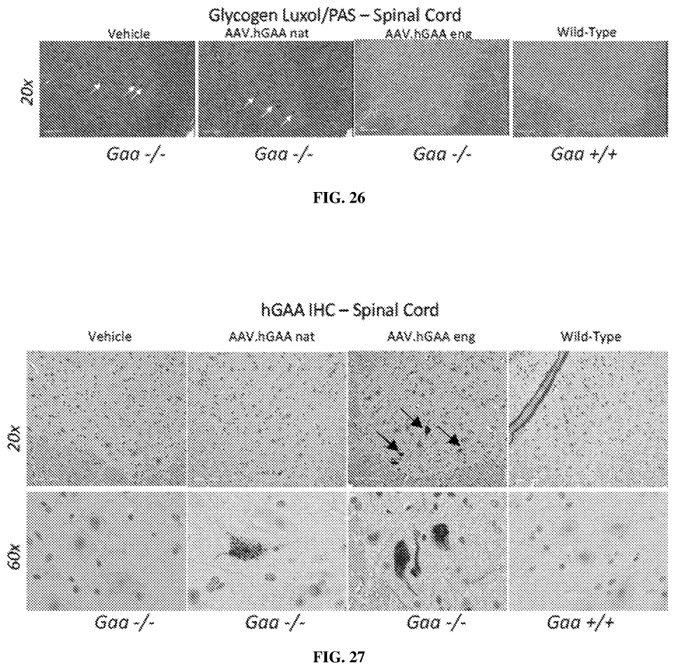

GAA activity, glycogen content, and glycogen storage/cytoplasmic vacuolization were assessed in normal (wild type) mice and treated GAA KO mice (FIG. 23). GAA activity in the brain was about 5-fold lower than wildtype. Immunohistochemistry and glycogen PAS were also assessed (FIG. 24, FIG. 25, FIG. 26, FIG. 27). Immunohistochemistry indicated that there may be a direct transduction of some cells. However, little to no glycogen clearance was obtained with the natural construct. Glycogen levels were close to wild type levels for the engineered construct even though activity was only 20% of wild type. PAS staining in the spinal cord shows little to no glycogen clearance with the natural construct. Glycogen levels close to wild type for engineered construct was observed in the ventral horn including motor neurons. Immunohistochemistry demonstrated direct transduction in spinal cord neurons. Engineered hGAA produced by the choroid plexus and neuronal cells was able to reduce glycogen by cross correction in the spinal cord while little glycogen reduction was observed for natural hGAA.

### CONCLUSIONS

Overall, the data in this example demonstrated that the engineered gene therapy constructs have dramatically better uptake into tissues and glycogen reduction than the wildtype GAA used in conventional treatments, including effects in the brain and spinal cord.

### Example 8: Animal Study Protocols

AAVhu68 vectors were produced and titrated by the Penn Vector Core as described. (Lock, Alvira et al. 2010, “Rapid, simple, and versatile manufacturing of recombinant adeno-associated viral vectors at scale.” Hum Gene Ther 21(10): 1259-1271).

Mus musculus, Pompe mice Gaa knock-out, in a C57BL/6/129 background founders were purchased at Jackson Labs (stock #004154, also known as 6neo mice).

Mice received 5×10¹¹ GCs (approximately 2.5×10¹³ GC/kg) of AAVhu68.CAG.hGAA (comprising either natural hGAA (SEQ ID NO: 189) or engineered hGAA (SEQ ID NO: 190) in 0.1 mL via the lateral tail vein, were bled on Day 7 and Day 21 post vector dosing for serum isolation, and were terminally bled (for plasma isolation) and euthanized by exsanguination 28 days post injection. Tissues were promptly collected, starting with brain.

GAA Activity

Plasma was mixed with 5.6 mM 4-MU-α-glucopyranoside pH 4.0 and incubated for three hours at 37° C. The reaction was stopped with 0.4 M sodium carbonate, pH 11.5. Relative fluorescence units, RFUs were measured using a Victor3 fluorimeter, ex 355 nm and emission at 460 nm. Activity in units of nmol/mL/hr was calculated by interpolation from a standard curve of 4-MU. Activity in individual tissue samples were further normalized based on total protein content in the homogenate.

GAA Signature Peptide by LC/MS

Plasma was precipitated in 100% methanol and centrifuged. Supernatants were discarded. The pellet was spiked with a stable isotope-labeled peptide unique to hGAA as an internal standard and resuspended with trypsin and incubated at 37° C. for one hour. The digestion was stopped with 10% formic acid. Tryptic peptides were separated by C-18 reverse phase chromatography and Identified and quantified by ESI-mass spectroscopy. The total GAA concentration in plasma was calculated from the signature peptide concentration.

Cell Surface Receptor Binding Assay

A 96-well plate was coated with receptor, washed, and blocked with BSA. 28-day plasma from AAV treated mice was serially diluted to give a series of decreasing concentrations and incubated with coupled receptor. After incubation the plate was washed to remove any unbound hGAA and 4-MU-α-glucopyranoside added for one hour at 37° C. The reaction was stopped with 1.0 M glycine, pH 10.5 and RFUs were read by a Spectramax fluorimeter; ex 370, emission 460. RFU's for each sample were converted to activity (nmol/mL/hr) by interpolation from a standard curve of 4-MU. Nonlinear regression was done using GraphPad Prism.

Histology

Tissues were formalin fixed and paraffin embedded. Muscle slides were stained with PAS; CNS slides with luxol fast blue/Periodic Acid-Schiff (PAS). A board-certified veterinary pathologist (JH) blindly reviewed histological slides. A semi-quantitative estimation of the total percentage of cells with glycogen storage and cytoplasmic vacuolization was done on scanned slides. A score from 0 to 4 was attributed as described in table below.

TABLE 9
Histology Scoring

| | Storage/Vacuolization |
|---|---|
| 0 | 0 |
| 1 | 1 to 9% |
| 2 | 10 to 49% |
| 3 | 50 to 74% |
| 4 | 75 to 100% |

Immuno-Histochemistry (IHC)

We studied transgene expression and cellular localization from slides immunostained using an anti-human GAA antibody (Sigma HPA029126).

### Example 9: Histology-Tissue Processing—Protocols and Results in an Animal Model of Pompe Disease

All tissues were fixed in 10% NBF (neutral buffered formalin). The assays (PAS and IHC) are routinely used in the field.

PAS staining of quadriceps and triceps (FIG. 29 and FIG. 32)—Tissues were fixed in 10% NBF and embedded in paraffin. Sections were post-fixed in 1% periodic acid and stained with Schiff's reagent. Afterwards, sections were counterstained with hematoxylin. Glycogen appears as magenta aggregates (lysosomal bound) or diffused pink (cytosolic); nuclei are blue. Based on the images and assuming each is representative of a group, the ranking order in terms of glycogen clearance is: Engineered hGAA>Natural hGAA. The Engineered hGAA construct produced more staining across the entire image compared to the rest, showing an improved endocytosis of GAA protein mediated through the binding of vIGF2 to CI-MPR.

PAS staining of spinal cord (FIG. 26)—Tissues were fixed in 10% NBF. Post-fixation in 1% periodic acid could have been done prior to or after paraffin embedding. Sections were stained with Schiff's reagent and counterstained likely with methylene blue. Glycogen appears as magenta aggregates (lysosomal bound); nerve fibers appear blue. The images focused on the ventral horn of the spinal cord and glycogen accumulation in the motor neurons. Engineered hGAA appeared most effective in glycogen reduction among the constructs.

GAA IHC (FIG. 22, FIG. 25, FIG. 27, FIG. 30, and FIG. 35)—Tissues were fixed in 10% NBF and embedded in paraffin. Sections were incubated with an anti-GAA primary antibody, followed by a secondary antibody that recognizes the primary antibody and carries an enzyme tag—HRP. Subsequently, an enzymatic reaction was carried out and a brown-colored precipitating product was formed. Sections were then counterstained with hematoxylin. The constructs showed GAA uptake into muscle fibers (FIG. 31). Engineered hGAA>Natural hGAA. The BiP-vIGF2 construct had more diffused staining across the entire image compared to the rest.

Compared to other vectors, engineered hGAA produced more GAA IHC signals with a punctum-like appearance inside the muscle fibers, showing a much more efficient lysosomal targeting (FIG. 22).

In all, engineered hGAA consistently demonstrated superiority in tissue uptake, lysosomal targeting, and glycogen reduction in various tissues among the constructs.

### Example 10: Binding of Fusion Proteins to CIMPR

In this example, therapeutic enzymes were engineered to be targeted to the CI-MPR. Data in this example show that the fusion proteins bind better to CIMPR when they contain a vIGF2 tag. This was shown even for enzymes that are known to be well-phosphorylated, such as PPT1.

Each transgene was cloned into a pIREShyg3 plasmid and the DNA was transfected in suspension HEK 293K cells using PEI transfection reagent. Cells were grown in FreeStyle 293 expression media. The conditioned media was harvested from the cells three to four days post-transfection. The amount of secreted enzyme in the conditioned media was determined by activity assay or by Signature Peptide assay. These concentrations were used to set up CIMPR binding assays.

In the binding assay, a plate was first coated with CI-MPR. Next, a sample containing the enzyme of interest was incubated on the plate. The plate was washed so that only substances bound to CI-MPR remain on the plate. The amount of the enzyme of interest bound to the plate was determined by enzyme assay or by mass spec. The binding assay was performed at a range of concentrations of the enzyme of interest in order to obtain a binding curve.

The amount of tagged and untagged enzyme bound to the plate was determined in order to construct binding curves. In the case of AGA and TPP1, enzyme activity assays were performed to make this determination. In other cases, the Signature Peptide assay was performed to determine the amount of enzyme bound.

TPP1 activity assay is described at www.rndsystems.com/products/recombinant-human-tripeptidyl-peptidas e-i-tpp1-protein-cf_2237-se#product-details.

AGA activity assay described at YaV, et al. Applications of a new fluorometric enzyme assay for the diagnosis of aspartylglucosaminuria. J Inherit Metab Disease 1993 and Banning, et al. Identification of Small Molecule Compounds for Pharmacological Chaperone Therapy of Aspartylglucosaminuria. Sci Rep 2016.

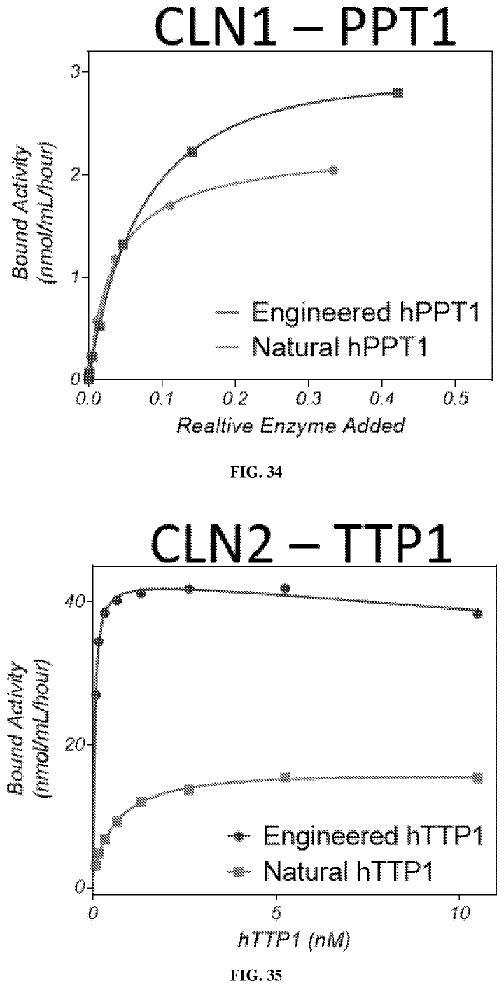

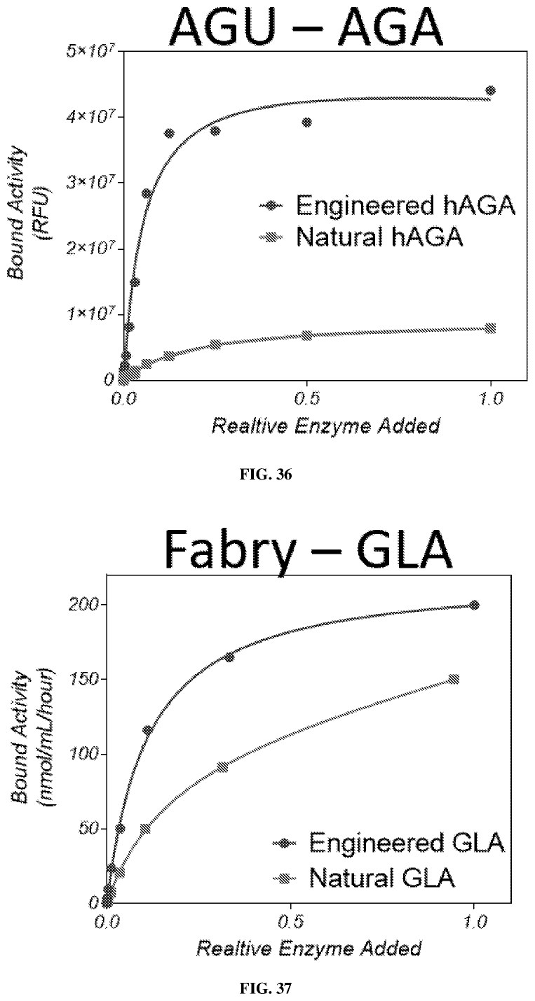

FIG. 34 shows increased binding of engineered PPT1 compared to wild type PPT1. FIG. 35 shows increased binding of engineered TPP1 compared to wild type TPP1. FIG. 36 shows increased binding of engineered AGA compared to wild type AGA. FIG. 37 shows increased binding of engineered GLA compared to wild type GLA.

### Example 11: Cloning of PPT1 Fusions

All PPT1 constructs were assembled into the pcDNA3.1 expression vector using the In-Fusion cloning kit from Takara Bio.

The linearized pcDNA3.1 vector and each PPT1 gene fragments were recombined via the InFusion reaction to yield the final pcDNA3.1 vector harboring the stated PPT1 constructs.

### Example 12: Cloning vIGF2 Mutants

All of the vIGF2 mutants were swapped into the pcDNA3.1-BiP-vIGF2-2GS-GAA expression vector using the In-Fusion cloning kit from Takara.

Recombination of the ordered vIGF2 fragment and the linearized pcDNA3.1-GAA vector via the InFusion reaction gave the final pcDNA3.1-BiP-vIGF2*-2GS-GAA circular expression vector.

### Example 13: Characterization of vIGF2-GAA Constructs

Transient Transfection of HEK293T Cells with pcDNA3.1-vIGF2-GAA Plasmids

HEK293T cells were transiently transfected with 1 μg of DNA using Fugene HD transfection reagent. The cultures were incubated for an additional 2-5 days at 37° C. supplemented with 5% CO₂ before harvesting the conditioned media and cell pellet.

Western Blot Analysis of vIGF2-GAA in Conditioned Media

Western blots were performed using a common standard method using the Licor Odyssey detection system. The primary antibody used for vIGF2-GAA detection was an in-house rabbit Anti-GAA antibody (FL059). The secondary antibodies used for GAA were goat anti-rabbit DyLight 800 (ThermoFisher cat #SA5-35571).

GAA Activity Assay

GAA activity was measured as described above.

CI-MPR Binding Assay

CI-MPR binding was measured as described above.

Cellular Uptake Assay

Results from the creation of 30+ IGF2-GAA constructs is as follows.

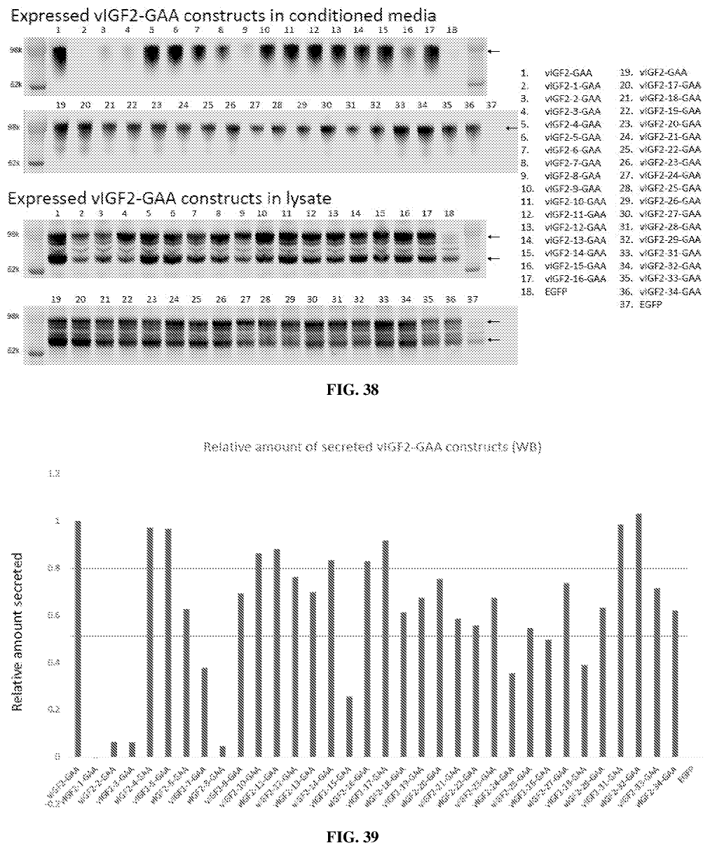

vIGF2-GAA constructs that exhibited secretion/expression level not less than 80% of the original vIGF2 are vIGF2-4, 5, 10, 11, 14, 16, 17, 31, and 32 (FIG. 38 and FIG. 39).

vIGF2-GAA constructs that exhibited secretion/expression level not less than 50% of the original vIGF2 are vIGF2-4, 5, 6, 9-14, 16-23, 25, 27, and 29-34 (FIG. 38 and FIG. 39).

All vIGF2-GAA constructs appeared to have processed correctly inside cells where the 70/76 KDa mature GAA peptide fragment was observed (FIG. 38).

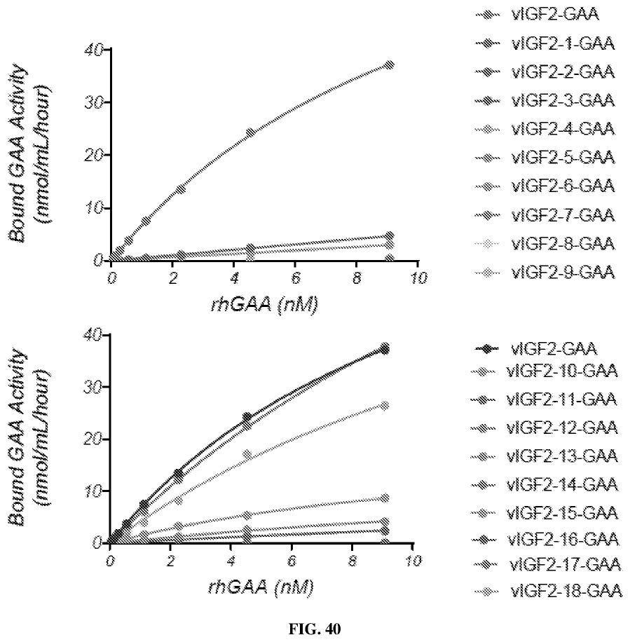

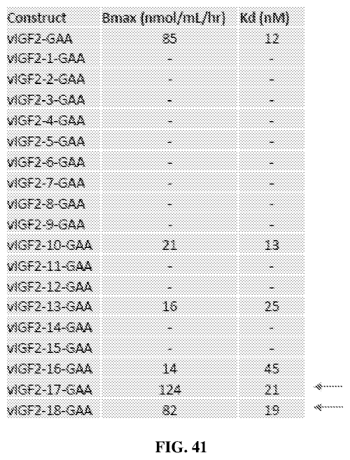

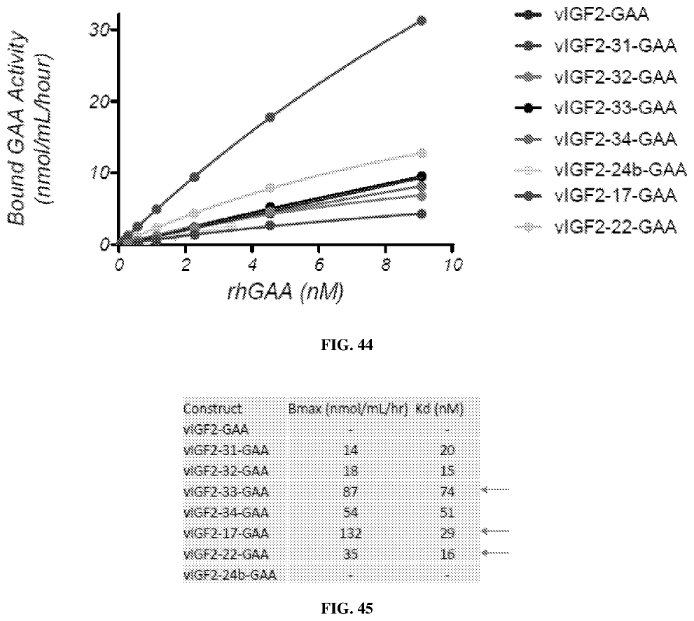

vIGF2-17 consistently gave a CI-MPR binding Bmax significantly higher than the original vIGF2 (FIG. 40, FIG. 41, FIG. 44, and FIG. 45).

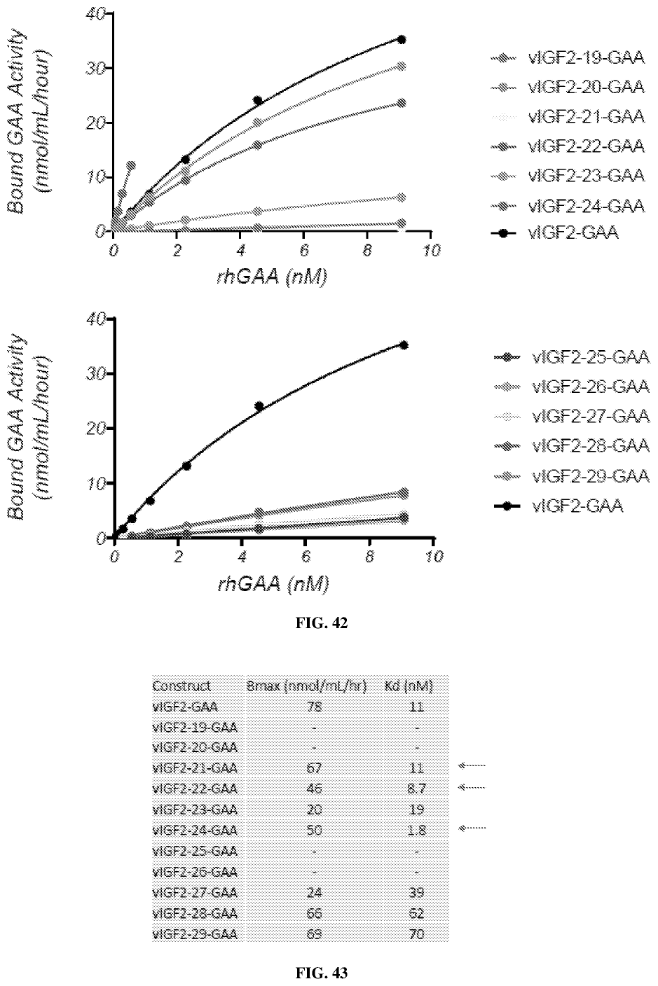

vIGF2-24 has binds CI-MPR significantly better than the original vIGF2 (FIG. 42 and FIG. 43).

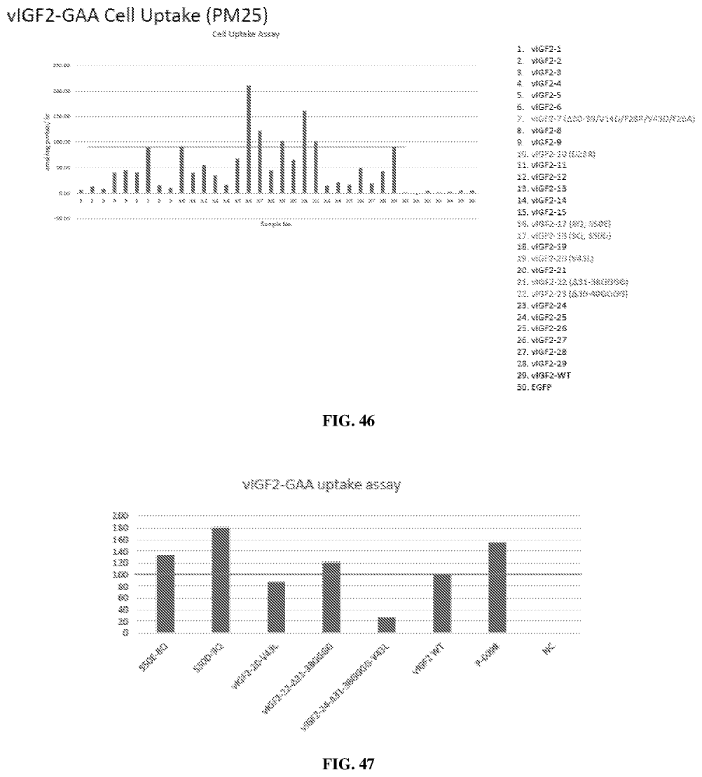

vIGF2-GAA constructs that have a comparable or better PM25 cellular uptake properties to the original vIGF2 include vIGF2-7, vIGF2-10, vIGF-17, vIGF2-18, vIGF2-20, vIGF2-22, & vIGF2-23 (FIG. 46 and FIG. 47).

### Example 14: Testing of PPT1 Constructs

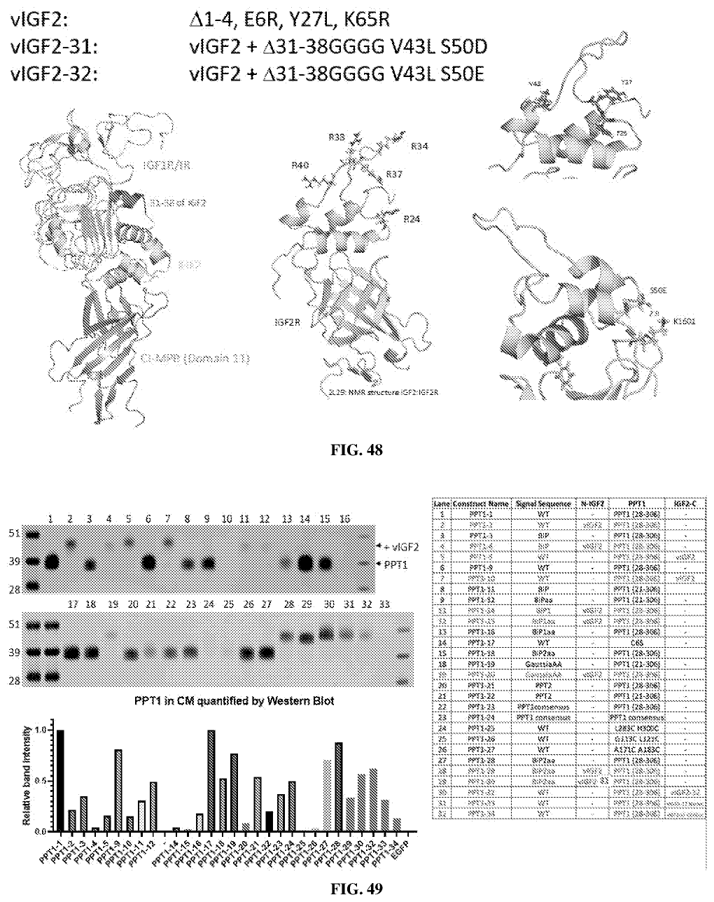

vIGF2 peptides were designed as discussed elsewhere herein. Variants were selected based on increased selective binding to CI-MPR and improved protein expression. Exemplary peptides and their structure are provided in FIG. 48.

Transient Transfection of HEK293T Cells with pcDNA3.1-PPT1 Plasmids

HEK293T cells grown to about 80% confluence in 1 mL OptiMEM media supplemented with 5% FBS in 12-well culture were transiently transfected with 1 μg of DNA using Fugene HD transfection reagent. The cultures were incubated for an additional 2-5 days at 37° C. supplemented with 5% CO₂ before harvesting the conditioned media and cell pellet.

Western Blot Analysis of PPT1 in Conditioned Media

Western blots were performed using a common standard method using the Licor Odyssey detection system. The primary antibody used for PPT1 detection was a mouse polyclonal antibody from Abcam (catalog cat #ab89022). The secondary antibodies used for PPT1 were goat anti-mouse DyLight 800 (ThermoFisher cat #SA5-35521).

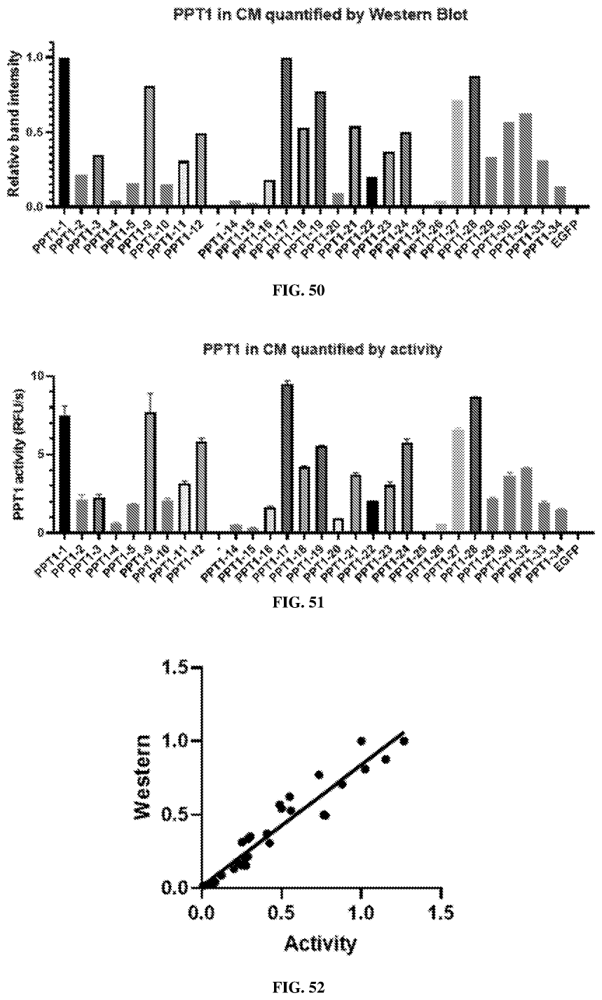

Western blots of PPT1 expression and a graph showing band intensity are shown in FIG. 49. A graph showing PPT1 in conditioned media quantified by Western blot is shown in FIG. 50.

PPT1 Activity Assay

The PPT1 activity assay used was essentially that described by Van Diggelen et al. (Mol Genet Metab. 66:240-244, 1999). Briefly in a typical PPT1 activity assay, 10 ul of conditioned media containing secreted PPT1 was mixed with 90 ul of reaction buffer containing 75 uM MU-6S-Palm-βGlc (4-methylumbelliferyl-6-thio-palmitate-β-D-glucopyranoside, Cayman Chemical; CAS 229644-17-1), 2 U/mL β-glucosidase (Sigma Chemicals; CAS 9001-22-3; G4511), 20 mM citrate pH 4.0, 5 mM DTT, 0.02% Triton X-100, and 50 mM NaCl in a 96-well black, clear bottom plate (Corning cat #3631). Using an excitation wavelength of 330 nm and emission wavelength of 450 nm, fluorescence was monitored at 30-second intervals over a 1 hr period at 25° C. using the SpectraMax M2. The rate of the PPT1 reaction was extracted by fitting the time course fluorescence data with a linear regression.

A graph showing PPT1 in conditioned media quantified by activity is shown in FIG. 51. Activity was found to have a strong correlation with the Western blot results. FIG. 52 shows the correlation between activity and Western blot quantification.

PPT1 Stability Assay

Briefly, in a typical stability assay, 180 μL of conditioned media containing PPT1 was diluted with 20 μL of 10×PBS, pH 7.4 and incubated at 37° C. At different time points, an aliquot of 15 μL was taken out and flashed frozen in ethanol cooled with dry ice. At the end of the time course experiment, the frozen samples were thawed and PPT1 activity was measured using the PPT1 activity assay.

CI-MPR Binding Assay

CI-MPR plate-binding assay performed as previously described, then amount bound was determined by PPT1 activity assay.

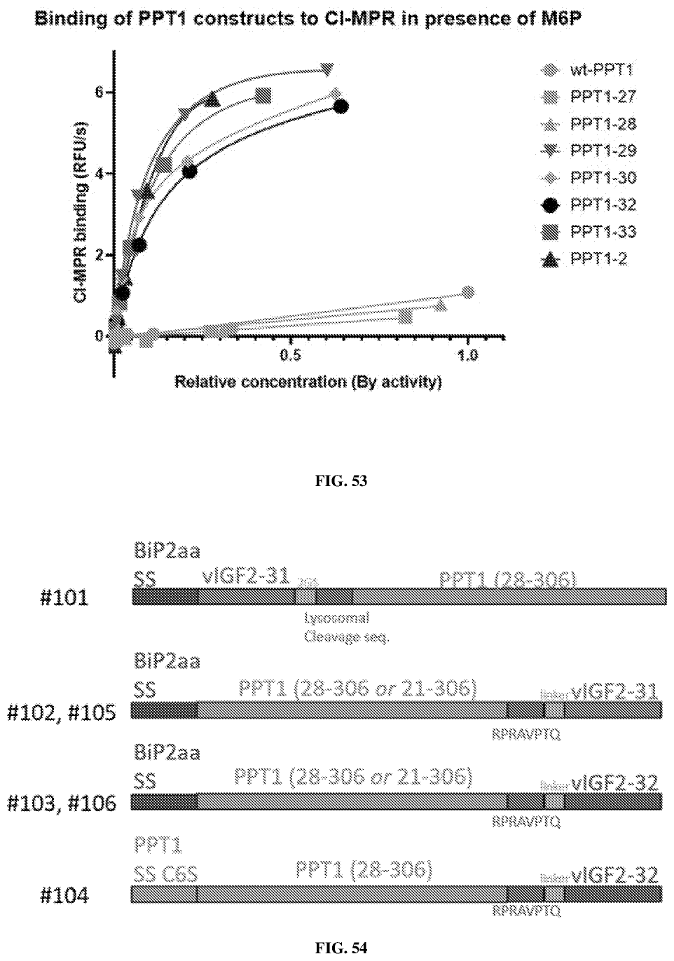

Binding of PPT1 constructs to CI-MPR in presence of M6P in the table below. Binding curves are shown in FIG. 53.

TABLE 10
Binding of PPT1 constructs to CI-MPR in presence of M6P

| | Bmax | Relative Kd |
|---|---|---|
| PPT1-9 | | ND |
| PPT1-27 | | ND |
| PPT1-28 | | ND |
| PPT1-29 | 8.98 | 0.107 |
| PPT1-30 | 4.88 | 0.056 |
| PPT1-32 | 6.05 | 0.121 |
| PPT1-33 | 9.19 | 0.143 |
| PPT1-2 | | ND |

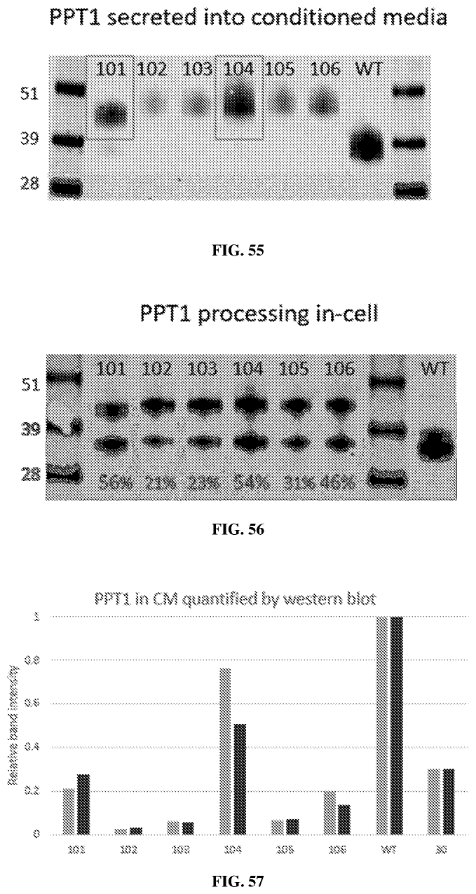

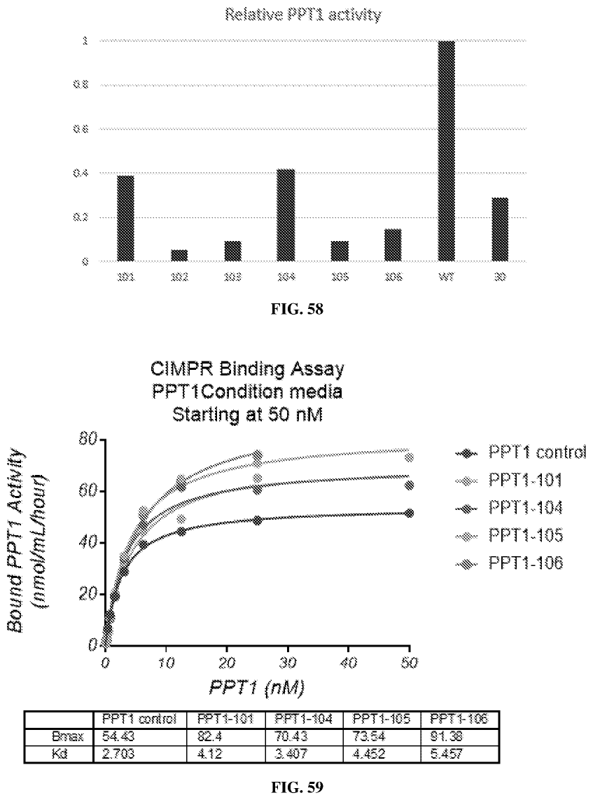

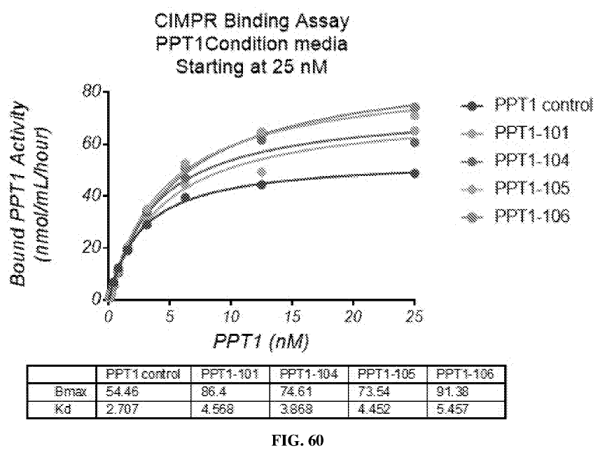

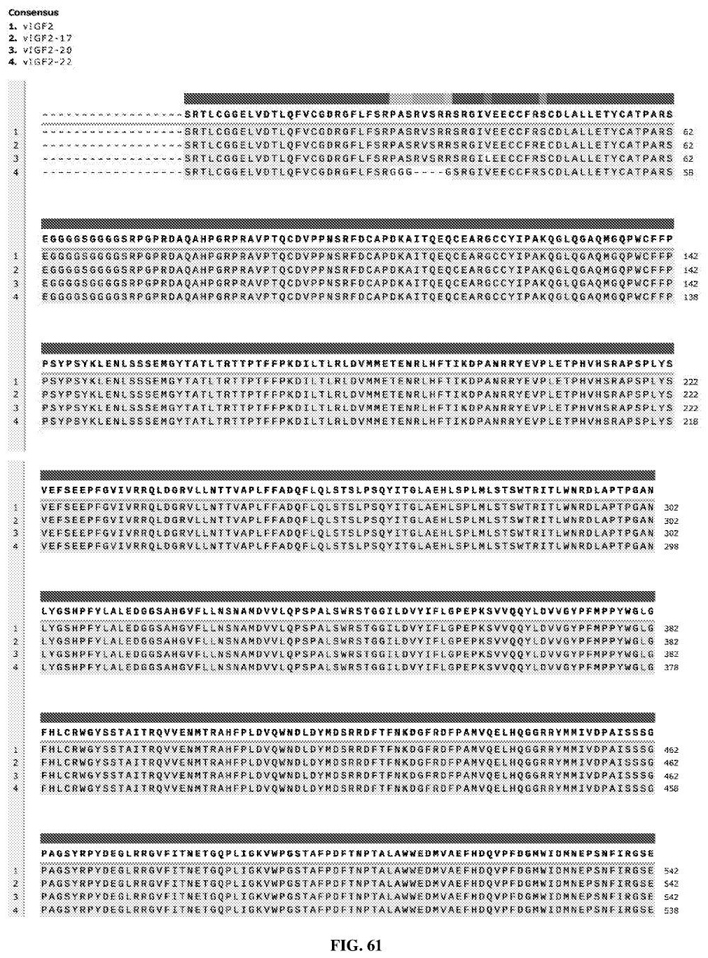

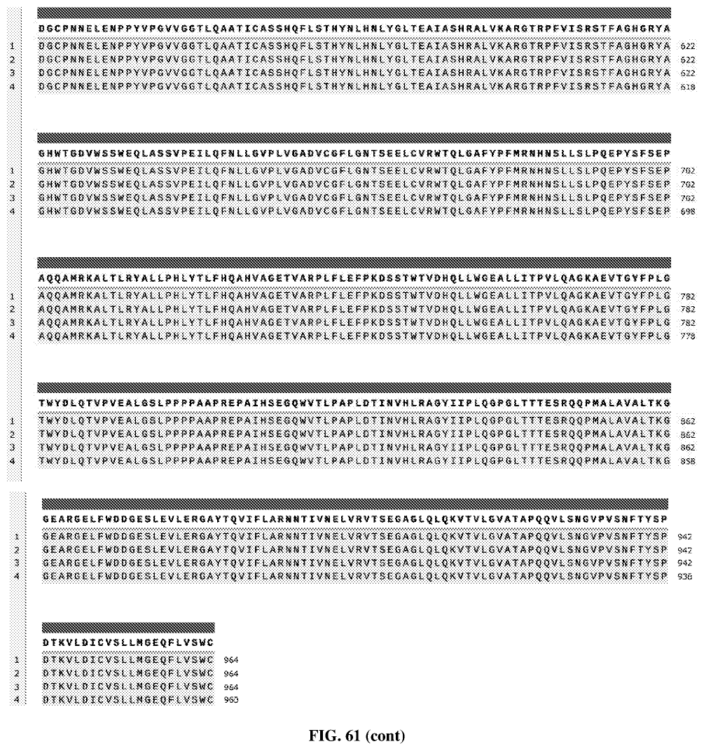

Six PPT1 constructs were selected for further analysis. These six constructs are shown in FIG. 54. PPT1 secretion into the media (FIG. 55), PPT1 processing in-cell (FIG. 56), PPT1 quantification by Western blot (FIG. 57) and activity (FIG. 58) were determined for these six constructs.

### Example 15 Engineering and Testing of Additional PPT1 IGF2 Fusion Constructs

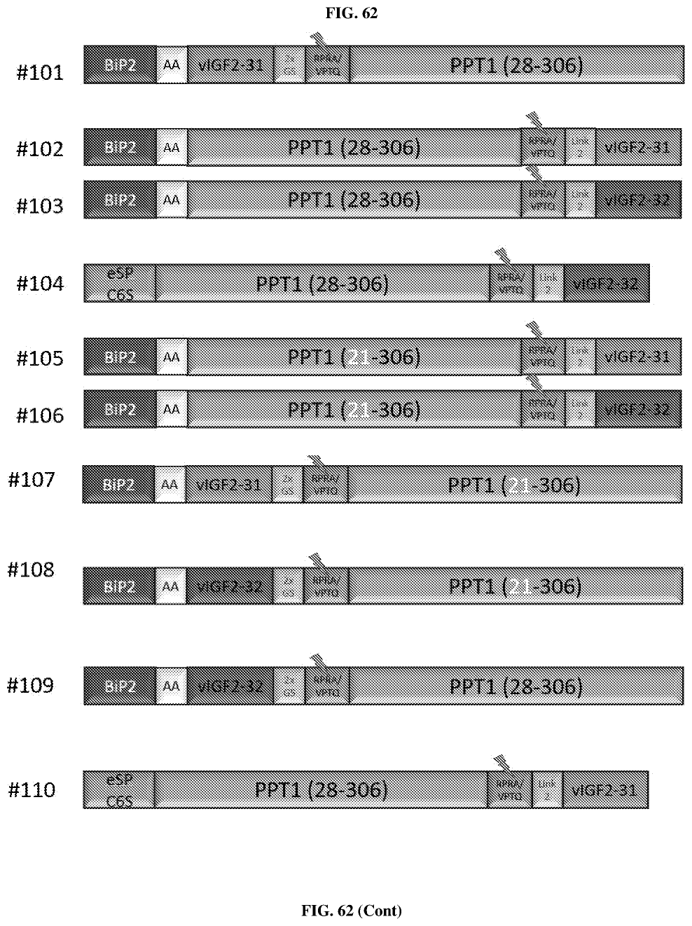

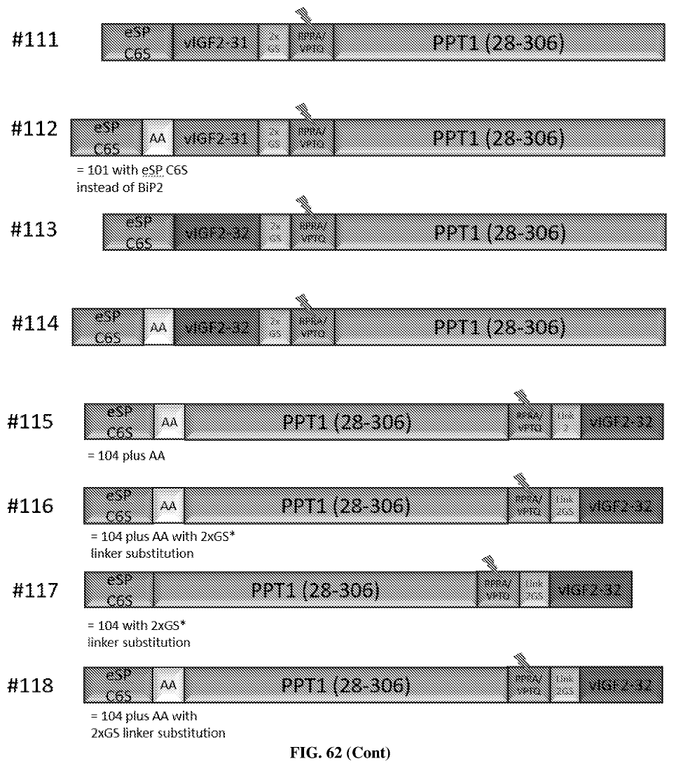

Additional PPT1 constructs were designed and cloned as shown in FIG. 62. These constructs contain either an endogenous signal sequence with a C6S mutation (SEQ ID NO:177), optionally with a two alanine extension to improve cleavage (SEQ ID NO:178), or a modified BiP signal peptide, BiP-2 (SEQ ID: 171), a PPT1 sequence comprising amino acid residues 21-306 or 28-306 of wild-type human PPT1 (SEQ ID NO: 4), a GS linker (SEQ ID NO:181-187), and a variant IGF2-31 or 32 (SEQ ID NOs:120 or 121), separated by a lysosomal cleavage site, RPRAVPTQA (SEQ ID NO: 188).

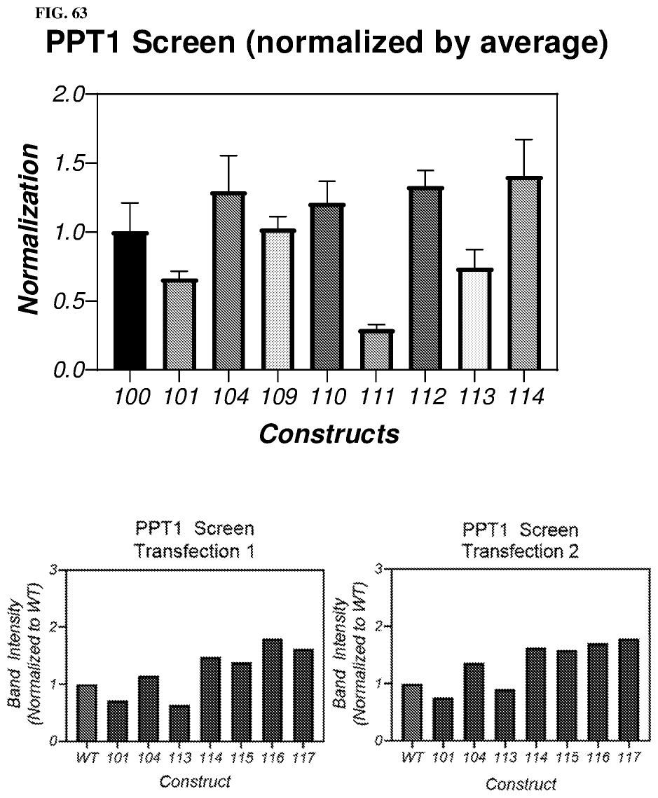

All PPT1 constructs (FIG. 62) were transiently expressed in FreeStyle 293 suspension cells. Briefly, FreeStyle 293 cells were transfected with each PPT1 construct in a pcDNA3.1 backbone, using polyethylenimine (PEI) as a transfection reagent. After four days of expression in FreeStyle 293 expression medium, the conditioned medium from each transfection was collected and run on western blots, using an anti-PPT1 primary antibody. Relative PPT1 levels in the medium were quantified from the band density on these western blots. FIG. 63 shows that several constructs tested have higher levels secreted into the medium than WT PPT1. Higher PPT1 levels in the conditioned medium are reflective of both good expression and efficient secretion from the cell. Although vIGF2-31 (SEQ ID NO:120) and vIGF2-32 (SEQ ID NO:32) were designed to improve CIMPR binding, the surprisingly enhanced the expression and secretion of PPT1 compared to an earlier IGF2 variant (SEQ ID NO:80).

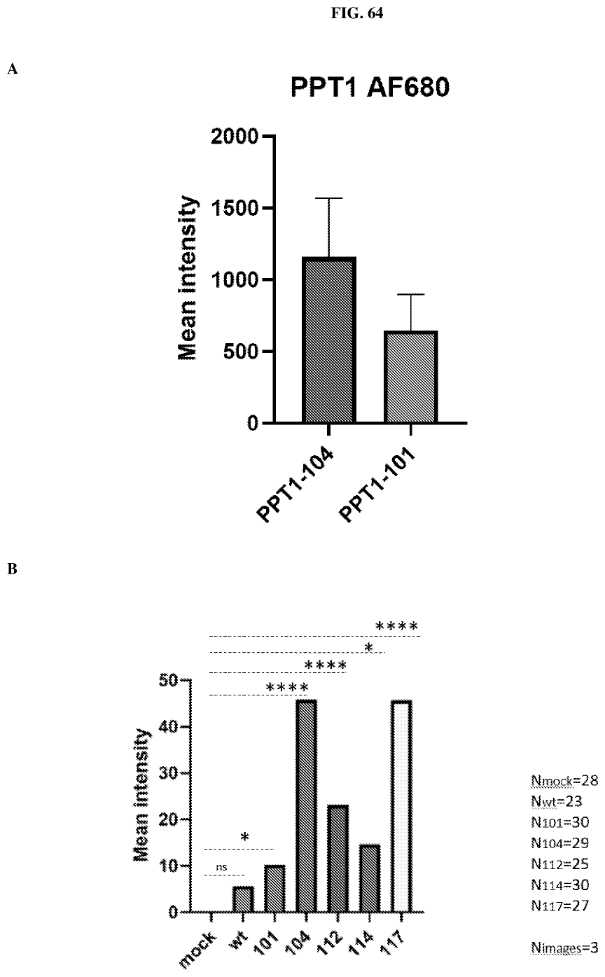

Neuronal uptake experiments with purified protein constructs PPT1-101 and PPT1-104 showed successful uptake of both proteins, with approximately twice as much PPT1-104 taken up as PPT1-101 (FIG. 64A). For this experiment, rat cortical neurons were cultured in NeuroCult medium and plated on poly-L-lysine coated cover slips. The neurons were treated with 5 ug/ml purified PPT1-101 or PPT1-104, which had been labeled with Alexa Fluor 680 fluorescent dye. After a one-hour incubation, the cells were fixed, permeabilized, and imaged using a Leica SP8 confocal microscope.

Neuronal uptake experiments with conditioned medium were performed using conditioned medium obtained from FreeStyle 293 cell transfections, as described above. The concentration of each PPT1 construct protein in the media was first determined via western blot, using a standard curve generated using a sample of PPT1 of known concentration. Each sample of conditioned media was concentrated before treating the neurons. Rat cortical neurons were cultured in Primary Neuron Growth Medium and plated on poly-L-lysine coated cover slips. The neurons were treated with the following concentrations of PPT1 protein in media:

| WT | PPT1-101 | PPT1-104 | PPT1-112 | PPT1-114 | PPT1-117 |
|---|---|---|---|---|---|
| 5.6 ug/ml | 6.8 ug/ml | 12.8 ug/ml | 14.8 ug/ml | 15.4 ug/ml | 17.8 ug/ml |

After a one-hour incubation, the cells were fixed, permeabilized, and imaged using a Leica SP8 confocal microscope. Uptake with all PPT1 variants was higher than with WT PPT1; PPT1-104 and PPT1-117 showed the highest levels of uptake (FIG. 64 B).

### Example 16 Analysis of NAGLU Constructs

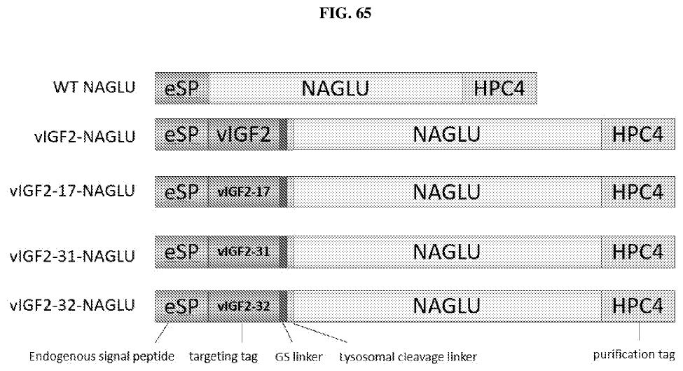

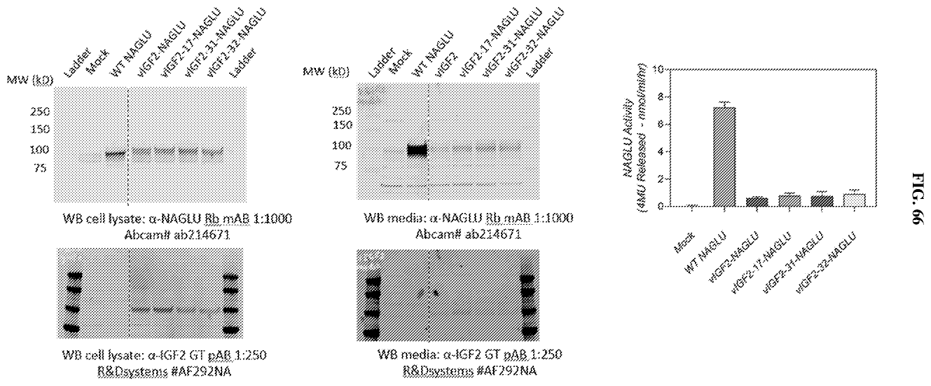

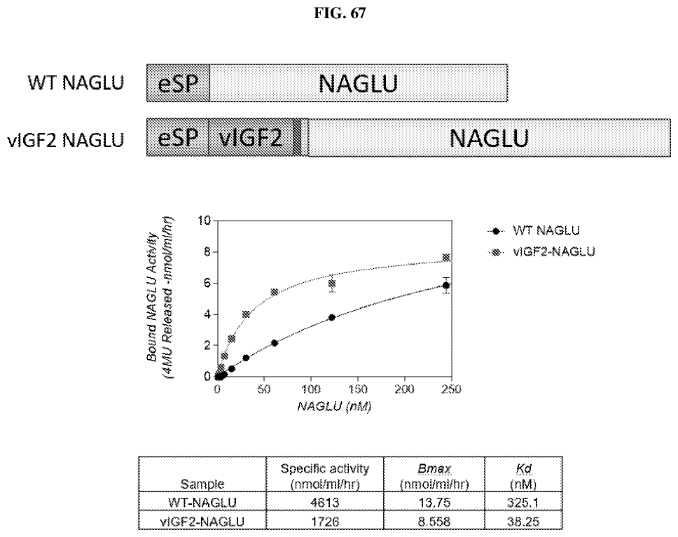

Mutant fusion proteins comprising recombinant human NAGLU protein having an N-terminal vIGF2 tag inserted between the signal peptide and the NAGLU protein were designed as shown in FIG. 65. Several variants were prepared including fusion proteins comprising vIGF2 (SEQ ID NO:80), vIGF2-17 (SEQ ID NO:106), vIGF2-31 (SEQ ID NO:120) and vIGF2-32 (SEQ ID NO:121). The fusion proteins were expressed in HEK293F cells. The NAGLU content as determined by Western blotting with ab214671 (R&Dsystems) is shown in the lysate and media fractions for each fusion protein tested. (FIG. 66A-B) Enzyme activity in conditioned media for each fusion protein was determined by a 4-MU assay. (FIG. 66 C) Protein amounts in conditioned media were not normalized/equalized and activity data represent relative secretion of constructs into conditioned media rather than relative specific activity of equal quantities of proteins. As seen in FIG. 66, the presence of variant IGF2 led to decreased expression and secretion as compared to untagged NAGLU. However, the CIMPR binding of IGF2-tagged NAGLU improved significantly compared to untagged NAGLU. (FIG. 67) Notably, −2.5-fold less IGF2-tagged NAGLU compared to WT was used as input for the binding assay, yet more of the tagged compared to WT bound to the immobilized receptor.

### Example 17 Analysis of TPP1 Constructs

A series of nucleic acid constructs for expressing TPP1 fusion proteins linked to IGF2 variants were designed and tested for expression, secretion and CIMPR binding. The fusion proteins comprise a signal peptide (SEQ ID NO:179, a variant IGF2 sequence (SEQ ID NOs:80, 106, 111, 133, 119-121), a GS linker (GGGGSGGGGS, SEQ ID NO:186), a lysosomal cleavage site (RPRAVPTQA, SEQ ID NO:188), a TPP1 propeptide (SEQ ID NO:45), and a TPP1 mature peptide (SEQ ID NO:46). Both N-terminally and C-terminally vIGF2 tagged constructs were generated and tested. Examples of PPT1 fusion proteins that were designed and tested are shown in Table 11.

TABLE 11
TPP1 Fusion Constructs

| PPT1 Construct | SEQ ID |
|---|---|
| pSvelte001- Native TPP1 Signal Peptide - vIGF2 - GS linker - | 47 |
| Lyso Cleave - TPP1 propeptide - TPP1 mature peptide | |
| pSvelte057 - Native TPP1 Signal Peptide - vIGF2v17 - GS | 48 |
| linker - Lyso Cleave - TPP1 propeptide - TPP1 mature peptide | |
| pSvelte059 - Native TPP1 Signal Peptide - vIGF2v22 - GS | 49 |
| linker - Lyso Cleave - TPP1 propeptide - TPP1 mature peptide | |
| pSvelte060 - Native TPP1 Signal Peptide - vIGF2v24- GS | 50 |
| linker - Lyso Cleave - TPP1 propeptide - TPP1 mature peptide | |
| pSvelte061 - Native TPP1 Signal Peptide - vIGF2v30 - GS | 51 |
| linker - Lyso Cleave - TPP1 propeptide - TPP1 mature peptide | |
| pSvelte062 - Native TPP1 Signal Peptide - vIGF2v31 - GS | 52 |
| linker - Lyso Cleave - TPP1 propeptide - TPP1 mature peptide | |
| pSvelte063- Native TPP1 Signal Peptide - vIGF2v32 - GS | 53 |
| linker - Lyso Cleave - TPP1 propeptide - TPP1 mature peptide | |

Expression & Secretion

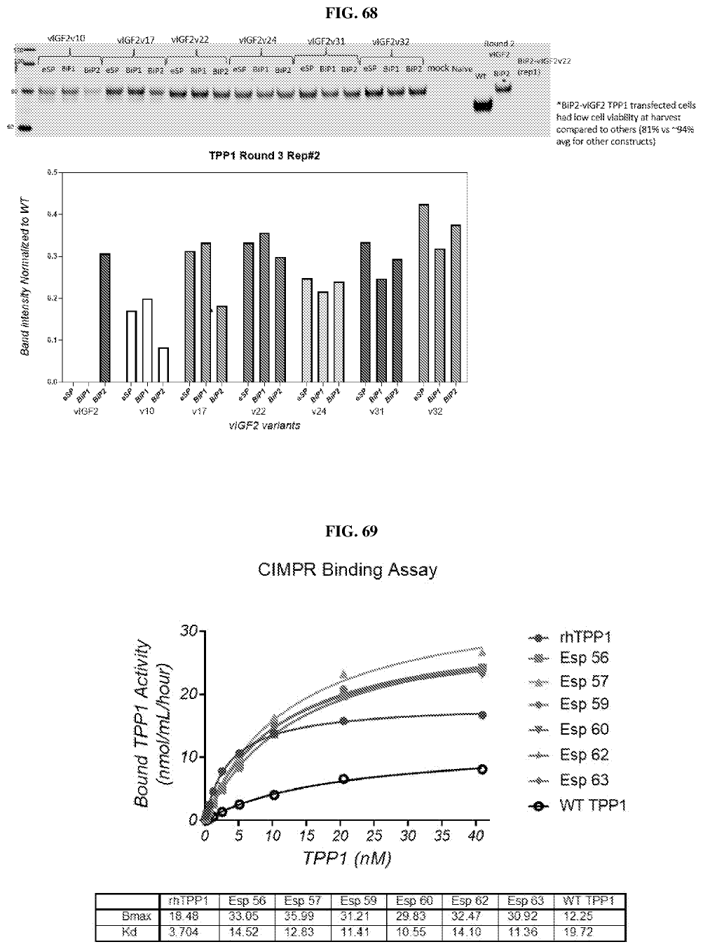

For each construct, Freestyle 293 cells (3.7 million cells in 1.5 ml of Freestyle 293 media) were transfected with 9 ul of 1 mg/ml PEI and 3 ug DNA and grown in 24-well deep well plates under shaking conditions (37 deg C., 5% CO2, 80% RH, 250 RPM). ˜24 hrs following transfection, valproic acid (final concentration 2.2 mM) and an additional 1.5 ml freestyle media was added to the transfection. Cultures were harvested 3 days post transfection and centrifuged to separate cells and conditioned media. Protein in conditioned media was separated on an SDS-PAGE gel and transferred to a nitrocellulose membrane. The membrane was blocked with 5% milk and probed with anti-TPP1 (abcam EPR16537) and Licor Anti-rabbit 800CW (926-32213). Blots were imaged and bands were quantified with a Licor Odyssey CLX as show in FIG. 68.

CIMPR Binding

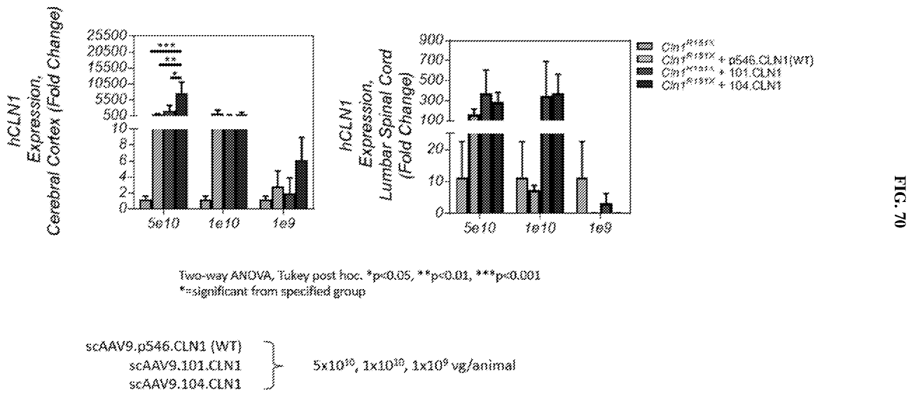

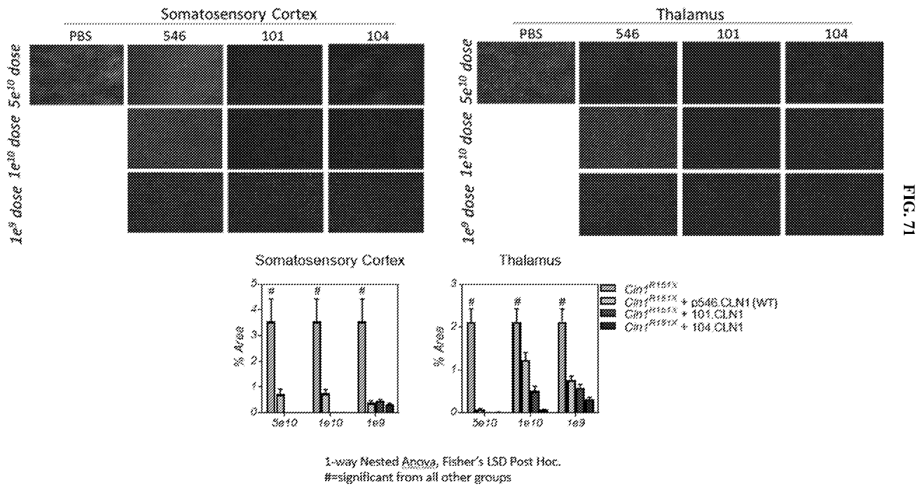

CIMPR binding was measured essentially as described in Example 10. The results are shown in FIG. 69. rhTPP1 (R&D system #2237-SE-010, expressed in Mouse myeloma NS0 cells) and WT TPP1 (SEQ ID NO:8) were included as controls. As shown in FIG. 69, the novel TPP1 constructs all showed improved binding compared to rhTPP1.

### Example 18 Testing of Novel PPT1 Variants CLN1 Mouse Model

The PPT1-101 (SEQ ID NO:60) and PPT1-104 (SEQ ID NO:61) constructs were tested in CLN1^{R151X} mouse model. (Miller, 2014, Human Molecular Genetics, 24(1)185-196). Gene therapy constructs comprising the coding sequences of PPT1-101 (SEQ ID NO:228) and PPT1-104 (SEQ ID NO:235) were prepared. Postnatal Day 1 (P1) mice were intracerebroventricularly injected with the viral constructs (or PBS control) at doses of 5×1010, 1×1010, or 1×109 vg/animal. Wild-type PPT1 (p546) was included as a control. The transgenes were introduced using an AAV9 vector. Outcomes were assessed at 2 months of age.

Transgene Expression

Human CLN1 transgene expression was detected by RT-qPCR. As seen in FIG. 70, brain and spinal cord extracts showed similar gene expression between the various constructs, with higher expression in the cortex.

Reduction in Autofluorescent Storage Material

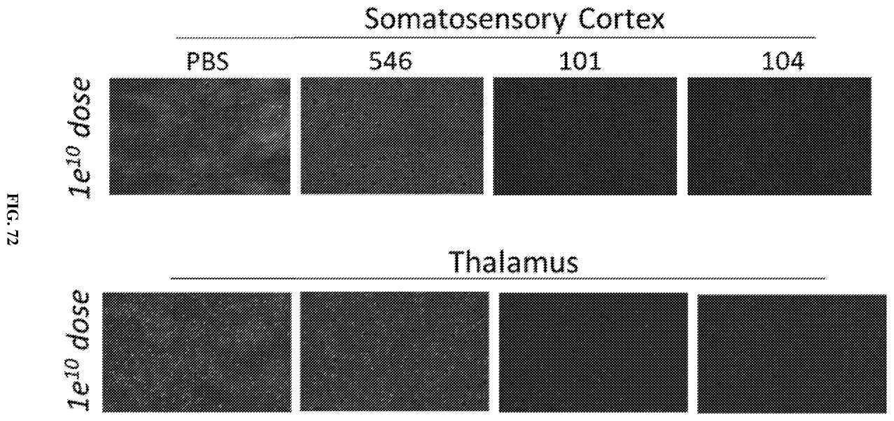

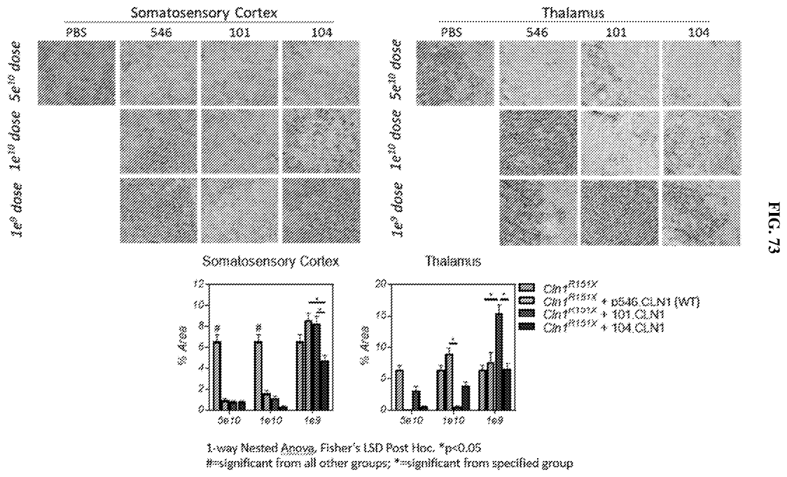

FIGS. 71-72 show the effect of each construct on brain autofluorescent storage material (ASM) accumulation, a correlate of lysosomal dysfunction. At the 5×10¹⁰ and 1×10¹⁰ doses in the cortex, and at the 1×10¹⁰ and 1×10⁹ doses in the thalamus, the 101 and 104 constructs trend towards greater reductions in ASM, as compared to the WT p546 construct.

Reduction in Glial Fibrillary Acidic Protein (GFAP)

FIG. 73 shows the effect of each construct on the glial fibrillary acidic protein (GFAP), a correlate of astrogliosis and neuroinflammation. At the 1×109 dose in the cortex, the 104 construct trended towards greater reductions in GFAP. At the 1×1010 dose in the thalamus, the 101 construct trended towards greater reductions in GFAP. GFAP-positive cells were morphologically consistent with a reactive astrocyte phenotype.

Thus, the novel PPT1 101 and 104 gene therapy constructs show improved cross-correction compared to wildtype PPT1 in a CLN1 mouse model, leading to greater reduction in both ASM and GFAP in the cortex and thalamus.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1. A nucleic acid construct comprising:
(a) a nucleic acid sequence encoding a therapeutic protein, and
(b) a nucleic acid sequence encoding a variant IGF2 (vIGF2) peptide that is at least 95% identical to at least one sequence selected from SEQ ID NO: 90-103.

2. The nucleic acid construct of claim 1, wherein the vIGF2 peptide has an amino acid sequence that is at least 98% identical to an IGF2 variant peptide selected from SEQ ID NOs:106, 109, 111, 119, 120, 121.

3. The nucleic acid construct of claim 1, wherein the vIGF2 peptide comprises an amino acid sequence that is at least 98% identical to an IGF2 variant peptide selected from the group consisting of SEQ ID NO:120 and SEQ ID NO:121.

4. The nucleic acid construct of claim 1, further comprising a sequence encoding a linker having a sequence that is at least 98% identical to a sequence selected from the group consisting of SEQ ID NOs: 181-188.

5. The nucleic acid construct of claim 1, wherein the vIGF2 peptide is capable of increasing expression and/or secretion of a therapeutic protein compared to a vIGF2 peptide having the amino acid sequence of SEQ ID NO:80.

6. The nucleic acid construct of claim 1, wherein the vIGF2 peptide has increased affinity for the CI-MPR as compared to a vIGF2 peptide having the amino acid sequence of SEQ ID NO:80.

7. The nucleic acid construct claim 1, wherein the vIGF2 peptide is capable of improving uptake of the therapeutic protein into a cell.

8. The nucleic acid construct of claim 1, wherein the therapeutic protein is capable of replacing a defective or deficient protein associated with a genetic disorder in a subject having the genetic disorder.

9. The nucleic acid construct of claim 8, wherein genetic disorder is a lysosomal storage disorder.

10. The nucleic acid construct of claim 8, wherein the genetic disorder is selected from the group consisting of aspartylglucosaminuria, neuronal ceroid lipofuscinosis, CLN1/PPT1 disease, CLN2/PPT1 disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, Schindler disease type II, adenosine deaminase severe combined immunodeficiency (ADA-SCID), and neuronal ceroid lipofuscinosis.

11. The nucleic acid construct of claim 1, wherein the genetic disorder is selected from the group consisting of CLN1/PPT1 disease, CLN2/PPT1 disease, Pompe disease and MPS IIIB disease.

12. The nucleic acid construct of claim 11, wherein the genetic disorder is CLN1/PPT1 disease or CLN2/PPT1 disease.

13. The nucleic acid construct of claim 1, wherein the therapeutic protein comprises a human enzyme selected from the group consisting of alpha-galactosidase (A or B), β-galactosidase, β-hexosaminidase (A or B), galactosylceramidase, arylsulfatase (A or B), β-glucocerebrosidase, glucocerebrosidase, lysosomal acid lipase, lysosomal enzyme acid sphingomyelinase, formylglycine-generating enzyme, iduronidase (e.g., alpha-L), acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, N-sulfoglucosamine sulfohydrolase, glycosaminoglycan N—acetylgalactosamine 4-sulfatase, β-glucuronidase, hyaluronidase, alpha-N-acetyl neuraminidase (sialidase), gangliosidesialidase, phosphotransferase, alpha-glucosidase, alpha-D-mannosidase, beta-D-mannosidase, aspartylglucosaminidase, alpha-L-fucosidase, battenin, PPT1, TPP1, and other Batten-related proteins (e.g., ceroid-lipofuscinosis neuronal protein 6), or an enzymatically active fragment thereof.

14. The nucleic acid construct of claim 13, wherein the therapeutic protein is alpha-glucosidase, or an enzymatically active fragment thereof.

15. The nucleic acid construct of claim 13, wherein the therapeutic protein is human PPT1.

16. The nucleic acid construct of claim 13, wherein the therapeutic protein is human TPP1

17. The nucleic acid construct of claim 13, wherein the therapeutic protein is human NAGLU.

18. The nucleic acid construct of claim 1, wherein the nucleic acid construct further comprises a sequence encoding a signal peptide.

19. The nucleic acid construct of claim 18, wherein the signal peptide is one of the sequences selected from the group consisting of SEQ ID NO:169-180.

20. The nucleic acid construct of claim 1, wherein the vIGF2 encoding nucleic acid sequence is 5′ to the nucleic acid sequence encoding a therapeutic protein.

21. The nucleic acid construct of claim 1, wherein the vIGF2 encoding nucleic acid sequence is 3′ to the nucleic acid sequence encoding a therapeutic protein.

22. A gene therapy vector comprising the nucleic acid construct of claim 1.

23. The gene therapy vector of claim 22, wherein the gene therapy vector is a virus vector.

24. The gene therapy vector of claim 23, wherein the virus vector is an adenovirus vector, an adeno-associated virus (AAV) vector, a retrovirus vector, a lentivirus vector, a pox virus vector, a vaccinia virus vector, an adenovirus vector, or a herpes virus vector.

25. The nucleic acid construct of claim 1, wherein the nucleic acid construct is a plasmid.

26. A pharmaceutical composition comprising a therapeutically effective amount of the nucleic acid construct of claim 1, and a pharmaceutically acceptable carrier or excipient.

27. The pharmaceutical composition of claim 25, wherein the excipient comprises a non-ionic, low-osmolar compound, a buffer, a polymer, a salt, or a combination thereof.

28. A method for treating a genetic disorder comprising administering to a subject in need thereof the nucleic acid construct of claim 1.

29. The method of claim 27, wherein the genetic disorder is a lysosomal storage disorder.

30-67. (canceled)


SEQUENCE LISTING (250 sequences)

SEQ ID NO: 1 - protein, 952 aa, Artificial Sequence
  Natural hGAA
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 2 - protein, 982 aa, Artificial Sequence
  Engineered hGAA (BiP-vIGF2-GAA)
MKLSLVAAMLLLLSAARASRTLCGGELVDTLQFVCGDRGFLFSRPASRVSRRSRGIVEECCFRSCDLALLETYCATPARSEGGGGSGGGGSRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 3 - protein, 892 aa, Artificial Sequence
  hGAA delta 1-60
SRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 4 - protein, 306 aa, Artificial Sequence
  wt-PPT1
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 5 - protein, 387 aa, Artificial Sequence
  PPT1-2 (vIGF2-PPT1)
MASPGCLWLLAVALLPWTCASRALQHLSRTLCGGELVDTLQFVCGDRGFLFSRPASRVSRRSRGIVEECCFRSCDLALLETYCATPARSEGGGGSGGGGSRPRAVPTQDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 6 - protein, 387 aa, Artificial Sequence
  PPT1-29 (BiP2aa-vIGF2-PPT1)
MKLSLVAAMLLLLWVALLLLSAARAAASRTLCGGELVDTLQFVCGDRGFLFSRPASRVSRRSRGIVEECCFRSCDLALLETYCATPARSEGGGGSGGGGSRPRAVPTQDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 7 - protein, 387 aa, Artificial Sequence
  PPT1 engineered
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLGRPRAVPTQGGSGSGSTSSSRTLCGGELVDTLQFVCGDRGFLFSRPASRVSRRSRGIVEECCFRECDLALLETYCATPARSE

SEQ ID NO: 8 - protein, 563 aa, Artificial Sequence
  TPP1 wildtype
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 9 - protein, 644 aa, Artificial Sequence
  TPP1 engineered
MGLQACLLGLFALILSGKCSRTLCGGELVDTLQFVCGDRGFLFSRPASRVSRRSRGIVEECCFRSCDLALLETYCATPARSEGGGGSGGGGSRPRAVPTQSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 10 - protein, 346 aa, Artificial Sequence
  AGA wildtype
MARKSNLPVLLVPFLLCQALVRCSSPLPLVVNTWPFKNATEAAWRALASGGSALDAVESGCAMCEREQCDGSVGFGGSPDELGETTLDAMIMDGTTMDVGAVGDLRRIKNAIGVARKVLEHTTHTLLVGESATTFAQSMGFINEDLSTTASQALHSDWLARNCQPNYWRNVIPDPSKYCGPYKPPGILKQDIPIHKETEDDRGHDTIGMVVIHKTGHIAAGTSTNGIKFKIHGRVGDSPIPGAGAYADDTAGAAAATGNGDILMRFLPSYQAVEYMRRGEDPTIACQKVISRIQKHFPEFFGAVICANVTGSYGAACNKLSTFTQFSFMVYNSEKNQPTEEKVDCI

SEQ ID NO: 11 - protein, 427 aa, Artificial Sequence
  AGA engineered (N-terminal fusion)
MARKSNLPVLLVPFLLCQALVRCSRTLCGGELVDTLQFVCGDRGFLFSRPASRVSRRSRGIVEECCFRSCDLALLETYCATPARSEGGGGSGGGGSRPRAVPTQSSPLPLVVNTWPFKNATEAAWRALASGGSALDAVESGCAMCEREQCDGSVGFGGSPDELGETTLDAMIMDGTTMDVGAVGDLRRIKNAIGVARKVLEHTTHTLLVGESATTFAQSMGFINEDLSTTASQALHSDWLARNCQPNYWRNVIPDPSKYCGPYKPPGILKQDIPIHKETEDDRGHDTIGMVVIHKTGHIAAGTSTNGIKFKIHGRVGDSPIPGAGAYADDTAGAAAATGNGDILMRFLPSYQAVEYMRRGEDPTIACQKVISRIQKHFPEFFGAVICANVTGSYGAACNKLSTFTQFSFMVYNSEKNQPTEEKVDCI

SEQ ID NO: 12 - protein, 429 aa, Artificial Sequence
  GLA wildtype
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 13 - protein, 517 aa, Artificial Sequence
  GLA engineered
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLLYIPAKQGLQGAQMGQPGGGGSGGGGSRTLCGGELVDTLQFVCGDRGFLFSRPASRVSRRSRGIVEECCFRSCDLALLETYCATPARSE

SEQ ID NO: 14 - protein, 982 aa, Artificial Sequence
  BiP-vIGF2-17-2GS-GAA
MKLSLVAAMLLLLSAARASRTLCGGELVDTLQFVCGDRGFLFSRPASRVSRRSRGIVEECCFRECDLALLETYCATPARSEGGGGSGGGGSRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 15 - protein, 982 aa, Artificial Sequence
  BiP-vIGF2-20-2GS-GAA
MKLSLVAAMLLLLSAARASRTLCGGELVDTLQFVCGDRGFLFSRPASRVSRRSRGILEECCFRSCDLALLETYCATPARSEGGGGSGGGGSRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 16 - protein, 978 aa, Artificial Sequence
  BiP-vIGF2-22-2GS-GAA
MKLSLVAAMLLLLSAARASRTLCGGELVDTLQFVCGDRGFLFSRGGGGSRGIVEECCFRSCDLALLETYCATPARSEGGGGSGGGGSRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 17 - protein, 297 aa, Artificial Sequence
  PPT1-3 (BiP-PPT1)
MKLSLVAAMLLLLSAARADPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 18 - protein, 378 aa, Artificial Sequence
  PPT1-4 (BiP-vIGF2-PPT1)
MKLSLVAAMLLLLSAARASRTLCGGELVDTLQFVCGDRGFLFSRPASRVSRRSRGIVEECCFRSCDLALLETYCATPARSEGGGGSGGGGSRPRAVPTQDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 19 - protein, 386 aa, Artificial Sequence
  PPT1-5 (wt-PPT1-vIGF2)
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLGRPRAVPTQGGGGSGGGGSRTLCGGELVDTLQFVCGDRGFLFSRPASRVSRRSRGIVEECCFRSCDLALLETYCATPARSE

SEQ ID NO: 20 - protein, 306 aa, Artificial Sequence; identical to SEQ ID NO: 4
  PPT1-9 (wt-PPT1)

SEQ ID NO: 21 - protein, 386 aa, Artificial Sequence
  PPT1-10 (wt-PPT1-vIGF2_2)
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLGRPRAVPTQGGSGSGSTSSRTLCGGELVDTLQFVCGDRGFLFSRPASRVSRRSRGIVEECCFRSCDLALLETYCATPARSE

SEQ ID NO: 22 - protein, 304 aa, Artificial Sequence
  PPT1-11 (BiP-PPT1_2)
MKLSLVAAMLLLLSAARASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 23 - protein, 304 aa, Artificial Sequence; identical to SEQ ID NO: 22
  PPT1-12 (BiPaa-PPT1_2)

SEQ ID NO: 24 - protein, 299 aa, Artificial Sequence
  PPT1-13 (BiPaa-PPT1)
MKLSLVAAMLLLLSAARAAADPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 25 - protein, 388 aa, Artificial Sequence
  PPT1-14 (BiP1-vIGF2-PPT1)
MKLSLVAAMLLLLSLVAAMLLLLSAARASRTLCGGELVDTLQFVCGDRGFLFSRPASRVSRRSRGIVEECCFRSCDLALLETYCATPARSEGGGGSGGGGSRPRAVPTQDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 26 - protein, 390 aa, Artificial Sequence
  PPT1-15 (BiP1aa-vIGF2-PPT1)
MKLSLVAAMLLLLSLVAAMLLLLSAARAAASRTLCGGELVDTLQFVCGDRGFLFSRPASRVSRRSRGIVEECCFRSCDLALLETYCATPARSEGGGGSGGGGSRPRAVPTQDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 27 - protein, 316 aa, Artificial Sequence
  PPT1-16 (BiP1aa-PPT1_2)
MKLSLVAAMLLLLSLVAAMLLLLSAARAAASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 28 - protein, 306 aa, Artificial Sequence
  PPT1-17(wt-PPT1-C6S)
MASPGSLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 29 - protein, 313 aa, Artificial Sequence
  PPT1-18 (BiP2aa-PPT1
MKLSLVAAMLLLLWVALLLLSAARAAASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 30 - protein, 305 aa, Artificial Sequence
  PPT1-19 (GaussiaAA-PPT1_2)
MGVKVLFALICIAVAEAAASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 31 - protein, 379 aa, Artificial Sequence
  PPT1-20 (GaussiaAA-vIGF2-PPT1)
MGVKVLFALICIAVAEAAASRTLCGGELVDTLQFVCGDRGFLFSRPASRVSRRSRGIVEECCFRSCDLALLETYCATPARSEGGGGSGGGGSRPRAVPTQDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 32 - protein, 306 aa, Artificial Sequence
  PPT1-21 (ppt2ss-PPT1)
MLGLWGQRLPAAWVLLLLPFLPLLLLADPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 33 - protein, 313 aa, Artificial Sequence
  PPT1-22 (ppt2ss-PPT1_2)
MLGLWGQRLPAAWVLLLLPFLPLLLLASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 34 - protein, 306 aa, Artificial Sequence
  PPT1-23 (consensusSS-PPT1)
MASPSCLWLLAVALLPWSCAARALGHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 35 - protein, 306 aa, Artificial Sequence
  PPT1-24 (consensus-PPT1)
MASPSCLWLLAVALLPWSCAARALGHLDPPAPLPLVIWHGMGDSCCNPLSMGAIKKMVEKKIPGIYVLSLEIGKTLMEDVENSFFLNVNSQVTTVCQILAKDPKLQQGYNAMGFSQGGQFLRAVAQRCPSPPMINLISVGGQHQGVFGLPRCPGESSHICDFIRKTLNAGAYSKAVQERLVQAEYWHDPIKEDVYRNHSIFLADINQERGVNESYKKNLMALKKFVMVKFLNDSIVDPVDSEWFGFYRSGQAKETIPLQETTLYTQDRLGLKEMDKAGQLVFLATEGDHLQLSEEWFYAHIIPFLE

SEQ ID NO: 36 - protein, 306 aa, Artificial Sequence
  PPT1-25 (wt-PPT1 L283C H300C)
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFCATEGDHLQLSEEWFYACIIPFLG

SEQ ID NO: 37 - protein, 306 aa, Artificial Sequence
  PPT1-26 (wt-PPT1 G113C L121C)
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMCFSQGGQFCRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 38 - protein, 306 aa, Artificial Sequence
  PPT1-27 (wt-PPT1 A171C A183C)
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGCYSKVVQERLVQCEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 39 - protein, 313 aa, Artificial Sequence; identical to SEQ ID NO: 29
  PPT1-28 (BiP2aa-PPT1)

SEQ ID NO: 40 - protein, 388 aa, Artificial Sequence; identical to SEQ ID NO: 25
  PPT1-31 (BiP1-vIGF2-PPT1

SEQ ID NO: 41 - protein, 383 aa, Artificial Sequence
  PPT1-32 (wt-PPT1-vIGF2-32)
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLGRPRAVPTQGGSGSGSTSSSRTLCGGELVDTLQFVCGDRGFLFSRGGGGSRGILEECCFRECDLALLETYCATPARSE

SEQ ID NO: 42 - protein, 387 aa, Artificial Sequence; identical to SEQ ID NO: 7
  PPT1-33 (wt-PPT1-vIGF2-8Q)

SEQ ID NO: 43 - protein, 387 aa, Artificial Sequence; identical to SEQ ID NO: 7
  PPT1-34 (wt-PPT1-vIGF2-8Q)

SEQ ID NO: 44 - protein, 387 aa, Artificial Sequence; identical to SEQ ID NO: 7
  PPT1-35 (wt-PPT1-vIGF2-8Q)

SEQ ID NO: 45 - protein, 176 aa, Artificial Sequence
  Human TPP1 Propeptide
SYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVG

SEQ ID NO: 46 - protein, 368 aa, Artificial Sequence
  Human TPP1 Mature Peptide
LHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 47 - protein, 645 aa, Artificial Sequence
  pSvelte001- Native TPP1 Signal Peptide - vIGF2 - GS linker - Lyso Cleave - TPP1 propeptide - TPP1 mature peptide
MGLQACLLGLFALILSGKCSRTLCGGELVDTLQFVCGDRGFLFSRPASRVSRRSRGIVEECCFRSCDLALLETYCATPARSEGGGGSGGGGSRPRAVPTQSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPG

SEQ ID NO: 48 - protein, 646 aa, Artificial Sequence
  Svelte057 - Native TPP1 Signal Peptide - vIGF2v17 - GS linker - Lyso Cleave - TPP1 propeptide - TPP1 mature peptide
MGLQACLLGLFALILSGKCSRTLCGGELVDTLQFVCGDRGFLFSRPASRVSRRSRGIVEECCFRECDLALLETYCATPARSEGGGGSGGGGSRPRAVPTQASYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPG

SEQ ID NO: 49 - protein, 642 aa, Artificial Sequence
  pSvelte059 -Native TPP1 Signal Peptide - vIGF2v22 - GS linker - Lyso Cleave - TPP1 propeptide - TPP1 mature peptide
MGLQACLLGLFALILSGKCSRTLCGGELVDTLQFVCGDRGFLFSRGGGGSRGIVEECCFRSCDLALLETYCATPARSEGGGGSGGGGSRPRAVPTQASYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPG

SEQ ID NO: 50 - protein, 642 aa, Artificial Sequence
  pSvelte060 - Native TPP1 Signal Peptide - vIGF2v24- GS linker - Lyso Cleave - TPP1 propeptide - TPP1 mature peptide
MGLQACLLGLFALILSGKCSRTLCGGELVDTLQFVCGDRGFLFSRGGGGSRGILEECCFRSCDLALLETYCATPARSEGGGGSGGGGSRPRAVPTQASYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPG

SEQ ID NO: 51 - protein, 646 aa, Artificial Sequence
  pSvelte061- Native TPP1 Signal Peptide - vIGF2v30 - GS linker - Lyso Cleave - TPP1 propeptide - TPP1 mature peptide
MGLQACLLGLFALILSGKCSRTLCGGELVDVLQFVCGRRGFLFSRPASRVSRRSRGIVEECCFRDCDLALLETYCATPARSEGGGGSGGGGSRPRAVPTQASYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPG

SEQ ID NO: 52 - protein, 642 aa, Artificial Sequence
  pSvelte062- Native TPP1 Signal Peptide - vIGF2v31 - GS linker - Lyso Cleave - TPP1 propeptide - TPP1 mature peptide
MGLQACLLGLFALILSGKCSRTLCGGELVDTLQFVCGDRGFLFSRGGGGSRGILEECCFRDCDLALLETYCATPARSEGGGGSGGGGSRPRAVPTQASYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPG

SEQ ID NO: 53 - protein, 642 aa, Artificial Sequence
  pSvelte063- Native TPP1 Signal Peptide - vIGF2v32 - GS linker - Lyso Cleave - TPP1 propeptide - TPP1 mature peptide
MGLQACLLGLFALILSGKCSRTLCGGELVDTLQFVCGDRGFLFSRGGGGSRGILEECCFRECDLALLETYCATPARSEGGGGSGGGGSRPRAVPTQASYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNPG

SEQ ID NO: 54 - protein, 743 aa, Artificial Sequence
  Wt-NAGLU
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 55 - protein, 764 aa, Artificial Sequence
  Wt NAGLU-HPC4
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGLEVLFQGPEDQVDPRLIDGK

SEQ ID NO: 56 - protein, 847 aa, Artificial Sequence
  vIGF2-NAGLU-HPC4
MEAVAVAAAVGVLLLAGAGGAAGDASRTLCGGELVDTLQFVCGDRGFLFSRPASRVSRRSRGIVEECCFRSCDLALLETYCATPARSEGGGGSGGGGSRPRAVPTQAEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGLEVLFQGPEDQVDPRLIDGK

SEQ ID NO: 57 - protein, 847 aa, Artificial Sequence
  vIGF2-17-NAGLU
MEAVAVAAAVGVLLLAGAGGAAGDASRTLCGGELVDTLQFVCGDRGFLFSRPASRVSRRSRGIVEECCFRECDLALLETYCATPARSEGGGGSGGGGSRPRAVPTQAEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGLEVLFQGPEDQVDPRLIDGK

SEQ ID NO: 58 - protein, 843 aa, Artificial Sequence
  vIGF2-31-NAGLU-HPC4
MEAVAVAAAVGVLLLAGAGGAAGDASRTLCGGELVDTLQFVCGDRGFLFSRGGGGSRGILEECCFRDCDLALLETYCATPARSEGGGGSGGGGSRPRAVPTQAEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGLEVLFQGPEDQVDPRLIDGK

SEQ ID NO: 59 - protein, 843 aa, Artificial Sequence
  vIGF2-32-NAGLU
MEAVAVAAAVGVLLLAGAGGAAGDASRTLCGGELVDTLQFVCGDRGFLFSRGGGGSRGILEECCFRECDLALLETYCATPARSEGGGGSGGGGSRPRAVPTQAEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGLEVLFQGPEDQVDPRLIDGK

SEQ ID NO: 60 - protein, 383 aa, Artificial Sequence
  PPT1-101
MKLSLVAAMLLLLWVALLLLSAARAAASRTLCGGELVDTLQFVCGDRGFLFSRGGGGSRGILEECCFRDCDLALLETYCATPARSEGGGGSGGGGSRPRAVPTQDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 61 - protein, 383 aa, Artificial Sequence
  PPT1-104
MASPGSLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLGRPRAVPTQGGSGSGSTSSSRTLCGGELVDTLQFVCGDRGFLFSRGGGGSRGILEECCFRECDLALLETYCATPARSE

SEQ ID NO: 62 - protein, 385 aa, Artificial Sequence
  PPT1-112
MASPGSLWLLAVALLPWTCASRALQHLAASRTLCGGELVDTLQFVCGDRGFLFSRGGGGSRGILEECCFRDCDLALLETYCATPARSEGGGGSGGGGSRPRAVPTQDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 63 - protein, 385 aa, Artificial Sequence
  PPT1-114
MASPGSLWLLAVALLPWTCASRALQHLAASRTLCGGELVDTLQFVCGDRGFLFSRGGGGSRGILEECCFRECDLALLETYCATPARSEGGGGSGGGGSRPRAVPTQDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 64 - protein, 385 aa, Artificial Sequence
  PPT1-115
MASPGSLWLLAVALLPWTCASRALQHLAADPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLGRPRAVPTQGGSGSGSTSSSRTLCGGELVDTLQFVCGDRGFLFSRGGGGSRGILEECCFRECDLALLETYCATPARSE

SEQ ID NO: 65 - protein, 387 aa, Artificial Sequence
  PPT1-116
MASPGSLWLLAVALLPWTCASRALQHLAADPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLGRPRAVPTQGGGGSGSGGGGSSRTLCGGELVDTLQFVCGDRGFLFSRGGGGSRGILEECCFRECDLALLETYCATPARSE

SEQ ID NO: 66 - protein, 385 aa, Artificial Sequence
  PPT1-117
MASPGSLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLGRPRAVPTQGGGGSGSGGGGSSRTLCGGELVDTLQFVCGDRGFLFSRGGGGSRGILEECCFRECDLALLETYCATPARSE

SEQ ID NO: 67 - protein, 385 aa, Artificial Sequence
  PPT1-118
MASPGSLWLLAVALLPWTCASRALQHLAADPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLGRPRAVPTQGGGGSGGGGSSRTLCGGELVDTLQFVCGDRGFLFSRGGGGSRGILEECCFRECDLALLETYCATPARSE

SEQ ID NO: 68 - protein, 67 aa, Artificial Sequence
  Wildtype IGF2
AYRPSETLCGGELVDTLQFVCGDRGFYFSRPASRVSRRSRGIVEECCFRSCDLALLETYCATPAKSE

SEQ ID NO: 69 - protein, 67 aa, Artificial Sequence
  IGF2 F26S
AYRPSETLCGGELVDTLQFVCGDRGSYFSRPASRVSRRSRGIVEECCFRSCDLALLETYCATPAKSE

SEQ ID NO: 70 - protein, 67 aa, Artificial Sequence
  IGF2 Y27L
AYRPSETLCGGELVDTLQFVCGDRGFLFSRPASRVSRRSRGIVEECCFRSCDLALLETYCATPAKSE

SEQ ID NO: 71 - protein, 67 aa, Artificial Sequence
  IGF2 V43L
AYRPSETLCGGELVDTLQFVCGDRGFYFSRPASRVSRRSRGILEECCFRSCDLALLETYCATPAKSE

SEQ ID NO: 72 - protein, 67 aa, Artificial Sequence
  IGF2 F48T
AYRPSETLCGGELVDTLQFVCGDRGFYFSRPASRVSRRSRGIVEECCTRSCDLALLETYCATPAKSE

SEQ ID NO: 73 - protein, 67 aa, Artificial Sequence
  IGF2 R49S
AYRPSETLCGGELVDTLQFVCGDRGFYFSRPASRVSRRSRGIVEECCFSSCDLALLETYCATPAKSE

SEQ ID NO: 74 - protein, 67 aa, Artificial Sequence
  IGF2 S50I
AYRPSETLCGGELVDTLQFVCGDRGFYFSRPASRVSRRSRGIVEECCFRICDLALLETYCATPAKSE

SEQ ID NO: 75 - protein, 67 aa, Artificial Sequence
  IGF2 A54R
AYRPSETLCGGELVDTLQFVCGDRGFYFSRPASRVSRRSRGIVEECCFRSCDLRLLETYCATPAKSE

SEQ ID NO: 76 - protein, 67 aa, Artificial Sequence
  L55R
AYRPSETLCGGELVDTLQFVCGDRGFYFSRPASRVSRRSRGIVEECCFRSCDLARLETYCATPAKSE

SEQ ID NO: 77 - protein, 67 aa, Artificial Sequence
  IGF2 F26S, Y27L, V43L, F48T, R49S, S50I, A54R, L55R
AYRPSETLCGGELVDTLQFVCGDRGSLFSRPASRVSRRSRGILEECCTSICDLRRLETYCATPAKSE

SEQ ID NO: 78 - protein, 61 aa, Artificial Sequence
  IGF2 delta 1-6, Y27L, K65R
TLCGGELVDTLQFVCGDRGFLFSRPASRVSRRSRGIVEECCFRSCDLALLETYCATPARSE

SEQ ID NO: 79 - protein, 60 aa, Artificial Sequence
  IGF2 delta 1-7, Y27L, K65R
LCGGELVDTLQFVCGDRGFLFSRPASRVSRRSRGIVEECCFRSCDLALLETYCATPARSE

SEQ ID NO: 80 - protein, 63 aa, Artificial Sequence
  IGF2 delta 1-4, E6R, Y27L, K65R
SRTLCGGELVDTLQFVCGDRGFLFSRPASRVSRRSRGIVEECCFRSCDLALLETYCATPARSE

SEQ ID NO: 81 - protein, 63 aa, Artificial Sequence
  IGF2 delta 1-4, E6R, Y27L
SRTLCGGELVDTLQFVCGDRGFLFSRPASRVSRRSRGIVEECCFRSCDLALLETYCATPAKSE

SEQ ID NO: 82 - protein, 67 aa, Artificial Sequence
  IGF2 E6R
AYRPSRTLCGGELVDTLQFVCGDRGFYFSRPASRVSRRSRGIVEECCFRSCDLALLETYCATPAKSE

SEQ ID NO: 83 - protein, 63 aa, Artificial Sequence
  IGF2 delta 1-4, E6R, Y27L, S50E, K65R
SRTLCGGELVDTLQFVCGDRGFLFSRPASRVSRRSRGIVEECCFRECDLALLETYCATPARSE

SEQ ID NO: 84 - protein, 18 aa, Artificial Sequence
  Cleavable IGF2 variant-N terminal
GGGGSGGGGSRPRAVPTQ

SEQ ID NO: 85 - protein, 25 aa, Artificial Sequence
  Cleavable IGF2 variant-C terminal
YIPAKQGLQGAQMGQPGGGGSGGGG

SEQ ID NO: 86 - protein, 18 aa, Artificial Sequence
  Cleavable IGF2 variant-C terminal
RPRAVPTQGGSGSGSTSS

SEQ ID NO: 87 - protein, 81 aa, Artificial Sequence
  Cleavable IGF2 variant-N terminal
SRTLCGGELVDTLQFVCGDRGFLFSRPASRVSRRSRGIVEECCFRSCDLALLETYCATPARSEGGGGSGGGGSRPRAVPTQ

SEQ ID NO: 88 - protein, 88 aa, Artificial Sequence
  Cleavable IGF2 variant-C terminal
YIPAKQGLQGAQMGQPGGGGSGGGGSRTLCGGELVDTLQFVCGDRGFLFSRPASRVSRRSRGIVEECCFRSCDLALLETYCATPARSE

SEQ ID NO: 89 - protein, 81 aa, Artificial Sequence
  Cleavable IGF2 variant-C terminal
RPRAVPTQGGSGSGSTSSSRTLCGGELVDTLQFVCGDRGFLFSRPASRVSRRSRGIVEECCFRECDLALLETYCATPARSE

SEQ ID NO: 90 - protein, 63 aa, Artificial Sequence
  vIGF2-1 (vIGF2_1_NGGWGMG)
SRTLCGGELVDTNQFVCGDRGFLFSRGASRVSRGSRGIVEWCCFRGCDLALLETYCATPMRGE

SEQ ID NO: 91 - protein, 63 aa, Artificial Sequence
  vIGF2-2 (vIGF2_2_GGWGMG)
SRTLCGGELVDTLQFVCGDRGFLFSRGASRVSRGSRGIVEWCCFRGCDLALLETYCATPMRGE

SEQ ID NO: 92 - protein, 63 aa, Artificial Sequence
  vIGF2-3 (vIGF2_3_NGGGMG)
SRTLCGGELVDTNQFVCGDRGFLFSRGASRVSRGSRGIVEECCFRGCDLALLETYCATPMRGE

SEQ ID NO: 93 - protein, 53 aa, Artificial Sequence
  vIGF2-4 (vIGF2_4_delta 32-41, 53aa)
SRTLCGGELVDTLQFVCGDRGFLFSRPIVEECCFRSCDLALLETYCATPARSE

SEQ ID NO: 94 - protein, 53 aa, Artificial Sequence
  vIGF2-5 (vIGF2 delta 30-39, 53aa)
SRTLCGGELVDTLQFVCGDRGFLFSRGIVEECCFRSCDLALLETYCATPARSE

SEQ ID NO: 95 - protein, 55 aa, Artificial Sequence
  vIGF2-6 (vIGF2 delta 33-40, 55aa)
SRTLCGGELVDTLQFVCGDRGFLFSRPAGIVEECCFRSCDLALLETYCATPARSE

SEQ ID NO: 96 - protein, 53 aa, Artificial Sequence
  vIGF2-7 (vIGF2 delta 30-39/V14D/F28R/V43D/F26A)
SRTLCGGELDDTLQFVCGDRGALRSRGIDEECCFRSCDLALLETYCATPARSE

SEQ ID NO: 97 - protein, 63 aa, Artificial Sequence
  vIGF2-8 (vIGF2_8_REE)
SRTLCGGELVDTLQFVCGRRGELFSRPASRVSRRSRGIVEECCFRECDLALLETYCATPARSE

SEQ ID NO: 98 - protein, 55 aa, Artificial Sequence
  vIGF2-9 (vIGF2_9_ delta 30-39-REE; vIGF2)
SRTLCGGELVDTLQFVCGRRGELFSRPAGIVEECCFRECDLALLETYCATPARSE

SEQ ID NO: 99 - protein, 63 aa, Artificial Sequence
  vIGF2-10 (vIGF2_1Q; vIGF2 D23R)
SRTLCGGELVDTLQFVCGRRGFLFSRPASRVSRRSRGIVEECCFRSCDLALLETYCATPARSE

SEQ ID NO: 100 - protein, 63 aa, Artificial Sequence
  vIGF2-11 (vIGF2_2Q; vIGF2 F19W)
SRTLCGGELVDTLQWVCGDRGFLFSRPASRVSRRSRGIVEECCFRSCDLALLETYCATPARSE

SEQ ID NO: 101 - protein, 63 aa, Artificial Sequence
  vIGF2-12 (vIGF2_3Q; vIGF2 T16W)
SRTLCGGELVDWLQFVCGDRGFLFSRPASRVSRRSRGIVEECCFRSCDLALLETYCATPARSE

SEQ ID NO: 102 - protein, 63 aa, Artificial Sequence
  vIGF2-13 (vIGF2_4Q; vIGF2 D23K)
SRTLCGGELVDTLQFVCGKRGFLFSRPASRVSRRSRGIVEECCFRSCDLALLETYCATPARSE

SEQ ID NO: 103 - protein, 63 aa, Artificial Sequence
  vIGF2-14 (vIGF2_5Q; vIGF2 T16Y)
SRTLCGGELVDYLQFVCGDRGFLFSRPASRVSRRSRGIVEECCFRSCDLALLETYCATPARSE

SEQ ID NO: 104 - protein, 63 aa, Artificial Sequence
  vIGF2-15 (vIGF2_6Q; vIGF2 F26E)
SRTLCGGELVDTLQFVCGDRGELFSRPASRVSRRSRGIVEECCFRSCDLALLETYCATPARSE

SEQ ID NO: 105 - protein, 63 aa, Artificial Sequence
  vIGF2-16 (vIGF2_7Q; vIGF2 T16V)
SRTLCGGELVDVLQFVCGDRGFLFSRPASRVSRRSRGIVEECCFRSCDLALLETYCATPARSE

SEQ ID NO: 106 - protein, 63 aa, Artificial Sequence; identical to SEQ ID NO: 83
  vIGF2-17 (vIGF2_8Q; vIGF2 S50E)

SEQ ID NO: 107 - protein, 63 aa, Artificial Sequence
  vIGF2-18 (vIGF2_9Q; vIGF2 S50D)
SRTLCGGELVDTLQFVCGDRGFLFSRPASRVSRRSRGIVEECCFRDCDLALLETYCATPARSE

SEQ ID NO: 108 - protein, 63 aa, Artificial Sequence
  vIGF2-19 (vIGF2 F26S V43L)
SRTLCGGELVDTLQFVCGDRGSLFSRPASRVSRRSRGILEECCFRSCDLALLETYCATPARSE

SEQ ID NO: 109 - protein, 63 aa, Artificial Sequence
  vIGF2-20 (vIGF2 V43L)
SRTLCGGELVDTLQFVCGDRGFLFSRPASRVSRRSRGILEECCFRSCDLALLETYCATPARSE

SEQ ID NO: 110 - protein, 63 aa, Artificial Sequence
  vIGF2-21 (vIGF2_ESRE; vIGF2 V14E F26S F28R V43E)
SRTLCGGELEDTLQFVCGDRGSLRSRPASRVSRRSRGIEEECCFRSCDLALLETYCATPARSE

SEQ ID NO: 111 - protein, 59 aa, Artificial Sequence
  vIGF2-22 (vIGF2 delta 31-38GGGG)
SRTLCGGELVDTLQFVCGDRGFLFSRGGGGSRGIVEECCFRSCDLALLETYCATPARSE

SEQ ID NO: 112 - protein, 56 aa, Artificial Sequence
  vIGF2-23 (vIGF2 delta 30-40GGGG)
SRTLCGGELVDTLQFVCGDRGFLFSGGGSGIVEECCFRSCDLALLETYCATPARSE

SEQ ID NO: 113 - protein, 59 aa, Artificial Sequence
  vIGF2-24 (vIGF2 delta 31-38GGGG V43L)
SRTLCGGELVDTLQFVCGDRGFLFSRGGGGSRGILEECCFRSCDLALLETYCATPARSE

SEQ ID NO: 114 - protein, 63 aa, Artificial Sequence
  vIGF2-25 (vIGF2 L8A)
SRTACGGELVDTLQFVCGDRGFLFSRPASRVSRRSRGIVEECCFRSCDLALLETYCATPARSE

SEQ ID NO: 115 - protein, 63 aa, Artificial Sequence
  vIGF2-26 (vIGF2 R6Q T7A L8A)
SQAACGGELVDTLQFVCGDRGFLFSRPASRVSRRSRGIVEECCFRSCDLALLETYCATPARSE

SEQ ID NO: 116 - protein, 63 aa, Artificial Sequence
  vIGF2-27 (vIGF2 R24E R34E R37E R38E)
SRTLCGGELVDTLQFVCGDEGFLFSRPASEVSEESRGIVEECCFRSCDLALLETYCATPARSE

SEQ ID NO: 117 - protein, 63 aa, Artificial Sequence
  vIGF2-28 (vIGF2 R24E R34E)
SRTLCGGELVDTLQFVCGDEGFLFSRPASEVSRRSRGIVEECCFRSCDLALLETYCATPARSE

SEQ ID NO: 118 - protein, 63 aa, Artificial Sequence
  vIGF2-29 (vIGF2 D23R S40D)
SRTLCGGELVDTLQFVCGRRGFLFSRPASRVSRRSRGIVEECCFRDCDLALLETYCATPARSE

SEQ ID NO: 119 - protein, 63 aa, Artificial Sequence
  vIGF2-30 (vIGF2 T16V D23R S50D)
SRTLCGGELVDVLQFVCGRRGFLFSRPASRVSRRSRGIVEECCFRDCDLALLETYCATPARSE

SEQ ID NO: 120 - protein, 59 aa, Artificial Sequence
  vIGF2-31 (vIGF2 delta 31-38GGGG V43L S50D)
SRTLCGGELVDTLQFVCGDRGFLFSRGGGGSRGILEECCFRDCDLALLETYCATPARSE

SEQ ID NO: 121 - protein, 59 aa, Artificial Sequence
  vIGF2-32 (vIGF2 delta 31-38GGGG V43L S50E)
SRTLCGGELVDTLQFVCGDRGFLFSRGGGGSRGILEECCFRECDLALLETYCATPARSE

SEQ ID NO: 122 - protein, 67 aa, Artificial Sequence
  vIGF2-33 (vIGF2-N1)
SRTLCGGELVDTLQFVCGDRGFLFRLPSRPVSRHSHRRSRGIVEECCFQRCNLALLETYCATPARSE

SEQ ID NO: 123 - protein, 67 aa, Artificial Sequence
  vIGF2-34 (vIGF2-N1 V43L S50E)
SRTLCGGELVDTLQFVCGDRGFLFRLPSRPVSRHSHRRSRGILEECCFQECNLALLETYCATPARSE

SEQ ID NO: 124 - protein, 63 aa, Artificial Sequence
  vIGF2-1 R38G
SRTLCGGELVDTLQFVCGDRGFLFSRPASRVSRGSRGIVEECCFRSCDLALLETYCATPARSE

SEQ ID NO: 125 - protein, 63 aa, Artificial Sequence
  vIGF2-2 R38G, E45W
SRTLCGGELVDTLQFVCGDRGFLFSRPASRVSRGSRGIVEWCCFRSCDLALLETYCATPARSE

SEQ ID NO: 126 - protein, 63 aa, Artificial Sequence
  vIGF2-3 R38G, E45W, S50G
SRTLCGGELVDTLQFVCGDRGFLFSRPASRVSRGSRGIVEWCCFRGCDLALLETYCATPARSE

SEQ ID NO: 127 - protein, 63 aa, Artificial Sequence
  vIGF2-4 P31G, R38G, E45W, S50G
SRTLCGGELVDTLQFVCGDRGFLFSRGASRVSRGSRGIVEWCCFRGCDLALLETYCATPARSE

SEQ ID NO: 128 - protein, 63 aa, Artificial Sequence
  vIGF2-5 L17N, P31G, R38G, E45W, S50G
SRTLCGGELVDTNQFVCGDRGFLFSRGASRVSRGSRGIVEWCCFRGCDLALLETYCATPARSE

SEQ ID NO: 129 - protein, 63 aa, Artificial Sequence
  vIGF2-6 L17N, P31G, R38G, E45W, S50G, S66G
SRTLCGGELVDTNQFVCGDRGFLFSRGASRVSRGSRGIVEWCCFRGCDLALLETYCATPARGE

SEQ ID NO: 130 - protein, 63 aa, Artificial Sequence; identical to SEQ ID NO: 90
  vIGF2-7 L17N, P31G, R38G, E45W, S50G, A64M, S66G

SEQ ID NO: 131 - protein, 63 aa, Artificial Sequence
  vIGF2-8 S5L, L17N, P31G, R38G, E45W, S50G, A64M, S66G
LRTLCGGELVDTNQFVCGDRGFLFSRGASRVSRGSRGIVEWCCFRGCDLALLETYCATPMRGE

SEQ ID NO: 132 - DNA, 201 bp, Artificial Sequence
  Mature WT IGF2
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

SEQ ID NO: 133 - DNA, 189 bp, Artificial Sequence
  vIGF2 delta 1-4, E6R, Y27L, K65R
tctagaacactgtgcggaggggagcttgtagacactcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaa

SEQ ID NO: 134 - DNA, 189 bp, Artificial Sequence
  vIGF2 delta 1-4, E6R, Y27L, S50E, K65R
tctagaacactgtgcggaggggagcttgtagacactcttcagttcgtgtgtggagatcgcgggttcctcttctctcgccccgcttccagagtttcacggaggtctaggggtatagtagaggagtgttgtttcagggagtgtgacttggcgctcctcgagacctattgcgcgacgccagccaggtccgaa

SEQ ID NO: 135 - DNA, 189 bp, Artificial Sequence
  vIGF2-1 (vIGF2_1_NGGWGMG)
tctagaacactgtgcggaggggagcttgtagacactaaccagttcgtgtgtggagatcgcgggttcctcttctctcgcggcgcttccagagtttcacggggctctaggggtatagtagagtggtgttgtttcaggggctgtgacttggcgctcctcgagacctattgcgcgacgccaatgaggggcgaa

SEQ ID NO: 136 - DNA, 189 bp, Artificial Sequence
  vIGF2-2 (vIGF2_2_GGWGMG)
tctagaacactgtgcggaggggagcttgtagacactcttcagttcgtgtgtggagatcgcgggttcctcttctctcgcggcgcttccagagtttcacggggctctaggggtatagtagagtggtgttgtttcaggggctgtgacttggcgctcctcgagacctattgcgcgacgccaatgaggggcgaa

SEQ ID NO: 137 - DNA, 189 bp, Artificial Sequence
  vIGF2-3 (vIGF2_3_NGGGMG)
tctagaacactgtgcggaggggagcttgtagacactaaccagttcgtgtgtggagatcgcgggttcctcttctctcgcggcgcttccagagtttcacggggctctaggggtatagtagaggagtgttgtttcaggggctgtgacttggcgctcctcgagacctattgcgcgacgccaatgaggggcgaa

SEQ ID NO: 138 - DNA, 159 bp, Artificial Sequence
  vIGF2-4 (vIGF2_4_delta 32-41, 53aa)
tctagaacactgtgcggaggggagcttgtagacactcttcagttcgtgtgtggagatcgcgggttcctcttctctcgccccatagtagaggagtgttgtttcaggtcctgtgacttggcgctcctcgagacctattgcgcgacgccagccaggtccgaa

SEQ ID NO: 139 - DNA, 159 bp, Artificial Sequence
  vIGF2-5 (vIGF2 delta 30-39, 53aa)
tctagaacactgtgcggaggggagcttgtagacactcttcagttcgtgtgtggagatcgcgggttcctcttctctaggggtatagtagaggagtgttgtttcaggtcctgtgacttggcgctcctcgagacctattgcgcgacgccagccaggtccgaa

SEQ ID NO: 140 - DNA, 165 bp, Artificial Sequence
  vIGF2-6 (vIGF2 delta 33-40, 55aa)
tctagaacactgtgcggaggggagcttgtagacactcttcagttcgtgtgtggagatcgcgggttcctcttctctcgccccgctggtatagtagaggagtgttgtttcaggtcctgtgacttggcgctcctcgagacctattgcgcgacgccagccaggtccgaa

SEQ ID NO: 141 - DNA, 159 bp, Artificial Sequence
  vIGF2-7 (vIGF2 delta 30-39/V14D/F28R/V43D/F26A)
tctagaacactgtgcggaggggagcttgacgacactcttcagttcgtgtgtggagatcgcggggccctcagatctaggggtatagacgaggagtgttgtttcaggtcctgtgacttggcgctcctcgagacctattgcgcgacgccagccaggtccgaa

SEQ ID NO: 142 - DNA, 189 bp, Artificial Sequence
  vIGF2-8 (vIGF2_8_REE)
tctagaacactgtgcggaggggagcttgtagacactcttcagttcgtgtgtggaagacgcggggagctcttctctcgccccgcttccagagtttcacggaggtctaggggtatagtagaggagtgttgtttcagggagtgtgacttggcgctcctcgagacctattgcgcgacgccagccaggtccgaa

SEQ ID NO: 143 - DNA, 165 bp, Artificial Sequence
  vIGF2-9 (vIGF2_9_ delta 30-39-REE; vIGF2 Homerun)
tctagaacactgtgcggaggggagcttgtagacactcttcagttcgtgtgtggaagacgcggggagctcttctctcgccccgctggtatagtagaggagtgttgtttcagggagtgtgacttggcgctcctcgagacctattgcgcgacgccagccaggtccgaa

SEQ ID NO: 144 - DNA, 189 bp, Artificial Sequence
  vIGF2-10 (vIGF2_1Q; vIGF2 D23R)
tctagaacactgtgcggaggggagcttgtagacactcttcagttcgtgtgtggacgtcgcgggttcctcttctctcgccccgcttccagagtttcacggaggtctaggggtatagtagaggagtgttgtttcaggtcctgtgacttggcgctcctcgagacctattgcgcgacgccagccaggtccgaa

SEQ ID NO: 145 - DNA, 189 bp, Artificial Sequence
  vIGF2-11 (vIGF2_2Q; vIGF2 F19W)
tctagaacactgtgcggaggggagcttgtagacactcttcagtgggtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaa

SEQ ID NO: 146 - DNA, 189 bp, Artificial Sequence
  vIGF2-12 (vIGF2_3Q; vIGF2 T16W)
tctagaacactgtgcggaggggagcttgtagactggcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaa

SEQ ID NO: 147 - DNA, 189 bp, Artificial Sequence
  vIGF2-13 (vIGF2_4Q; vIGF2 D23K)
tctagaacactgtgcggaggggagcttgtagacactcttcagttcgtgtgtggaaagcgcgggttcctcttctctcgccccgcttccagagtttcacggaggtctaggggtatagtagaggagtgttgtttcaggtcctgtgacttggcgctcctcgagacctattgcgcgacgccagccaggtccgaa

SEQ ID NO: 148 - DNA, 189 bp, Artificial Sequence
  vIGF2-14 (vIGF2_5Q; vIGF2 T16Y)
tctagaacactgtgcggaggggagcttgtagactatcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaa

SEQ ID NO: 149 - DNA, 189 bp, Artificial Sequence
  vIGF2-15 (vIGF2_6Q; vIGF2 F26E)
tctagaacactgtgcggaggggagcttgtagacactcttcagttcgtgtgtggagatcgcggggagctcttctctcgccccgcttccagagtttcacggaggtctaggggtatagtagaggagtgttgtttcaggtcctgtgacttggcgctcctcgagacctattgcgcgacgccagccaggtccgaa

SEQ ID NO: 150 - DNA, 189 bp, Artificial Sequence
  vIGF2-16 (vIGF2_7Q; vIGF2 T16V)
tctagaacactgtgcggaggggagcttgtagacgttcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaa

SEQ ID NO: 151 - DNA, 189 bp, Artificial Sequence; identical to SEQ ID NO: 134
  vIGF2-17 (vIGF2_8Q; vIGF2 S50E)

SEQ ID NO: 152 - DNA, 189 bp, Artificial Sequence
  vIGF2-18 (vIGF2_9Q; vIGF2 S50D)
tctagaacactgtgcggaggggagcttgtagacactcttcagttcgtgtgtggagatcgcgggttcctcttctctcgccccgcttccagagtttcacggaggtctaggggtatagtagaggagtgttgtttcagggactgtgacttggcgctcctcgagacctattgcgcgacgccagccaggtccgaa

SEQ ID NO: 153 - DNA, 189 bp, Artificial Sequence
  vIGF2-19 (vIGF2 F26S V43L)
tctagaacactgtgcggaggggagcttgtagacactcttcagttcgtgtgtggagatcgcgggagcctcttctctcgccccgcttccagagtttcacggaggtctaggggtatactggaggagtgttgtttcaggtcctgtgacttggcgctcctcgagacctattgcgcgacgccagccaggtccgaa

SEQ ID NO: 154 - DNA, 189 bp, Artificial Sequence
  vIGF2-20 (vIGF2 V43L)
tctagaacactgtgcggaggggagcttgtagacactcttcagttcgtgtgtggagatcgcgggttcctcttctctcgccccgcttccagagtttcacggaggtctaggggtatactggaggagtgttgtttcaggtcctgtgacttggcgctcctcgagacctattgcgcgacgccagccaggtccgaa

SEQ ID NO: 155 - DNA, 189 bp, Artificial Sequence
  vIGF2-21 (vIGF2_ESRE; vIGF2 V14E F26S F28R V43E)
tctagaacactgtgcggaggggagcttgaggacactcttcagttcgtgtgtggagatcgcgggagcctcagatctcgccccgcttccagagtttcacggaggtctaggggtatagaggaggagtgttgtttcaggtcctgtgacttggcgctcctcgagacctattgcgcgacgccagccaggtccgaa

SEQ ID NO: 156 - DNA, 177 bp, Artificial Sequence
  vIGF2-22 (vIGF2 delta 31-38GGGG)
tctagaacactgtgcggaggggagcttgtagacactcttcagttcgtgtgtggagatcgcgggttcctcttctctcgcggaggtggaggttctaggggtatagtagaggagtgttgtttcaggtcctgtgacttggcgctcctcgagacctattgcgcgacgccagccaggtccgaa

SEQ ID NO: 157 - DNA, 168 bp, Artificial Sequence
  vIGF2-23 (vIGF2 delta 30-40GGGG)
tctagaacactgtgcggaggggagcttgtagacactcttcagttcgtgtgtggagatcgcgggttcctcttctctggtggaggttctggtatagtagaggagtgttgtttcaggtcctgtgacttggcgctcctcgagacctattgcgcgacgccagccaggtccgaa

SEQ ID NO: 158 - DNA, 177 bp, Artificial Sequence
  vIGF2-24 (vIGF2 delta 31-38GGGG V43L)
tctagaacactgtgcggaggggagcttgtagacactcttcagttcgtgtgtggagatcgcgggttcctcttctctcgcggaggtggaggttctaggggtatactggaggagtgttgtttcaggtcctgtgacttggcgctcctcgagacctattgcgcgacgccagccaggtccgaa

SEQ ID NO: 159 - DNA, 189 bp, Artificial Sequence
  vIGF2-25 (vIGF2 L8A)
tctcaggccgcgtgcggaggggagcttgtagacactcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaa

SEQ ID NO: 160 - DNA, 189 bp, Artificial Sequence; identical to SEQ ID NO: 159
  vIGF2-26 (vIGF2 R6Q T7A L8A)

SEQ ID NO: 161 - DNA, 189 bp, Artificial Sequence
  vIGF2-27 (vIGF2 R24E R34E R37E R38E)
tctagaacactgtgcggaggggagcttgtagacactcttcagttcgtgtgtggagatgaggggttcctcttctctcgccccgcttccgaggtttcagaggaatctaggggtatagtagaggagtgttgtttcaggtcctgtgacttggcgctcctcgagacctattgcgcgacgccagccaggtccgaa

SEQ ID NO: 162 - DNA, 189 bp, Artificial Sequence
  vIGF2-28 (vIGF2 R24E R34E)
tctagaacactgtgcggaggggagcttgtagacactcttcagttcgtgtgtggagatgaggggttcctcttctctcgccccgcttccgaggtttcacggaggtctaggggtatagtagaggagtgttgtttcaggtcctgtgacttggcgctcctcgagacctattgcgcgacgccagccaggtccgaa

SEQ ID NO: 163 - DNA, 189 bp, Artificial Sequence
  vIGF2-29 (vIGF2 D23R S40D)
tctagaacactgtgcggaggggagcttgtagacactcttcagttcgtgtgtggaagacgcgggttcctcttctctcgccccgcttccagagtttcacggaggtctaggggtatagtagaggagtgttgtttcagggactgtgacttggcgctcctcgagacctattgcgcgacgccagccaggtccgaa

SEQ ID NO: 164 - DNA, 189 bp, Artificial Sequence
  vIGF2-30 (vIGF2 T16V D23R S50D)
tctagaacactgtgcggaggggagcttgtagacgtgcttcagttcgtgtgtggaagacgcgggttcctcttctctcgccccgcttccagagtttcacggaggtctaggggtatagtagaggagtgttgtttcagggactgtgacttggcgctcctcgagacctattgcgcgacgccagccaggtccgaa

SEQ ID NO: 165 - DNA, 177 bp, Artificial Sequence
  vIGF2-31 (vIGF2 delta 31-38GGGG V43L S50D)
tctagaacactgtgcggaggggagcttgtagacactcttcagttcgtgtgtggagatcgcgggttcctcttctctcgcggaggtggaggttctaggggtatactggaggagtgttgtttcagggactgtgacttggcgctcctcgagacctattgcgcgacgccagccaggtccgaa

SEQ ID NO: 166 - DNA, 177 bp, Artificial Sequence
  vIGF2-32 (vIGF2 delta 31-38GGGG V43L S50E)
tctagaacactgtgcggaggggagcttgtagacactcttcagttcgtgtgtggagatcgcgggttcctcttctctcgcggaggtggaggttctaggggtatactggaggagtgttgtttcagggagtgtgacttggcgctcctcgagacctattgcgcgacgccagccaggtccgaa

SEQ ID NO: 167 - DNA, 201 bp, Artificial Sequence
  vIGF2-33 (vIGF2-N1)
tctagaacactgtgcggaggggagcttgtagacactcttcagttcgtgtgtggagatcgcgggttcttgtttcgattgccgtccaggcccgtgtcccggcacagtcaccgcaggtcaagggggatagttgaagaatgttgctttcagaggtgtaatttggcgctcctcgagacctattgcgcgacgccagccaggtccgaa

SEQ ID NO: 168 - DNA, 201 bp, Artificial Sequence
  vIGF2-34 (vIGF2-N1 V43L S50E)
tctagaacactgtgcggaggggagcttgtagacactcttcagttcgtgtgtggagatcgcgggttcttgtttcgattgccgtccaggcccgtgtcccggcacagtcaccgcaggtcaagggggatactggaagaatgttgctttcaggagtgtaatttggcgctcctcgagacctattgcgcgacgccagccaggtccgaa

SEQ ID NO: 169 - protein, 18 aa, Artificial Sequence
  Native human BiP
MKLSLVAAMLLLLSAARA

SEQ ID NO: 170 - protein, 28 aa, Artificial Sequence
  Modified BiP-1
MKLSLVAAMLLLLSLVAAMLLLLSAARA

SEQ ID NO: 171 - protein, 25 aa, Artificial Sequence
  Modified BiP-2
MKLSLVAAMLLLLWVALLLLSAARA

SEQ ID NO: 172 - protein, 26 aa, Artificial Sequence
  Modified BiP-3
MKLSLVAAMLLLLSLVALLLLSAARA

SEQ ID NO: 173 - protein, 26 aa, Artificial Sequence
  Modified BiP-4
MKLSLVAAMLLLLALVALLLLSAARA

SEQ ID NO: 174 - protein, 17 aa, Artificial Sequence
  Gaussia
MGVKVLFALICIAVAEA

SEQ ID NO: 175 - protein, 27 aa, Artificial Sequence
  Native PPT1 Signal Peptide (eSP)
MASPGCLWLLAVALLPWTCASRALQHL

SEQ ID NO: 176 - protein, 29 aa, Artificial Sequence
  Native PPT1 Signal Peptide (eSP AA)
MASPGCLWLLAVALLPWTCASRALQHLAA

SEQ ID NO: 177 - protein, 27 aa, Artificial Sequence
  Native PPT1 Signal Peptide C6S (eSP C6S)
MASPGSLWLLAVALLPWTCASRALQHL

SEQ ID NO: 178 - protein, 29 aa, Artificial Sequence
  Native PPT1 Signal Peptide C6S (eSP C6S AA)
MASPGSLWLLAVALLPWTCASRALQHLAA

SEQ ID NO: 179 - protein, 19 aa, Artificial Sequence
  Native TPP1 Signal Peptide
MGLQACLLGLFALILSGKC

SEQ ID NO: 180 - protein, 24 aa, Artificial Sequence
  Native NAGLU Signal Peptide
MEAVAVAAAVGVLLLAGAGGAAGD

SEQ ID NO: 181 - protein, 9 aa, Artificial Sequence
  Linker Sequence
GGGGSGGGG

SEQ ID NO: 182 - protein, 5 aa, Artificial Sequence
  Linker Sequence
GGGGS

SEQ ID NO: 183 - protein, 9 aa, Artificial Sequence
  Linker Sequence
GGGSGGGGS

SEQ ID NO: 184 - protein, 9 aa, Artificial Sequence
  Linker Sequence
GGGGSGGGS

SEQ ID NO: 185 - protein, 9 aa, Artificial Sequence
  Linker Sequence
GGSGSGSTS

SEQ ID NO: 186 - protein, 10 aa, Artificial Sequence
  Linker Sequence
GGGGSGGGGS

SEQ ID NO: 187 - protein, 12 aa, Artificial Sequence
  Linker Sequence
GGGGSGSGGGGS

SEQ ID NO: 188 - protein, 9 aa, Artificial Sequence
  Lysosomal cleaveage linker
RPRAVPTQA

SEQ ID NO: 189 - DNA, 2864 bp, Artificial Sequence
  Kozak- hGAA (Natural GAA)
gcaagatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagtagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagcttttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 190 - DNA, 2954 bp, Artificial Sequence
  Kozak BiP-vIGF2-GAA (Engineered hGAA)
gcaagatgaagctctccctggtggccgcgatgctgctgctgctcagcgcggcgcgggcctctagaacactgtgcggaggggagcttgtagacactcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaagggggcggtggctcaggtggtggaggtagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcaccggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 191 - DNA, 3139 bp, Artificial Sequence
  Cricket Paralysis Virus IRES -BiP-vIGF2-GAA
aaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgctatgaagctctccctggtggccgcgatgctgctgctgctcagcgcggcgcgggcctctagaacactgtgcggaggggagcttgtagacactcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaagggggcggtggctcaggtggtggaggtagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 192 - DNA, 921 bp, Artificial Sequence
  wt-PPT1 IDT codon optimized
atggcatcaccgggttgcctctggttgttggccgttgcgttgcttccgtggacatgtgcatcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggagcttatagtaaagtcgtccaagaacggcttgttcaagcggagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggctaa

SEQ ID NO: 193 - DNA, 1164 bp, Artificial Sequence
  PPT1-2 (wt-vIGF2-PPT1; Codon optimized by IDT codon optimization tool)
atggcatcccccggatgtttgtggctgctggcggttgcgcttctgccatggacgtgcgcctcccgagccctccaacacctgtccaggacactttgcggcggagagttggtcgatacgcttcaattcgtgtgtggggatagaggcttccttttttctcggcccgctagccgcgtgtcccgaaggtcccggggtatcgttgaggaatgctgtttccggtcctgcgatcttgcactgttggagacatactgtgctacgcctgcgagaagcgagggtggagggggttctggaggtggagggagccggcctcgggcggttcccacccaggatcctccagctcctctgcctctggtcatctggcatgggatgggggactcatgttgtaacccgctgagtatgggggcaattaaaaaaatggttgaaaagaaaattccaggtatttatgtcctctctcttgaaatcggtaagacacttatggaggatgtggaaaactcctttttccttaatgtcaattctcaggtcacaacagtttgtcaggctctggcgaaggatcctaagctgcagcaaggctacaacgccatgggtttttcccagggaggccaatttctcagagcggtagctcagcgatgtccatcaccaccgatgataaatctgatcagtgtcggcggacaacaccagggagttttcgggctgcccaggtgtccgggggaatctagtcacatatgtgacttcattcgcaagacccttaacgccggcgcttactcaaaggtggttcaagaacggcttgtgcaggctgaatactggcacgatcccatcaaggaagatgtatataggaaccacagtatctttctggcagacataaatcaggaaaggggtattaacgaaagctacaagaaaaatctcatggccctgaagaaatttgtaatggttaagtttttgaacgattctatagtagatcctgttgactccgagtggttcgggttctatcgatctggtcaagccaaggagacgattccgcttcaggaaacttcactgtacacacaggatcggctgggactcaaggagatggacaatgcgggccagttggtgtttctggctacagagggagaccatctccagttgagtgaagaatggttctatgcacatattatcccattcctcggctaa

SEQ ID NO: 194 - DNA, 1164 bp, Artificial Sequence
  PPT1-29 (BiP2aa-vIGF2-PPT1; native human sequence)
atgaagctctccctggtggccgcgatgctgctgctgctctgggtggcactgctgctgctcagcgcggcgagggccgccgcgagtcgcacgttgtgtggaggtgaactcgtcgacacccttcagttcgtatgtggagatcgcggtttcctcttctcacgcccagcttccagagtttcccgaagatcacgaggaatagttgaggagtgctgttttcggtcttgtgatctggctctcctcgagacttattgtgctacgccggcccgctctgaaggaggtggtggcagtggaggaggagggagtcggcctagggcagtcccaacccaggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 195 - DNA, 1164 bp, Artificial Sequence
  PPT1 engineered
atggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggaagacctagagcagtgcctacgcagggagggagtgggagtggatccacttcatcctctagaacactgtgcggaggggagcttgtagacactcttcagttcgtgtgtggagatcgcgggttcctcttctctcgccccgcttccagagtttcacggaggtctaggggtatagtagaggagtgttgtttcagggagtgtgacttggcgctcctcgagacctattgcgcgacgccagccaggtccgaatga

SEQ ID NO: 196 - DNA, 1692 bp, Artificial Sequence
  TPP1 wildtype
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctga

SEQ ID NO: 197 - DNA, 1935 bp, Artificial Sequence
  TPP1 engineered
atgggactccaagcctgcctcctagggctctttgccctcatcctctctggcaaatgctctagaacactgtgcggaggggagcttgtagacactcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaaggaggtggtggcagtggaggaggagggagtagacctagagcagtgcctacgcagagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctga

SEQ ID NO: 198 - DNA, 1041 bp, Artificial Sequence
  AGA wildtype
atggcgcggaagtcgaacttgcctgtgcttctcgtgccgtttctgctctgccaggccctagtgcgctgctccagccctctgcccctggtcgtcaacacttggccctttaagaatgcaaccgaagcagcgtggagggcattagcatctggaggctctgccctggatgcagtggagagcggctgtgccatgtgtgagagagagcagtgtgacggctctgtaggctttggaggaagtcctgatgaacttggagaaaccacactagatgccatgatcatggatggcactactatggatgtaggagcagtaggagatctcagacgaattaaaaatgctattggtgtggcacggaaagtactggaacatacaacacacacacttttagtaggagagtcagccaccacatttgctcaaagtatggggtttatcaatgaagacttatctaccactgcttctcaagctcttcattcagattggcttgctcggaattgccagccaaattattggaggaatgttataccagatccctcaaaatactgcggaccctacaaaccacctggtatcttaaagcaggatattcctatccataaagaaacagaagatgatcgtggtcatgacactattggcatggttgtaatccataagacaggacatattgctgctggtacatctacaaatggtataaaattcaaaatacatggccgtgtaggagactcaccaatacctggagctggagcctatgctgacgatactgcaggggcagccgcagccactgggaatggtgatatattgatgcgcttcctgccaagctaccaagctgtagaatacatgagaagaggagaagatccaaccatagcttgccaaaaagtgatttcaagaatccagaagcattttccagaattctttggggctgttatatgtgccaatgtgactggaagttacggtgctgcttgcaataaactttcaacatttactcagtttagtttcatggtttataattccgaaaaaaatcagccaactgaggaaaaagtggactgcatctaa

SEQ ID NO: 199 - DNA, 1284 bp, Artificial Sequence
  AGA engineered (N-terminal fusion)
atggcgcggaagtcgaacttgcctgtgcttctcgtgccgtttctgctctgccaggccctagtgcgctgctctagaacactgtgcggaggggagcttgtagacactcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaaggaggtggtggcagtggaggaggagggagtagacctagagcagtgcctacgcagtccagccctctgcccctggtcgtcaacacttggccctttaagaatgcaaccgaagcagcgtggagggcattagcatctggaggctctgccctggatgcagtggagagcggctgtgccatgtgtgagagagagcagtgtgacggctctgtaggctttggaggaagtcctgatgaacttggagaaaccacactagatgccatgatcatggatggcactactatggatgtaggagcagtaggagatctcagacgaattaaaaatgctattggtgtggcacggaaagtactggaacatacaacacacacacttttagtaggagagtcagccaccacatttgctcaaagtatggggtttatcaatgaagacttatctaccactgcttctcaagctcttcattcagattggcttgctcggaattgccagccaaattattggaggaatgttataccagatccctcaaaatactgcggaccctacaaaccacctggtatcttaaagcaggatattcctatccataaagaaacagaagatgatcgtggtcatgacactattggcatggttgtaatccataagacaggacatattgctgctggtacatctacaaatggtataaaattcaaaatacatggccgtgtaggagactcaccaatacctggagctggagcctatgctgacgatactgcaggggcagccgcagccactgggaatggtgatatattgatgcgcttcctgccaagctaccaagctgtagaatacatgagaagaggagaagatccaaccatagcttgccaaaaagtgatttcaagaatccagaagcattttccagaattctttggggctgttatatgtgccaatgtgactggaagttacggtgctgcttgcaataaactttcaacatttactcagtttagtttcatggtttataattccgaaaaaaatcagccaactgaggaaaaagtggactgcatctaa

SEQ ID NO: 200 - DNA, 1251 bp, Artificial Sequence
  GLA wildtype
atgaagctctccctggtggccgcgatgctgctgctgctcagcgcggcgcgggccctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 201 - DNA, 1515 bp, Artificial Sequence
  GLA engineered
atgaagctctccctggtggccgcgatgctgctgctgctcagcgcggcgcgggccctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttacatccctgcaaagcaggggctgcagggagcccagatggggcagcccgggggcggtggctcaggtggtggaggttcaagaacactgtgcggaggggagcttgtagacactcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaataa

SEQ ID NO: 202 - DNA, 2949 bp, Artificial Sequence
  BiP-vIGF2-17-2GS-GAA
atgaagctctccctggtggccgcgatgctgctgctgctcagcgcggcgcgggcctctagaacactgtgcggaggggagcttgtagacactcttcagttcgtgtgtggagatcgcgggttcctcttctctcgccccgcttccagagtttcacggaggtctaggggtatagtagaggagtgttgtttcagggagtgtgacttggcgctcctcgagacctattgcgcgacgccagccaggtccgaagggggcggtggctcaggtggtggaggtagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 203 - DNA, 2949 bp, Artificial Sequence
  BiP-vIGF2-20-2GS-GAA
atgaagctctccctggtggccgcgatgctgctgctgctcagcgcggcgcgggcctctagaacactgtgcggaggggagcttgtagacactcttcagttcgtgtgtggagatcgcgggttcctcttctctcgccccgcttccagagtttcacggaggtctaggggtatactggaggagtgttgtttcaggtcctgtgacttggcgctcctcgagacctattgcgcgacgccagccaggtccgaagggggcggtggctcaggtggtggaggtagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 204 - DNA, 2937 bp, Artificial Sequence
  BiP-vIGF2-22-2GS-GAA
atgaagctctccctggtggccgcgatgctgctgctgctcagcgcggcgcgggcctctagaacactgtgcggaggggagcttgtagacactcttcagttcgtgtgtggagatcgcgggttcctcttctctcgcggaggtggaggttctaggggtatagtagaggagtgttgtttcaggtcctgtgacttggcgctcctcgagacctattgcgcgacgccagccaggtccgaagggggcggtggctcaggtggtggaggtagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 205 - DNA, 894 bp, Artificial Sequence
  PPT1-3 (BiP-PPT1; Codon optimized IDT)
atgaaactgtctctggttgcagcaatgctcttgctgttgagtgcggcccgcgcggatccacctgctcccctgcccctcgttatatggcatggcatgggagattcctgttgtaatcccctcagcatgggggccatcaaaaaaatggtggaaaaaaaaatacctggcatatatgtactctcacttgaaatcggtaagacccttatggaagacgtcgaaaattccttctttttgaacgtgaactcacaagttacgaccgtctgtcaagctctcgcgaaagaccctaagctccagcaaggttataatgcaatgggcttctcacagggaggtcagttcttgcgagcggtagcccagaggtgtccgtctccgccaatgatcaacttgatctcagtggggggtcagcaccaaggcgtttttggactccctagatgccctggagagagctctcacatttgcgattttatacggaagacgctgaatgccggcgcgtattcaaaggtcgttcaagagcgactcgtccaggctgaatactggcacgatccgattaaggaagacgtgtatcgaaaccattctatctttcttgccgacattaaccaggagcgagggatcaacgaaagttataaaaaaaacctgatggcactcaagaaatttgtaatggttaaattcctgaacgattcaatagttgatccggtggattccgagtggttcggcttctaccggtccggtcaggccaaggaaacaatcccattgcaagaaaccagtctctatactcaggaccgcctgggtctgaaagaaatggacaacgctggccaacttgtttttctggcaacggagggtgatcacttgcagctctctgaagaatggttttacgcacacatcattcctttccttggttaa

SEQ ID NO: 206 - DNA, 1137 bp, Artificial Sequence
  PPT1-4 (BiP-vIGF2-PPT1; Codon optimized IDT)
atgaagttgtccctcgtagctgcaatgttgctgctcctcagtgcagcgcgggcaagtcgcacgttgtgtggaggtgaactcgtcgacacccttcagttcgtatgtggagatcgcggtttcctcttctcacgcccagcttccagagtttcccgaagatcacgaggaatagttgaggagtgctgttttcggtcttgtgatctggctctcctcgagacttattgtgctacgccggcccgctctgaaggaggtggtggcagtggaggaggagggagtcggcctagggcagtcccaacccaggatcccccagcacccctccccctggtaatttggcatggaatgggtgattcctgctgtaacccactctcaatgggggcaattaagaaaatggtagagaaaaagatccctggcatttatgttctgtcactcgaaatcggtaaaacgctcatggaggacgtagaaaacagcttttttctgaatgttaattcacaggttaccacggtctgccaagcattggcaaaggacccgaaattgcaacaaggctataacgcgatggggttcagccaaggcgggcagtttcttcgagctgtggctcagcgctgcccttccccaccgatgataaatttgattagcgtagggggacaacatcaaggggttttcggtttgccaaggtgtcctggcgaatcttcacatatttgcgactttatacggaagaccttgaatgcgggggcgtatagtaaagtcgtccaggaacggcttgtccaagctgaatactggcacgatcccatcaaagaagatgtctatcggaatcacagcatttttctcgccgacataaaccaagaacgcggaattaatgagtcatacaagaagaacttgatggcacttaaaaaatttgtgatggttaagtttttgaatgatagtatcgtagatcccgtagatagtgaatggtttggtttctatcgatccggacaggctaaagaaacgataccattgcaggaaacctctttgtatactcaagataggttgggcctcaaggagatggataatgcggggcaacttgtcttcctcgcgactgagggtgaccacctccagctcagcgaggaatggttttacgcccacatcattcctttccttggttaa

SEQ ID NO: 207 - DNA, 1161 bp, Artificial Sequence
  PPT1-5 (wt-PPT1-vIGF2; Codon optimized IDT)
atggcaagtccagggtgtctttggttgctcgcggttgccttgctcccttggacgtgcgcgtcccgagcccttcaacacctcgatccaccagccccgcttcctctcgtgatatggcacggcatgggcgacagttgctgcaatcccttgtctatgggcgcaattaaaaagatggtggaaaagaaaatccctggtatctacgttttgagcctcgaaattgggaaaacgctcatggaggatgtcgagaacagcttctttcttaacgtcaattcccaagttaccacggtttgtcaagccttggcgaaagatcccaagcttcagcaagggtataacgctatgggatttagccagggcggacagttcctgagggcggtagcacagaggtgtcctagtccaccaatgataaatctcatctcagtcgggggccagcaccagggcgtcttcgggcttcctcgatgccccggcgaatccagccacatatgtgacttcattagaaaaactttgaatgcaggggcctacagtaaagtggttcaagaacgcctggtacaagcagagtattggcatgacccgattaaggaagatgtctacagaaatcactctatttttttggcggacatcaatcaggaacgaggcattaacgagtcttacaagaagaacctgatggcgctgaaaaagttcgtcatggtcaagttcttgaatgactccattgtcgatcctgtagacagcgagtggtttggcttctacaggtctggtcaagcaaaggagacaataccacttcaggaaaccagtctctatacacaagacagactgggtttgaaggaaatggacaatgcaggccaactggtattcctggctacagagggagatcatcttcaactgagcgaagagtggttttatgcccacataatcccctttctgggaagacctagagcagtgcctacgcagggtggtggtggctctggaggaggaggctccaggactctgtgtgggggcgagctggtggacaccttgcaattcgtgtgtggcgaccgaggatttctgttcagtcgacctgcctcaagagtaagccggaggagtcgggggatcgttgaagaatgctgtttccggagctgcgacttggcgttgctcgagacttattgtgccacacctgcaaggagtgagtga

SEQ ID NO: 208 - DNA, 921 bp, Artificial Sequence
  PPT1-9 (wt-PPT1; native human sequence)
atggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 209 - DNA, 1161 bp, Artificial Sequence
  PPT1-10 (wt-PPT1-vIGF2_2; Codon optimized IDT)
atggcatcaccgggttgcctctggttgttggccgttgcgttgcttccgtggacatgtgcatcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggagcttatagtaaagtcgtccaagaacggcttgttcaagcggagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggcagacctagagcagtgcctacgcagggagggagtgggagtggatccacttcatccaggactctgtgtgggggcgagctggtggacaccttgcaattcgtgtgtggcgaccgaggatttctgttcagtcgacctgcctcaagagtaagccggaggagtcgggggatcgttgaagaatgctgtttccggagctgcgacttggcgttgctcgagacttattgtgccacacctgcaaggagtgaatga

SEQ ID NO: 210 - DNA, 915 bp, Artificial Sequence
  PPT1-11 (BiP-PPT1_2; Codon optimized IDT)
atgaagctctccctggtggccgcgatgctgctgctgctcagcgcggcgcgggcctcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggagcttatagtaaagtcgtccaagaacggcttgttcaagcggagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggctaa

SEQ ID NO: 211 - DNA, 915 bp, Artificial Sequence; identical to SEQ ID NO: 210
  PPT1-12 (BiPaa-PPT1_2; Codon optimized IDT)

SEQ ID NO: 212 - DNA, 900 bp, Artificial Sequence
  PPT1-13 (BiPaa-PPT1; Codon optimized IDT)
atgaaactgtctctggttgcagcaatgctcttgctgttgagtgcggcccgcgcggcggccgatccacctgctcccctgcccctcgttatatggcatggcatgggagattcctgttgtaatcccctcagcatgggggccatcaaaaaaatggtggaaaaaaaaatacctggcatatatgtactctcacttgaaatcggtaagacccttatggaagacgtcgaaaattccttctttttgaacgtgaactcacaagttacgaccgtctgtcaagctctcgcgaaagaccctaagctccagcaaggttataatgcaatgggcttctcacagggaggtcagttcttgcgagcggtagcccagaggtgtccgtctccgccaatgatcaacttgatctcagtggggggtcagcaccaaggcgtttttggactccctagatgccctggagagagctctcacatttgcgattttatacggaagacgctgaatgccggcgcgtattcaaaggtcgttcaagagcgactcgtccaggctgaatactggcacgatccgattaaggaagacgtgtatcgaaaccattctatctttcttgccgacattaaccaggagcgagggatcaacgaaagttataaaaaaaacctgatggcactcaagaaatttgtaatggttaaattcctgaacgattcaatagttgatccggtggattccgagtggttcggcttctaccggtccggtcaggccaaggaaacaatcccattgcaagaaaccagtctctatactcaggaccgcctgggtctgaaagaaatggacaacgctggccaacttgtttttctggcaacggagggtgatcacttgcagctctctgaagaatggttttacgcacacatcattcctttccttggttaa

SEQ ID NO: 213 - DNA, 1167 bp, Artificial Sequence
  PPT1-14 (BiP1-vIGF2-PPT1; Codon optimized IDT)
atgaagctcagtctcgtggcagctatgctcctcctgctgtccctggttgcggcaatgttgctcttgctgagcgccgcgagagcaagtcgcacgttgtgtggaggtgaactcgtcgacacccttcagttcgtatgtggagatcgcggtttcctcttctcacgcccagcttccagagtttcccgaagatcacgaggaatagttgaggagtgctgttttcggtcttgtgatctggctctcctcgagacttattgtgctacgccggcccgctctgaaggaggtggtggcagtggaggaggagggagtcggcctagggcagtcccaacccaggatcccccagcacccctccccctggtaatttggcatggaatgggtgattcctgctgtaacccactctcaatgggggcaattaagaaaatggtagagaaaaagatccctggcatttatgttctgtcactcgaaatcggtaaaacgctcatggaggacgtagaaaacagcttttttctgaatgttaattcacaggttaccacggtctgccaagcattggcaaaggacccgaaattgcaacaaggctataacgcgatggggttcagccaaggcgggcagtttcttcgagctgtggctcagcgctgcccttccccaccgatgataaatttgattagcgtagggggacaacatcaaggggttttcggtttgccaaggtgtcctggcgaatcttcacatatttgcgactttatacggaagaccttgaatgcgggggcgtatagtaaagtcgtccaggaacggcttgtccaagctgaatactggcacgatcccatcaaagaagatgtctatcggaatcacagcatttttctcgccgacataaaccaagaacgcggaattaatgagtcatacaagaagaacttgatggcacttaaaaaatttgtgatggttaagtttttgaatgatagtatcgtagatcccgtagatagtgaatggtttggtttctatcgatccggacaggctaaagaaacgataccattgcaggaaacctctttgtatactcaagataggttgggcctcaaggagatggataatgcggggcaacttgtcttcctcgcgactgagggtgaccacctccagctcagcgaggaatggttttacgcccacatcattcctttccttggttaa

SEQ ID NO: 214 - DNA, 1173 bp, Artificial Sequence
  PPT1-15 (BiP1aa-vIGF2-PPT1; Codon optimized IDT)
atgaagctcagtctcgtggcagctatgctcctcctgctgtccctggttgcggcaatgttgctcttgctgagcgccgcgagagcagcagctagtcgcacgttgtgtggaggtgaactcgtcgacacccttcagttcgtatgtggagatcgcggtttcctcttctcacgcccagcttccagagtttcccgaagatcacgaggaatagttgaggagtgctgttttcggtcttgtgatctggctctcctcgagacttattgtgctacgccggcccgctctgaaggaggtggtggcagtggaggaggagggagtcggcctagggcagtcccaacccaggatcccccagcacccctccccctggtaatttggcatggaatgggtgattcctgctgtaacccactctcaatgggggcaattaagaaaatggtagagaaaaagatccctggcatttatgttctgtcactcgaaatcggtaaaacgctcatggaggacgtagaaaacagcttttttctgaatgttaattcacaggttaccacggtctgccaagcattggcaaaggacccgaaattgcaacaaggctataacgcgatggggttcagccaaggcgggcagtttcttcgagctgtggctcagcgctgcccttccccaccgatgataaatttgattagcgtagggggacaacatcaaggggttttcggtttgccaaggtgtcctggcgaatcttcacatatttgcgactttatacggaagaccttgaatgcgggggcgtatagtaaagtcgtccaggaacggcttgtccaagctgaatactggcacgatcccatcaaagaagatgtctatcggaatcacagcatttttctcgccgacataaaccaagaacgcggaattaatgagtcatacaagaagaacttgatggcacttaaaaaatttgtgatggttaagtttttgaatgatagtatcgtagatcccgtagatagtgaatggtttggtttctatcgatccggacaggctaaagaaacgataccattgcaggaaacctctttgtatactcaagataggttgggcctcaaggagatggataatgcggggcaacttgtcttcctcgcgactgagggtgaccacctccagctcagcgaggaatggttttacgcccacatcattcctttccttggttaa

SEQ ID NO: 215 - DNA, 951 bp, Artificial Sequence
  PPT1-16 (BiP1aa-PPT1_2; Codon optimized IDT)
atgaagctcagtctcgtggcagctatgctcctcctgctgtccctggttgcggcaatgttgctcttgctgagcgccgcgagagcagccgcgtcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggagcttatagtaaagtcgtccaagaacggcttgttcaagcggagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggctaa

SEQ ID NO: 216 - DNA, 921 bp, Artificial Sequence
  PPT1-17 (wt-PPT1-C6S; natural human sequence)
atggcgtcgcccggcagcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 217 - DNA, 942 bp, Artificial Sequence
  PPT1-18 (BiP2aa-PPT1; Codon optimized IDT)
atgaagctctccctggtggccgcgatgctgctgctgctctgggtggcactgctgctgctcagcgcggcgagggccgccgcgtcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggagcttatagtaaagtcgtccaagaacggcttgttcaagcggagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggctaa

SEQ ID NO: 218 - DNA, 918 bp, Artificial Sequence
  PPT1-19 (GaussiaAA-PPT1_2; Codon optimized IDT)
atgggtgtaaaggtgttgttcgctcttatctgcattgccgttgcagaagctgccgcgtcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggagcttatagtaaagtcgtccaagaacggcttgttcaagcggagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggctaa

SEQ ID NO: 219 - DNA, 1140 bp, Artificial Sequence
  PPT1-20 (GaussiaAA-vIGF2-PPT1; Codon optimized IDT)
atgggtgtaaaggtgttgttcgctcttatctgcattgccgttgcagaagctgcagctagtcgcacgttgtgtggaggtgaactcgtcgacacccttcagttcgtatgtggagatcgcggtttcctcttctcacgcccagcttccagagtttcccgaagatcacgaggaatagttgaggagtgctgttttcggtcttgtgatctggctctcctcgagacttattgtgctacgccggcccgctctgaaggaggtggtggcagtggaggaggagggagtcggcctagggcagtcccaacccaggatcccccagcacccctccccctggtaatttggcatggaatgggtgattcctgctgtaacccactctcaatgggggcaattaagaaaatggtagagaaaaagatccctggcatttatgttctgtcactcgaaatcggtaaaacgctcatggaggacgtagaaaacagcttttttctgaatgttaattcacaggttaccacggtctgccaagcattggcaaaggacccgaaattgcaacaaggctataacgcgatggggttcagccaaggcgggcagtttcttcgagctgtggctcagcgctgcccttccccaccgatgataaatttgattagcgtagggggacaacatcaaggggttttcggtttgccaaggtgtcctggcgaatcttcacatatttgcgactttatacggaagaccttgaatgcgggggcgtatagtaaagtcgtccaggaacggcttgtccaagctgaatactggcacgatcccatcaaagaagatgtctatcggaatcacagcatttttctcgccgacataaaccaagaacgcggaattaatgagtcatacaagaagaacttgatggcacttaaaaaatttgtgatggttaagtttttgaatgatagtatcgtagatcccgtagatagtgaatggtttggtttctatcgatccggacaggctaaagaaacgataccattgcaggaaacctctttgtatactcaagataggttgggcctcaaggagatggataatgcggggcaacttgtcttcctcgcgactgagggtgaccacctccagctcagcgaggaatggttttacgcccacatcattcctttccttggttaa

SEQ ID NO: 220 - DNA, 921 bp, Artificial Sequence
  PPT1-21 (ppt2ss-PPT1; Codon optimized IDT)
atgctggggctctgggggcagcggctccccgcggcgtgggtcctgcttctgttgcctttcctgccgctgctgctgcttgcagatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggagcttatagtaaagtcgtccaagaacggcttgttcaagcggagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggctaa

SEQ ID NO: 221 - DNA, 942 bp, Artificial Sequence
  PPT1-22 (ppt2ss-PPT1_2; Codon optimized IDT)
atgctggggctctgggggcagcggctccccgcggcgtgggtcctgcttctgttgcctttcctgccgctgctgctgcttgcatcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggagcttatagtaaagtcgtccaagaacggcttgttcaagcggagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggctaa

SEQ ID NO: 222 - DNA, 921 bp, Artificial Sequence
  PPT1-23 (concensusSS-PPT1; Codon optimized IDT)
atggcaagtccttcctgtctttggctgctggctgttgccttgcttccttggtcttgtgcggcgcgggcactcggccatttggacccaccagccccactgcccttggttatatggcatggaatgggagatagttgctgtaatccactgagcatgggagccataaagaaaatggttgagaaaaaaataccgggaatatatgttctgagcctggagataggtaagacactcatggaagacgttgaaaactcattttttttgaacgtgaatagtcaagtcacaacggtctgtcaagctctggctaaagatcctaagttgcaacagggttacaatgcgatgggatttagtcaaggtggacagttcctgcgggccgtcgcacagaggtgcccgagtccgccaatgataaatctcatttcagtaggcggacaacatcagggcgtgttcggtcttcctcgctgcccgggtgagtcttctcacatttgcgatttcatacgcaaaacacttaacgcgggggcttactccaaggtagttcaagaaaggctcgtgcaggccgaatactggcatgatccaatcaaagaagacgtctatagaaatcactctatattcttggccgacatcaaccaagagcgaggtataaatgaaagttacaagaaaaacctcatggctcttaaaaaatttgttatggtaaaatttcttaatgactctatcgttgacccggtcgatagtgagtggtttgggttttataggagcggacaggccaaagagacaattccgttgcaggagacaagtttgtacacgcaggataggcttggtcttaaggagatggacaacgcgggccaacttgtatttttggctactgaaggtgatcacctccaattgtctgaagagtggttttatgcgcatattattcctttcctcggctaa

SEQ ID NO: 223 - DNA, 921 bp, Artificial Sequence
  PPT1-24 (consensus-PPT1; Codon optimized IDT)
atggcaagcccttcctgcctctggttgcttgctgttgctttgcttccttggtcttgtgctgcaagagcacttggccaccttgatcctcctgcacctctcccgctcgttatatggcacggcatgggggatagctgttgtaatccactgtcaatgggggctattaagaaaatggtggagaagaaaattccgggaatttatgtgctctccctggagataggcaaaacgcttatggaagacgtggagaacagtttttttcttaacgtaaattcacaggttaccaccgtctgtcaaattttggccaaagatcccaaactgcaacaagggtataacgctatgggcttcagtcaagggggtcaatttttgagggcggttgcgcaacgctgccctagtccgcccatgataaacttgatcagtgttgggggacagcaccagggagtatttggtctgccgaggtgtccaggcgagtcttcacacatctgtgactttattcgcaagaccttgaacgcgggcgcttattccaaggctgtgcaggaaaggcttgtgcaagcggaatattggcacgatcctataaaggaagatgtgtatcgcaaccactctatcttcctggcggatatcaatcaagaacgaggagtcaatgagtcctacaagaaaaatctgatggcgcttaaaaagttcgtaatggtcaagttcctgaatgacagcatagtagatccggtggattctgaatggttcggattctaccggtcaggacaggccaaggagacaatcccccttcaagagacgaccctgtacacacaagatagattgggactgaaagaaatggataaggccggtcaattggtcttcttggccacagaaggggaccatctccaactgagtgaagaatggttttatgcacatataattcccttcctggagtaa

SEQ ID NO: 224 - DNA, 921 bp, Artificial Sequence
  PPT1-25 (wt-PPT1 L283C H300C; Codon optimized IDT)
atggcatcaccgggttgcctctggttgttggccgttgcgttgcttccgtggacatgtgcatcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggagcttatagtaaagtcgtccaagaacggcttgttcaagcggagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttctgcgctacggaaggcgatcatctccaactctccgaagagtggttctatgcctgcataatcccgttcctgggctaa

SEQ ID NO: 225 - DNA, 921 bp, Artificial Sequence
  PPT1-26 (wt-PPT1 G113C L121C; Codon optimized IDT)
atggcatcaccgggttgcctctggttgttggccgttgcgttgcttccgtggacatgtgcatcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatgtgctttagccagggcggacagttttgcagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggagcttatagtaaagtcgtccaagaacggcttgttcaagcggagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggctaa

SEQ ID NO: 226 - DNA, 921 bp, Artificial Sequence
  PPT1-27 (wt-PPT1 A171C A183C; Codon optimized IDT)
atggcatcaccgggttgcctctggttgttggccgttgcgttgcttccgtggacatgtgcatcaagagctcttcaacatctggatcccccagctcccctgccgctcgtaatctggcacgggatgggggattcatgttgtaacccgttgtcaatgggcgcgataaaaaagatggttgaaaagaagattccaggcatctacgttctgtccctggaaatcggtaagacactgatggaagacgtggagaactccttctttctcaacgtcaatagtcaggtcactaccgtctgtcaagcattggcaaaggaccctaaacttcagcaggggtacaatgcgatggggtttagccagggcggacagtttcttagagccgtcgcacagcgctgtccatctcccccgatgattaaccttatatctgtcgggggacaacaccagggtgtttttggtcttcctcgctgtcctggtgaaagctcccacatctgtgatttcatacgcaaaacgttgaacgcaggatgctatagtaaagtcgtccaagaacggcttgttcaatgcgagtattggcatgacccaataaaagaagacgtttataggaatcactctatcttcttggccgatatcaaccaagaacgcggaatcaacgaaagctacaaaaagaatcttatggctctcaagaaatttgttatggtgaaattccttaatgactctatagtagatcctgtcgattcagaatggttcgggttctacaggtctggccaggcgaaggagactattcccctccaagaaacgtctctctatacacaagacagactcggactgaaagagatggataatgcgggccagttggtcttcttggctacggaaggcgatcatctccaactctccgaagagtggttctatgcccatataatcccgttcctgggctaa

SEQ ID NO: 227 - DNA, 942 bp, Artificial Sequence
  PPT1-28 (BiP2aa-PPT1; native human sequence)
atgaaacttagtctcgtcgcagcaatgttgcttctcctgtgggttgccctcctgttgctcagcgcagctagggctgctgcgtctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 228 - DNA, 1152 bp, Artificial Sequence
  PPT1-101
atgaagctctccctggtggccgcgatgctgctgctgctctgggtggcactgctgctgctcagcgcggcgagggccgccgcgtctagaacactgtgcggaggggagcttgtagacactcttcagttcgtgtgtggagatcgcgggttcctcttctctcgcggaggtggaggttctaggggtatactggaggagtgttgtttcagggactgtgacttggcgctcctcgagacctattgcgcgacgccagccaggtccgaaggaggtggtggcagtggaggaggagggagtcggcctagggcagtcccaacccaggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 229 - DNA, 1167 bp, Artificial Sequence
  PPT1-31 (BiP1-vIGF2-PPT1; native human sequence)
atgaagctcagtctcgtggcagctatgctcctcctgctgtccctggttgcggcaatgttgctcttgctgagcgccgcgagagcaagtcgcacgttgtgtggaggtgaactcgtcgacacccttcagttcgtatgtggagatcgcggtttcctcttctcacgcccagcttccagagtttcccgaagatcacgaggaatagttgaggagtgctgttttcggtcttgtgatctggctctcctcgagacttattgtgctacgccggcccgctctgaaggaggtggtggcagtggaggaggagggagtcggcctagggcagtcccaacccaggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 230 - DNA, 1152 bp, Artificial Sequence
  PPT1-32 (wt-PPT1-vIGF2-32; native human sequence)
atggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggaagacctagagcagtgcctacgcagggagggagtgggagtggatccacttcatcctctagaacactgtgcggaggggagcttgtagacactcttcagttcgtgtgtggagatcgcgggttcctcttctctcgcggaggtggaggttctaggggtatactggaggagtgttgtttcagggagtgtgacttggcgctcctcgagacctattgcgcgacgccagccaggtccgaatga

SEQ ID NO: 231 - DNA, 1164 bp, Artificial Sequence; identical to SEQ ID NO: 195
  PPT1-33 (wt-PPT1-vIGF2-8Q; native human sequence)

SEQ ID NO: 232 - DNA, 1164 bp, Artificial Sequence
  PPT1-34 (wt-PPT1-vIGF2-8Q; Codon optimized GENEius)
atggcctccccaggctgcttatggttgctggccgtagcacttttaccatggacatgtgctagtcgagctttacaacacttagacccgccagcgcctcttcctttagttatctggcacggcatgggcgactcgtgttgtaacccgctcagtatgggtgccataaagaagatggtggagaagaaaattcccggaatctatgtgcttagcctcgaaatcggcaaaacacttatggaggacgtagagaactcattcttcctgaatgtaaatagccaagtcaccacggtatgtcaagctctagcgaaggaccctaaactccagcaggggtataacgcaatgggattttctcagggcggccagtttctgcgtgctgtcgcacagcgttgcccttctccgcctatgataaacttaatttccgtaggagggcaacaccaaggggtattcggcttaccgaggtgtccaggcgaatcttcacatatatgcgacttcatccgaaagacccttaatgccggggcctattccaaggtggtacaggaacggttggtgcaagctgagtattggcacgaccctataaaggaagatgtgtatcggaatcactcaatctttcttgcggatataaatcaagagcgcggcattaacgagagctacaagaagaacctcatggctcttaagaaattcgtcatggtcaaattcctcaacgacagtatagttgatcccgtcgattcggagtggtttggattctaccgctctgggcaagccaaagagaccataccactacaggaaacatcgctatatacccaagatcgcttgggtttgaaagaaatggataacgccggtcagcttgtgttcttagcgacagagggtgatcatctccagctgtcggaagaatggttctatgcccacataatacctttccttggacgaccccgtgcggtcccaacgcagggtggatcaggtagcggctcaactagttccagccgtacgttgtgcggcggagaactagtagacactcttcaattcgtttgtggggatcggggcttcctcttcagcaggccagcgtcacgcgtgtcgcgtcggagccgaggtatagtggaagaatgctgcttccgcgaatgtgatctagcactccttgaaacctactgcgcgacgcctgcccgaagtgaatga

SEQ ID NO: 233 - DNA, 1164 bp, Artificial Sequence
  PPT1-35 (wt-PPT1-vIGF2-8Q; Codon optimized COOL)
atggcttcccctggctgcctgtggctgctcgctgtggccctcctgccctggacctgtgcttctcgggcccttcagcatctggaccctccagcccccctccccttggtcatctggcacggcatgggcgacagctgctgcaaccctctgtccatgggggccatcaagaaaatggttgagaagaagatcccaggcatctacgtgctgagcctggaaattggcaagacactgatggaggatgtggaaaacagcttcttcctgaatgtgaactcccaggtgaccaccgtgtgccaggctctggccaaagatcccaagctgcagcagggctacaatgccatgggattcagccaggggggccagtttctgcgggctgttgcccagaggtgccccagcccccccatgatcaatctcatctctgtgggcgggcagcaccagggtgtgtttggcctgcctcgctgccctggagaaagcagccacatttgtgatttcatcaggaagaccttaaatgctggagcctacagcaaggtggtccaggaaaggctggtgcaggcagagtactggcatgaccccatcaaagaggacgtgtacagaaaccacagcatcttcctggctgacatcaaccaggagagaggaattaatgagagctacaagaagaacctcatggccttgaaaaagtttgtgatggtgaagttcttgaatgactccatcgtggatcctgtggacagtgaatggtttgggttctaccgctctggacaggccaaggaaaccatccccctgcaagaaacatccctgtacacccaggaccgcctggggctgaaggagatggacaacgccggccaactggtcttccttgccacagaaggagaccacctgcagctgtctgaggagtggttctatgcccacatcatccccttcctgggccggcccagggccgtgcccacacagggaggcagtggcagcggctccaccagctccagcaggaccctgtgtggcggcgagctggttgacaccctccagttcgtgtgtggggacagaggcttcctcttctccaggcccgccagccgggtgagccgccgctcccggggcattgtggaggaatgttgcttccgggagtgtgacctggccctgctggagacctactgtgccacccctgcccggagtgagtga

SEQ ID NO: 234 - DNA, 1152 bp, Artificial Sequence; identical to SEQ ID NO: 228
  PPT1-101

SEQ ID NO: 235 - DNA, 1152 bp, Artificial Sequence
  PPT1-104
atggcgtcgcccggcagcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggaagacctagagcagtgcctacgcagggagggagtgggagtggatccacttcatcctctagaacactgtgcggaggggagcttgtagacactcttcagttcgtgtgtggagatcgcgggttcctcttctctcgcggaggtggaggttctaggggtatactggaggagtgttgtttcagggagtgtgacttggcgctcctcgagacctattgcgcgacgccagccaggtccgaatga

SEQ ID NO: 236 - DNA, 1158 bp, Artificial Sequence
  PPT-112
atggcgtcgcccggcagcctgtggctcttggctgtggctctcctgccatggacctgcgcttctcgggcgctgcagcatctggccgcgtctagaacactgtgcggaggggagcttgtagacactcttcagttcgtgtgtggagatcgcgggttcctcttctctcgcggaggtggaggttctaggggtatactggaggagtgttgtttcagggactgtgacttggcgctcctcgagacctattgcgcgacgccagccaggtccgaaggaggtggtggcagtggaggaggagggagtcggcctagggcagtcccaacccaggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 237 - DNA, 1158 bp, Artificial Sequence
  PPT-114
atggcgtcgcccggcagcctgtggctcttggctgtggctctcctgccatggacctgcgcttctcgggcgctgcagcatctggccgcgtctagaacactgtgcggaggggagcttgtagacactcttcagttcgtgtgtggagatcgcgggttcctcttctctcgcggaggtggaggttctaggggtatactggaggagtgttgtttcagggagtgtgacttggcgctcctcgagacctattgcgcgacgccagccaggtccgaaggaggtggtggcagtggaggaggagggagtcggcctagggcagtcccaacccaggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 238 - DNA, 1158 bp, Artificial Sequence
  PPT1-115
atggcgtcgcccggcagcctgtggctcttggctgtggctctcctgccatggacctgcgcttctcgggcgctgcagcatctggctgccgacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggaagacctagagcagtgcctacgcagggagggagtgggagtggatccacttcatcctctagaacactgtgcggaggggagcttgtagacactcttcagttcgtgtgtggagatcgcgggttcctcttctctcgcggaggtggaggttctaggggtatactggaggagtgttgtttcagggagtgtgacttggcgctcctcgagacctattgcgcgacgccagccaggtccgaatga

SEQ ID NO: 239 - DNA, 1164 bp, Artificial Sequence
  PPT-116
atggcgtcgcccggcagcctgtggctcttggctgtggctctcctgccatggacctgcgcttctcgggcgctgcagcatctggctgccgacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggaagacctagagcagtgcctacgcagggagggggtggcagtggcagtggaggcggcggttcctctagaacactgtgcggaggggagcttgtagacactcttcagttcgtgtgtggagatcgcgggttcctcttctctcgcggaggtggaggttctaggggtatactggaggagtgttgtttcagggagtgtgacttggcgctcctcgagacctattgcgcgacgccagccaggtccgaatga

SEQ ID NO: 240 - DNA, 1158 bp, Artificial Sequence
  PPT1-117
atggcgtcgcccggcagcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggaagacctagagcagtgcctacgcagggagggggtggcagtggcagtggaggcggcggttcctctagaacactgtgcggaggggagcttgtagacactcttcagttcgtgtgtggagatcgcgggttcctcttctctcgcggaggtggaggttctaggggtatactggaggagtgttgtttcagggagtgtgacttggcgctcctcgagacctattgcgcgacgccagccaggtccgaatga

SEQ ID NO: 241 - DNA, 1158 bp, Artificial Sequence
  PPT-118
atggcgtcgcccggcagcctgtggctcttggctgtggctctcctgccatggacctgcgcttctcgggcgctgcagcatctggctgccgacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggaagacctagagcagtgcctacgcagggagggggtggcagtggaggcggcggttcctctagaacactgtgcggaggggagcttgtagacactcttcagttcgtgtgtggagatcgcgggttcctcttctctcgcggaggtggaggttctaggggtatactggaggagtgttgtttcagggagtgtgacttggcgctcctcgagacctattgcgcgacgccagccaggtccgaatga

SEQ ID NO: 242 - DNA, 81 bp, Artificial Sequence
  BiP2AA
atgaagctctccctggtggccgcgatgctgctgctgctctgggtggcactgctgctgctcagcgcggcgagggccgccgcg

SEQ ID NO: 243 - DNA, 81 bp, Artificial Sequence
  eSP C6S
atggcgtcgcccggcagcctgtggctcttggctgtggctctcctgccatggacctgcgcttctcgggcgctgcagcatctg

SEQ ID NO: 244 - DNA, 87 bp, Artificial Sequence
  eSP C6S AA (used in PPT1-112 and PPT1-114)
atggcgtcgcccggcagcctgtggctcttggctgtggctctcctgccatggacctgcgcttctcgggcgctgcagcatctggccgcg

SEQ ID NO: 245 - DNA, 87 bp, Artificial Sequence
  eSP C6S AA (used in PPT1-115, PPT1-116, and PPT1-118) - different codon usage for AA portion
atggcgtcgcccggcagcctgtggctcttggctgtggctctcctgccatggacctgcgcttctcgggcgctgcagcatctggctgcc

SEQ ID NO: 246 - DNA, 2295 bp, Artificial Sequence
  WT NAGLU-HPC4
atggaggccgtggccgtggccgccgccgtgggcgtgctgctgctggccggcgccggcggcgccgccggcgacgaggcccgggaggccgccgccgtgcgggccctggtggcccggctgctgggccccggccccgccgccgacttcagcgttagcgtggagcgggccctggccgccaagcccggcctggacacctacagcctgggcggcggcggcgccgcccgggtgcgggtgcggggcagcaccggcgtggccgccgccgccggcctgcaccggtatctgcgggacttctgcggctgccacgtggcctggagcggcagccagctgcggctgccccggcccctgcccgccgtgcccggcgagctgaccgaggccacccccaaccggtatcggtactaccagaacgtgtgcacccagagctacagcttcgtgtggtgggactgggcccggtgggagcgggagatcgactggatggccctgaacggcatcaacctggccctggcctggagcggccaggaggccatctggcagcgggtgtacctggccctgggcctgacccaggccgagatcaacgagttcttcaccggccccgccttcctggcctggggccggatgggcaacctgcacacctgggacggccccctgcctccaagctggcacatcaagcagctgtacctgcagcaccgggtgctggaccagatgcggagcttcggcatgacccccgtgctgcccgccttcgccggccacgtgcccgaggccgtgacccgggtgttcccccaagttaacgtgaccaagatgggcagctggggccacttcaactgcagctacagctgcagcttcctgctggcccccgaggaccccatcttccccatcatcggcagcctgttcctgcgggagctgatcaaggagttcggcaccgaccacatctacggcgccgacaccttcaacgagatgcagcctccaagcagcgagcccagctacctggccgccgccaccaccgccgtgtacgaggccatgaccgccgtggacaccgaggccgtgtggctgctgcagggctggctgttccagcaccagccccagttctggggacctgcccagatccgggccgtgctgggcgccgtgcctagaggacggctgctggtgctggacctgttcgccgagagccagcccgtgtacacccggaccgccagcttccagggccagcccttcatctggtgcatgctgcacaacttcggcggcaaccacggcctgttcggcgccctggaggccgtgaacggcggccccgaggccgcccggctgttccccaacagcaccatggtgggcaccggcatggcccccgagggcatcagccagaacgaggtggtgtacagcctgatggccgagctgggctggcggaaggaccccgtgcccgacctggccgcctgggtgaccagcttcgccgcccggcggtacggcgtgagccaccccgacgccggcgccgcctggcggctgctgctgcggagcgtgtacaactgcagcggcgaggcctgccggggccacaaccggagccccctggtgcggcggcccagcctgcagatgaacaccagcatctggtacaaccggagcgacgtgttcgaggcctggcggctgctgctgaccagcgcccccagcctggccaccagccccgccttcagatacgacctgctggacctgacccggcaggccgtgcaggagctggtgagcctgtactacgaggaggcccggagcgcctacctgagcaaggagctggccagcctgctgcgggccggcggcgtgctggcctacgagctgctgcccgccctggacgaggtgctggccagcgacagccggttcctgctgggcagctggctggagcaggcccgggccgccgccgtgagcgaggccgaggccgacttctacgagcagaacagccggtatcagctgaccctgtggggacctgagggcaacatcctggactacgccaacaagcagctggccggcctggtggccaactactacaccccaaggtggcggctgttcctggaggccctggtggacagcgtggcccagggcatccccttccagcagcaccagttcgacaagaacgtgttccagctggagcaggccttcgtgctgagcaagcagcggtatcccagccagcctagaggagacaccgtggacctggccaagaagatcttcctgaagtactacccccggtgggtggccggcagctggggacttgaggtactgttccaagggcccgaggaccaggtagacccacgactcattgatggaaaatag

SEQ ID NO: 247 - DNA, 2544 bp, Artificial Sequence
  vIGF2-NAGLU-HPC4
atggaggctgtggctgtggcagctgcggtgggggtccttctcctggccggggccgggggcgcggcaggcgacgcttctaggacgttgtgtggtggggaacttgtcgacacactgcagtttgtctgcggcgaccgaggatttcttttttccaggcctgcctcaagagtatctaggaggtcccgcggtattgttgaagagtgctgttttaggtcatgcgaccttgcgttgttggagacatattgtgctacccctgcacgctctgaaggtggaggtggttcaggtggtggaggttccaggccaagggcggtccctactcaggccgaggcccgggaggccgccgccgtgcgggccctggtggcccggctgctgggccccggccccgccgccgacttcagcgttagcgtggagcgggccctggccgccaagcccggcctggacacctacagcctgggcggcggcggcgccgcccgggtgcgggtgcggggcagcaccggcgtggccgccgccgccggcctgcaccggtatctgcgggacttctgcggctgccacgtggcctggagcggcagccagctgcggctgccccggcccctgcccgccgtgcccggcgagctgaccgaggccacccccaaccggtatcggtactaccagaacgtgtgcacccagagctacagcttcgtgtggtgggactgggcccggtgggagcgggagatcgactggatggccctgaacggcatcaacctggccctggcctggagcggccaggaggccatctggcagcgggtgtacctggccctgggcctgacccaggccgagatcaacgagttcttcaccggccccgccttcctggcctggggccggatgggcaacctgcacacctgggacggccccctgcctccaagctggcacatcaagcagctgtacctgcagcaccgggtgctggaccagatgcggagcttcggcatgacccccgtgctgcccgccttcgccggccacgtgcccgaggccgtgacccgggtgttcccccaagttaacgtgaccaagatgggcagctggggccacttcaactgcagctacagctgcagcttcctgctggcccccgaggaccccatcttccccatcatcggcagcctgttcctgcgggagctgatcaaggagttcggcaccgaccacatctacggcgccgacaccttcaacgagatgcagcctccaagcagcgagcccagctacctggccgccgccaccaccgccgtgtacgaggccatgaccgccgtggacaccgaggccgtgtggctgctgcagggctggctgttccagcaccagccccagttctggggacctgcccagatccgggccgtgctgggcgccgtgcctagaggacggctgctggtgctggacctgttcgccgagagccagcccgtgtacacccggaccgccagcttccagggccagcccttcatctggtgcatgctgcacaacttcggcggcaaccacggcctgttcggcgccctggaggccgtgaacggcggccccgaggccgcccggctgttccccaacagcaccatggtgggcaccggcatggcccccgagggcatcagccagaacgaggtggtgtacagcctgatggccgagctgggctggcggaaggaccccgtgcccgacctggccgcctgggtgaccagcttcgccgcccggcggtacggcgtgagccaccccgacgccggcgccgcctggcggctgctgctgcggagcgtgtacaactgcagcggcgaggcctgccggggccacaaccggagccccctggtgcggcggcccagcctgcagatgaacaccagcatctggtacaaccggagcgacgtgttcgaggcctggcggctgctgctgaccagcgcccccagcctggccaccagccccgccttcagatacgacctgctggacctgacccggcaggccgtgcaggagctggtgagcctgtactacgaggaggcccggagcgcctacctgagcaaggagctggccagcctgctgcgggccggcggcgtgctggcctacgagctgctgcccgccctggacgaggtgctggccagcgacagccggttcctgctgggcagctggctggagcaggcccgggccgccgccgtgagcgaggccgaggccgacttctacgagcagaacagccggtatcagctgaccctgtggggacctgagggcaacatcctggactacgccaacaagcagctggccggcctggtggccaactactacaccccaaggtggcggctgttcctggaggccctggtggacagcgtggcccagggcatccccttccagcagcaccagttcgacaagaacgtgttccagctggagcaggccttcgtgctgagcaagcagcggtatcccagccagcctagaggagacaccgtggacctggccaagaagatcttcctgaagtactacccccggtgggtggccggcagctggggacttgaggtactgttccaagggcccgaggaccaggtagacccacgactcattgatggaaaatag

SEQ ID NO: 248 - DNA, 2544 bp, Artificial Sequence
  vIGF2-17-NAGLU-HPC4
atggaggctgtggctgtggcagctgcggtgggggtccttctcctggccggggccgggggcgcggcaggcgacgctagcagaacactttgtggcggagagctggtggacaccctgcagtttgtgtgtggcgacagaggcttcctgttcagcagacctgcatccagagttagcaggcggtccagaggaatcgtggaagagtgctgcttcagagaatgcgatctggccctgctggaaacctactgtgccacaccagccagatctgaaggtggaggtggttcaggtggtggaggttccaggccaagggcggtccctactcaggccgaggcccgggaggccgccgccgtgcgggccctggtggcccggctgctgggccccggccccgccgccgacttcagcgttagcgtggagcgggccctggccgccaagcccggcctggacacctacagcctgggcggcggcggcgccgcccgggtgcgggtgcggggcagcaccggcgtggccgccgccgccggcctgcaccggtatctgcgggacttctgcggctgccacgtggcctggagcggcagccagctgcggctgccccggcccctgcccgccgtgcccggcgagctgaccgaggccacccccaaccggtatcggtactaccagaacgtgtgcacccagagctacagcttcgtgtggtgggactgggcccggtgggagcgggagatcgactggatggccctgaacggcatcaacctggccctggcctggagcggccaggaggccatctggcagcgggtgtacctggccctgggcctgacccaggccgagatcaacgagttcttcaccggccccgccttcctggcctggggccggatgggcaacctgcacacctgggacggccccctgcctccaagctggcacatcaagcagctgtacctgcagcaccgggtgctggaccagatgcggagcttcggcatgacccccgtgctgcccgccttcgccggccacgtgcccgaggccgtgacccgggtgttcccccaagttaacgtgaccaagatgggcagctggggccacttcaactgcagctacagctgcagcttcctgctggcccccgaggaccccatcttccccatcatcggcagcctgttcctgcgggagctgatcaaggagttcggcaccgaccacatctacggcgccgacaccttcaacgagatgcagcctccaagcagcgagcccagctacctggccgccgccaccaccgccgtgtacgaggccatgaccgccgtggacaccgaggccgtgtggctgctgcagggctggctgttccagcaccagccccagttctggggacctgcccagatccgggccgtgctgggcgccgtgcctagaggacggctgctggtgctggacctgttcgccgagagccagcccgtgtacacccggaccgccagcttccagggccagcccttcatctggtgcatgctgcacaacttcggcggcaaccacggcctgttcggcgccctggaggccgtgaacggcggccccgaggccgcccggctgttccccaacagcaccatggtgggcaccggcatggcccccgagggcatcagccagaacgaggtggtgtacagcctgatggccgagctgggctggcggaaggaccccgtgcccgacctggccgcctgggtgaccagcttcgccgcccggcggtacggcgtgagccaccccgacgccggcgccgcctggcggctgctgctgcggagcgtgtacaactgcagcggcgaggcctgccggggccacaaccggagccccctggtgcggcggcccagcctgcagatgaacaccagcatctggtacaaccggagcgacgtgttcgaggcctggcggctgctgctgaccagcgcccccagcctggccaccagccccgccttcagatacgacctgctggacctgacccggcaggccgtgcaggagctggtgagcctgtactacgaggaggcccggagcgcctacctgagcaaggagctggccagcctgctgcgggccggcggcgtgctggcctacgagctgctgcccgccctggacgaggtgctggccagcgacagccggttcctgctgggcagctggctggagcaggcccgggccgccgccgtgagcgaggccgaggccgacttctacgagcagaacagccggtatcagctgaccctgtggggacctgagggcaacatcctggactacgccaacaagcagctggccggcctggtggccaactactacaccccaaggtggcggctgttcctggaggccctggtggacagcgtggcccagggcatccccttccagcagcaccagttcgacaagaacgtgttccagctggagcaggccttcgtgctgagcaagcagcggtatcccagccagcctagaggagacaccgtggacctggccaagaagatcttcctgaagtactacccccggtgggtggccggcagctggggacttgaggtactgttccaagggcccgaggaccaggtagacccacgactcattgatggaaaatag

SEQ ID NO: 249 - DNA, 2532 bp, Artificial Sequence
  vIGF2-31-NAGLU-HPC4
atggaggctgtggctgtggcagctgcggtgggggtccttctcctggccggggccgggggcgcggcaggcgacgctagcagaacactttgtggcggagagctggtggacaccctgcagtttgtgtgtggcgacagaggcttcctgttcagcagaggtggaggtggatctagaggaatcctggaagagtgctgcttcagagattgcgatctggccctgctggaaacctactgtgccacaccagccagatctgaaggtggaggtggttcaggtggtggaggttccaggccaagggcggtccctactcaggccgaggcccgggaggccgccgccgtgcgggccctggtggcccggctgctgggccccggccccgccgccgacttcagcgttagcgtggagcgggccctggccgccaagcccggcctggacacctacagcctgggcggcggcggcgccgcccgggtgcgggtgcggggcagcaccggcgtggccgccgccgccggcctgcaccggtatctgcgggacttctgcggctgccacgtggcctggagcggcagccagctgcggctgccccggcccctgcccgccgtgcccggcgagctgaccgaggccacccccaaccggtatcggtactaccagaacgtgtgcacccagagctacagcttcgtgtggtgggactgggcccggtgggagcgggagatcgactggatggccctgaacggcatcaacctggccctggcctggagcggccaggaggccatctggcagcgggtgtacctggccctgggcctgacccaggccgagatcaacgagttcttcaccggccccgccttcctggcctggggccggatgggcaacctgcacacctgggacggccccctgcctccaagctggcacatcaagcagctgtacctgcagcaccgggtgctggaccagatgcggagcttcggcatgacccccgtgctgcccgccttcgccggccacgtgcccgaggccgtgacccgggtgttcccccaagttaacgtgaccaagatgggcagctggggccacttcaactgcagctacagctgcagcttcctgctggcccccgaggaccccatcttccccatcatcggcagcctgttcctgcgggagctgatcaaggagttcggcaccgaccacatctacggcgccgacaccttcaacgagatgcagcctccaagcagcgagcccagctacctggccgccgccaccaccgccgtgtacgaggccatgaccgccgtggacaccgaggccgtgtggctgctgcagggctggctgttccagcaccagccccagttctggggacctgcccagatccgggccgtgctgggcgccgtgcctagaggacggctgctggtgctggacctgttcgccgagagccagcccgtgtacacccggaccgccagcttccagggccagcccttcatctggtgcatgctgcacaacttcggcggcaaccacggcctgttcggcgccctggaggccgtgaacggcggccccgaggccgcccggctgttccccaacagcaccatggtgggcaccggcatggcccccgagggcatcagccagaacgaggtggtgtacagcctgatggccgagctgggctggcggaaggaccccgtgcccgacctggccgcctgggtgaccagcttcgccgcccggcggtacggcgtgagccaccccgacgccggcgccgcctggcggctgctgctgcggagcgtgtacaactgcagcggcgaggcctgccggggccacaaccggagccccctggtgcggcggcccagcctgcagatgaacaccagcatctggtacaaccggagcgacgtgttcgaggcctggcggctgctgctgaccagcgcccccagcctggccaccagccccgccttcagatacgacctgctggacctgacccggcaggccgtgcaggagctggtgagcctgtactacgaggaggcccggagcgcctacctgagcaaggagctggccagcctgctgcgggccggcggcgtgctggcctacgagctgctgcccgccctggacgaggtgctggccagcgacagccggttcctgctgggcagctggctggagcaggcccgggccgccgccgtgagcgaggccgaggccgacttctacgagcagaacagccggtatcagctgaccctgtggggacctgagggcaacatcctggactacgccaacaagcagctggccggcctggtggccaactactacaccccaaggtggcggctgttcctggaggccctggtggacagcgtggcccagggcatccccttccagcagcaccagttcgacaagaacgtgttccagctggagcaggccttcgtgctgagcaagcagcggtatcccagccagcctagaggagacaccgtggacctggccaagaagatcttcctgaagtactacccccggtgggtggccggcagctggggacttgaggtactgttccaagggcccgaggaccaggtagacccacgactcattgatggaaaatag

SEQ ID NO: 250 - DNA, 2532 bp, Artificial Sequence
  vIGF2-32-NAGLU-HPC4
atggaggctgtggctgtggcagctgcggtgggggtccttctcctggccggggccgggggcgcggcaggcgacgctagcagaacactttgtggcggagagctggtggacaccctgcagtttgtgtgtggcgacagaggcttcctgttcagcagaggtggaggtggatctagaggaatcctggaagagtgctgcttcagagaatgcgatctggccctgctggaaacctactgtgccacaccagccagatctgaaggtggaggtggttcaggtggtggaggttccaggccaagggcggtccctactcaggccgaggcccgggaggccgccgccgtgcgggccctggtggcccggctgctgggccccggccccgccgccgacttcagcgttagcgtggagcgggccctggccgccaagcccggcctggacacctacagcctgggcggcggcggcgccgcccgggtgcgggtgcggggcagcaccggcgtggccgccgccgccggcctgcaccggtatctgcgggacttctgcggctgccacgtggcctggagcggcagccagctgcggctgccccggcccctgcccgccgtgcccggcgagctgaccgaggccacccccaaccggtatcggtactaccagaacgtgtgcacccagagctacagcttcgtgtggtgggactgggcccggtgggagcgggagatcgactggatggccctgaacggcatcaacctggccctggcctggagcggccaggaggccatctggcagcgggtgtacctggccctgggcctgacccaggccgagatcaacgagttcttcaccggccccgccttcctggcctggggccggatgggcaacctgcacacctgggacggccccctgcctccaagctggcacatcaagcagctgtacctgcagcaccgggtgctggaccagatgcggagcttcggcatgacccccgtgctgcccgccttcgccggccacgtgcccgaggccgtgacccgggtgttcccccaagttaacgtgaccaagatgggcagctggggccacttcaactgcagctacagctgcagcttcctgctggcccccgaggaccccatcttccccatcatcggcagcctgttcctgcgggagctgatcaaggagttcggcaccgaccacatctacggcgccgacaccttcaacgagatgcagcctccaagcagcgagcccagctacctggccgccgccaccaccgccgtgtacgaggccatgaccgccgtggacaccgaggccgtgtggctgctgcagggctggctgttccagcaccagccccagttctggggacctgcccagatccgggccgtgctgggcgccgtgcctagaggacggctgctggtgctggacctgttcgccgagagccagcccgtgtacacccggaccgccagcttccagggccagcccttcatctggtgcatgctgcacaacttcggcggcaaccacggcctgttcggcgccctggaggccgtgaacggcggccccgaggccgcccggctgttccccaacagcaccatggtgggcaccggcatggcccccgagggcatcagccagaacgaggtggtgtacagcctgatggccgagctgggctggcggaaggaccccgtgcccgacctggccgcctgggtgaccagcttcgccgcccggcggtacggcgtgagccaccccgacgccggcgccgcctggcggctgctgctgcggagcgtgtacaactgcagcggcgaggcctgccggggccacaaccggagccccctggtgcggcggcccagcctgcagatgaacaccagcatctggtacaaccggagcgacgtgttcgaggcctggcggctgctgctgaccagcgcccccagcctggccaccagccccgccttcagatacgacctgctggacctgacccggcaggccgtgcaggagctggtgagcctgtactacgaggaggcccggagcgcctacctgagcaaggagctggccagcctgctgcgggccggcggcgtgctggcctacgagctgctgcccgccctggacgaggtgctggccagcgacagccggttcctgctgggcagctggctggagcaggcccgggccgccgccgtgagcgaggccgaggccgacttctacgagcagaacagccggtatcagctgaccctgtggggacctgagggcaacatcctggactacgccaacaagcagctggccggcctggtggccaactactacaccccaaggtggcggctgttcctggaggccctggtggacagcgtggcccagggcatccccttccagcagcaccagttcgacaagaacgtgttccagctggagcaggccttcgtgctgagcaagcagcggtatcccagccagcctagaggagacaccgtggacctggccaagaagatcttcctgaagtactacccccggtgggtggccggcagctggggacttgaggtactgttccaagggcccgaggaccaggtagacccacgactcattgatggaaaatag